(19) United States
(12) Patent Application Publication
(10) Pub. No.: US 2024/0201200 A1
(43) Pub. Date: Jun. 20, 2024

(54) Miniaturized Proteomic Sample Preparation

(71) Applicants: Northeastern University, Boston, MA (US); Scienion GmbH, Berlin (DE)

(72) Inventors: Nikolai Slavov, Cambridge, MA (US); Andrew Leduc, Hingham, MA (US); Richard Huffman, Boston, MA (US); Aileen Murphy, Madison, WI (US); Joshua Cantlon-Bruce, Phoenix, AZ (US)

(21) Appl. No.: 18/556,655

(22) PCT Filed: Apr. 22, 2022
(86) PCT No.: PCT/US2022/071883

§ 371 Date: Oct. 20, 2023

Related U.S. Application Data
(60) Provisional application No. 63/179,035, filed on Apr. 23, 2021.
(60) Provisional application No. 63/179,184, filed on Apr. 23, 2021.

(51) Int. Cl. G01N 33/68; G01N 1/34

(52) U.S. Cl. CPC G01N 33/6827; G01N 1/34; G01N 33/6848; G01N 2333/976; G01N 2458/15; G01N 2570/00

(57) ABSTRACT

The disclosure provides methods of forming one or more single-cell proteomic samples, such as by: dispensing n droplets of lysis buffer onto a substantially planar solid surface, wherein n>2: dispensing a single cell into each of the n droplets of lysis buffer to produce n droplets with a lysed single cell: dispensing digestion buffer into each of the n droplets to digest proteins from each lysed single cell to produce n droplets comprising peptides: dispensing a chemical tag into at least a subset of the n droplets comprising the peptides to produce labeled peptides, thereby enabling the labeled peptides in a given droplet to be distinguishable from labeled peptides in at least one other droplet: and applying a fluid to merge at least a subset of the droplets into a combined droplet on the substantially planar surface, thereby combining the labeled peptides to form a single-cell proteomic sample.

### RELATED APPLICATIONS

This application claims the benefit of U.S. Provisional Application No. 63/179,035, filed on Apr. 23, 2021 and U.S. Provisional Application No. 63/179,184, filed on Apr. 23, 2021. The entire teachings of the above applications are incorporated herein by reference.

### GOVERNMENT SUPPORT

This invention was made with government support under Grant No. GM123497 awarded by the National Institutes of Health. The government has certain rights in the invention.

### Common Ownership Under Joint Research Agreement 35 U.S.C. 102(C)

The subject matter disclosed in this application was developed, and the claimed invention was made by, or on behalf of, one or more parties to a joint Research Agreement that was in effect on or before the effective filing date of the claimed invention. The parties to the Joint Research Agreement are as follows Northeastern University and SCIENION GmbH.

### BACKGROUND

Single-cell measurements are essential for understanding biological systems composed of different cell types. Recent advances in single-cell RNA and protein methods have allowed analyzing single-cell heterogeneity at unprecedented scale and depth. These emerging single-cell methods have the potential to go beyond classifying cell types and to help characterize intrinsically single-cell processes, such as the cell division cycle (CDC) and its coordination with metabolism and cell growth. Crucial aspects of the CDC are regulated post-transcriptionally by protein synthesis and degradation and their characterization demands single-cell protein analysis. There is a need to improve single-cell proteomic sample preparation toward, for example, improved quantification of proteins and/or protein variabilities.

### SUMMARY

Embodiments of the present invention include methods of single-cell proteomic sample preparation for analyzing peptides in samples with a low abundance of proteins.

In one aspect, the disclosure provides a method of forming a single-cell proteomic sample, said method comprising:
a) dispensing n droplets of lysis buffer onto a substantially planar solid surface, wherein n≥2;
b) dispensing a single cell into each of the n droplets of lysis buffer to produce n droplets, each comprising a lysed single cell;
c) dispensing digestion buffer into each of the n droplets to digest proteins from each lysed single cell to produce n droplets comprising peptides;
d) dispensing a chemical tag into each of the n droplets comprising the peptides to produce labeled peptides, wherein at least one droplet of the n droplets receives a different chemical tag from at least one other droplet of the n droplets, thereby enabling the labeled peptides in the at least one droplet to be distinguishable from the labeled peptides in the at least one other droplet; and
e) applying a fluid to merge at least a subset of the n droplets into a combined droplet on the substantially planar surface, thereby combining the labeled peptides to form a single-cell proteomic sample.

In some embodiments, each of the n droplets in step a), b), c), and/or d) has a volume of about 25 nanoliters (nl) or less. In particular embodiments, each of the n droplets in step a), b), c) and d) has a volume of about 25 nanoliters (nl) or less.

In some embodiments, the substantially planar solid surface is provided by a uniform glass slide. In certain embodiments, the substantially planar solid surface is etched with a geometric pattern. In particular embodiments, the substantially planar solid surface is fluorocarbon-coated.

In certain embodiments, n is ≥10.

In some embodiments, the lysis buffer comprises about 4-8 nanoliters of 90-100% dimethyl sulfoxide (DMSO).

In some embodiments, step b) comprises dispensing the single cell in a cell suspension buffer with a volume of about 100-1,000 picoliters. In particular embodiments, step b) comprises dispensing the single cell in a cell suspension buffer with a volume of about 300 picoliters.

In certain embodiments, the single cell is lysed in a total volume of about 4-10 nl for about 10-20 minutes.

In some embodiments, step c) comprises:
dispensing about 15-25 nl of about 120 ng/μl trypsin to each of the n droplets; and
digesting the proteins from each lysed single cell at about 1ºC above the dew point and a relative humidity of about 75% for about 4-5 hours.

In certain embodiments, the chemical tag comprises a “light” version of TMT label reagents dissolved in DMSO. In other embodiments, the chemical tag comprises a “heavy” version of TMT label reagents dissolved in DMSO.

In some embodiments, step d) comprises dispensing about 18-22 nl of a chemical tag into each of the n droplets comprising the peptides; and enabling the chemical tag to react with the peptides at room temperature and a relative humidity of about 75% for about 1 hour to produce the labeled peptides. In certain embodiments, each droplet of the n droplets receives a unique chemical tag, thereby enabling the labeled peptides in each droplet to be distinguishable from the labeled peptides in each other droplet.

In certain embodiments, the fluid is water. In particular embodiments, the fluid has a volume of about 1 μl.

In some embodiments, steps a) to e) are repeated at least once to form two or more single-cell proteomic samples on the substantially planar solid surface.

In certain embodiments, at least 100 droplets of lysis buffer are dispensed onto the substantially planar solid surface.

In some embodiments, at least 500-3,000 droplets of lysis buffer are dispensed onto the substantially planar solid surface.

In certain embodiments, the two or more single-cell proteomic samples comprises peptides from at least 100 cells. In particular embodiments, the two or more single-cell proteomic samples comprises peptides from about 100-10,000 cells.

In some embodiments, the disclosed methods further comprise performing at least one proteomic analysis on the single-cell proteomic sample. In particular embodiments, the at least one single-cell proteomic analysis enables identifying and/or quantifying protein covariation across the single cells.

In another aspect, the disclosure provides a method of performing a proteomic analysis comprising analyzing a single-cell proteomic sample formed by any of the methods described herein. In some embodiments, the analyzing comprises identifying and/or quantifying protein covariation across the single cells.

In another aspect, the disclosure provides a single-cell proteomic sample, for example, one formed by any one of the methods of single-cell proteomic sample formation described herein.

In another aspect, the disclosure provides kits and systems comprising reagents described herein (for example, one or more buffers) and/or an element that provides for a substantially planar surface and/or devices described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1D show parallel preparation of thousands of single cells by nPOP. FIG. 1A is schematic of a non-limiting example of nano-Proteomic-sample Preparation (nPOP) method illustrating the steps of cell lysis, protein digestion, peptide labeling with tandem mass tags (TMT), quenching of labeling reaction, and sample collection. These steps are performed for each single cell (corresponding to a single droplet). FIG. 1B shows that after barcoding, single-cell samples are automatically pooled into set samples, and the set samples are transferred into a 384-well plate, which is then placed into an autosampler for automated injection for LC-MS/MS. Any system that support 384-well plate injection (such as Dionex™ 3000) can implement this example workflow. FIG. 1C shows that the flat surface allows programming different droplet layouts, such as the 4 examples shown in the picture. FIG. 1D shows four slides with 2,016 single cells from an nPOP experiment using droplet configuration AL-01. Samples are surrounded by a perimeter of water for local humidity control. Slides are placed on a cooling surface to further prevent evaporation.

FIGS. 2A-2E depict proteome coverage and quality controls. FIG. 2A shows the number of proteins and peptides quantified per single cell from multiplexed samples prepared by nPOP and analyzed using 60 min active gradients on Q Exactive™ classic Mass Spectrometer. FIG. 2B shows the distributions of reporter ion (RI) intensities for all melanoma, monocyte, and negative controls. Intensities were mostly absent from negative control wells, which contained all reagents but not a single cell. FIG. 2C plots the average reporter ion intensity against the measured diameter of cells. A strong correlation between the two metrics shows that larger cells had increased protein contents. FIG. 2D shows that the mean quantitative variability per cell was tightly distributed, suggesting high consistency of sample preparation. The consistency of protein quantification was estimated as the coefficient of variation (CV) of the relative levels of peptides originated from the same protein. FIG. 2E Principal component analysis separates single-cell samples corresponding to melanoma cells or to U937 monocytes. 200 cell bulk samples were projected onto PCA to demonstrate agreement between bulk and single cell measurements.

FIGS. 3A-3C show protein correlations with joint distributions. The points represent the expression levels of two proteins in a single cell. FIG. 3A shows proteins that correlate in a similar manner within both cell types. FIG. 3B shows proteins that correlate with the opposite trend. FIG. 3C shows distributions of Euclidean distances of several complexes plotted along with the distribution for all proteins.

FIGS. 4A-4J identify functional protein groups that covary with cell division cycle (CDC)-markers. FIGS. 4A and 4F show proteins whose abundance varies with CDC phases identified using distributions of DNA content for Fluorescence-activated cell sorting (FACS) sorted cells. FIGS. 4B and 4G show correlations between CDC protein markers computed within the single cells from each type. FIGS. 4C and 4H depict Principal Component Analysis (PCA) of melanoma and monocyte cells in the space of CDC periodic genes. Cells in each PCA plot are colored by the mean abundance of proteins annotated to the marked phase. FIGS. 4D and 4I show boxplots display distributions for correlations between the CDC-phase markers and proteins from the proteins from the polyubiquitination gene ontology (GO) term. The difference between these distributions was evaluated by one-way ANOVA analysis to estimate statistical significance, FDR <5%. The distributions for other GO terms that covary in a similar way between the two cell lines are summarized with their medians plotted as a heatmap. FIGS. 4E and 4J show a similar analysis and display as in FIGS. 4D and 41 and are used to visualize GO terms whose covariation with the CDC is cell-type specific.

FIGS. 5A-5G show melanoma subpopulations. FIGS. 5A and 5F show PCA of melanoma cells which indicates two distinct clusters. Single cells were colored based on the protein abundances corresponding to transcripts previously identified as markers of primed cells (Emert et al., Nat Biotechnol. 39(7):865-76 (2021)). The single cells were also colored by the average abundance of protein sets exhibiting significant enrichment clusters A and B. FIG. 5B shows distributions of cells by CDC-phase for cells from cluster A and B. CDC-phases were determined from marker proteins from FIGS. 3A-3C. FIGS. 5C and 5G are protein sets showing distinct covariation in subpopulation A and B. The analysis and display are as in FIGS. 4E and 4J. FIG. 5D shows marginal distributions of protein abundances differentiating clusters A and B. FIG. 5E shows joint distributions of protein abundances differentiating clusters A and B.

FIGS. 6A-6E show functional protein covariation identified at the single-cell level by nPOP. Closely related pancreatic adenocarcinoma cell lines analyzed at the single-cell level by nPOP are easily clustered by time (FIG. 6A) and result in highly consistent protein quantification based on different peptides (FIG. 6B). The data allow identifying functional protein covariation (FIGS. 6C-6E).

FIGS. 7A-7D evaluate the efficiency of protein extraction by DMSO cell lysis. FIG. 7A Equal number of U-937 cells labeled with “Light” and “Heavy” isotopes via SILAC (stable isotope labeling by amino acids in cell culture) were lysed with urea or DMSO, diluted, and combined for digestion. FIG. 7B shows that the SILAC ratios for proteins from different cellular compartments show comparable protein recovery for DMSO and urea cell lysis. FIG. 7C Equal number of SILAC labeled “Light” Jurkat and “Heavy” U-937 cells were combined, and the mixed sample was then divided for cell lysis either by urea or by DMSO. FIG. 7C shows agreement between the SILAC ratios from the two methods which supports the use of DMSO lysis for quantitative protein analysis.

FIGS. 8A-8C depict another non-limiting example of workflow of nano-Proteomic sample Preparation (nPOP). FIG. 8A is a schematic of nPOP sample preparation method illustrating the steps of cell lysis, protein digestion, peptide labeling with isobaric chemical tags (TMT), and quenching with two additions of hydroxylamine. These steps are performed in parallel for all single cells and take place in small droplets. FIG. 8B is a representative field of droplets post trypsin addition. Droplets with single cells are clustered in groups of 13, and the number of cells are labeled and combined into one SCOPE2 sets using TMTpro. The single-cell droplets are surrounded by a perimeter of water droplets for maintaining high local humidity. FIG. 8C shows total ion current chromatograms from three runs demonstrating low contaminants and consistent chromatography.

FIGS. 9A-9D show reporter ion intensities in single cells and in negative controls. FIGS. 9A-9B show the reporter ion intensities for two representative Single Cell ProtEomics 2 (SCOPE2) sets prepared with nPOP. The panels show distributions of reporter ion intensities relative to the corresponding isobaric carrier for the set. RI intensities are mostly absent from negative control wells, which contains all reagents but not a single cell. FIG. 9C estimates the consistency of protein quantification using the coefficient of variation (CV) of the relative levels of peptides originating from the same protein. The median CVs per cells form a tight distribution, suggesting high consistency of sample preparation. FIG. 9D PCA separates samples corresponding to HeLa cells or to monocytes. The single cells cluster with bulk samples of 100 cells, indicating consistent relative protein quantitation.

FIGS. 10A-10H cluster cells based on cell type and cell cycle phase. PCA of HeLa cells in the space of proteins whose abundance is periodic with the cell cycle. Cells in each PCA plot are colored by the mean abundance of proteins annotated to the M/G1, G1/S, S, and G2 phases.

### DETAILED DESCRIPTION

A description of example embodiments follows.

Traditionally, single-cell proteomic analyses have been performed by using fluorescent proteins or affinity reagents. While these approaches are powerful, mass spectrometry (MS) has the potential to increase the specificity and depth of single-cell protein quantification. For decades, MS has been a powerful tool for quantitative measurements of thousands of proteins in bulk samples consisting of thousands of cells or more.

Bulk samples are often prepared for liquid chromatography tandem MS analysis by using relatively large volumes (hundreds of microliters) and chemicals (detergents or chaotropic agents like urea) that are incompatible with MS analysis and require removal by cleanup procedures. The large volumes and cleanup procedures entail sample losses that may be prohibitive for small samples, such as single mammalian cells.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

When introducing elements disclosed herein, the articles “a,” “an,” “the,” and “said” are intended to mean that there are one or more of the elements. Further, the one or more elements may be the same or different.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

### Methods of the Disclosure

In various aspects, the disclosure provides methods of forming single-cell proteomic samples.

In one aspect, the disclosure provides a method of forming a single-cell proteomic sample, said method comprising:
a) dispensing n droplets of lysis buffer onto a substantially planar solid surface, wherein n≥2;
b) dispensing a single cell into each of the n droplets of lysis buffer to produce n droplets, each comprising a lysed single cell;
c) dispensing digestion buffer into each of the n droplets to digest proteins from each lysed single cell to produce n droplets comprising peptides;
d) dispensing a chemical tag into each of the n droplets comprising the peptides to produce labeled peptides, wherein at least one droplet of the n droplets receives a different chemical tag from at least one other droplet of the n droplets, thereby enabling the labeled peptides in the at least one droplet to be distinguishable from the labeled peptides in the at least one other droplet; and
e) applying a fluid to merge at least a subset of the n droplets into a combined droplet on the substantially planar surface, thereby combining the labeled peptides to form a single-cell proteomic sample.

In some embodiments, each of the n droplets in step a), b), c), and/or d) has a volume of less than 100 nanoliters (nl or nL), for example, less than 80, 60, 50, 40, 35, 30, 25, 22 or 20 nl. In certain embodiments, each of the n droplets in step a), b), c), and/or d) has a volume of about 100 nl or less, for example, about: 80, 60, 50, 40, 35, 30, 25, 22 or 20 nl or less. In particular embodiments, each of the n droplets in step a), b), c), and/or d) has a volume of about 25 nanoliters (nl) or less. In more particular embodiments, each of the n droplets in step a), b), c), and d) has a volume of about 25 nl or less.

In certain embodiments, the lysis buffer, the digestion buffer, the chemical tag, or a combination thereof is dispensed in a volume of about 1-20 nl per droplet, for example, about: 1-18, 1-16, 1-14, 1-12, 1-10, 1-8, I-6, I-4, 2-18, 2-16, 2-14, 2-12, 2-10, 2-8, 2-6, 2-4, 4-20, 4-18, 4-16, 4-14, 4-12, 4-10, 4-8, 4-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 12-20, 12-18, 12-16, 12-14, 14-20, 14-18, 14-16, 16-20, 16-18 or 18-20 nl.

In some embodiments, the disclosure provides a method of forming at least two single-cell proteomic samples, wherein steps a) to e) are repeated at least once to form two or more single-cell proteomic samples. In certain embodiments, steps a) to e) are repeated at least 3 times, for example, at least 5, 10, 20, 30, 50, 80, 100, 120, 150, 180, 200, 250, 300, 350, 400, 500 or 1,000 times. In particular embodiments, steps a) to e) are repeated about 200 times.

### Substantially Planar Solid Surfaces

As used herein, the term “substantially planar solid surface” refers to a surface that is substantially flat. In some embodiments, a substantially planar solid surface is a smooth surface. In certain embodiments, a substantially planar solid surface comprises etching, one or more (e.g., arrays of) very shallow dimples, or a combination thereof. A substantially planar solid surface enables small droplets (e.g., about 10-200 nl) of liquids to merge into a combined droplet when applying a fluid of a discrete volume (e.g., about 1 microliter (μl or μL)). A member (such as a multi-well plate or a microfuge tube) where its contents are closed off or surrounded, for example, by a wall, does not have a substantially planar solid surface. In particular embodiments, the substantially planar solid surface is provided by a slide, for example, a uniform glass slide.

In some embodiments, at least 90% of the points in the substantially planar surface are located on one of or between a pair of planes which are parallel and which are spaced from each other by a distance of not more than 5% of the largest dimension of the surface. In certain embodiments, the radius of curvature of the space is much greater than the cross-sectional dimensions, and the curvature does not substantially alter the function of the space. In particular embodiments, the substantially planar surface has a generally uniform thickness and having surface dimensions that are both much larger (e.g., ten to 100 times or more) than the thickness.

In certain embodiments, the substantially planar solid surface is etched, for example, with a laser. An “etched surface” refers to a surface that is made by etching.

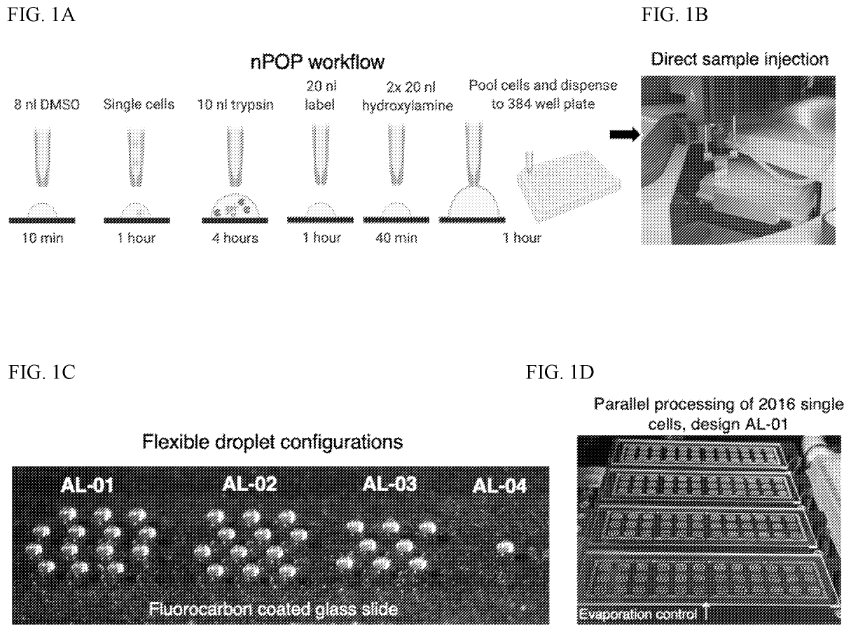

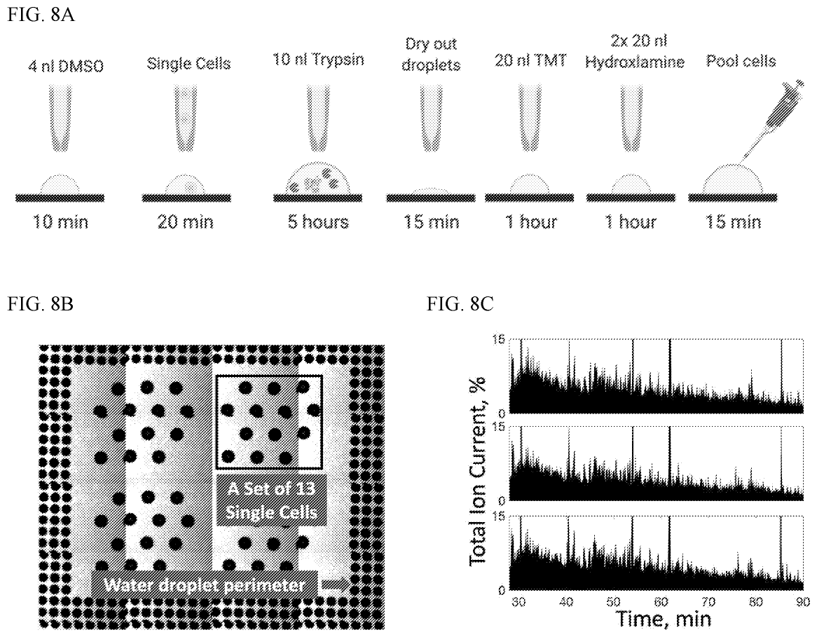

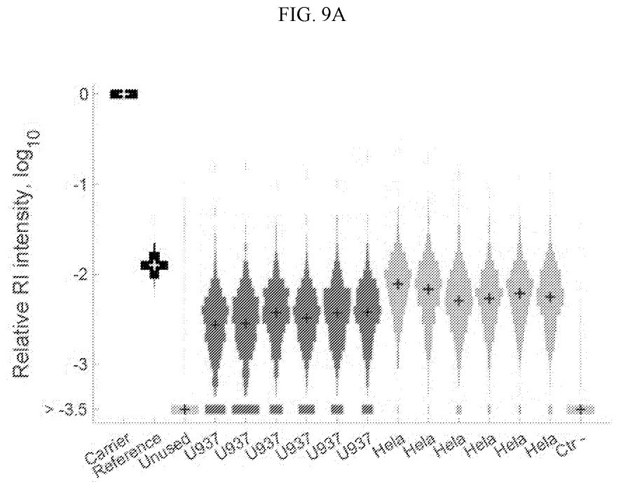

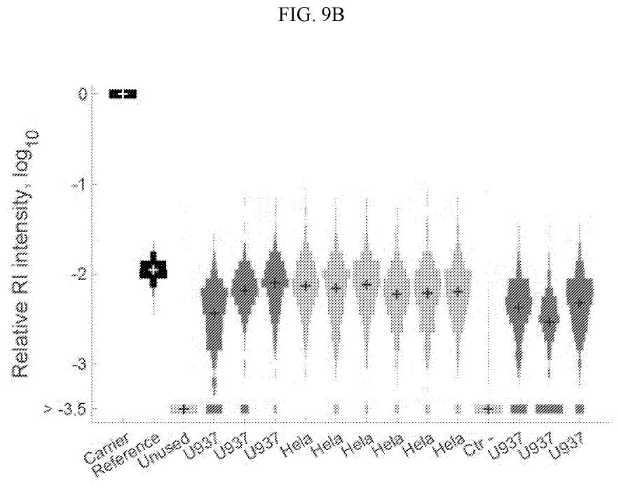

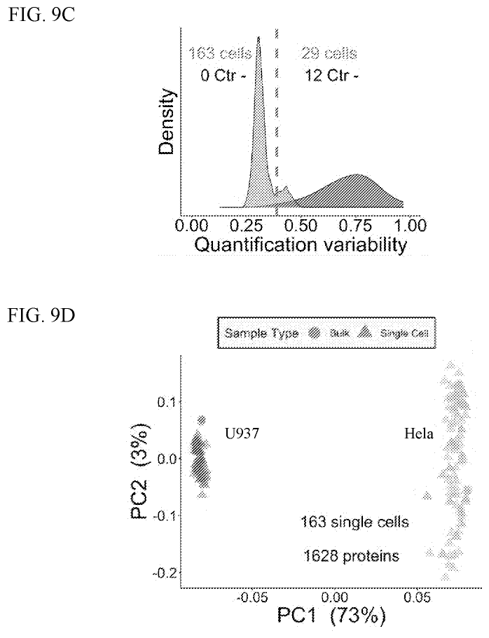

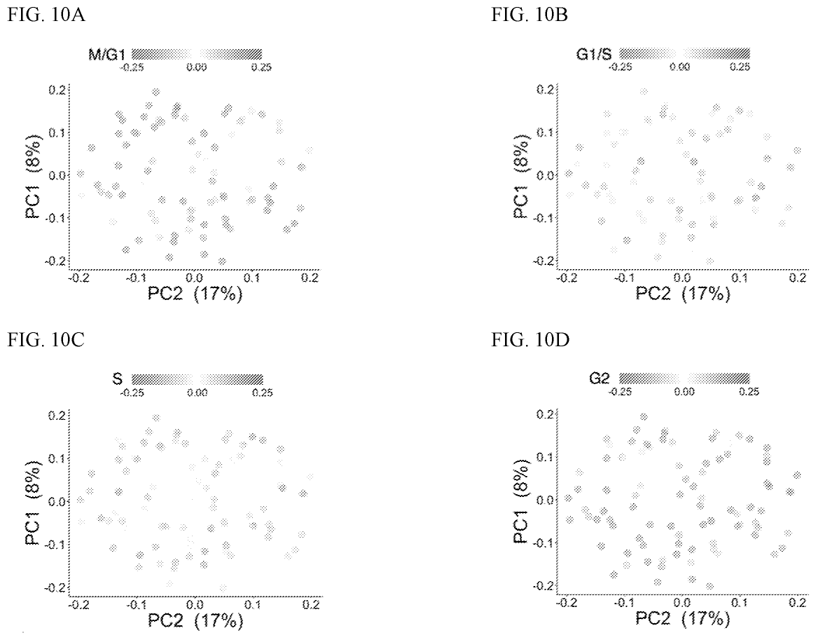

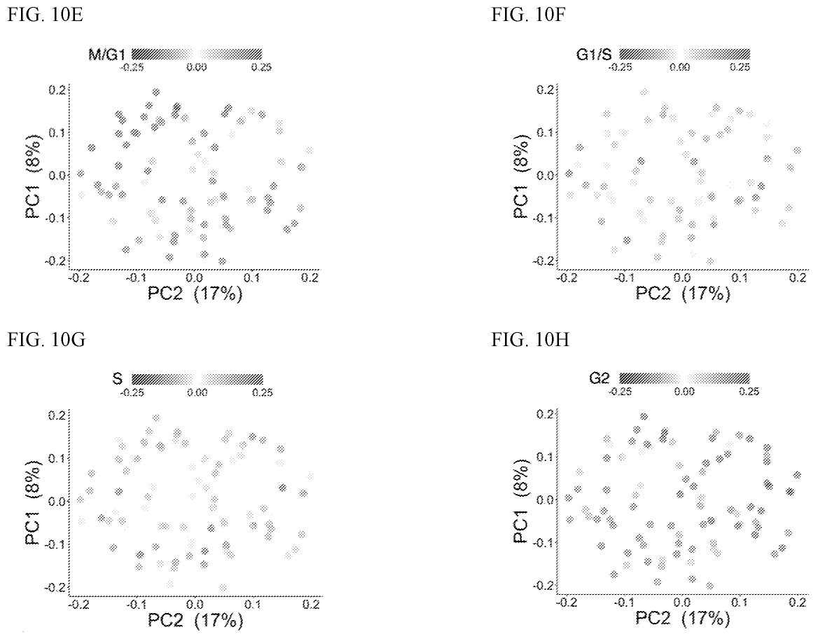

In some embodiments, the substantially planar solid surface comprises etchings arranged in spaced relation to each other (e.g., into clusters of a discrete number of spots (see, e.g., FIGS. 1C and 8B)). In some embodiments, the substantially planar solid surface comprises etchings with a geometric pattern. The arrangement and/or geometric pattern may be programmable. For example, a geometric pattern may be designed by a person of ordinary skill in the art based on the goal of the proteomic analysis, sample multiplexing strategy, etc., or a combination thereof. Suitable geometric patterns may include about 1-120 clusters per substantially planar solid surface, for example, about: 18, 36, 54, 72, 90 or 108 clusters per surface; and each cluster may include about 1-20 spots, for example, about: 1, 2, 3, 4, 5, 6, 7, 8, 9, 10, 11, 12, 13, 14, 15, 16, 17, 18, 19 or 20 spots. In some embodiments, each cluster has at least 14 spots. In certain embodiments, the geometric pattern includes 36 clusters with at least 14 spots per cluster. In particular embodiments, the geometric pattern includes 36 clusters with at least 16 spots per cluster.

In some embodiments, the substantially planar solid surface is unetched.

In some embodiments, the distance between two spots (e.g., two closest spots) within a cluster is about 0.1-10.0 mm, for example, about: 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10.0 mm. In certain embodiments, the distance between two spots (e.g., two closest spots) within a cluster is about: 0.1-9.5, 0.15-9.5, 0.15-9.0, 0.2-9.0, 0.2-8.5, 0.25-8.5, 0.25-8.0, 0.3-8.0, 0.3-7.5, 0.35-7.5, 0.35-7.0, 0.4-7.0, 0.4-6.5, 0.45-6.5, 0.45-6.0, 0.5-6.0, 0.5-5.5, 0.55-5.5, 0.55-5.0, 0.6-5.0, 0.6-4.5, 0.65-4.5, 0.65-4.0, 0.7-4.0, 0.7-3.5, 0.75-3.5, 0.75-3.0, 0.8-3.0, 0.8-2.5, 0.85-2.5, 0.85-2.0, 0.9-2.0, 0.9-1.5, 0.95-1.5 or 0.95-1.0. In particular embodiments, the distance between two spots (e.g., two closest spots) within a cluster is about 1.0 mm.

In some embodiments, the distance between the centers of two clusters (e.g., two neighboring clusters) is about 3.0-50 mm, for example, about: 3.5, 4.0, 4.5, 5.0, 5.5, 6.0, 8.0, 10, 15, 20, 30, 40 or 50 mm. In certain embodiments, the distance between the centers of two clusters (e.g., two neighboring clusters) is about: 3.0-40, 3.5-40, 3.5-30, 4.0-30, 4.0-20, 4.5-20, 4.5-15, 5.0-15, 5.0-10, 5.5-10, 5.5-8 or 6-8. In particular embodiments, the distance between the centers of two clusters (e.g., two neighboring clusters) is about 6 mm.

The distance between two spots (e.g., two closest spots) within a cluster and/or the distance between the centers of two clusters (e.g., two neighboring clusters) may be designed by a person of ordinary skill in the art based on the goal of the proteomic analysis, sample multiplexing strategy and/or desired throughput.

Methods disclosed herein can be compatible with many types of substantially planar solid surfaces with a wide range of sizes. In some embodiments, the length of the substantially planar solid surface is about 10 mm to 50 cm, for example, about: 20 mm to 50 cm, 20 mm to 25 cm, 40 mm to 25 cm, 40 mm to 12 cm, 50 mm to 12 cm, 50 mm to 10 cm, 100 mm to 10 cm, 100 mm to 5 cm, 200 mm to 5 cm, 200 mm to 2.5 cm, 500 mm to 2.5 cm or 500 mm to 1.0 cm.

In certain embodiments, the width of the substantially planar solid surface is about 5.0 mm to 30 cm, for example, about: 10 mm to 30 cm, 20 mm to 30 cm, 20 mm to 15 cm, 50 mm to 15 cm, 50 mm to 10 cm, 100 mm to 10 cm, 100 mm to 5.0 cm, 200 mm to 5.0 cm, 200 mm to 2.5 cm, 500 mm to 2.5 cm, 500 mm to 2.0 cm or 1.0 to 2.0 cm.

In particular embodiments, the substantially planar solid surface is provided by microscopic glass slides with dimensions of 75 mm by 25 mm (3″ by 1″) and about 1 mm thickness.

In certain embodiments, the substantially planar solid surface is coated with a compound (e.g., a compound that is neither hydrophobic nor hydrophilic) to stabilize the individual droplets. In particular embodiments, the substantially planar solid surface is fluorocarbon-coated. The term “fluorocarbon” refers to a compound formed by replacing one or more of the hydrogen atoms in a hydrocarbon with fluorine atoms.

In certain embodiments, movement of the substantially planar solid surface is minimized.

### Lysing Single Cells

Lysing single cells comprises dispensing n droplets of lysis buffer onto the substantially planar solid surface (e.g., etched or unetched uniformed glass slide), wherein n≥2; and dispensing a single cell into each of the n droplets of lysis buffer to produce n droplets, each comprising a lysed single cell.

As used herein, the term “liquid droplet” refers to a very small drop of a liquid. In some embodiments, each individual droplet comprising the lysis buffer has a volume of about 1.0-10.0 nl, for example, about: 1.0, 2.0, 3.0, 4.0, 5.0, 6.0, 7.0, 8.0, 9.0, 10.0, 1.0-4.0, 1.0-6.0, 1.0-8.0, 2.0-4.0, 2.0-6.0, 2.0-8.0, 2.0-10.0, 4.0-6.0, 4.0-8.0, 4.0-10.0, 6.0-8.0, 6.0-10.0 or 8.0-10.0 nl. In certain embodiments, each individual droplet comprising the lysis buffer has a volume of about 10.0 nl or less, for example, about: 9.5, 9.0, 8.5, 8.0, 7.5, 7.0, 6.5, 6.0, 5.5, 5.0, 4.5 or 4.0 nl or less. In some embodiments, each individual droplet comprising the lysis buffer has a volume of about 4 nl. In particular embodiments, each individual droplet comprising the lysis buffer has a volume of about 8 nl.

In certain embodiments, the individual droplets of lysis buffer are dispensed using a first piezo dispensing capillary (PDC), for example, that of cellenONER (SCIENION GmbH, Berlin, Germany). In some embodiments, the first PDC is dedicated for handling organic solvents, protein solutions, or a combination thereof. In other embodiments, the individual droplets of lysis buffer are dispensed with MANTISR Liquid Handler (FORMULATRIXR, Bedford, MA) or HP D300e Digital Dispenser (Hewlett-Packard, Palo Alto, CA).

In some embodiments, n is >3, for example, >4, >5, >6, >7, >8, >9, >10, ≥11, ≥12, ≥13, >14, ≥15, ≥16, ≥17, >18, >19 or >20. In certain embodiments, n is about 2-20, for example, 2, 3, 4, 5, 6, 7, 8, 9, 10, 11, 12, 13, 14, 15, 16, 17, 18, 19 or 20, or 2-18, 3-18, 3-16, 4-16, 4-14, 5-14, 5-12, 6-12 or 6-10. In particular embodiments, n is about 12-20. In more particular embodiments, n is about 14-18. In some embodiments, the n droplets are arranged in spaced relation to each other (e.g., into a cluster (see, e.g., FIGS. 1C and 8B)).

In some embodiments, the method comprises dispensing m times n droplets of lysis buffer onto a substantially planar solid surface, wherein n (corresponding to the number of droplets per subgroup/cluster)>2, and m (corresponding to the number of subgroups/clusters)≥2.

For example, a multiplexing format may be designed by a person of ordinary skill in the art based on the goal of the proteomic analysis, sample multiplexing strategy, etc., or a combination thereof. A suitable multiplexing format may include about 1-120 clusters per substantially planar solid surface, for example, about: 18, 36, 54, 72, 90 or 108 clusters per substantially planar solid surface; and each cluster may include about 1-20 droplets, for example, about: 1, 2, 3, 4, 5, 6, 7, 8, 9, 10, 11, 12, 13, 14, 15, 16, 17, 18, 19 or 20 droplets. In certain embodiments, the multiplexing format comprises at least about 10 clusters, for example, at least about: 15, 20, 25, 30, 35, 40, 45, 50, 55, 60, 65, 70, 75, 80, 90 or 100 clusters.

In some embodiments, each cluster has at least about 6 droplets, for example, at least about: 7, 8, 9, 10, 11, 12, 13, 14, 15 or 16 droplets. In particular embodiments, the multiplexing format includes about 14 droplets per cluster. In more particular embodiments, the multiplexing format includes about 16 droplets per cluster.

In certain embodiments, the multiplexing format includes at least 10 clusters, and each cluster comprising at least 10 droplets (e.g., 14-16 droplets). In particular embodiments, the multiplexing format includes 36 clusters with 14 droplets per cluster.

In some embodiments, a total of about 100-10,000 individual droplets comprising the lysis buffer are dispensed onto the substantially planar solid surface, for example, about: 100-9,000, 150-9,000, 150-8,000, 200-8,000, 200-6,000, 300-6,000, 300-5,000, 500-5,000, 500-4,000, 750-4,000, 750-3,000, 1,000-3,000, 1,500-3,000, 1,500-2,000 or 2,000-3,000 individual droplets. In certain embodiments, about 2,000 (e.g., 2016) individual droplets are dispensed onto the substantially planar solid surface.

In some embodiments, the lysis buffer is devoid of any compound incompatible with the proteomic analysis (e.g., mass spectrometry (MS)). In certain embodiments, the method is devoid of one or more steps for removing one or more incompatible compounds (“cleanup steps”).

In certain embodiments, the lysis buffer comprises a mass-spec compatible organic solvent and/or detergent, such as acetonitrile, n-Dodecyl-ß-D-maltopyranoside (DDM), n-Decyl-B-D-maltopyranoside (DM) and Rapigest.

In some embodiments, the lysis buffer comprises a compound compatible with the intended proteomic analysis (e.g., MS). In certain embodiments, the compound has a vapor pressure of about 0.500-0.700 mm Hg or less at 25° C. In particular embodiments, the compound has a vapor pressure of about 0.600 mm Hg at 25° C. In more particular embodiments, the compound is an organosulfur compound, for example, dimethyl sulfoxide (DMSO).

In some embodiments, the lysis buffer comprises 33-100% DMSO, for example, 40-100%, 50-100%, 60-100%, 70-100%, 80-100%, 90-100%, 92-100%, 94-100%, 95-100%, 96-100%, 97-100%, 98-100% or 99-100% DMSO. In certain embodiments, the lysis buffer comprises about 4.0-8.0 nl of 90-100% DMSO. In particular embodiments, the lysis buffer comprises (e.g., consists of) about 4.0 nl 90-100% DMSO. In more particular embodiments, the lysis buffer comprises (e.g., consists of) about 8.0 nl 90-100% DMSO.

In some embodiments, a perimeter of water (e.g., mass spectrometry grade water) droplets is dispensed in a perimeter surrounding each grid (see, e.g., FIGS. 1D and 8B) to provide local humidity and, thus, reaction volume control. In particular embodiments, the system is set to refresh the water droplet perimeter to control local humidity, e.g., periodically (e.g., every 40 minutes).

### Single-Cell Dispensation

In some embodiments, the single cell is a prokaryotic cell. In certain embodiments, the single cell is a eukaryotic cell (e.g., an animal cell, a plant cell, a fungus cell, or a protist cell). Non-limiting examples of animals include humans, domestic animals, such as laboratory animals (e.g., cats, dogs, monkeys, pigs, rats, mice, etc.), household pets (e.g., cats, dogs, rabbits, etc.), livestock (e.g., pigs, cattle, sheep, goats, horses, etc.), and non-domestic animals. In particular embodiments, the single cell is a mammalian cell (e.g., a human cell).

In some embodiments, the single cell is a germ-line cell. In certain embodiments, the single cell is a somatic cell. Non-limiting examples of somatic cells include stem cells, red blood cells, white blood cells (e.g., neutrophils, eosinophils, basophils, or lymphocytes), platelets, nerve cells, neuroglial cells, muscle cells (e.g., skeletal muscle cells, cardiac muscle cells, or smooth muscle cells), cartilage cells, and skin cells. In certain embodiments, the individual cells comprise tumor cells (e.g., melanoma cells).

In some embodiments, the single cell has a diameter of less than 100 μm. In certain embodiments, the single cell has a diameter of about 10-20 μm. In particular embodiments, the single cell has a diameter of about 10-15 μm.

In some embodiments, the single-cell proteomic sample comprises peptides from at least two cells, for example, from at least about: 10, 15, 20, 30, 50, 80, 100, 150, 200, 250, 300, 500, 750, 1,000, 1,500, 2,000, 2,500, 3,000, 4,000, 5,000, 6,000, 7,000, 8,000 or 10,000 cells. In certain embodiments, the single-cell proteomic sample comprises peptides from about 10-10,000 cells, for example, about: 10-9,000, 15-9,000, 15-8,000, 30-8,000, 30-6,000, 50-6,000, 50-5,000, 100-5,000, 100-4,000, 150-4,000 or 150-3,000 cells. In some embodiments, the single-cell proteomic sample comprises peptides from at least 100 cells. In certain embodiments, the single-cell proteomic sample comprises peptides from at least 1,000 cells. In particular embodiments, the single-cell proteomic sample comprises peptides from at least 1,500 cells.

In some embodiments, the cells are a homogenous cell population (of the same cell type). In other embodiments, two or more cell types are dispensed into the n droplets of lysis buffer, for example, 3, 4, 5, 6, 7, 8, 9 or 10 or more cell types. Each cell type may comprise multiple subpopulations based on certain characteristics, for example, cell division cycle (CDC). In particular embodiments, the method further comprises enriching a subpopulation of cells, for example, with Fluorescence-activated cell sorting (FACS) (e.g., based on size, DNA content, cellular state, and/or surface marker), culture condition, reporter-based selection, or a combination thereof.

In some embodiments, (isolating and) dispensing the single cell uses a second piezo dispensing capillary (PDC), for example, that of cellenONER (SCIENION GmbH, Berlin, Germany). In some embodiments, the second PDC is dedicated to handling cell suspensions. In other embodiments, (isolating and) dispensing the single cell uses MANTISR Liquid Handler (FORMULATRIXR, Bedford, MA) or HP D300e Digital Dispenser (Hewlett-Packard, Palo Alto, CA).

In some embodiments, step b) comprises dispensing the single cell in a buffer (e.g., phosphate buffered saline (PBS)) with a measured volume. In certain embodiments, the measured volume is from about 30 picoliters to about 3,000 picoliters, for example, about: 30-2,400, 45-2,400, 45-1,800, 60-1,800, 60-1,200, 90-1,200, 90-900, 100-1,000, 120-900, 120-600, 150-600, 150-450, 200-450, 200-400, 200-300, 250-350, 260-340, 270-330, 280-320, 290-310 or 300-450 picoliters. In certain embodiments, the measured volume is less than 3,000 picoliters, for example, less than: 2,500, 2,400, 2,000, 1,800, 1,500, 1,200, 1,000, 800, 500, 450 or 400 picoliters. In particular embodiments, the measured volume is about 300 picoliters.

In certain embodiments, step b) comprises dispensing the single cell in a cell suspension buffer with a volume of about 100-1,000 picoliters. In particular embodiments, step b) comprises dispensing the single cell in a cell suspension buffer with a volume of about 300 picoliters.

In some embodiments, the method further comprises dispensing a cell suspension buffer devoid of any cell into one or more droplets of lysis buffer, for example, as a negative control for detecting background noise, contamination, etc., or a combination thereof.

### Cell Lysis

In some embodiments, step b) enables lysing the single cell in a total volume of about 5.0-12.0 nl, for example, of about: 5.0, 5.5, 6.0, 6.5, 7.0, 7.5, 8.0, 8.5, 9.0, 9.5, 10.5, 11.0, 11.5, 12.0, 5.5-12.0, 5.5-11.5, 6.0-11.5, 6.0-11.0, 6.5-11.0, 6.5-10.5, 7.0-10.5, 7.0-10.0, 7.5-10.0, 7.5-9.5, 8.0-9.5 or 8.0-8.5 nl. In particular embodiments, step b) enables lysing the single cell in a total volume of about 7.5-8.5 nl. In particular embodiments, step b) enables lysing the single cell in a total volume of about 8.0-8.5 nl.

In certain embodiments, step b) enables lysing the single cell for about 10-20 minutes. In some particular embodiments, step b) enables lysing the single cell in a total volume of about 8-8.5 nl for about 10-20 minutes.

In some embodiments, 5.0-12.0 μl is the sum of the volume of the lysis buffer plus the volume of the single cell in its dispensing solution, for example, about: 5.0, 5.5, 6.0, 6.5, 7.0, 7.5, 8.0, 8.5, 9.0, 9.5, 10.5, 11.0, 11.5, 12.0, 5.5-12.0, 5.5-11.5, 6.0-11.5, 6.0-11.0, 6.5-11.0, 6.5-10.5, 7.0-10.5, 7.0-10.0, 7.5-10.0, 7.5-9.5, 8.0-9.5 or 8.0-8.5 nl. In particular embodiments, 4-10 μl is the sum of the volume of the lysis buffer plus the volume of the single cell in its dispensing solution.

### Protein Digestion

In certain embodiments, the digestion buffer is a trypsin buffer, and dispensing digestion buffer into each of the n droplets produces a solution comprising about 100-150 ng/μl trypsin. In particular embodiments, dispensing digestion buffer into each of the n droplets produces a solution comprising about 120 ng/μl trypsin in about 5 mM HEPES buffer.

In certain embodiments, dispensing digestion buffer into each of the n droplets produces a solution with a volume of about 15-25 nl, for example, about: 15, 16, 17, 18, 19, 20, 21, 22, 23, 24, 25, 15-20, 16-20, 16-19, 17-19 or 18-19 nl. In particular embodiments, dispensing digestion buffer into each of the n droplets produces a solution with a volume of about 18 nl.

In some embodiments, step c) comprises enabling the proteins from each lysed single cell to be digested at about 1ºC above the dew point, for example, about: 0.4-1.6° C., 0.5-1.5° C., 0.6-1.4° C., 0.7-1.3ºC, 0.8-1.2° C. or 0.9-1.1° C. above the dew point. In certain embodiments, step c) comprises enabling the proteins from each lysed single cell to be digested at a relative humidity of about 70-80%, for example, about: 70%, 71%, 72%, 73%, 74%, 75%, 76%, 77%, 78%, 79%, 80%, 71-79%, 72-78%, 73-77%, 74-76%, or 74.5-75.5%. As used herein, the term “relative humidity” refers to the amount of water vapor present in air expressed as a percentage of the amount needed for saturation at the same temperature. In particular embodiments, step c) comprises enabling the proteins from each lysed single cell to be digested at a relative humidity of about 75%. In more particular embodiments, step c) comprises enabling the proteins from each lysed single cell to be digested at about 1ºC above the dew point and at a relative humidity of about 75%. In some embodiments, the temperature, the relative humidity, or both are dynamically regulated.

In some embodiments, step c) comprises enabling the proteins from each lysed single cell to be digested for about 3-5 hours, for example, for about: 3, 3.5, 4, 4.5, 5, 3.1-4.9, 3.2-4.8, 3.3-4.7, 3.4-4.6, 3.5-4.5, 3.6-4.4, 3.7-4.3, 3.8-4.2 or 3.9-4.1 hours.

In some embodiments, step c) comprises:
dispensing about 15-25 nl of about 120 ng/μl trypsin into each of the n droplets; and
enabling the proteins from each lysed single cell to be digested at about 1° C. above the dew point and a relative humidity of about 75% for about 4-5 hours.

In certain embodiments, the digestion buffer is dispensed using the first piezo dispensing capillaries (PDC), for example, that of cellenONER (Lyon, France). In other embodiments, the digestion buffer is dispensed using MANTISR Liquid Handler (FORMULATRIXR, Bedford, MA) or HP D300e Digital Dispenser (Hewlett-Packard, Palo Alto, CA).

### Single-Cell Proteomics

In some embodiments, the one or more single-cell proteomic samples are intended for tandem mass spectrometry. Tandem mass spectrometry, also referred to herein as MS/MS or MS2, involves multiple steps of mass spectrometry selection, with some form of fragmentation occurring in between the stages. In a tandem mass spectrometer, ions are formed in the ion source and separated by mass-to-charge ratio in the first stage of mass spectrometry (MS1). Ions of a particular mass-to-charge ratio (precursor ions) are selected and fragment ions (product ions) are created by collision-induced dissociation, ion-molecule reaction, photodissociation, or other processes known to those skilled in the art. The resulting ions are then separated and detected in a second stage of mass spectrometry (MS2). A common use is for analysis of proteins and peptides.

In certain embodiments, the one or more single-cell proteomic samples are intended for quantitative proteomics. Quantitative proteomics can be used, for example, to determine the relative or absolute amount of proteins in a sample.

Several quantitative proteomics methods are based on MS/MS. One method commonly used for quantitative proteomics is isobaric tag labeling. Isobaric tag labeling enables simultaneous identification and quantification of proteins from multiple samples in a single analysis. To quantify proteins, peptides are labeled with chemical tags that have the same structure and nominal mass, but vary in the distribution of heavy isotopes in their structure. These tags, commonly referred to as tandem mass tags (TMT), are designed so that the mass tag is cleaved at a specific linker region upon higher-energy collisional-induced dissociation during tandem mass spectrometry, yielding reporter ions of different masses. Protein quantitation is accomplished by comparing the intensities of the reporter ions in the MS/MS spectra.

MS/MS can also be used for protein sequencing, as is understood by those skilled in the art. When intact proteins are introduced to a mass analyzer, it is termed “top-down proteomics,” and when proteins are digested into smaller peptides and subsequently introduced into the mass spectrometer, it is termed “bottom-up proteomics”. Shotgun proteomics is a variant of bottom-up proteomics in which proteins in a mixture are digested prior to separation and tandem mass spectrometry.

In some embodiments, the one or more single-cell proteomic samples are generated for cell classification, uncovering a regulatory process, associating a regulatory process with a functional outcome, or a combination thereof. In particular embodiments, the one or more single-cell proteomic samples are generated for understanding cell cycle regulation. In some embodiments, the one or more single-cell proteomic samples are generated for identifying proteins whose abundance differs in G1, S, and/or G2/M phase for two or more cell types.

In some embodiments, the one or more single-cell proteomic samples comprise 10 or more cells of the same cell type to minimize batch effects, background noise, or a combination thereof. In certain embodiments, the one or more single-cell proteomic samples comprise at least 10 cells of the same cell type, for example, at least: 15 cells, 20 cells, 30 cells, 50 cells, 80 cells, 100 cells, 150 cells, 200 cells, 250 cells, 300 cells, 500 cells, 750 cells, 1,000 cells, 1,500 cells, 2,000 cells, 2,500 cells or 3,000 cells of the same cell type. In certain embodiments, the one or more single-cell proteomic samples comprise about 10-10,000 cells of the same cell type, for example, about: 10-9,000 cells, 15-9,000 cells, 15-8,000 cells, 30-8,000 cells, 30-6,000 cells, 50-6,000 cells, 50-5,000 cells, 100-5,000 cells, 100-4,000 cells, 150-4,000 cells, 150-3,000, 500-3,000, 1,000-3,000, 1,000-2,500, 1,000-2,000, 1,500-3,000, 1,500-2,500 or 1,500-2,000 cells of the same cell type. In particular embodiments, the one or more single-cell proteomic samples comprise about 1,500-2,000 cells.

In some embodiments, the disclosed methods enable performing parallel sample preparation of multiple (e.g., hundreds or thousands of) single cells; obviating sample cleanup and associated losses; minimizing bias for cellular compartments; supporting accurate relative protein quantification, or a combination thereof.

In some embodiments, the disclosed methods further comprise performing at least one proteomic analysis on the single-cell proteomic sample. In particular embodiments, the at least one single-cell proteomic analysis enables identifying and/or quantifying protein covariation across the single cells.

In certain embodiments, the single-cell proteomic analysis is performed on a non-substantially planar solid surface, for example, in a multi-well plate or in a tube (such as a microfuge tube).

### Peptide Labeling

Peptide labeling comprises dispensing a chemical tag into each of the n droplets comprising the peptides to produce labeled peptides, wherein at least one droplet of the n droplets receives a different chemical tag from at least one other droplet of the n droplets, thereby enabling the labeled peptides in the at least one droplet to be distinguishable from the labeled peptides in the at least one other droplet (e.g., to be distinguishable from labeled peptides in any other droplet within a cluster/subgroup).

In particular embodiments, each droplet of the n droplets receives a unique chemical tag, thereby enabling the labeled peptides in each droplet to be distinguishable from the labeled peptides in each other droplet.

In isobaric labeling for tandem mass spectrometry, proteins are extracted from cells, digested, and labeled with tags of the same mass. When fragmented during MS/MS, the reporter ions show the relative amount of the peptides in the samples.

In some embodiments, the chemical tag comprises (e.g., consists of) an isobaric tag. Two commercially available isobaric tags are iTRAQR and tandem mass tag (TMT) reagents. A TMT comprises four regions: mass reporter, cleavable linker, mass normalization, and protein reactive group. TMT reagents can be used to simultaneously analyze, e.g., 2-18 different peptide samples prepared from individual cells. TMT reagents include three types: (1) a reactive NHS ester functional group for labeling primary amines (e.g., TMTduplex™, TMTTMsixplex™, TMT10plex plus™, TMT11-131C™, TMTpro 16plex, TMTpro 18plex,), (2) a reactive iodoacetyl functional group for labeling free sulfhydryls (e.g., iodoTMT™) and (3) reactive alkoxyamine functional group for labeling of carbonyls (e.g., aminoxyTMT™).

In certain embodiments, the peptides are labeled by isobaric mass tags (e.g., TMT or TMTpro) for multiplexed analysis. In particular embodiments, the chemical tag comprises (e.g., consists of) TMTpro 16plex or TMTpro 18plex.

In certain embodiments, the chemical tag comprises (e.g., consists of) an isobaric tag for relative and absolute quantitation (iTRAQR). ITRAQR is a reagent for tandem mass spectrometry that is used to determine the amount of proteins from different sources in a single experiment. iTRAQ® uses stable isotope labeled molecules that can form a covalent bond with the N-terminus and side chain amines of proteins. The iTRAQR reagents are used to label peptides from different samples that are pooled and analyzed by liquid chromatography and tandem mass spectrometry. The fragmentation of the attached tag generates a low molecular mass reporter ion that can be used to relatively quantify the peptides and the proteins from which they originated.

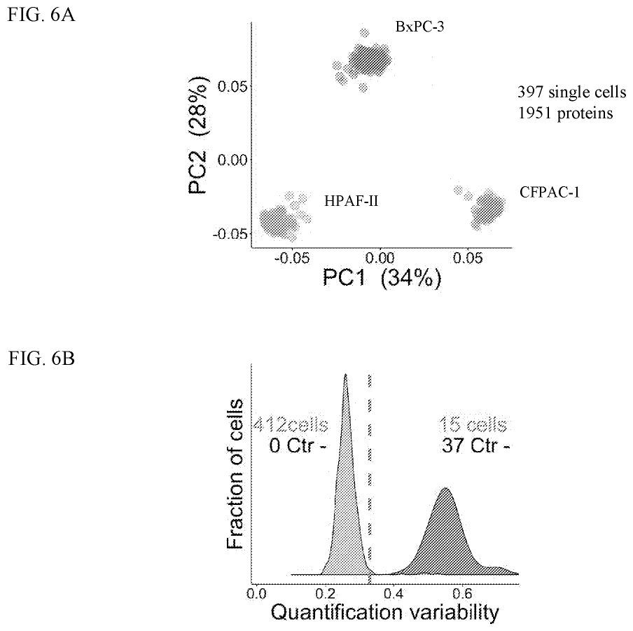

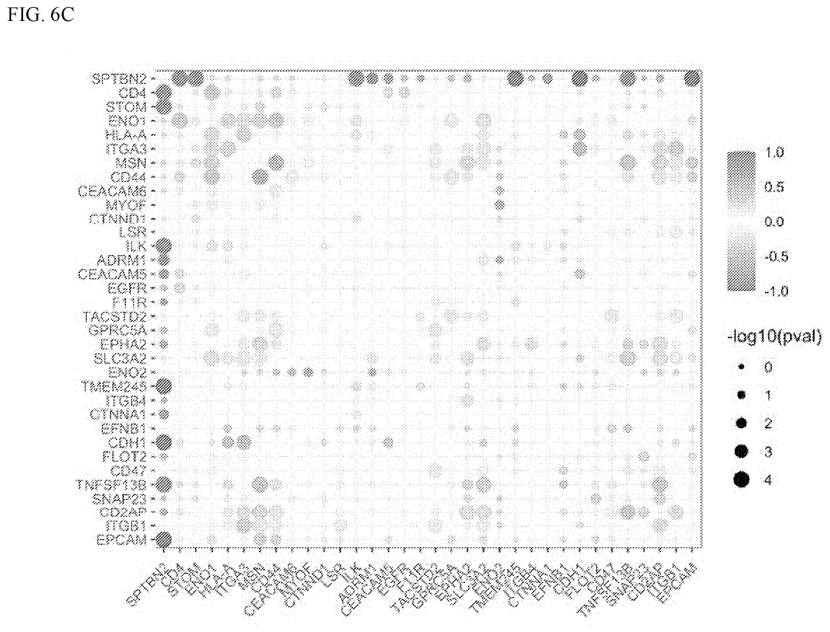

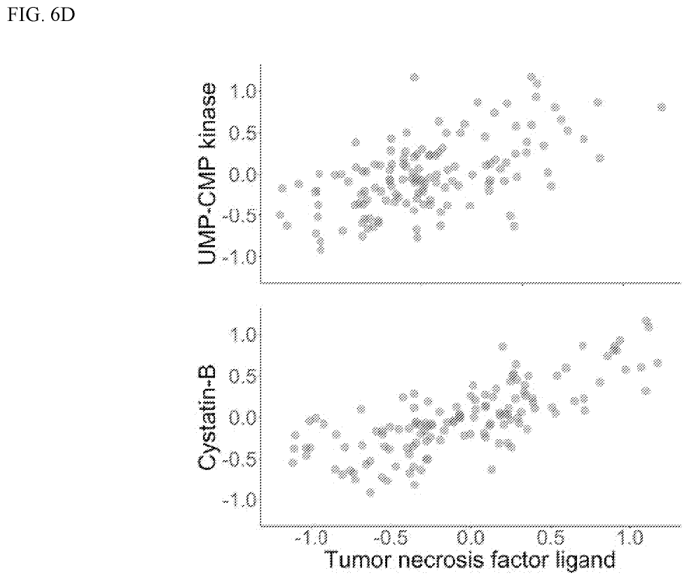

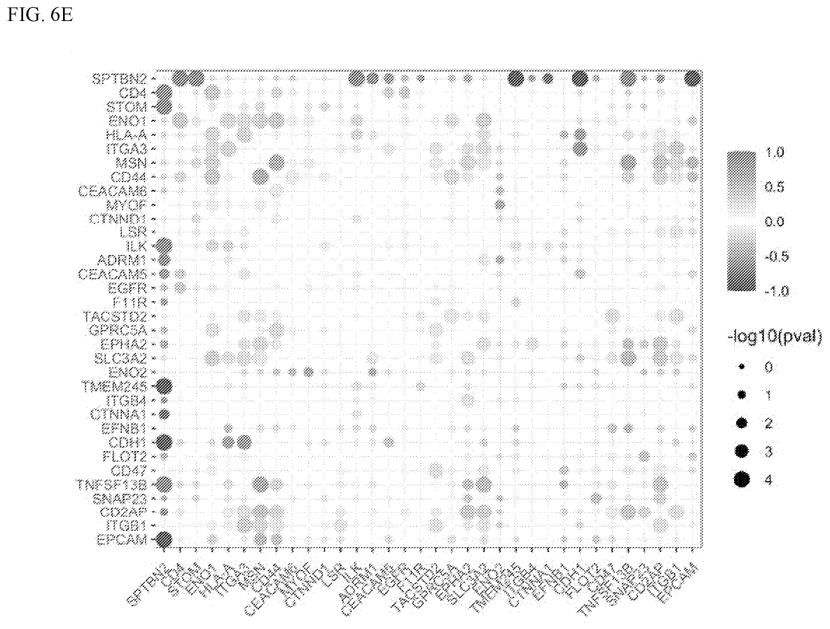

This sample preparation methods described herein are also compatible with non-isobaric mass tags, for example, as demonstrated with mTRAQ (FIG. 6 of Derks et al., bioRxiv 467007 (doi.org/10.1101/2021.11.03.467007) (2021).

In some embodiments, the methods further comprise reducing the volumes of the individual droplets before labeling (e.g., by drying down the individual droplets). In certain embodiments, the volumes of the individual droplets are reduced to about 3-5 nl before dispensing the chemical tag into the corresponding droplet comprising the peptides, for example, about 3.0, 3.5, 4.0, 4.5, 5.0, 3.1-4.9, 3.2-4.8, 3.3-4.7, 3.4-4.6, 3.5-4.5, 3.6-4.4, 3.7-4.3, 3.8-4.2 or 3.9-4.1 nl. In particular embodiments, the volumes of the individual droplets are reduced to about 4 nl before dispensing the chemical tag into the corresponding droplet comprising the peptides.

In certain embodiments, step d) comprises dispensing a chemical tag in a volume of about 15-25 nl into each of the n droplets comprising the peptides, for example, the volume is about: 15, 16, 17, 18, 19, 20, 21, 22, 23, 24, 25, 16-24, 17-23, 18-22, 19-21, 19.5-20.5, 19.6-20.4, 19.7-20.3, 19.8-20.2 or 19.9-20.1 nl. In some embodiments, step d) comprises dispensing a chemical tag in a volume of about 20 nl into each of the n droplets comprising the peptides. In particular embodiments, step d) comprises dispensing TMTpro™ (e.g., “light” version of TMTpro™ 14plex or TMTpro™ 16plex) in a volume of about 20 nl into each of the n droplets comprising the peptides.

In some embodiments, the chemical tag (e.g., TMT) is dissolved in DMSO. In certain embodiments, the chemical tag comprises TMT label reagents (such as of TMTpro™ 14plex or TMTpro™ 16plex) dissolved in DMSO. In particular embodiments, the chemical tag comprises a “light” version of TMT label reagents, also known as TMTO, dissolved in DMSO. In certain embodiments, the chemical tag comprises a “heavy” version of TMT label reagents, also known as TMT super heavy TMTsh, dissolved in DMSO.

In some embodiments, the concentration of the chemical label (e.g., TMTpro™ 14plex) is about 28 mM.

In some embodiments, step d) comprises enabling the chemical tag to react with the peptides at room temperature. In certain embodiments, step d) comprises enabling the chemical tag to react with the peptides at about 18-25° C., for example, at about: 18, 18.5, 19, 19.5, 20, 20.5, 21, 21.5, 22, 22.5, 23, 23.5, 24, 24.5, 25, 18.5-25, 19-24.5, 19.5-24, 20-23.5, 20.5-23, 21-22.5 or 21.5-22° C. In particular embodiments, step d) comprises enabling the chemical tag to react with the peptides at about 20-23.5° C.

In certain embodiments, step d) comprises enabling the chemical tag to react with the peptides at in a total volume of about 18-30 nl, for example, about: 18, 19, 20, 21, 22, 23, 24, 25, 26, 27, 28, 29, 30, 19-29, 20-28, 21-27, 22-26, 23-25, 23-24 or 24-25 nl. In particular embodiments, step d) comprises enabling the chemical tag to react with the peptides in a total volume of about 24 nl.

In certain embodiments, dispensing a chemical tag into each of the n droplets comprising the peptides uses the first piezo dispensing capillaries (PDC), for example, that of cellenONER (SCIENION GmbH, Berlin, Germany). In other embodiments, dispensing a chemical tag into each of the n droplets uses MANTIS® Liquid Handler (FORMULATRIX®, Bedford, MA) or HP D300e Digital Dispenser (Hewlett-Packard, Palo Alto, CA).

In certain embodiments, greater than 90.0% of all peptides are labeled with the (corresponding) chemical tag, for example, greater than: 92.5%, 95.0%, 96.0%, 97.0%, 98.0%, 99.0%, 99.5%, 99.8% or 99.9% of all peptides are labeled. In particular embodiments, greater than 99% of all peptides are labeled.

### Quenching the Labeling Reactions

In some embodiments, the methods of the disclosure further comprise dispensing a quenching reagent into each of the n droplets to quench unconjugated chemical tag.

In certain embodiments, the quenching reagent comprises about 20-30 nl of 5% hydroxylamine.

In particular embodiments, step d) further comprises:
dispensing 20 nl of 5% hydroxylamine into each of the n droplets, and quenching unconjugated chemical tag for about 20 minutes; and
dispensing 30 nl of 5% hydroxylamine into each of the n droplets, and quenching unconjugated chemical tag for about 20 minutes.

In some embodiments, step d) further comprises enabling unconjugated chemical tag to be quenched at about 1ºC above the dew point, for example, about: 0.4-1.6° C., 0.5-1.5° C., 0.6-1.4ºC, 0.7-1.3ºC, 0.8-1.2° ° C. or 0.9-1.1ºC above the dew point. In certain embodiments, step d) further comprises enabling unconjugated chemical tag to be quenched at a relative humidity of about 70-80%, for example, about: 70%, 71%, 72%, 73%, 74%, 75%, 76%, 77%, 78%, 79%, 80%, 71-79%, 72-78%, 73-77%, 74-76%, or 74.5-75.5%. In particular embodiments, step d) further comprises enabling unconjugated chemical tag to be quenched at about 1ºC above the dew point and at a relative humidity of about 75%. In some embodiments, the temperature, the relative humidity, or both are dynamically regulated.

### Pooling (Merging)

Pooling comprises applying a fluid to merge at least a subset the n droplets into a combined droplet on the substantially planar surface, thereby combining the labeled peptides to form a single-cell proteomic sample. In some embodiments, the fluid is water. In certain embodiments, the fluid has a volume of about 1 μl.

In some embodiments, the at least a subset the n droplets comprise n droplets. In certain embodiments, the at least a subset the n droplets comprise ≤n-1 droplets. In particular embodiments, the at least a subset the n droplets comprise ≤n-2 droplets.

In certain embodiments, step e) further comprises aspirating each combined droplet off the substantially planar solid surface in an acetonitrile solution. In some embodiments, the acetonitrile solution comprises about 100% acetonitrile, for example, about: 99.0-100%, 99.5-100%, 99.8-100% or 99.9-100% acetonitrile. In particular embodiments, the acetonitrile solution has a volume of about 5-15 μl, for example, about 5, 6, 7, 8, 9, 10, 11, 12, 13, 14, 15, 9.0-11.0, 9.1-10.9, 9.2-10.8, 9.3-10.7, 9.4-10.6, 9.5-10.5, 9.6-10.4, 9.7-10.3, 9.8-10.2 or 9.9-10.1 μl. In more particular embodiments, the total volume for aspirating each combined droplet is about 10 μl. Each single-cell proteomic sample can be transferred into a single well of a multi-well (e.g., a 384-well) plate.

In some embodiments, the combined droplet (comprising the labeled peptides) is transferred onto a non-substantially planar solid surface. In certain embodiments, the combined droplet (comprising the labeled peptides) is transferred into a container (e.g., a well within a multi-well plate, a tube such as a microfuge tube).

### Drying

In some embodiments, the method further comprises drying the single-cell proteomic samples, for example in a speed-vacuum.

In some embodiments, the one or more single-cell proteomic sample are stored (for example, frozen at −80° C.) for future proteomic analysis. In certain embodiments, the one or more single-cell proteomic sample are reconstituted (for example, each in about 1.1 μl of 0.1% formic acid) for proteomic analysis (e.g., mass spectrometry analysis).

In another aspect, the disclosure provides a single-cell proteomic sample formed with any one of the methods described herein.

Many biological processes and regulatory dynamics, such as the cell division cycle, are reflected in protein covariation across single cells. Variabilities within a cell type are challenging to analyze with existing single-cell omics methods. In some embodiments, the sample preparation methods described herein enable quantifying and interpreting the covariations by single-cell proteomics with sufficiently high throughput and accuracy. As shown below, the sample preparation methods have been used to prepare 1,888 single cells and 128 negative controls in a single batch. Their analysis enabled quantifying the covariation among thousands of proteins and cell-cycle protein markers. The results demonstrate that protein covariation across single cells may reveal functionally concerted biological differences between closely related cell states.

A substantially planar solid surface enables parallel processing of a large number of multiplexed single-cell samples at a high density, thereby significantly increasing the throughput of single-cell proteomic analysis. Said surface also enables efficient merging of each multiplexed single-cell proteomic sample, thereby significantly reducing sample loss and sample processing time. A substantially planar solid surface also enables precise dispensing of very small volumes of single cells and reagents and keeping the droplets separated.

Single cells are isolated in very small volumes (e.g., about 300 picoliter), and all preparation steps, including cell lysing, protein digesting, and peptide labeling are performed in droplets of small volumes (e.g., below about 20 nl) on a substantially planar surface. Reduced volumes during sample preparation and increased throughput result in reductions in background signal, increased sample consistencies, and increased sensitivities.

### EXAMPLES

Single-cell measurements are commonly used to identify different cell types from tissues composed of diverse cells (Regev et al., Elife 6:e27041 (2017) and Specht & Slavov, J Proteome Res. 17(8):2565-71 (2018)). This analysis is powering the construction of cell atlases, which can pinpoint cell types affected by various physiological processes. This cell classification requires analyzing a large number of cells and may tolerate measurement errors (Regev et al., Elife 6:e27041 (2017), Ziegenhain et al., Mol Cell 65(4):631-43 (2017), and Slavov, Science 367(6477):512-13 (2020)). In addition to classifying cells by type, single-cell measurements may reveal regulatory processes within a cell type and even associate them with different functional outcomes (Slavov, PLOS Biol. 20(1):e3001512 (2022), Shaffer et al., Nature. 546(7658):431-35 (2017) and Emert et al., Nat Biotechnol. 39(7):865-76 (2021)). For example, the covariation among proteins across single cells from the same type may reflect cell intrinsic dynamics, such as the cell division cycle (Slavov, PLOS Biol. 20(1):e3001512 (2022) and Mahdessian et al., Nature 590(7847):649-54 (2021)). Furthermore, protein covariation may reflect protein interactions within complexes or cellular states, such as senescence (Slavov, PLOS Biol. 20(1):e3001512 (2022)). However, estimating and interpreting protein covariation within a cell type requires high quantitative accuracy and high throughput (Slavov, PLOS Biol. 20(1):e3001512 (2022) and Slavov, Mol Cell Proteomics 21(1):100179 (2022)). Indeed, protein differences within a cell type are smaller than differences across cell types and can be easily swamped by batch effects and measurement noise. A goal is to minimize measurement noise to levels consistent with estimating and interpreting protein covariation across single cells from the same cell type. Towards this goal, an aim was to reduce batch effects and background noise, since these factors undermine the accuracy of single-cell proteomics by mass spectrometry (MS) (Slavov, Curr Opin Chem Biol. 60:1-9 (2021), Vanderaa & Gatto, Expert Rev Proteomics 18(10):835-43 (2021), Kelly, Mol Cell Proteomics 19(11): 1739-48 (2020), and Specht et al., Genome Biol. 22(1):50 (2021)). Specifically, an aim was to develop a widely accessible, robust, and automated sample preparation method that reduces volumes to a few nanoliters. A goal was to perform parallel sample preparation of thousands of single cells to increase the size of experimental batches and thus reduce batch effects (Vanderaa & Gatto, Expert Rev Proteomics 18(10):835-43 (2021), Klein et al., Cell 161(5):1187-201 (2015) and Macosko et al., Cell 161(5):1202-14 (2015)). To achieve high precision, an aim is to avoid any movement of the samples during the sample preparation stage, so that 1-10 nl volumes of reagents can be repeatedly dispensed to each droplet containing a single cell. The CellenONE cell sorting and liquid handling system was used to develop nano-Proteomic sample Preparation (nPOP), which allowed a 100-fold reduction of the sample volumes over the Minimal ProteOmic sample Preparation (mPOP) method (Specht et al., Genome Biol. 22(1):50 (2021), Harrison et al., bioRxiv 399774 (2018), Petelski et al., Nat Protoc. 16(12):5398-25 (2021) and Marx, Nat Methods 16(9):809-12 (2019)). nPOP enabled analysis of protein covariation within two cell lines, monocytes and melanoma. This enabled classifying cells by cell division cycle (CDC) phase and identifying a sub-population of melanoma cells. Comparative analysis between the cell lines identifies both similar and differential patterns of CDC associated protein covariation. Further, this analysis was applied within melanoma sub-populations, and differences in CDC associated protein covariation as well as a differential distribution of cells throughout phases of the CDC were identified.

### Example 1. Methods

### Cell Culture

U-937 and Jurkat cells were grown as suspension cultures in RPMI medium (HyClone 16777-145, Cytiva, Marlborough, MA) supplemented with 10% fetal bovine serum and 1% penicillin-streptomycin (pen/strep) (15140122, ThermoFisher, Waltham, MA). Cells were passaged when a density of 10⁶ cells/ml was reached, approximately every two days.

The melanoma cells (WM989-A6-G3, a gift from Arjun Raj, University of Pennsylvania) were grown as adherent cultures in TU2% media which is composed of 80% MCDB 153 (M7403, Sigma-Aldrich, St. Louis, MO), 10% Leibovitz L-15 (11415064, ThermoFisher, Waltham, MA), 2% fetal bovine serum, 0.5% penicillin-streptomycin and 1.68 mM Calcium Chloride (499609, Sigma-Aldrich, St. Louis, MO). Cells were passaged at 80% confluence (approximately every 3-4 days) in T75 flasks (Z707546, MilliporeSigma, Burlington, MA) using 0.25% Trypsin-EDTA (25200072, ThermoFisher, Waltham, MA) and re-plated at 30% confluence.

HPAF-II cells (CRL-1997™, ATCC, Manassas, VA) were cultured in EMEM (30-2003, ATCC, Manassas, VA), CFPAC-I cells (CRL-1918™, ATCC, Manassas, VA) were cultured in IMDM (30-2005), and BxPC-3 cells (CRL-1687™, ATCC, Manassas, VA) were cultured in RPMI 1640 (30-2001, ATCC, Manassas, VA). All media were supplemented with 10% fetal bovine serum (FBS) (F4135, MilliporeSigma, Burlington, MA) and 1% penicillin-streptomycin. Cells were passaged at 70% confluence.

### Lysis Validation Experiment

Jurkat cells and U-937 cells cultured in heavy SILAC media (containing+10 Da Arg and +8 Da Lys) were washed and re-suspended in PBS at 20,000 cells per μl. Two solutions of equal cell count containing Jurkat and U-937 cells were made mixed in 1:1 ratios. One sample was lysed by diluting cells in 90% DMSO and the other was lysed in 6M urea. The DMSO cell lysate was diluted to a concentration of 33% DMSO and urea lysate was diluted to 0.5 M. Both solutions were digested in 15 ng/μl of trypsin for 12 hours. Each sample was then desalted using C18 stage tips and run using data dependent acquisition.

### Carrier and Reference Channel Preparation in Bulk

The isobaric carrier consisting of a 1:1 mixture of melanoma and monocyte cells was prepared in bulk and aliquoted into carriers corresponding to 200 cells each. A single cell suspension of 22,000 cells was transferred to a 200 μl PCR tube (1402-3900, USA Scientific, Inc., Ocala, FL) and then processed via the mPOP sample preparation method (Harrison et al. bioRxiv 399774 (2018)). The reference channel was made from the same sample.

### Bulk Melanoma and Monocyte Samples

Additional bulk samples of melanoma and monocyte cells were prepared for validating quantification of single cells. Cell pellets of 100,000 monocyte and melanoma cells were suspended in 50 μl of mass spectrometry grade water and lysed and digested via mPOP sample preparation (Harrison et al. bioRxiv 399774 (2018)). Samples were then labeled with TMT-16plex, combined, and diluted down to a concentration of 400 cells/μl for analysis by LC-MS.

Reagent Handling with CellenONE

The CellenONE (see, e.g., www.cellenion.com/technology/) was equipped with two piezo dispensing capillaries (PDC). One PDC was dedicated to handle cell suspensions. The other PDC was dedicated for all other reagent handling including organic solvents and protein solutions. Reagents were loaded into a 384-well plate in volumes of 30 μl. When aspirating protein solutions, 20 μl was aspirated to ensure the mixture was not diluted with system water. When dispensing DMSO, it was important to deactivate the humidifier. This allowed residual DMSO left on the tip of the PDC to evaporate quickly so dispensing was not affected. After each sample preparation, PDCs were washed with ethanol and cleaned under sonication to remove any built-up of material from inside of the PDC and ensure optimal performance.

### Sample Preparation and Experimental Design

nPOP reactions were carried out on the surface of a fluorocarbon coated glass slide. The array layout was very flexible and adjustable to the experimental parameters. The droplets used for single-cell sample preparation were arranged in clusters, and the number of droplets per cluster equals the number of single cells per SCOPE2 (Single Cell ProtEomics 2) set. TMTpro 18plex and 14 droplets per cluster, corresponding to the 14 isobaric labels used for single cells, were used. The design allowed fitting 36 clusters per slide and 4 glass slides on the temperature controlled target holder, which enabled simultaneous processing of up to 14×36×4=2,016 single cells. Reducing the space between clusters can further increase the number of clusters per slide and thus the number of simultaneously prepared single cells. The array layout was optimized to keep droplets from the same set close in proximity but prevent reaction volumes from merging. Once an array layout was selected, 8 nl of DMSO was dispensed to each location of the array, forming the initial reaction volume for each single cell reaction. Lysis began when cells were dispensed inside a droplet of about 300 pl of PBS into these reaction volumes of DMSO. After lysis, 10 nl of solution containing trypsin and HEPES buffer was added to each reaction volume, for a final concentration of 120 ng/μl of trypsin and 5 mM HEPES and total volume of 18 nl.

The humidifier and cooling system were then turned on to prevent droplet evaporation. Relative humidity inside the CellenONE was set to 75%, and the chiller temperature was set to dynamically chase one degree above the dew point. Mass spectrometry grade water was dispensed in a perimeter surrounding each grid to provide further control for the local humidity of the reaction volumes. The system was set to refresh the water droplet perimeter to control local humidity every 40 minutes for 5 hours as proteins digest.

After proteins were digested for 5 hours, the humidity and cooling controls were turned off. 20 nl of TMT labels suspended in DMSO and concentrated at 28 mM were then dispensed to each reaction volume using the organic dispensing tip. When dispensing labels, humidifier was turned off to assist with dispensing. After single cells were left to label for 1 hour, 20 nl of 5% hydroxylamine solution was added to each reaction volume to quench labeling reaction. Humidity and cooling controls were returned to previous settings for quenching labeling reaction. After 20 minutes, another addition of 30 nl of 5% hydroxylamine was added.

After quenching proceeds for another 20 minutes, sample clusters were pooled by aspirating them off the slide surface in 10 μl of a 100% acetonitrile solution via CellenONE PDC and syringe pump controls. Pooled samples were then transferred into a 384-well plate (AB1384, ThermoFisher, Waltham, MA) and dried down to dryness in a speed-vacuum (Eppendorf, Germany) and either frozen at −80ºC for later analysis or immediately reconstituted in 1.1 μl of 0.1% formic acid (85178, ThermoFisher, Waltham, MA) for mass spectrometry analysis.

### DNA Sorting for Bulk CDC Analysis

Melanoma and monocyte cells were incubated using Vy-17 brand Dye Cycle (V35003, ThermoFisher, Waltham, MA) following manufacturer's instructions. Cells were sorted via the Beckman CytoFLEX SRT (Beckman Coulter, Brea, CA). Post sorting, cells were pelleted and washed with Mass Spectrometry grade water and resuspended in water at a concentration of 2000 cells/μl. Cells were then frozen at −80ºC for 10 minutes and then heated to 90ºC for 10 minutes for lysis. Proteins were then digested overnight in a solution of 15 ng/μl of trypsin. Samples were analyzed via data independent acquisition.

### LC-MS Platform

MS analysis was designed and performed according to the SCOPE2 guidelines and protocol (Specht et al., Genome Biol. 22(1):50 (2021), Petelski et al., Nat Protoc. 16(12):5398-425 (2021) and Specht & Slavov, J Proteome Res. 20(1):880-87 (2021)). Specifically, the single cells pooled into SCOPE2 sets were separated via online nlC on a Dionex UltiMate 3000 UHPLC; 1 μl out of 1.1 μl of sample was picked up out of a 384-well plate (AB1384, ThermoFisher, Waltham, MA) placed on an auto sampler height adjuster for PCR plates (6820.4089, ThermoFisher, Waltham, MA) and loaded onto a 25 cm×75 μl IonOpticks Aurora Series UHPLC column (AUR2-25075C18A). Buffer A was 0.1% formic acid in water and buffer B was 0.1% formic acid in 80 acetonitrile/20% water. A constant flow rate of 200 nl/min was used throughout sample loading and separation. Samples were loaded onto the column for 20 minutes at 1% B buffer, then ramped to 5 B buffer over two minutes. The active gradient then ramped from 5% B buffer to 25% B buffer over 53 minutes. The gradient was then ramped to 95% B buffer over 2 minutes and stayed at that level for 3 minutes. The gradient then dropped to 1% B buffer over 0.1 minutes and stayed at that level for 4.9 minutes. Loading and separating each sample took 95 minutes total. All samples were analyzed by a Thermo Scientific Q-Exactive mass spectrometer from minute 20 to 95 of the LC loading and separation process. Electrospray voltage was set to 1.8 V, applied at the end of the analytical column. To reduce atmospheric background ions and enhance the peptide signal-tonoise ratio, an Active Background Ion Reduction Device (ABIRD, ESI Source Solutons, LLC, Woburn, MA) was used at the nanospray interface. The temperature of ion transfer tube was 250° C. and the S-lens RF level was set to 80.

### Single-Cell MS Data Acquisition

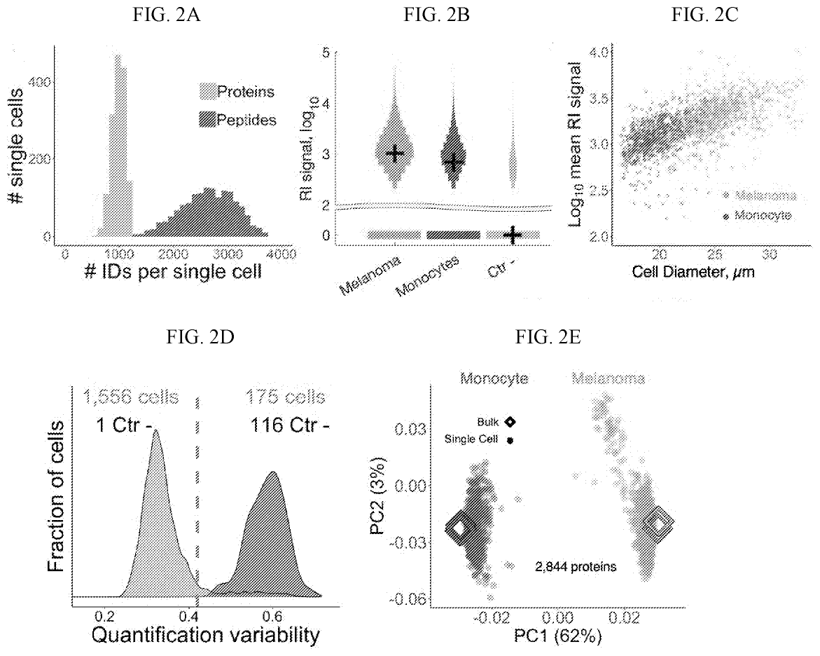

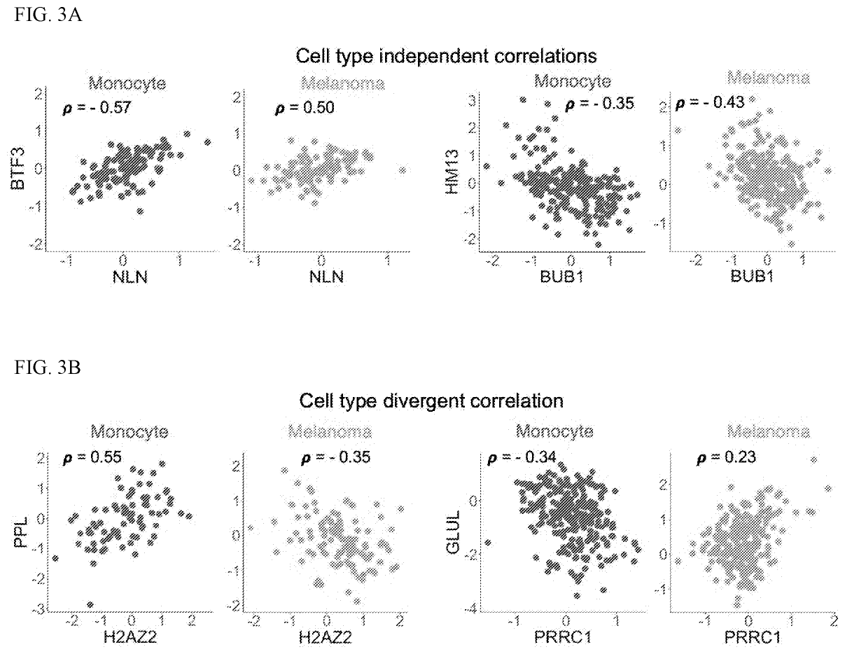

A prioritized analysis workflow (Huffman et al., bioRxiv 484655 (2022)) was used to increase consistency of identification and depth of coverage for the nPOP-prepared single-cell data shown in FIGS. 2A-2E, FIGS. 3A-3C, FIGS. 4A-4I, and FIGS. 5A-5G. A spectral library was built from two injections of a 10× concentrated aliquot of combined carrier and reference sample analyzed by DIA instrument methods 1 and 2, as well as an injection of a 5× concentrated aliquot of combined carrier and reference sample analyzed by DIA method 1. Both of these instrument methods are detailed in the methods section of Huffman, et al. (Huffman et al., bioRxiv 484655 (2022)). A subsequent injection of a 1× concentrated aliquot of carrier and reference sample was analyzed by DIA instrument method 1 to serve as a retention-time-calibration run. The results from this retention-time-calibration run were searched with Spectronaut to generate a prioritized inclusion list for subsequent scout runs and prioritized single-cell analyses. The prioritized inclusion lists were then imported into MaxQuant. Live (v. 2.0.3 with priority tiers) and used to analyze 1× concentrated carrier and reference samples or nPOP prepared single-cell samples, with settings detailed below.

LC-Settings for pSCOPE-Associated Experiments

Samples were analyzed using a 95-minute method with the following gradient characteristics: samples were loaded onto the column at 4% B; the gradient was then ramped to 8% at minute 12, 35% at minute 75, 95% at minute 77, 4% from minute 80.1 onward.

### Inclusion-List Generation for Scout Experiments

Spectronaut search results of the retention-timecalibration run were filtered to EG.PEP≤ 0.02 and EG.Qvalue≤ 0.05. Additionally, precursors without TMTPro modifications (+304.2071 Da) on the peptide n-terminus or lysine residue were filtered out. The distribution of precursor intensities for the remaining precursors was then subset into tertiles for use in priority tier assignment. These precursors were then filtered such that a maximum of four peptides per protein were selected, with the most intense peptides per protein being selected. Filtered peptides with precursor intensities in the top intensity tertile were placed on the top priority tier, peptides with intensities in the middle intensity tertile were placed on the middle priority tier, and peptides with intensities in the bottom intensity tertile were placed on the bottom priority tier. All species matching the original EG.PEP and EG.Qvalue filtration characteristics that were not previously selected for a priority tier were assigned a priority below the previous bottom tier. These priority-tier-assigned peptides were then enabled for participation in MaxQuant.Live's realtime-retention-time-alignment algorithm, as well as MS2 upon detection. Any remaining PSMs outside of the original filtration criteria (EG.PEP≤ 0.02 and the EG.Qvalue≤ 0.05) were enabled for participation in MaxQuant.Live's realtime-retention-time-alignment algorithm, but not sent for MS2 upon detection.

### Scout Experiment Instrument Method and Raw Data Analysis

1 μl injections of a 1× concentrated aliquot of mixed carrier-reference material were analyzed using the instrument method detailed in the prioritized acquisition parameters section and MaxQuant.Live parameters indicated in the associated table. The two raw files associated with these experiments were then searched using MaxQuant (v. 1.6.17.0) using a FASTA containing all entries from the human SwissProt database (swissprot_human_20211005. fasta, 20,386 proteins). TMTPro 16plex was enabled as a fixed modification on peptide n-termini and lysines via the reporter ion MS2 submenu. Methionine oxidation (+15.99492 Da) and protein n-terminal acetylation (+42.01056 Da) were enabled as variable modifications, and trypsin was selected for in silico digestion with enzyme mode set to specific. Up to 2 missed cleavages were allowed per peptide with a minimum length of 7 amino acids. Second peptide identifications were disabled, calculate peak properties was enabled, and msScans was enabled as an output file. PSM FDR and protein FDR were set to 1.

### Pre-Prioritization Shotgun Experiment Instrument Method and Raw Data Analysis

One lul injection of a 1× concentrated aliquot of mixed carrier-reference material was analyzed using the LC settings indicated above. The following MS1 settings were used: 70k resolution, le6 AGC target, 100 ms maximum injection time, and a scan range of 450Th to 1600Th. MS2 scans were acquired with the following settings: 70k resolution, le6 AGC target, 300 ms maximum injection time, loop count (i.e., top-n) of 7, Isolation window of 0.7Th with a 0.3Th offset, fixed first mass of 100 m/z, NCE of 33, and a centroid spectrum data type. The minimum AGC target was 2e4, apex triggering was disabled, and charge exclusion was enabled for unassigned charge states, as well as charge states greater than 6. The peptide match setting was disabled, exclude isotopes was enabled, and dynamic exclusion was set to 30 seconds. Voltage was set to 0 for the first 25 minutes, sweep gas was applied from minute 24.6 to 25 to dislodge any accumulated droplets from the capillary tip. From minute 25 to 80, voltage was set to 1.7 kV, capillary temp to 250° C., and the S-lens RF level to 80. From minute 94.20 to 94.60, sweep gas was applied to dislodge any accumulated droplets from the capillary tip.

The raw file generated by this analysis was searched using the same maxquant settings as indicated in the Scout experiment instrument method and raw data analysis section.

### Prioritized Inclusion List Generation

The PSMs generated from the scout runs using intensity dependent-tiers (wAL00191 and wAL00192) were partitioned into three categories: PSMs at PEP≤ 0.02 (set a), PSMs with 0.02<PEP≤ 0.05 (set B), and PSMs with PEPs >0.05 (set γ). Then the same set of PEP filters defined above for wAL00191 and wAL00192 were applied to the results of a DDA analysis conducted on an injection of a 1× concentrated aliquot of carrier and reference material to generate sets δ, ∈, and ξ. Furthermore, these last three precursor sets were assembled such that they each contained a unique set of precursors with respect to one another and the preceding set of precursors.

Sets α and δ were combined and filtered such that a maximum of 4 peptides per protein were selected, choosing those precursors with the highest precursor intensities, to form the top priority tier candidates. The excluded precursors from this filtration were then combined with sets β and ∈ to make up the middle priority tier candidates. Peptides from sets γ and ξ were then combined to form the bottom priority tier candidates.

The results from the retention-time-calibration experiment were then intersected with the priority tier sets, and the PSMs matching each set were given a corresponding priority index for use by MaxQuant. Live. Up to 8,600 of the most abundant remaining retention-time-calibrationexperiment-associated PSMs were then added to the bottom priority tier to provide additional identifiable precursors when higher priority precursors were not detected. All selected precursors were then enabled for participation in the MaxQuant.live real-time-retention-time-alignment algorithm, and for MS2 upon detection. All remaining PSMs that were not part of the priority tiers were then selected for participation in the MaxQuant.live real-time-retention-time-alignment algorithm, but not for MS2 upon detection.

### Prioritized Acquisition Parameters, Scout Runs and Single-Cell Samples

All single-cell samples were resuspended in 1 μl of 0.1% formic acid (85178, Thermo Fisher, Waltham, MA) and injected from a 384-well plate (AB1384, Thermo Fisher, Waltham, MA). All 1× concentrated carrier and reference samples were resuspended in 1 μl of 0.1% formic acid (85178, Thermo Fisher, Waltham, MA) and injected from a glass HPLC insert (C4010-630, Thermo Fisher, Waltham, MA). LC settings indicated above were used in these analyses. Scan parameters were implemented following the MQ.live listening scan guidelines: Two Full MS-SIM scans were applied from minute 25 to 30 to trigger MaxQuant.live. Both MS-SIM scans had the following parameters in common: 70k resolution, le6 AGC target, and a 300 ms maximum injection time. The first MS-SIM scan covered 908 to 1070Th, since the acquisition started at minute 25 and ended at minute 95. The second MS-SIM scan covered the scan space from 909Th to the numeric MaxQuant.live method index to call. The total Xcalibur MS method time was 95 minutes. Tune files governing voltage and sweep gas were implemented as in the pre-prioritization shotgun method.

### Limited FASTA File Generation for Raw Data Analysis Corresponding to Prioritized Samples

The swissprot_human_20211005. fasta was read into the R environment using the seqinr (Charif & Lobry, Biological and Medical Physics, Biomedical Engineering 207-32 (2007)) package, and only those proteins with peptides present on the inclusion list were retained to generate the AndrewsnPOP_FASTA_v2.fasta file, containing 3535 proteins, used to search the resulting prioritized single-cell experiments.

### DDA MS Acquisition

After a precursor scan from 450 to 1600 m/z at 70,000 resolving power, the top 7 most intense precursor ions with charges 2 to 4 and above the AGC min threshold of 20,000 were isolated for MS2 analysis via a 0.7 Th isolation window with a 0.3 Th offset. These ions were accumulated for at most 300 ms before being fragmented via HCD at a normalized collision energy of 33 eV (normalized to m/z 500, z=1). The fragments were analyzed by an MS2 scan with 70,000 resolution. Dynamic exclusion was used with a duration of 30 seconds with a mass tolerance of 10 ppm.

### DIA MS Acquisition of Bulk CDC Populations

Samples were run using the VI method from Derks et al. (Derks et al., bioRxiv 467007 (2021)). This method contains 140k resolution MS1 scans for improved MS1 level quantification.

### Analysis of DDA MS Data

Raw data were searched by MaxQuant (Cox et al., Nat Biotechnol. 26(12): 1367-72 (2008) and Cox et al., J Proteome Res. 10(4):1794-805 (2011)). 1.6.17.0 against a protein sequence database including entries from the appropriate human SwissProt database (dow nloaded Jul. 30, 2018) and known contaminants such as human keratins and common lab contaminants. Fasta was limited to proteins which were included on prioritization list. MaxQuant searches were performed using the standard work flow (Tyanova et al., Nat Protoc. 11(12):2301-19 (2016)). Trypsin specificity was specified and up to two missed cleavages for peptides having from 5 to 26 amino acids were allowed. Methionine oxidation (+15.99492 Da) and protein N-terminal acetylation (+42.01056 Da) were set as variable modifications. Carbamidomethylation was disabled as a fixed modification. All peptide-spectrummatches (PSMs) and peptides found by MaxQuant were exported in the msms.txt and the evidence. txt files.

### Analysis of Data Independent Acquisition MS Data

Data Independent Acquisition runs were searched with DIA-NN v1.8.0 (Demichev et al., Nat Methods. 17(1):41-44 (2020)) using an in silico fasta generated library enabled by deep learning.

### SILAC Data Analysis

When comparing relative protein levels in Jurkat and U-937 cells, SILAC ratios for peptides were computed by taking dividing each channel by its median, and then taking the ratio of the light and heavy channels. When comparing absolute abundances between heavy and light U-937 cells to measure efficiency of extraction, label swap experiments were run so that both lysis conditions were measured with both heavy and light labels. The raw intensities for corresponding lysis methods were averaged and the ratio between different lysis methods was plotted.

### Single-Cell Filtering and Normalization

The single-cell data were processed and normalized by the SCOPE2 pipeline (Specht et al., Genome Biol. 22(1):50 (2021) and Petelski et al., Nat Protoc. 16(12):5398-425 (2021)). This pipeline is also implemented by the scp Bioconductor package (Vanderaa & Gatto, Expert Rev Proteomics 18(10):835-43 (2021) and Vanderaa & Gatto, Bioconductor (2020)). Briefly, single cells with suboptimal quantification were removed prior to data normalization and analysis based on objective criteria: The internal consistency of protein quantification for each single cell was evaluated by calculating the coefficient of variation (CV) for proteins (leading razor proteins) identified with over 5 peptides for that cell. The coefficient of variation is defined as the standard deviation divided by the mean. The CVs were computed for the relative reporter ion intensities, i.e., the RI reporter ion intensities of each peptide were divided by their mean resulting in a vector of fold changes relative to the mean. Cells that fell outside the distribution were removed from analysis with a threshold of 0.41. Data was normalized as by procedure outlined by Specht et al. (Specht et al., Genome Biol. 22(1):50 (2021) and Specht et al., Genome Biol. 22(1):50 (2021)).

### Principal Component Analysis for Single Cell Data Sets

From the protein x single cell matrix, all pairwise protein correlations (Pearson) were computed. Thus, for each protein, there was a computed vector of correlations with a length the same as the number of rows in the matrix (number of proteins). The dot product of this vector with itself was used to weight each protein prior to principal component analysis. The principal component analysis was performed on the correlation matrix of the weighted data.

### Melanoma Sub Population Protein Set Enrichment Analysis

Protein set enrichment analysis was performed by t-test between Cluster A and B on the un-imputed data. It was required that a given gene set had at least 4 proteins measured in the single cells and that each population had at least 80% of cells with protein observations. The distribution of p-values was corrected for multiple hypothesis testing with the BH method. O nly GO terms were reported with Q value less than 0.0001 were reported.

### Constructing CDC Phase Markers

Phase markers were constructed from proteins identify with differential abundant each CDC phase in both monocyte and melanoma cells. These proteins were first identified on the bulk level. To further narrow the list of proteins used to create phase markers, proteins that contained multiple, positively correlated peptides in the single cell samples were used. Phase markers were then constructed by averaging the abundances of all possible combinations of 2 or 3 proteins corresponding to each phase of the cell cycle. Groups of two markers for each CDC phase that were positively correlated were selected. This served as validation as it was expected that proteins that are highly abundant in same phase would positively covary. Groups of protein markers were then further filtered.

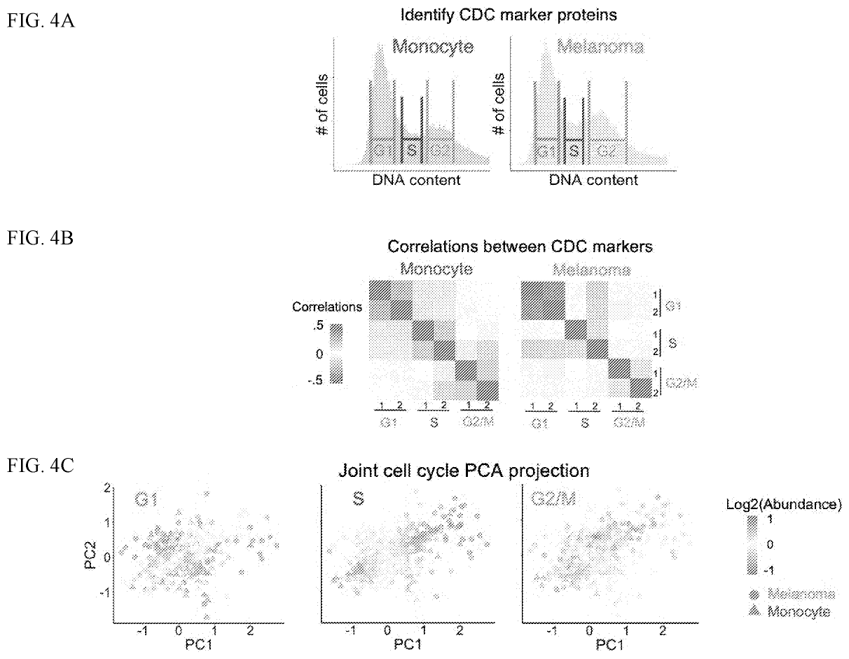

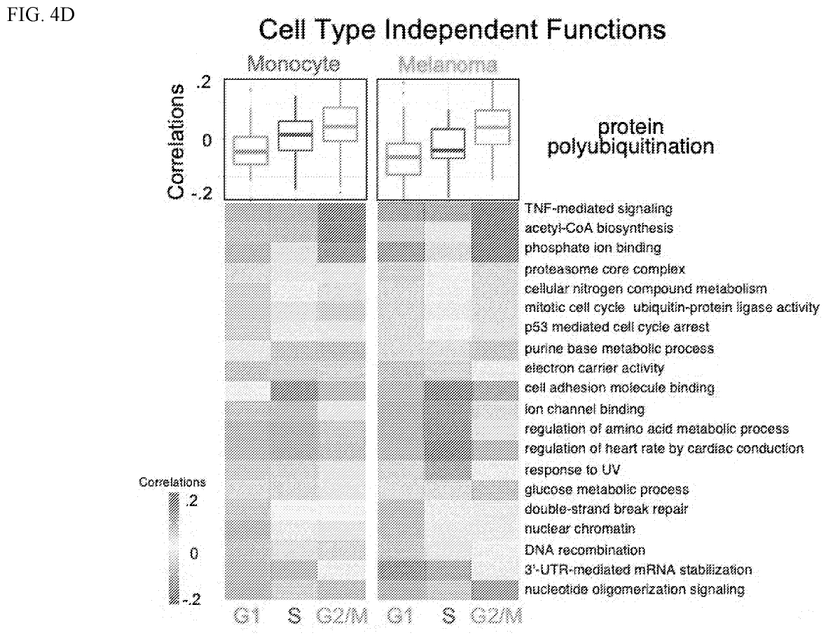

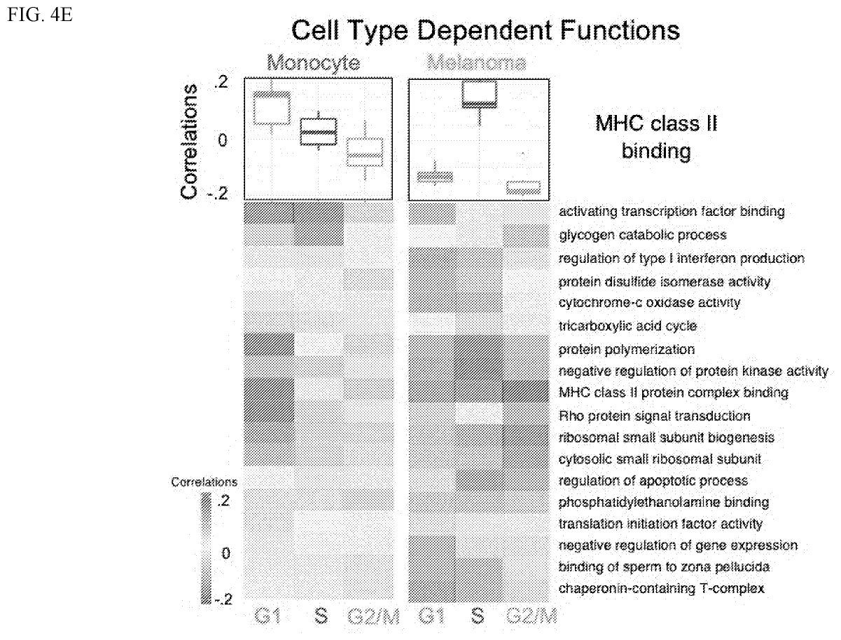

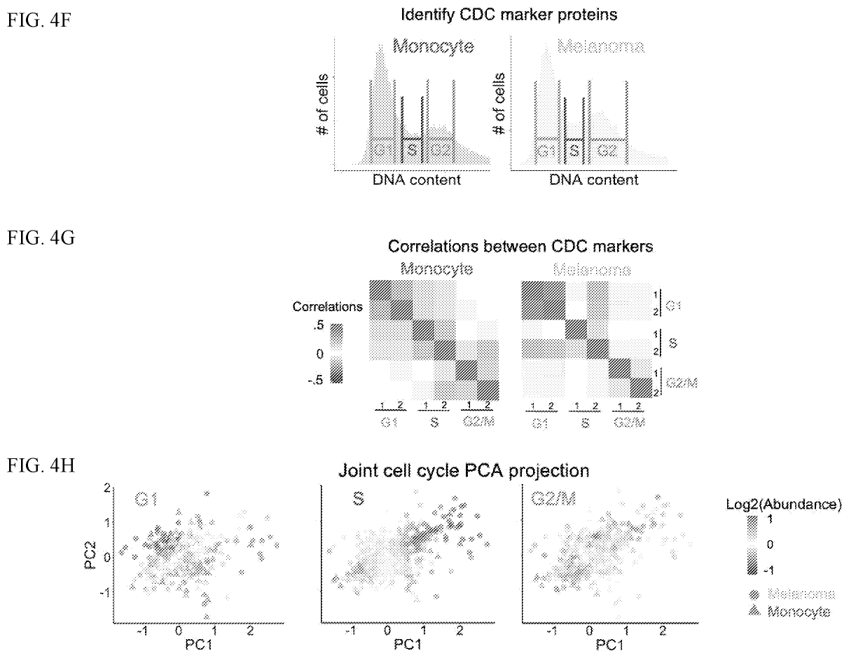

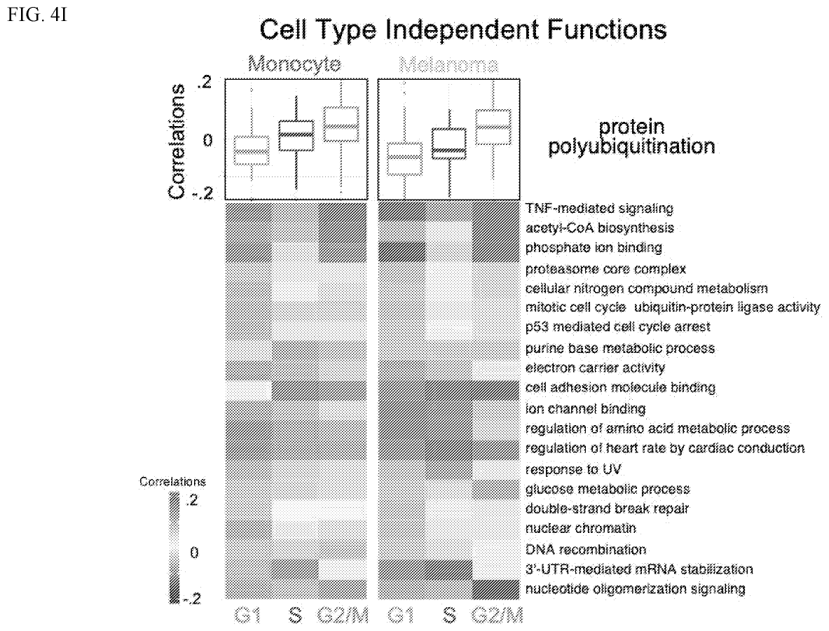

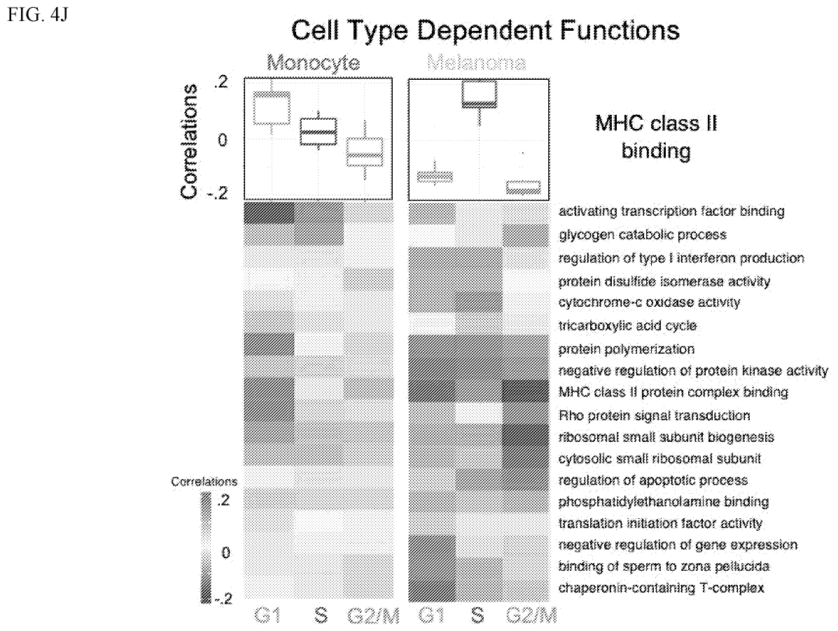

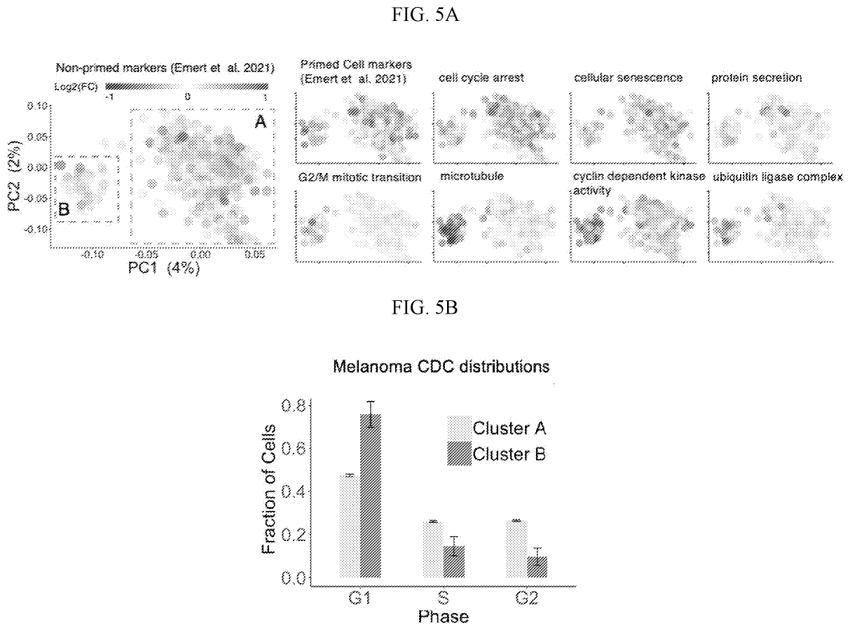

Markers were first constructed in the space of monocyte cells, and correlations between markers were validated in melanoma cells FIGS. 4B and 4G. Having validated the protein markers, protein markers within phase were averaged for downstream analysis.

Identifying Proteins that Covary with CDC Markers

To identify proteins that covary with the phase marker vectors, the phase marker vectors to the measured protein levels of each protein were correlated using Spearman correlation. The distribution of p-values obtained from the Spearman correlation test was adjusted using the BH method and the results were filtered at 1% FDR.

### Cell Cycle Protein Set Enrichment Analysis

To identify functionally coherent sets of proteins that covary with the CDC phase markers, each protein was correlated to the median abundance of CDC proteins that showed similarity between melanoma and monocyte cells as plotted in FIGS. 4A and 4F. The resulting correlation vectors were analyzed by protein set enrichment analysis similar to previously reported analysis (Franks et al., PLOS Comput Biol. 13(5):e1005535 (2017)). In the case of cell-type specific co-variation, empirical bootstrapping was also used to estimate the Z-score corresponding to each correlation, and the distributions of Z-scores were then compared via ANOVA for estimating the statistical significance. O nly GO Terms having least 4 proteins were analyzed. ANOVA was used to estimate if the variance among the correlations of the proteins from the GO term, and the CDC phase markers can be explained by the CDC. The Benjamini-Hochberg method was then used to estimate the corresponding q values (FDR; false discovery rare) for each GO term. Among the set of GO terms within 5% FDR, the 20 GO terms whose correlations to the CDC phase markers was most similar or most different between the 2 cell lines were displayed in FIGS. 4A-4J.

### Assigning Cells to CDC Phase

A greedy approach was taken to assign cells to a CDC phase. First, a vector comprised of length 3X the number of cells was created, where each value was the average abundance of G1, S, or G2 marker proteins. This vector was then sorted from highest to lowest. Subsequently iterated down the list and sorted cells into the G1, S, or G2 bin based off the phase of each value. 50% of cells were sorted into the G1 bin, 25% of cells were sorted into the S and G2 bins based off the distribution observed from the bulk FACS CDC sorting.

### Protein Complex Analysis

A null distribution that consists of all pairwise Euclidean distances was computed for each protein. Euclidean distances were o nly calculated between observed values, and vectors were subsequently normalized to the number of pairwise observed values in each vector. Euclidean distances were then calculated in the same fashion from all proteins within complexes from the CORUM protein database (Giurgiu et al., Nucleic Acids Res. 47(D1):D559-D563 (2019)).

TABLE 1
MaxQuant.live settings for prioritized analysis

| | Scout exp. | | nPOP samples | |
|---|---|---|---|---|
| Global settings: Survey scan | | | | |
| ScanDataAsProfile | True | | True | |
| PositiveMode | True | | True | |
| MaxIT | 100 | ms | 100 | ms |
| Resolution | 70,000 | | 70,000 | |
| AgcTarget | 1,000,000 | | 1,000,000 | |
| MzRange | (450, 1258) | | (450, 1258) | |
| BoxCarScans | 0 | | 0 | |
| Global settings: TopN | | | | |
| NumOfMS2Scans | 0 | | 0 | |
| RealtimeCorrection | | | | |
| MzTolerances | (4.5, 5) | | (4.5, 5) | |
| RetentionTimeTolerances | (0.01, 2) | | (0.01, 2) | |
| SigmaScaleFactorRt | 3 | | 3 | |
| PeptideHistoryLength | 2 | | 2 | |
| MinUsedCorrectionPeptides | 15 | | 15 | |
| IntensityPeakRatioThreshold | .01 | | .01 | |
| PeptideDetectionIsoPeaks | 2 | | 2 | |
| IsotopeTolerance | 9 | | 9 | |
| Ms2DetectionNeeded | False | | False | |
| Ms2ExcludeDetectedPeptides | False | | False | |
| Ms2MinNormIntensity | 0.1 | | 0.1 | |
| Ms2MzTolerance | 20 | | 20 | |
| TargetedMs2 | | | | |
| BatMode | False | | False | |
| AutoPriority | True | | True | |
| DefaultPriority | 0 | | 0 | |
| MaxNumOfScans | 1 | | 1 | |
| WindowAndOffsetInDalton | False | | False | |
| ScanDataAsProfile | False | | False | |
| WindowSize | 0.5 | | 0.5 | |
| MzOffset | 0 | | 0 | |
| LowerMzBound | 100 | | 100 | |
| CollisionEnergy | 33 | | 33 | |
| LifeTime | 2,100 | ms | 2,100 | ms |
| Resolution | 70,000 | | 70,000 | |
| MaxIT | 300 | ms | 300 | ms |

| | | |
|---|---|---|
| AgcTarget | 1,000,000 | 1,000,000 |
| PositiveMode | True | True |

### Example 2. Sample Preparation

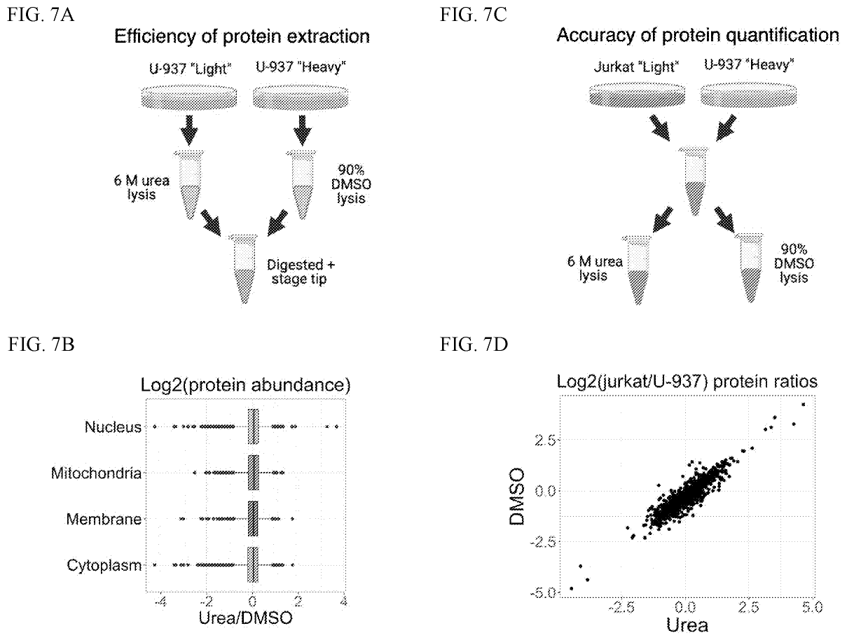

To reduce batch effects and background signal, the goal was to maximize the number of single cells prepared in parallel while minimizing the volumes of sample preparation. To this end, the idea of performing all sample preparation steps in droplets on the surface of a uniform glass slide was explored (FIGS. 1A-1D). This allows the freedom to arrange single cells in any geometry that best fits the experimental design (FIGS. 1A-1C). To facilitate this idea, clean reagents, compatible both with analysis by LC-MS and an open surface design, were needed. To this end, the use of 100% dimethyl sulfoxide (DMSO) was introduced as a reagent for cell lysis and protein extraction. Its low vapor pressure enables nanoliter droplets to persist on the surface of the open glass slide. Furthermore, its compatibility with MS analysis allows to obviate sample cleanup and associated losses and workflow complications. Control experiments indicate that DMSO efficiently delivers proteins to MS analysis without detectable bias for cellular compartments (FIG. 7B) and supports accurate relative protein quantification (FIG. 7D).

These data supported the use of DMSO for cell lysis performed by first dispensing an experimenter defined regular array of DMSO droplets, and subsequently adding a cell to each droplet for lysis (FIG. 1A). After lysis, proteins are digested for 4 hours by adding the protease trypsin dissolved in aqueous buffer. To control evaporation throughout the digestion step, slide temperature and internal humidity were controlled. Furthermore, a perimeter of water droplets was dispensed around the samples (FIG. 1D). See Example 1 for details.

For the next step, labeling peptides, it was found that the commonly used approach of dissolving labels in acetonitrile was unreliable due to low density and low surface tension of acetonitrile. To overcome this problem, DMSO dissolved labels were introduced, and robust performance of sub-nanoliter droplets over hundreds of samples were observed. This approach was validated by measuring labeling efficiency in pooled samples, and over 99% of all possible peptides were found to be TMT labeled. The final step of nPOP entailed collecting the samples and delivering them for LC-MS analysis. Clusters of labeled single cells were pooled into a single set, aspirated, and dispensed into a 384-well plate in a fully automated fashion for streamline sample injection (FIGS. 1A-1B).

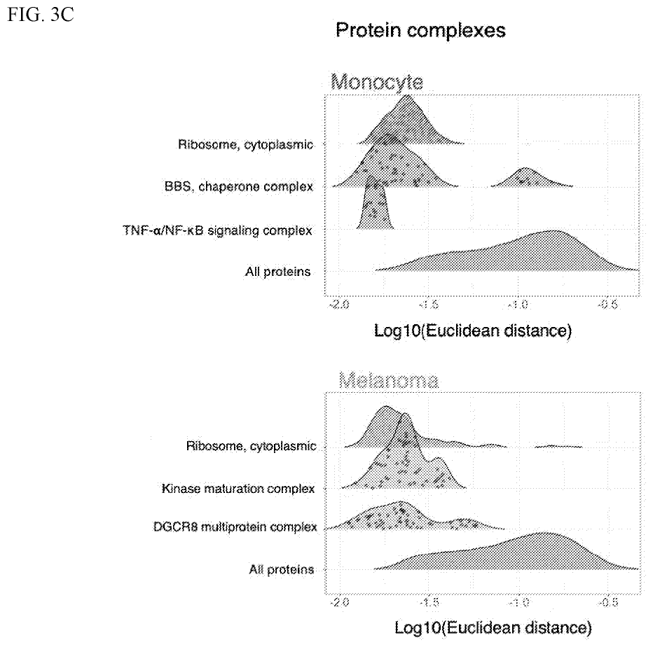

The nPOP sample preparation was combined with prioritized quantification of proteins introduced by Huffman et al. (Huffman et al., bioRxiv 484655 (2022) and followed the guidelines of the SCOPE2 protocol (Specht et al., Genome Biol. 22(1):50 (2021) and Petelski et al., Nat Protoc. 16(12):5398-25 (2021)). The AL-01 sample layout design, which prepares 2,016 single cells in one day, was employed (FIG. 1D). Using this design, 1,556 single cells were successfully analyzed (FIG. 2A) as part of a single batch. This is lower than the 2,016 capacity due to: 1) including 128 negative controls, 2) having 175 single cells excluded from analysis (FIG. 2A) and 3) 15 sets lost because of LC malfunctions. To increase the depth and consistency of proteome coverage, the single-cell samples were analyzed by prioritized Single Cell ProtEomics (pSCOPE) introduced by Huffman et al. (Huffman et al., bioRxiv 484655 (2022), following the guidelines of the SCOPE2 protocol (Specht et al., Genome Biol. 22(1):50 (2021) and Petelski et al., Nat Protoc. 16(12):5398-25 (2021)).

### Example 3. Single-Cell Data Quality Controls

To evaluate nPOP's ability to analyze protein covariation within and across cell types, two cell lines, WM989 melanoma and U-937 monocyte cells, were analyzed. The average number of proteins and peptides per single cell were 997 proteins and 2,630 peptides, with 2,844 proteins quantified across the 1,543 single cells prepared by nPOP (FIG. 2A). To quantify the extent of background noise in these measurements, the intensity of signal in negative controls was evaluated. The negative controls correspond to droplets that did not receive single cells, and their intensities reflect crosslabeling and nonspecific background noise (Specht et al., Genome Biol. 22(1):50 (2021) and Petelski et al., Nat Protoc. 16(12):5398-25 (2021)). The intensities in the negative controls, shown in FIG. 2B, were mostly absent or very low, indicating that background noise is low for samples prepared with nPOP. The intensities for single cells also show that peptides from melanoma cells were more abundant than peptides from monocyte cells, reflecting the different cell sizes (FIG. 2B). To further test the extent to which higher reporter ion signal in the melanoma cells reflects larger cell size, the measured diameter for single cells was plotted against the average reporter ion signal (FIG. 2C). Good agreement between diameter and average intensity, p=0.81, both between cell types and within cell types supports the differences in distributions for all melanoma and monocyte cells.

As an additional QC metric, the agreement in relative quantification derived from different peptides originating from the same protein was evaluated. The agreement was significantly higher in the single cells than the negative controls (FIG. 2D). Furthermore, the small spread of the distribution for the quantitative variability suggests high consistency of the automated sample preparation technique.

In addition to the increased throughput, nPOP reduced sample preparation batch effects that could introduce technical artifacts. Indeed, because all single cells were prepared on the same day, no sample preparation batch corrections needed to be applied to the data.

Next, principal component analysis (PCA) of the single-cell protein dataset was performed using all quantified proteins (FIG. 2E). The PCA indicates three distinct clusters of cells. The clusters correspond the cell types, with two sub populations of melanoma cells. The cell types separate along the first principal component (PC1), which accounts for 59% of the variance. To evaluate whether this separation reflects technical artifacts, such as differences in cell size or missing data, or biological differences between cell types, the proteomes of 200-cell samples of melanoma and monocyte cells analyzed by established bulk methods were projected (FIG. 2D).

The first step towards identifying within cell type protein covariation was to identify proteins that correlate significantly within monocyte and melanoma cells. Computing all pairwise correlations, 5,089 significant correlations were found in monocyte, and 4,679 correlations were found in melanoma cells at FDR <5%. 2,353 of these correlations were between the same pair of proteins. While most of these correlations shared the same trend, interestingly, 15 proteins showed opposite correlation trends. The joint distributions for proteins from these two cases were plotted in FIG. 3A and FIG. 3B, respectively.

A primary factor for observed protein covariation within a cell type may reflect proteins belonging to a complex. The goal was to identify whether observed protein covariation could be explained by proteins belonging to complexes. To this end, all pairwise Euclidean distances between proteins in know complexes from the CORUM database were computed (Giurgiu et al., Nucleic Acids Res. 47(D1):D559-D563 (2019)), and the distribution against all pairwise distances was tested. 96 protein complexes were identified in melanoma cells, and 89 were identified in monocytes at FDR <10%. Both cell types had similar agreement between Ribosomal proteins (FIG. 3C). A full list of differential protein complexes can be found in Table 4.

### Example 4. Cell Cycle Analysis

A more challenging problem was quantifying CDC-related protein covariation within a cell type. As a first step towards this analysis, the potential to classify individual cells by their cell cycle phase was evaluated. To obtain a list of proteins whose abundance varies periodically with the cell division cycle, populations of each cell type were first sorted based off their DNA content (FIGS. 4A and 4F). The proteomes of the sorted cells were quantified, and proteins whose abundance differs in G1, S, and G2/M phase for both cell types were identified.

To construct robust markers for each phase, the abundances of groups of proteins corresponding to each phase of the cell cycle were averaged. For each CDC phase, two markers from non-overlapping sets of proteins were constructed. Positive correlation between markers from the same phase served as internal validation based on the expectation that proteins peaking in the same phase positively covary. Conversely, markers for different phases were expected to negatively correlate to each other (FIGS. 4B and 4G). Markers were first constructed in the space of monocyte cells, and correlations between markers were cross-validated in melanoma cells (FIGS. 4A and 4F). Having validated the protein markers, protein markers within phase were averaged for downstream analysis.

The proteomes of both melanoma and monocyte cells were then projected into a joint 2-dimensional space of the CDC marker proteins defined by principal component analysis (FIGS. 4B and 4G). Each cell was then color-coded based on the mean abundance of a given protein marker in the PCA plots for their respective phase (FIGS. 4C and 4H). The cells from both cell types cluster by CDC phase, which further suggests that the data capture CDC related protein dynamics.

To identify proteins that covary with the CDC periodic markers, the phase marker vectors were correlated to the measured protein abundances of all proteins quantified across many single cells. For 121 of these proteins in the melanoma and 113 in the monocyte, the correlations were statistically significant, FDR <0.01, suggesting that these proteins are CDC periodic. Specifically, NPM1 which facilitates ribosome biogenesis positively correlated with G1 phase in both melanoma and monocyte populations, p<10-15, p<10-8, respectively.

To increase the statistical power and identify functional covariation with the CDC, the next focus was the covariation of phase markers and proteins with similar functions as defined by the gene ontology (GO). The distributions of correlations between the 3 phase marker vectors and all quantified proteins from a GO term were compared (see the boxplots in FIGS. 4D and 41). For protein polyubiquitination, the distributions of correlations differed significantly between the CDC phases, and this phase-specific covariation was similar for the two cell types. Many other GO terms showed covariation to the phase markers that was similar for the two cell types. Instead of displaying the boxplot distributions for all of them, the distributions of correlations were summarized with their medians and displayed as a heatmap. Such functions with shared covariation included proteolysis in G2/M phase which implicate the role of protein degradation in cell cycle progression. Additionally, terms related to DNA repair and translation were correlated with G1 markers, confirming the role of cell growth and DNA repair post mitosis.

In addition to finding groups of proteins that showed similar cell cycle covariation between cell types, several GO terms also varied differential with CDC markers (FIGS. 4E and 4J). Such GO terms included terms related to cell signaling, metabolism and immune system related processes which may reflect the role of the monocyte as an immune cell. However, a larger majority of the 117 significant GO terms (Table 3) showed concerted trends between the two cell types highlighting the conservation CDC related processes.

### Example 5. Melanoma Sub Population

Next, the two distinct clusters of melanoma cells observed in FIG. 2D were analyzed. Recent studies of these melanoma cells identified two populations with distinct transcriptomes (Emert et al., Nat Biotechnol. 39(7):865-76 (2021) and Fallahi-Sichani et al., Mol Syst Biol. 13(1):905 (2017)). The larger population is susceptible to treatment by the cancer drug vemurafenib, while the smaller one is primed to develop drug resistance (Emert et al., Nat Biotechnol. 39(7):865-76 (2021)).

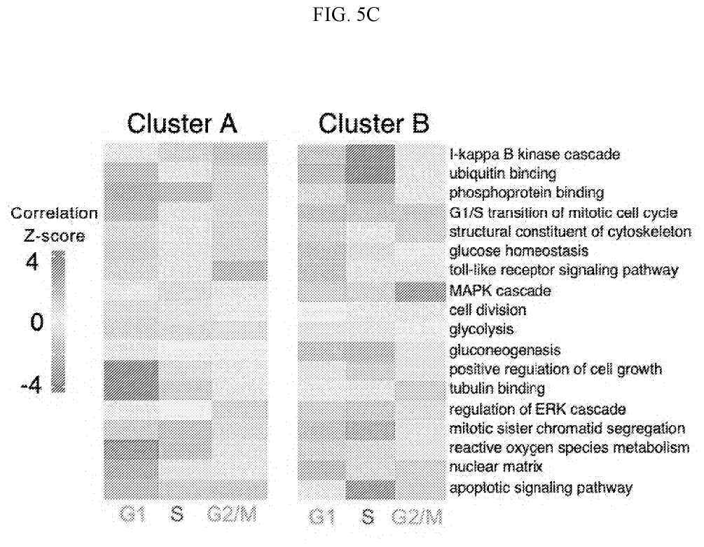

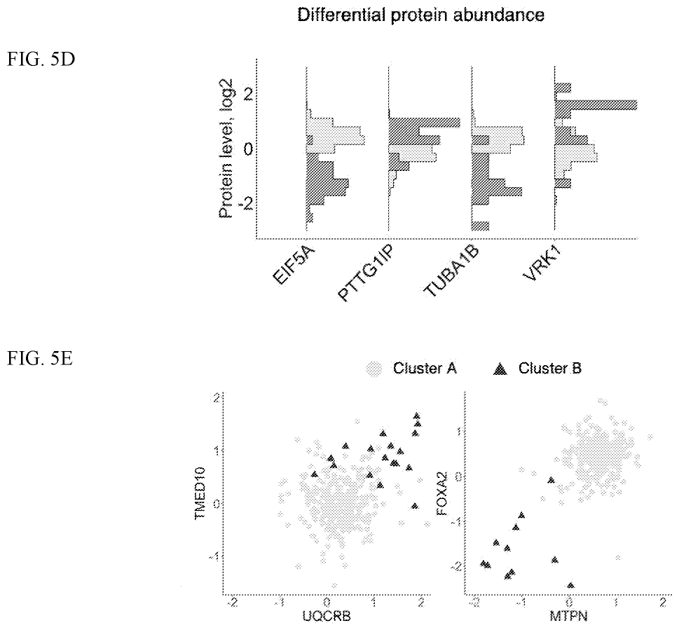

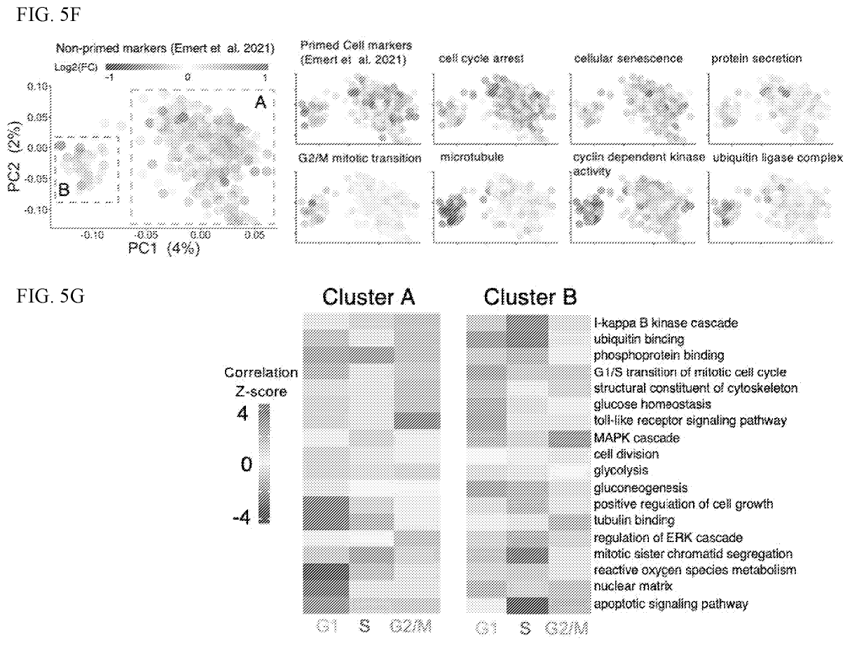

To test if the clusters mapped to the same distinct cell states previously identified, the cells were color coded by the abundance of proteins whose transcripts were reported (Emert et al., Nat Biotechnol. 39(7):865-76 (2021)) to mark either the non-primed population (Cluster A) or the primed sub-population (Cluster B) (FIG. 5A). Primed markers were significantly more abundant in cluster B, p=2e-4, while non-primed had greater abundance in cluster A, p<le-15. Having established correspondence between the populations, additional protein differences between the two clusters were identified by performing PSEA. It resulted in 200 sets of functionally related proteins exhibiting differential abundance at FDR <1% (Table 5). Some of these sets were displayed by color coding the single cells from the PCA plot with the mean protein abundances for the set (FIG. 5A). Protein sets related to G2/M transition of mitosis, cyclin dependent kinase activity and protein degradation were more abundant in cluster A. In contrast, protein sets with increased abundance in cluster B related to senescence and cell cycle arrest. These results suggest that Cluster A cells are more proliferative than cluster B cells, consistent with prior report (Fallahi-Sichani et al., Mol Syst Biol. 13(1):905 (2017)).

To explore CDC differences further, the distribution of cells in each CDC phase across the two sub-populations were quantified. A substantially larger fraction of cells were found in cluster B in G1 phase, 78%, while only 4% of cells were found to be assigned to G2 phase (FIG. 5B). This result further bolsters the conclusion that cluster B cells divide slower than cluster A cells. The next goal was to identify additional groups of proteins that co-vary with CDC phase between the two populations. Upon repeating the analysis from FIGS. 4C and 4D on both melanoma populations, several sets of proteins were found to correlate significantly to the CDC markers. Many of these sets correlated deferentially to the markers within each cluster. Specifically, many terms for glucogenesis and signaling cascades displayed different correlation profiles to the CDC markers.

Lastly, 234 additional proteins were differential between cluster A and cluster B cells at FDR <1%. Some of these proteins were displayed in FIG. 5D, as distributions of abundances for individual proteins, and in FIG. 5E, as joint distributions for abundances of two proteins. Notably, increased abundance of the surface protein Transmembrane emp24 domain-containing protein 10, and decreased abundance of the transcription factor Hepatocyte nuclear factor 3-beta in cluster B were found (FIG. 5E). The remaining list of differential proteins can be found in Table 6.

### Example 6. Surface protein analysis

NPOP was also applied to specifically study surface proteins in an additional experimental system, pancreatic ducal adenocarcinoma (PDAC) (FIGS. 6A and 6B). Identifying co-abundant surface proteins has valuable potential for therapeutics that utilize bi-specific antibodies or receptors (Dahlén et al., Ther Adv Vaccines Immunother. 6(1):3-17 (2018)). Thus, single cell proteomics may emerge as a useful tool for suggesting such pairs of proteins. To this end, the analysis of 34 different surface proteins including well known markers of PDAC, such as CEACAM5 and CEACAM6, was prioritized (Gebauer et al., PLOS One. 9(11):e113023 (2014)). Hierarchical clustering in the space of all pairwise protein protein correlations revealed co-abundant clusters of surface proteins (FIGS. 6C and 6E). Correlations were found between proteins such as CD44 and CEACAM6. Additionally, correlations between surface proteins and other intracellular proteins were computed, and 120 significant correlations at FDR <1% were found (FIG. 6D).

Existing single-cell omics methods excel at classifying cells by cell type. However, the regulatory dynamics resulting in cell to cell variability within a cell type are more challenging to analyze. To support such analysis, a highly parallel sample preparation that enables preparation of hundreds to thousands of single cells in a given experiment was introducee. It allows for reduced volumes and increased consistency of single-cell proteomic sample preparation. Furthermore, it can enable processing thousands of single cells in parallel and thus empower high-throughput, high-power biological analysis (Slavov, Nat Biotechnol. 39(7):809-10 (2021)).

To maximize access and flexibility, nPOP used only commercially available equipment and prepared single cells on an open surface that could be pragmatically reconfigured and adopted to different experimental designs. The open environment also obviated all sample movements and maximized the consistency and precision of the sample preparation. The open layout using a hydrophobic slide can be scaled up to simultaneously prepare thousands of single cells. Furthermore, nPOP is amenable to different coatings or hydrophobic surfaces which have the potential to further improve recovery.

NPOP allowed for deeper single cell proteomic analysis of the cell division cycle than the CDC analysis using the minimal sample preparation method (mPOP) (Specht et al., bioRxiv. 399774 (2018)). The data allowed identification of new proteins and functional groups of proteins associated with the cell cycle without the artifacts associated with synchronizing cell cultures (Cooper, FEBS J. 286(23):4650-56 (2019)). Furthermore, functional groups of proteins associated with the cell cycle were determined in an identified subpopulation of cells within the melanomas. These initial results demonstrate the feasibility of inferring co-regulation of biological processes from single-cell proteomics measurements.

### Example 7. nPOP Workflow

A non-limiting example of an overall work flow for nPOP sample preparation includes cell isolation, cell lysis, protein digestion, peptide labeling, and pooling as illustrated in FIG. 8A. Sample preparation starts with dispensing droplets of 4 nl DMSO for cell lysis. The droplets are organized as regular grids (e.g., a cluster, see, e.g., FIG. 8B) to facilitate their automating deposition, regular additions during sample preparation and pooling at the end of the experiment. The second step of nPOP is the isolation and dispensing of single cells into the DMSO droplets. Each single cell is isolated in a 0.3 nl droplet and added to a DMSO droplet for lysis (FIG. 8A). After 20 minutes for cell lysis, a perimeter of 12 nl droplets of water (for maintaining high local humidity) is deposited around the perimeter of the four samples. The next nPOP step is the addition of trypsin with HEPES buffer for digesting the proteins into peptides. This step brings the total volume to 13.5 nl. Samples are digested at a 75 ng/μl of trypsin for 5 hours on slide. To further control evaporation, nPOP uses a humidifier to keep relative humidity inside the CellenONER at 75%. The temperature of the slide is set to dynamically adjust to one degree above the dew point inside the CellenONER and stays around 17ºC for digestion. After digestion, humidity is reduced, and the slide is brought to room temperature for labeling. The single cell droplets dry down on the slide to volumes of approximately 4 nl before labeling. TMT labels dissolved in DMSO are dispensed in volumes of 20 nl to the single cell droplets. Dissolving labels in DMSO is a distinctive and required aspect of nPOP that allows for easy handling of tiny droplets with TMT solution. The most commonly used solvent for TMT, acetonitrile, is difficult to handle with CellenONER. After samples are labeled for one hour at room temperature, labeling is quenched with the addition of 20 nl 5% hydroxylamine for 20 minutes. A second addition of hydroxylamine is then added and sample quenches for an additional 20 minutes.

To pool all single-cell samples into a set, 1 μl of water is pipetted by hand onto each array of labeled samples. Samples are then pipetted directly into glass inserts containing carrier and reference previously prepared using the mPOP protocol (Specht & Slavov, J Proteome Res. 17(8):2565-71 (2018)) for injection vials. To improve the recovery of labeled peptides, the footprint of each array can be washed by 4 μl of acetonitrile, which is collected and added to the corresponding combined set. This wash is optional and is used to maximize the recovery of labeled peptides from the slide.

### Example 8. Single-Cell Protein Analysis with nPOP

nPOP is a general sample preparation method that can be used for either label-free MS analysis or multiplexed MS analysis as part of existing workflows reviewed by Slavov, Curr Opin Chem Biol. 60:1-9 (2021) and Kelly, Mol Cell Proteomics 19(11): 1739-48 (2020). Here, sample preparation by nPOP as part of the SCOPE2 protocol (Specht et al., Genome Biol. 22(1):50 (2021) and Petelski et al., Nat Protoc. 16(12):5398-25 (2021)) is demonstrated. Specifically, Minimal ProteOmic sample Preparation (mPOP) module (Specht & Slavov, J Proteome Res. 17(8):2565-71 (2018)) was replaced with nPOP and used all other modules of the SCOPE2 workflow, including an isobaric carrier (Specht & Slavov, J Proteome Res. 20(1):880-87 (2021)), Data-Driven Optimization of Mass Spectrometry (DO-MS) (Huffman et al., bioRxiv. 512152 (2019)), Data-driven Alignment of Retention Times for IDentification (DART-ID) (Chen et al., PLOS Comput Biol. 15(7):e1007082 (2019)), and the SCOPE2 data analysis pipeline (Specht et al., Genome Biol. 22(1):50 (2021) and Vanderaa et al., Bioconductor (2020)).

To evaluate the performance of nPOP for single-cell sample preparation, proteins in 176 single cells of two distinct cell types, Hela cells and U-937 monocytes, were measured. The sample preparation was done on two different days so that the data may reflect day-specific batch effects. The resulting SCOPE2 sets were run using less than 24 hours of instrument time. Samples were analyzed and data processed via the SCOPE pipeline (Specht et al., Genome Biol. 22(1):50 (2021)). To evaluate the single-cell data, the pipeline calculated the coefficient of variation (CV) of relative peptide levels belonging to the same protein. The relatively low CV values indicate that protein quantification from different peptides was internally consistent (FIG. 8C). Furthermore, the small spread of the distribution for the median CVs indicates that each cell is treated consistently by the automated sample preparation technique.

Next, principal component analysis (PCA) of the single-cell protein dataset was performed using all quantified proteins (FIG. 8D). The PCA indicates two distinct clusters of cells. The clusters corresponded with the cell type and separated along the first principal component (PC1), which accounted for 73% of the variance (FIG. 8D).

To validate further that the cell type separation was driven by accurate quantification of proteins (rather than by secondary factors such as cell size or missing data), bulk samples of Hela cells and monocytes were included in the PCA. Similar to previous analysis (Specht et al., Genome Biol. 22(1):50 (2021), Petelski et al., Nat Protoc. 16(12):5398-25 (2021) and Budnik et al., Genome Biol. 19(1):161 (2018)), the bulk samples clustered with the corresponding single cells. This clustering indicated that the single cell protein quantification was consistent with the proteomic measurements of established bulk methods.

### Example 9. Cell Cycle Analysis

To test further the quantitative accuracy of the data, the heterogeneity within a cell type was studied. Differences in cell state were measured by analyzing the variation in known cell division cycle proteins. To do this, the data for CDC proteins were filtered and cells along the first two principal components were plotted. Each cell was then color coded based on the mean abundance of markers for M/G1 and G2/S phases in the cell. The color-coded cells clustered along the first and second principal component, indicating the feasibility of inferring cell cycle phase from the cells analyzed with nPOP.

A method that prepares single cells in 4-15 nanoliter volumes using only commercially available equipment is demonstrated. The current method prepares single cells in an open environment without a need to move samples in the process to maximize the consistency and precision of the sample preparation. The open layout using a glass slide is scalable to preparing hundreds of single cells at a time. Furthermore, the current method is amenable to different coatings or hydrophobic surfaces which have the potential to further improve recovery.

TABLE 2
Protein set enrichment analysis based on protein levels in cells isolated based on DNA content from FIGs. 4A-4J

| | | | numberOf | fractionOfDB_{—} | | | | |
|---|---|---|---|---|---|---|---|---|
| | GO_term | pVal | Matches | Observed | G1 | S | G2 | FDR |
| 32 | regulation of cellular amino acid metabolic process | 2.45E−29 | 43 | 0.843137 | −0.16421 | 0.062426 | 0.083261 | 7.88E−26 |
| 34 | cellular nitrogen compound metabolic process | 4.58E−26 | 114 | 0.616216 | −0.11926 | 0.064725 | 0.035002 | 7.37E−23 |
| 162 | respiratory electron transport chain | 7.39E−25 | 80 | 0.761905 | 0.005534 | 0.07522 | −0.10121 | 7.93E−22 |
| 141 | proteasome accessory complex | 1.16E−24 | 17 | 0.62963 | 0.16543 | 0.036435 | 0.109147 | 9.35E−22 |
| 37 | regulation of ubiquitin-protein ligase activity involved in mitotic cell cycle | 3.75E−21 | 62 | 0.826667 | −0.15151 | 0.044838 | 0.109049 | 2.41E−18 |
| 144 | proteasome complex | 9.28E−20 | 51 | 0.836066 | −0.15017 | 0.032138 | 0.083261 | 4.98E−17 |
| 36 | positive regulation of ubiquitin-protein ligase activity involved in mitotic cell | 2.80E−19 | 58 | 0.816901 | −0.15387 | 0.049323 | 0.107033 | 1.29E−16 |
| | cycle | | | | | | | |
| 223 | negative regulation of ubiquitin-protein ligase activity involved in mitotic cell | 4.44E−19 | 55 | 0.846154 | −0.14787 | 0.047445 | 0.107033 | 1.78E−16 |
| | cycle | | | | | | | |
| 33 | DNA damage response, signal transduction by p53 class mediator resulting in | 5.96E−17 | 51 | 0.728571 | −0.15151 | 0.06121 | 0.083893 | 2.13E−14 |
| | cell cycle arrest | | | | | | | |
| 192 | ATP-dependent chromatin remodeling | 2.79E−16 | 21 | 0.84 | 0.093528 | −0.192 | 0.027195 | 8.98E−14 |
| 204 | mitochondrial ribosome | 6.82E−15 | 23 | 0.69697 | −0.0765 | 0.163773 | −0.0818 | 2.00E−12 |
| 14 | protein polyubiquitination | 3.50E−14 | 73 | 0.51773 | −0.12806 | 0.042758 | 0.065777 | 9.39E−12 |
| 128 | tricarboxylic acid cycle | 1.35E−13 | 26 | 0.52 | −0.04468 | 0.118258 | −0.09065 | 3.35E−11 |
| 256 | mitochondrial small ribosomal subunit | 3.04E−13 | 18 | 1 | −0.08978 | 0.187256 | −0.07391 | 6.99E−11 |
| 165 | G1/S transition of mitotic cell cycle | 1.17E−12 | 102 | 0.563536 | −0.15017 | 0.05532 | 0.084525 | 2.51E−10 |
| 71 | melanosome | 1.65E−12 | 84 | 0.823529 | 0.021763 | 0.045153 | −0.08822 | 3.33E−10 |
| 246 | DNA strand elongation involved in DNA replication | 2.25E−12 | 27 | 0.870968 | −0.1821 | 0.008833 | 0.10294 | 4.25E−10 |
| 49 | nucleosomal DNA binding | 9.79E−12 | 20 | 0.526316 | 0.093528 | −0.17623 | 0.01104 | 1.68E−09 |
| 131 | SWI/SNF complex | 9.90E−12 | 12 | 0.545455 | 0.191848 | −0.19741 | 0.023347 | 1.68E−09 |
| 30 | viral infectious cycle | 1.69E−11 | 100 | 0.847458 | −0.06343 | 0.064388 | −0.03382 | 2.72E−09 |
| 65 | spindle | 1.84E−11 | 76 | 0.520548 | −0.09735 | −0.0331 | 0.068958 | 2.82E−09 |
| 222 | nucleobase-containing small molecule metabolic process | 4.61E−11 | 56 | 0.717949 | −0.12346 | 0.037102 | 0.053552 | 6.74E−09 |
| 84 | mitochondrial respiratory chain complex I | 9.69E−11 | 41 | 0.719298 | 0.009603 | 0.07337 | −0.1029 | 1.36E−08 |
| 150 | chromatin remodeling | 1.02E−10 | 45 | 0.463918 | 0.131442 | −0.16481 | 0.032692 | 1.37E−08 |
| 132 | mitochondrial electron transport, NADH to ubiquinone | 1.27E−10 | 39 | 0.78 | 0.010877 | 0.076719 | −0.10643 | 1.64E−08 |
| 252 | viral transcription | 1.61E−10 | 74 | 0.902439 | −0.06417 | 0.066076 | −0.04127 | 1.99E−08 |
| 130 | translational termination | 2.99E−10 | 76 | 0.76 | −0.06417 | 0.065467 | −0.04096 | 3.57E−08 |
| 74 | RNA polymerase II distal enhancer sequence-specific DNA binding | 7.92E−10 | 25 | 0.555556 | 0.087479 | −0.16763 | 0.041553 | 9.10E−08 |
| 123 | translational elongation | 9.35E−10 | 84 | 0.651163 | −0.06146 | 0.056745 | −0.04154 | 1.04E−07 |
| 15 | integral to endoplasmic reticulum membrane | 9.80E−10 | 32 | 0.340426 | 0.145693 | 0.0592 | −0.11897 | 1.05E−07 |
| 24 | proteasome core complex | 1.15E−09 | 18 | 0.418605 | −0.16359 | 0.067345 | 0.070964 | 1.20E−07 |
| 110 | npBAF complex | 1.43E−09 | 10 | 0.4 | 0.11687 | −0.19075 | −0.00053 | 1.44E−07 |
| 2 | RNA polymerase II core promoter proximal region sequence-specific DNA | 1.65E−09 | 47 | 0.315436 | 0.097175 | −0.15762 | 0.027474 | 1.61E−07 |
| | binding | | | | | | | |
| 8 | ion transport | 1.74E−09 | 27 | 0.156977 | −0.04792 | 0.140563 | −0.1736 | 1.65E−07 |
| 219 | nuclear-transcribed mRNA catabolic process, nonsense-mediated decay | 3.07E−09 | 97 | 0.815126 | −0.06446 | 0.041942 | −0.01568 | 2.82E−07 |
| 120 | NADH dehydrogenase (ubiquinone) activity | 3.81E−09 | 39 | 0.58209 | 0.018774 | 0.067717 | −0.10535 | 3.41E−07 |
| 179 | SRP-dependent cotranslational protein targeting to membrane | 5.56E−09 | 98 | 0.830508 | −0.05122 | 0.061788 | −0.0524 | 4.83E−07 |
| 97 | nuclear inner membrane | 6.17E−09 | 20 | 0.571429 | 0.118928 | −0.16447 | −0.00109 | 5.22E−07 |
| 100 | regulation of translational initiation | 8.04E−09 | 33 | 0.611111 | −0.12036 | 0.036123 | 0.042677 | 6.63E−07 |
| 111 | nBAF complex | 9.58E−09 | 8 | 0.275862 | 0.168085 | −0.21357 | 0.01725 | 7.70E−07 |
| 211 | de novo' posttranslational protein folding | 1.02E−08 | 30 | 0.769231 | −0.15019 | 0.088715 | 0.007556 | 8.04E−07 |
| 195 | chromatin organization | 1.28E−08 | 68 | 0.561983 | 0.102038 | −0.13131 | 0.032564 | 9.77E−07 |
| 243 | catalytic step 2 spliceosome | 1.36E−08 | 77 | 0.9625 | 0.011309 | −0.03822 | 0.041113 | 1.01E−06 |
| 12 | cellular lipid metabolic process | 1.44E−08 | 87 | 0.524096 | 0.037441 | 0.029152 | −0.08104 | 1.05E−06 |
| 94 | pyruvate metabolic process | 1.56E−08 | 19 | 0.542857 | −0.04449 | 0.099324 | −0.11296 | 1.12E−06 |
| 31 | antigen processing and presentation of exogenous peptide antigen via MHC | 2.06E−08 | 63 | 0.446809 | −0.13681 | 0.063575 | 0.057411 | 1.44E−06 |
| | class I, TAP-dependent | | | | | | | |
| 5 | mediator complex | 5.46E−08 | 26 | 0.309524 | 0.143333 | −0.16837 | 0.014175 | 3.74E−06 |
| 35 | antigen processing and presentation of exogenous peptide antigen via MHC | 5.64E−08 | 66 | 0.452055 | −0.12806 | 0.063575 | 0.053209 | 3.78E−06 |
| | class I | | | | | | | |
| 88 | electron carrier activity | 6.05E−08 | 47 | 0.516484 | −0.02651 | 0.077971 | −0.07193 | 3.98E−06 |
| 63 | translation initiation factor activity | 6.93E−08 | 43 | 0.286667 | −0.1238 | 0.02009 | 0.077118 | 4.46E−06 |
| 99 | eukaryotic translation initiation factor 3 complex | 9.76E−08 | 15 | 0.223881 | −0.18804 | 0.079906 | 0.08019 | 6.16E−06 |
| 6 | phagocytic vesicle membrane | 1.31E−07 | 32 | 0.273504 | 0.04696 | 0.085917 | −0.17535 | 8.13E−06 |
| 158 | lipid particle | 1.58E−07 | 29 | 0.402778 | 0.068452 | −0.07888 | −0.07974 | 9.58E−06 |
| 101 | eukaryotic 43S preinitiation complex | 2.06E−07 | 14 | 0.56 | −0.19032 | 0.081462 | 0.08019 | 1.23E−05 |
| 182 | snRNA processing | 2.38E−07 | 11 | 0.37931 | 0.142306 | −0.10625 | −0.03667 | 1.37E−05 |
| 183 | integrator complex | 2.38E−07 | 11 | 0.366667 | 0.142306 | −0.10625 | −0.03667 | 1.37E−05 |
| 75 | glucose metabolic process | 2.80E−07 | 66 | 0.437086 | −0.05355 | 0.054665 | −0.02263 | 1.58E−05 |
| 53 | purine base metabolic process | 3.01E−07 | 24 | 0.666667 | −0.11526 | 0.045347 | 0.02116 | 1.67E−05 |
| 98 | formation of translation preinitiation complex | 3.45E−07 | 13 | 0.52 | −0.19032 | 0.081462 | 0.08019 | 1.85E−05 |
| 102 | eukaryotic 48S preinitiation complex | 3.45E−07 | 13 | 0.541667 | −0.19032 | 0.081462 | 0.08019 | 1.85E−05 |
| 148 | sperm protein complex | 5.16E−07 | 8 | 0.363636 | −0.13997 | 0.065658 | 0.060558 | 2.72E−05 |
| 127 | protein disulfide isomerase activity | 6.05E−07 | 16 | 0.516129 | 0.128265 | −0.01208 | −0.09251 | 3.13E−05 |
| 149 | chaperonin-containing T-complex | 6.13E−07 | 9 | 0.428571 | −0.13997 | 0.065658 | 0.060558 | 3.13E−05 |
| 126 | proteasome core complex, alpha-subunit complex | 7.16E−07 | 7 | 0.269231 | −0.18048 | 0.11554 | 0.058944 | 3.60E−05 |
| 118 | ER-associated protein catabolic process | 8.02E−07 | 24 | 0.571429 | 0.136369 | −0.04677 | −0.09942 | 3.97E−05 |
| 64 | spindle pole | 9.00E−07 | 43 | 0.307143 | −0.05084 | −0.03465 | 0.101614 | 4.39E−05 |
| 81 | cytosolic large ribosomal subunit | 9.38E−07 | 44 | 0.785714 | −0.06298 | 0.066455 | −0.04536 | 4.50E−05 |
| 216 | ER membrane protein complex | 1.00E−06 | 9 | 0.692308 | 0.163404 | −0.06521 | −0.14167 | 4.71E−05 |
| 161 | chromosome segregation | 1.01E−06 | 42 | 0.477273 | −0.08845 | −0.0467 | 0.094289 | 4.71E−05 |
| 106 | DNA methylation | 1.30E−06 | 12 | 0.387097 | 0.213756 | −0.17843 | −0.0265 | 5.98E−05 |
| 27 | tRNA binding | 1.33E−06 | 31 | 0.62 | −0.09439 | 0.030347 | 0.052023 | 6.05E−05 |
| 232 | positive regulation of innate immune response | 1.40E−06 | 5 | 0.384615 | −0.3905 | 0.144071 | 0.219182 | 6.25E−05 |
| 230 | unsaturated fatty acid metabolic process | 1.77E−06 | 9 | 0.818182 | 0.120954 | 0.034063 | −0.18453 | 7.69E−05 |
| 231 | alpha-linolenic acid metabolic process | 1.77E−06 | 9 | 0.818182 | 0.120954 | 0.034063 | −0.18453 | 7.69E−05 |
| 186 | protein N-linked glycosylation via asparagine | 1.83E−06 | 62 | 0.632653 | 0.093838 | −0.04186 | −0.07781 | 7.83E−05 |
| 11 | triglyceride biosynthetic process | 1.85E−06 | 21 | 0.396226 | 0.089435 | 0.024401 | −0.16332 | 7.83E−05 |
| 226 | regulation of proteasomal protein catabolic process | 2.10E−06 | 7 | 0.583333 | −0.16189 | −0.01281 | 0.132206 | 8.76E−05 |
| 188 | Ino80 complex | 2.20E−06 | 13 | 0.448276 | 0.147167 | −0.17969 | 0.014416 | 9.04E−05 |
| 124 | tRNA aminoacylation for protein translation | 2.24E−06 | 42 | 0.608696 | −0.05202 | 0.050645 | −0.01268 | 9.04E−05 |
| 136 | mitochondrial membrane | 2.25E−06 | 39 | 0.423913 | −0.0135 | 0.061614 | −0.085 | 9.04E−05 |
| 95 | vitamin D receptor binding | 2.34E−06 | 13 | 0.8125 | 0.147668 | −0.15714 | −0.01552 | 9.28E−05 |
| 155 | dolichyl-diphosphooligosaccharide-protein glycotransferase activity | 2.41E−06 | 7 | 0.5 | 0.0568 | 0.013482 | −0.14572 | 9.44E−05 |
| 201 | mitochondrial nucleoid | 2.68E−06 | 37 | 0.902439 | −0.00445 | 0.055354 | −0.05293 | 0.000104 |
| 253 | mitochondrial large ribosomal subunit | 3.11E−06 | 14 | 0.933333 | −0.04843 | 0.173239 | −0.09096 | 0.000119 |
| 215 | RNA polymerase II repressing transcription factor binding | 3.28E−06 | 10 | 0.285714 | 0.090858 | −0.12685 | 0.013096 | 0.000124 |
| 225 | lysosomal lumen | 3.39E−06 | 36 | 0.507042 | 0.077563 | 0.009343 | −0.10295 | 0.000127 |
| 25 | antigen processing and presentation of peptide antigen via MHC class I | 4.14E−06 | 81 | 0.455056 | −0.1132 | 0.0572 | 0.049277 | 0.000153 |
| 142 | NuRD complex | 4.57E−06 | 13 | 0.361111 | 0.0613 | −0.16927 | 0.065695 | 0.000167 |
| 119 | mitotic cell cycle spindle assembly checkpoint | 5.02E−06 | 24 | 0.571429 | −0.07541 | −0.06166 | 0.166742 | 0.000182 |
| 206 | proteasome binding | 6.02E−06 | 8 | 0.727273 | −0.1886 | −0.04456 | 0.170854 | 0.000215 |
| 85 | midbody | 6.60E−06 | 69 | 0.534884 | −0.04933 | −0.00339 | 0.032295 | 0.000234 |
| 56 | kinetochore | 7.63E−06 | 52 | 0.530612 | −0.03572 | −0.04522 | 0.091451 | 0.000267 |
| 13 | axonogenesis | 7.80E−06 | 28 | 0.224 | 0.018824 | 0.087952 | −0.16282 | 0.00027 |
| 22 | antigen processing and presentation | 8.00E−06 | 16 | 0.258065 | 0.04884 | 0.078843 | −0.16837 | 0.000274 |
| 217 | histone H4-K16 acetylation | 8.43E−06 | 14 | 0.608696 | 0.121237 | −0.18519 | 0.069265 | 0.000285 |
| 50 | nuclear pore | 8.75E−06 | 51 | 0.451327 | 0.042184 | −0.08528 | 0.011917 | 0.000293 |
| 67 | ATP hydrolysis coupled proton transport | 9.46E−06 | 14 | 0.157303 | 0.001607 | 0.078724 | −0.087 | 0.000314 |
| 143 | gluconeogenesis | 1.02E−05 | 26 | 0.376812 | −0.04908 | 0.072529 | −0.03359 | 0.000335 |
| 167 | long-chain fatty acid transport | 1.09E−05 | 4 | 0.307692 | 0.360173 | −0.21507 | −0.19082 | 0.000355 |
| 20 | cytokinesis after mitosis | 1.16E−05 | 14 | 0.424242 | −0.19888 | −0.01315 | 0.094845 | 0.000371 |
| 77 | G2/M transition of mitotic cell cycle | 1.17E−05 | 81 | 0.536424 | −0.07129 | −0.00286 | 0.055686 | 0.000371 |
| 1 | histone acetyltransferase complex | 1.29E−05 | 12 | 0.363636 | 0.131663 | −0.2084 | 0.051313 | 0.000407 |
| 154 | long-chain fatty acid-CoA ligase activity | 1.31E−05 | 6 | 0.222222 | 0.234518 | −0.1022 | −0.1933 | 0.000408 |
| 52 | septin complex | 1.39E−05 | 7 | 0.145833 | 0.083409 | −0.11239 | 0.044319 | 0.000429 |
| 244 | phagocytic vesicle | 1.46E−05 | 23 | 0.793103 | 0.038123 | 0.024386 | −0.11006 | 0.000446 |
| 151 | regulation of acetyl-CoA biosynthetic process from pyruvate | 1.49E−05 | 10 | 0.5 | −0.0432 | 0.083934 | −0.11644 | 0.000453 |
| 218 | spindle microtubule | 1.61E−05 | 22 | 0.578947 | −0.14636 | −0.105 | 0.169429 | 0.000486 |
| 23 | threonine-type endopeptidase activity | 1.64E−05 | 18 | 0.321429 | −0.16359 | 0.087207 | 0.039216 | 0.000487 |
| 152 | calcium ion-dependent exocytosis | 1.68E−05 | 6 | 0.26087 | −0.02642 | 0.184774 | −0.25201 | 0.000495 |
| 79 | condensed chromosome kinetochore | 1.71E−05 | 52 | 0.742857 | −0.00514 | −0.06144 | 0.099829 | 0.000499 |
| 153 | long-chain fatty acid metabolic process | 1.73E−05 | 9 | 0.204545 | 0.205768 | −0.08773 | −0.18052 | 0.0005 |
| 60 | protein polymerization | 1.79E−05 | 10 | 0.212766 | −0.17849 | 0.172902 | 0.022303 | 0.000515 |
| 193 | oligosaccharyltransferase complex | 1.81E−05 | 9 | 0.529412 | 0.113519 | 0.007354 | −0.14441 | 0.000517 |
| 68 | proton-transporting ATPase activity, rotational mechanism | 1.96E−05 | 11 | 0.366667 | 0.001805 | 0.088199 | −0.09178 | 0.000549 |
| 4 | RNA polymerase II transcription cofactor activity | 1.96E−05 | 25 | 0.342466 | 0.127641 | −0.14458 | 0.014519 | 0.000549 |
| 117 | proteasome activator complex | 1.99E−05 | 3 | 0.1875 | −0.19317 | 0.114138 | 0.053545 | 0.000551 |
| 220 | proteasome regulatory particle | 2.01E−05 | 8 | 0.571429 | −0.15151 | 0.04543 | 0.064738 | 0.000554 |
| 17 | positive regulation of interferon-beta production | 2.08E−05 | 13 | 0.565217 | −0.17264 | 0.106172 | 0.079535 | 0.000566 |
| 103 | protein N-linked glycosylation | 2.10E−05 | 12 | 0.363636 | 0.099357 | 0.036393 | −0.20665 | 0.000567 |
| 228 | nucleosome disassembly | 2.32E−05 | 12 | 0.705882 | 0.110704 | −0.16429 | −0.01081 | 0.000621 |
| 28 | kinase activity | 2.37E−05 | 19 | 0.131034 | −0.18302 | −0.01083 | 0.092884 | 0.000631 |
| 163 | fatty acid beta-oxidation | 2.40E−05 | 25 | 0.568182 | 0.014322 | 0.059874 | −0.08516 | 0.000634 |
| 59 | microtubule-based process | 2.42E−05 | 16 | 0.158416 | −0.17205 | 0.090011 | 0.034098 | 0.000634 |
| 9 | transmembrane transporter activity | 2.49E−05 | 14 | 0.215385 | 0.005268 | 0.074119 | −0.10636 | 0.000646 |
| 42 | negative regulation of catalytic activity | 2.54E−05 | 32 | 0.235294 | −0.09192 | 0.029677 | 0.027552 | 0.000655 |
| 180 | endoderm development | 2.59E−05 | 9 | 0.225 | 0.264604 | −0.24097 | −0.08909 | 0.000662 |
| 115 | chromatin modification | 2.76E−05 | 59 | 0.5 | 0.076637 | −0.11972 | 0.028932 | 0.000698 |
| 174 | lamin binding | 2.77E−05 | 7 | 0.466667 | 0.118928 | −0.22315 | −0.07637 | 0.000698 |
| 47 | cytosolic small ribosomal subunit | 2.96E−05 | 33 | 0.804878 | −0.07318 | 0.069572 | −0.03488 | 0.000739 |
| 187 | structural constituent of nuclear pore | 3.15E−05 | 8 | 0.615385 | 0.059971 | −0.11294 | −0.0033 | 0.00078 |
| 40 | peroxisomal membrane | 3.22E−05 | 34 | 0.53125 | 0.053474 | 0.034593 | −0.10099 | 0.00079 |
| 194 | purine ribonucleoside monophosphate biosynthetic process | 3.55E−05 | 11 | 0.785714 | −0.15026 | 0.102894 | 0.02011 | 0.000865 |
| 166 | viral receptor activity | 3.70E−05 | 10 | 0.285714 | −0.01626 | 0.169814 | −0.16554 | 0.000895 |
| 156 | protein N-terminus binding | 3.79E−05 | 57 | 0.491379 | 0.067428 | −0.08954 | −0.00992 | 0.00091 |
| 248 | mitochondrial ATP synthesis coupled proton transport | 4.03E−05 | 14 | 0.875 | 0.013494 | 0.062777 | −0.08601 | 0.00096 |
| 10 | hydrogen peroxide catabolic process | 4.12E−05 | 8 | 0.4 | −0.09879 | 0.076328 | −0.00322 | 0.000975 |
| 245 | transcription export complex | 4.23E−05 | 13 | 1 | 0.100418 | −0.11607 | 0.02015 | 0.000994 |
| 177 | cytochrome-c oxidase activity | 4.41E−05 | 14 | 0.264151 | −0.03129 | 0.125649 | −0.13295 | 0.001029 |
| 16 | extrinsic to membrane | 4.63E−05 | 18 | 0.264706 | −0.03703 | 0.129824 | −0.1151 | 0.001072 |
| 29 | integral to nuclear inner membrane | 4.74E−05 | 6 | 0.222222 | 0.233519 | −0.2111 | −0.09488 | 0.001091 |
| 172 | pyrimidine base metabolic process | 4.87E−05 | 15 | 0.517241 | −0.13504 | −0.02491 | 0.146294 | 0.001112 |
| 224 | MLL1 complex | 4.96E−05 | 25 | 0.925926 | 0.116412 | −0.12195 | 0.01688 | 0.001125 |
| 210 | RNA polymerase II carboxy-terminal domain kinase activity | 5.17E−05 | 14 | 0.823529 | 0.093156 | −0.15945 | 0.071303 | 0.001163 |
| 197 | chaperone-mediated protein complex assembly | 5.37E−05 | 8 | 0.5 | −0.14572 | 0.069709 | −0.00641 | 0.00119 |
| 146 | L-methionine salvage from methylthioadenosine | 5.38E−05 | 9 | 0.692308 | −0.18348 | 0.031838 | 0.088304 | 0.00119 |
| 145 | membrane protein ectodomain proteolysis | 5.46E−05 | 15 | 0.6 | 0.054086 | 0.019713 | −0.14771 | 0.00119 |
| 203 | mRNA transcription from RNA polymerase II promoter | 5.46E−05 | 3 | 0.214286 | 0.125953 | −0.15307 | −0.01308 | 0.00119 |
| 38 | nucleosome | 5.54E−05 | 12 | 0.144578 | 0.125919 | −0.22708 | −0.03249 | 0.00119 |
| 104 | DNA-dependent ATPase activity | 5.54E−05 | 25 | 0.396825 | 0.125031 | −0.139 | −0.05421 | 0.00119 |
| 237 | termination of RNA polymerase I transcription | 5.55E−05 | 15 | 0.625 | 0.091394 | −0.11934 | −0.03859 | 0.00119 |
| 208 | intracellular steroid hormone receptor signaling pathway | 5.86E−05 | 10 | 0.454545 | 0.143333 | −0.15993 | 0.014863 | 0.001249 |
| 249 | polyamine metabolic process | 6.59E−05 | 8 | 0.5 | −0.18348 | 0.104104 | 0.083016 | 0.001395 |
| 66 | steroid biosynthetic process | 6.98E−05 | 10 | 0.27027 | 0.122999 | −0.06091 | −0.17027 | 0.001468 |
| 19 | prefoldin complex | 7.32E−05 | 9 | 0.375 | −0.12995 | 0.183968 | −0.07403 | 0.00153 |
| 57 | spliceosomal complex | 7.64E−05 | 73 | 0.62931 | 0.008675 | −0.04912 | 0.047342 | 0.001587 |
| 39 | nuclear-transcribed mRNA catabolic process, deadenylation-dependent decay | 7.98E−05 | 43 | 0.781818 | −0.07308 | 0.005716 | 0.070863 | 0.001646 |
| 62 | glycolysis | 8.03E−05 | 26 | 0.19697 | −0.06513 | 0.077414 | −0.01372 | 0.001646 |
| 45 | Rab GTPase activator activity | 8.15E−05 | 20 | 0.151515 | −0.14442 | −0.0419 | 0.155899 | 0.00165 |
| 46 | positive regulation of Rab GTPase activity | 8.15E−05 | 20 | 0.151515 | −0.14442 | −0.0419 | 0.155899 | 0.00165 |
| 213 | L-serine transmembrane transporter activity | 8.28E−05 | 3 | 0.3 | −0.20839 | 0.292347 | −0.12203 | 0.001654 |
| 214 | L-serine transport | 8.28E−05 | 3 | 0.3 | −0.20839 | 0.292347 | −0.12203 | 0.001654 |
| 69 | ribose phosphate diphosphokinase activity | 8.92E−05 | 4 | 0.2 | −0.19295 | −0.19221 | 0.34583 | 0.00175 |
| 140 | ribose phosphate diphosphokinase complex | 8.92E−05 | 4 | 0.235294 | −0.19295 | −0.19221 | 0.34583 | 0.00175 |
| 239 | histone H4-K5 acetylation | 8.97E−05 | 11 | 0.733333 | 0.128957 | −0.14589 | 0.069265 | 0.00175 |
| 240 | histone H4-K8 acetylation | 8.97E−05 | 11 | 0.733333 | 0.128957 | −0.14589 | 0.069265 | 0.00175 |
| 108 | MCM complex | 0.000103 | 8 | 0.421053 | −0.28921 | 0.076952 | 0.09496 | 0.001973 |
| 255 | Nup107-160 complex | 0.000103 | 10 | 1 | 0.046194 | −0.10112 | 0.060993 | 0.001973 |
| 238 | endoplasmic reticulum-Golgi intermediate compartment membrane | 0.000103 | 22 | 0.758621 | 0.07661 | 0.051038 | −0.13193 | 0.001973 |
| 114 | R-SMAD binding | 0.000119 | 9 | 0.346154 | 0.132935 | −0.14438 | −0.04764 | 0.002273 |
| 169 | protein kinase C activity | 0.000122 | 7 | 0.155556 | −0.07321 | −0.13457 | 0.187185 | 0.002301 |
| 48 | aminopeptidase activity | 0.000126 | 22 | 0.392857 | −0.10693 | 0.020834 | 0.007109 | 0.002362 |
| 113 | sphingolipid metabolic process | 0.000133 | 32 | 0.380952 | 0.051022 | −0.02366 | −0.06775 | 0.00248 |
| 138 | binding of sperm to zona pellucida | 0.000133 | 11 | 0.211538 | −0.16374 | 0.069709 | −0.00641 | 0.002482 |
| 254 | telomere maintenance via semi-conservative replication | 0.000142 | 19 | 0.863636 | −0.16631 | −0.01194 | 0.141727 | 0.002612 |
| 105 | regulation of glucose transport | 0.000144 | 28 | 0.875 | 0.048286 | −0.10247 | 0.049905 | 0.002612 |
| 229 | S100 protein binding | 0.000144 | 8 | 0.727273 | 0.008799 | 0.229014 | −0.24767 | 0.002612 |
| 137 | ATP-dependent helicase activity | 0.000144 | 32 | 0.359551 | −0.00537 | −0.06924 | 0.055416 | 0.002612 |
| 87 | kinesin complex | 0.000144 | 19 | 0.118012 | −0.13828 | −0.05856 | 0.084166 | 0.002612 |
| 198 | neutral amino acid transmembrane transporter activity | 0.000145 | 3 | 0.230769 | −0.15789 | 0.344988 | −0.22054 | 0.002612 |
| 233 | branched chain family amino acid catabolic process | 0.000151 | 17 | 0.944444 | −0.07479 | 0.088313 | −0.05722 | 0.002701 |
| 82 | hydrogen ion transmembrane transporter activity | 0.000152 | 13 | 0.1625 | −0.00818 | 0.08546 | −0.07369 | 0.002701 |
| 41 | CCR4-NOT complex | 0.000157 | 10 | 0.588235 | −0.23048 | 0.046281 | 0.106251 | 0.002785 |
| 90 | protein homotetramerization | 0.000158 | 36 | 0.461538 | −0.05337 | 0.051517 | −0.03821 | 0.002786 |
| 89 | small ribosomal subunit | 0.000169 | 16 | 0.347826 | −0.07187 | 0.073501 | −0.02462 | 0.002946 |
| 242 | U12-type spliceosomal complex | 0.000169 | 17 | 0.708333 | 0.02057 | −0.04352 | 0.057469 | 0.002946 |
| 202 | negative regulation of nuclear mRNA splicing, via spliceosome | 0.000176 | 14 | 0.875 | −0.00441 | 0.08028 | −0.13313 | 0.003041 |
| 212 | morphogenesis of embryonic epithelium | 0.000181 | 2 | 0.111111 | 0.039417 | 0.168697 | −0.19555 | 0.003113 |
| 122 | ER to Golgi vesicle-mediated transport | 0.000182 | 43 | 0.401869 | 0.06241 | −0.07115 | −0.01915 | 0.003113 |
| 221 | Cajal body | 0.000203 | 31 | 0.688889 | −0.07604 | −0.00639 | 0.052887 | 0.00346 |
| 196 | peroxisome organization | 0.00021 | 9 | 0.375 | 0.084063 | −0.01852 | −0.09929 | 0.003557 |
| 78 | GPI anchor biosynthetic process | 0.000214 | 5 | 0.083333 | 0.294805 | −0.10397 | −0.31349 | 0.003606 |
| 116 | Ran GTPase binding | 0.000217 | 22 | 0.333333 | −0.10986 | 0.00611 | 0.111111 | 0.00363 |
| 86 | centrosome organization | 0.000228 | 16 | 0.290909 | −0.23218 | 0.15245 | 0.011865 | 0.003806 |
| 93 | protein targeting to mitochondrion | 0.000231 | 44 | 0.709677 | 0.002085 | 0.066315 | −0.04183 | 0.003834 |
| 107 | negative regulation of DNA binding | 0.000243 | 10 | 0.263158 | 0.161091 | −0.1102 | 0.019468 | 0.003979 |
| 164 | ferric iron binding | 0.000243 | 6 | 0.176471 | 0.022437 | 0.098371 | −0.16208 | 0.003979 |
| 3 | core promoter binding | 0.000244 | 13 | 0.25 | 0.092963 | −0.15894 | 0.004264 | 0.003979 |
| 83 | integrin complex | 0.00025 | 13 | 0.175676 | 0.01912 | 0.061454 | −0.10513 | 0.004067 |
| 73 | amino acid transport | 0.000263 | 8 | 0.205128 | −0.0588 | 0.270568 | −0.22054 | 0.004246 |
| 241 | interaction with host | 0.00027 | 17 | 0.5 | −0.01271 | 0.078724 | −0.04691 | 0.004347 |
| 235 | 1-acylglycerol-3-phosphate O-acyltransferase activity | 0.000285 | 9 | 0.75 | 0.192441 | −0.0867 | −0.19068 | 0.004558 |
| 191 | ceramide biosynthetic process | 0.000301 | 12 | 0.363636 | 0.098225 | −0.04928 | −0.04302 | 0.004802 |
| 54 | protein transporter activity | 0.000318 | 44 | 0.289474 | −0.07167 | 0.004818 | 0.032935 | 0.005045 |
| 168 | positive regulation of erythrocyte differentiation | 0.000321 | 7 | 0.269231 | 0.111565 | −0.15136 | 0.038154 | 0.005065 |
| 257 | exoribonuclease activity | 0.000323 | 11 | 0.916667 | −0.03415 | −0.09216 | 0.086495 | 0.005065 |
| 112 | removal of superoxide radicals | 0.000324 | 6 | 0.25 | −0.11763 | 0.149906 | −0.0077 | 0.005065 |
| 139 | cyclin binding | 0.000331 | 9 | 0.264706 | −0.04245 | −0.19008 | 0.149559 | 0.005143 |
| 91 | U1 snRNP | 0.000334 | 12 | 0.375 | −0.00596 | −0.04122 | 0.078437 | 0.005163 |
| 18 | sarcolemma | 0.000338 | 22 | 0.151724 | −0.07723 | 0.10522 | −0.11605 | 0.005198 |
| 184 | CDP-diacylglycerol biosynthetic process | 0.000345 | 7 | 0.5 | 0.192441 | −0.05254 | −0.19068 | 0.005285 |
| 43 | anchored to membrane | 0.000348 | 12 | 0.095238 | 0.008488 | 0.170286 | −0.26789 | 0.0053 |
| 72 | cytoplasmic vesicle membrane | 0.000352 | 52 | 0.436975 | −0.04855 | 0.06129 | −0.04708 | 0.00534 |
| 247 | mitochondrial proton-transporting ATP synthase complex | 0.000358 | 17 | 0.809524 | 0.009968 | 0.074119 | −0.12894 | 0.005401 |
| 58 | structural constituent of cytoskeleton | 0.000363 | 43 | 0.277419 | −0.07932 | 0.005999 | 0.003183 | 0.005455 |
| 250 | NuA4 histone acetyltransferase complex | 0.000367 | 10 | 0.526316 | 0.122766 | −0.14403 | 0.028307 | 0.005473 |
| 251 | histone H2A acetylation | 0.000367 | 10 | 0.588235 | 0.122766 | −0.14403 | 0.028307 | 0.005473 |
| 129 | mitotic spindle | 0.000373 | 19 | 0.575758 | −0.12863 | −0.01536 | 0.159479 | 0.005524 |
| 44 | histone deacetylase activity | 0.000386 | 8 | 0.170213 | 0.029522 | −0.1256 | 0.118448 | 0.005693 |
| 7 | chromosome, centromeric region | 0.000391 | 35 | 0.507246 | 0.029928 | −0.0804 | 0.077759 | 0.005745 |
| 159 | RNA polymerase II transcription factor binding | 0.000396 | 11 | 0.234043 | 0.141107 | −0.13948 | 0.017762 | 0.005796 |
| 189 | stress-activated MAPK cascade | 0.000399 | 27 | 0.457627 | −0.08651 | −0.03594 | 0.120591 | 0.005805 |
| 207 | antigen processing and presentation of exogenous peptide antigen via MHC | 0.0004 | 52 | 0.490566 | −0.01457 | −0.01579 | 0.012593 | 0.005805 |
| | class II | | | | | | | |
| 157 | 2 iron, 2 sulfur cluster binding | 0.000418 | 12 | 0.363636 | 0.016279 | 0.106304 | −0.0999 | 0.006039 |
| 134 | de novo' IMP biosynthetic process | 0.000436 | 6 | 0.6 | −0.14833 | 0.102894 | 0.02011 | 0.006257 |
| 190 | protein deacetylation | 0.000442 | 8 | 0.727273 | −0.03055 | 0.1145 | 0.177156 | 0.006325 |
| 109 | response to starvation | 0.000469 | 10 | 0.175439 | −0.03493 | 0.07223 | −0.07056 | 0.006683 |
| 26 | signalosome | 0.00049 | 24 | 0.428571 | −0.08648 | −0.00991 | 0.094622 | 0.006951 |
| 227 | nucleobase-containing small molecule interconversion | 0.000496 | 16 | 0.888889 | −0.12572 | 0.082345 | 0.089469 | 0.006997 |
| 61 | somitogenesis | 0.000501 | 12 | 0.162162 | 0.109622 | −0.15469 | 0.028021 | 0.007037 |
| 133 | thyroid hormone receptor binding | 0.000505 | 16 | 0.551724 | 0.137006 | −0.13891 | 0.030687 | 0.007062 |
| 160 | peroxisomal matrix | 0.00051 | 23 | 0.638889 | 0.013884 | 0.070204 | −0.10498 | 0.007104 |
| 185 | very long-chain fatty acid metabolic process | 0.000519 | 6 | 0.315789 | 0.12026 | −0.04046 | −0.18221 | 0.007201 |
| 178 | anaphase-promoting complex | 0.000528 | 16 | 0.432432 | −0.12885 | −0.052 | 0.122231 | 0.007292 |
| 209 | tRNA methylation | 0.000539 | 9 | 0.409091 | −0.18051 | 0.015363 | 0.117699 | 0.007414 |
| 205 | blastocyst hatching | 0.000555 | 3 | 0.166667 | 0.211324 | −0.10701 | −0.13018 | 0.007599 |
| 147 | signal peptide processing | 0.000568 | 6 | 0.26087 | 0.110347 | 0.00227 | −0.19979 | 0.007749 |
| 135 | small-subunit processome | 0.000596 | 9 | 0.473684 | 0.116951 | −0.11034 | −0.00289 | 0.008086 |
| 80 | protein dephosphorylation | 0.000605 | 35 | 0.244755 | −0.0257 | −0.01538 | 0.095514 | 0.008156 |
| 199 | cell adhesion molecule binding | 0.000606 | 14 | 0.297872 | −0.0367 | 0.114708 | −0.09618 | 0.008156 |
| 21 | collagen binding | 0.000618 | 23 | 0.157534 | 0.113374 | −0.03584 | −0.09703 | 0.008287 |
| 170 | neurogenesis | 0.000633 | 7 | 0.194444 | −0.20103 | 0.077332 | 0.086537 | 0.008431 |
| 173 | stress fiber assembly | 0.000634 | 3 | 0.214286 | −0.06578 | −0.1713 | 0.202157 | 0.008431 |
| 70 | nucleotide biosynthetic process | 0.000638 | 5 | 0.192308 | −0.19295 | −0.12374 | 0.251821 | 0.008447 |
| 200 | natural killer cell mediated cytotoxicity | 0.000647 | 6 | 0.315789 | −0.1951 | 0.149906 | 0.022303 | 0.008534 |
| 171 | cytoplasmic stress granule | 0.000666 | 27 | 0.55102 | −0.078 | 0.07114 | −0.0299 | 0.008727 |
| 125 | mitotic sister chromatid segregation | 0.000669 | 16 | 0.666667 | −0.12167 | −0.08992 | 0.152312 | 0.008727 |
| 51 | microtubule cytoskeleton | 0.00067 | 61 | 0.317708 | −0.02645 | −0.01958 | 0.045348 | 0.008727 |
| 236 | transcription elongation from RNA polymerase I promoter | 0.000678 | 13 | 0.619048 | 0.089633 | −0.11298 | −0.04193 | 0.008802 |
| 175 | grooming behavior | 0.000723 | 4 | 0.166667 | −0.07312 | −0.10665 | 0.167383 | 0.009319 |
| 55 | phospholipid biosynthetic process | 0.000726 | 10 | 0.217391 | 0.084369 | 0.032999 | −0.12047 | 0.009319 |
| 92 | microtubule bundle formation | 0.000727 | 11 | 0.34375 | −0.2003 | 0.01471 | 0.117994 | 0.009319 |
| 96 | microtubule associated complex | 0.000751 | 17 | 0.515152 | −0.05546 | 0.118891 | −0.04635 | 0.009594 |
| 76 | regulation of cell cycle | 0.000757 | 45 | 0.269461 | −0.01198 | −0.0694 | 0.075213 | 0.009626 |
| 181 | protein serine/threonine/tyrosine kinase activity | 0.000768 | 13 | 0.351351 | −0.14239 | −0.07836 | 0.208618 | 0.009725 |
| 234 | integral to mitochondrial inner membrane | 0.000771 | 13 | 0.866667 | 0.041683 | 0.097702 | −0.0773 | 0.009725 |
| 121 | NAD-dependent histone deacetylase activity (H3-K9 specific) | 0.000776 | 6 | 0.4 | 0.026897 | −0.12567 | 0.11708 | 0.009749 |
| 176 | clathrin binding | 0.000781 | 9 | 0.243243 | −0.01664 | 0.100249 | −0.12557 | 0.009785 |

TABLE 3
Protein set enrichment in the space of correlations between proteins and the CDC protein markers

| | G1_M | S_M | G2_M | blank | G1_U | S_U | G2_U | diff |
|---|---|---|---|---|---|---|---|---|
| regulation of acetyl-CoA biosynthetic process | −0.0173 | 0.1480 | −0.0578 | 0 | −0.0726 | 0.1684 | −0.0726 | 0.1686 |
| from pyruvate | | | | | | | | |
| purine base metabolic process | −0.0663 | 0.0265 | 0.0067 | 0 | −0.0534 | 0.0523 | 0.0069 | 0.1840 |
| cellular nitrogen compound metabolic process | −0.0308 | 0.0259 | 0.0081 | 0 | −0.0510 | 0.0281 | 0.0163 | 0.1906 |
| alternative nuclear mRNA splicing, via | 0.1823 | −0.0950 | −0.0950 | 0 | 0.1478 | −0.1259 | −0.0289 | 0.1949 |
| spliceosome | | | | | | | | |
| nucleosome | 0.3259 | −0.0833 | −0.0793 | 0 | 0.1835 | −0.0989 | −0.0487 | 0.2301 |
| retrograde vesicle-mediated transport, Golgi to ER | −0.0544 | 0.0513 | −0.0160 | 0 | −0.0795 | 0.0502 | 0.0239 | 0.2403 |
| protein polyubiquitination | −0.0308 | 0.0259 | 0.0286 | 0 | −0.0375 | 0.0221 | 0.0034 | 0.2411 |
| nucleobase-containing small molecule metabolic | −0.0550 | 0.0286 | 0.0299 | 0 | −0.0361 | 0.0268 | 0.0056 | 0.2467 |
| process | | | | | | | | |
| DNA damage response, signal transduction by p53 | −0.0321 | 0.0264 | 0.0240 | 0 | −0.0488 | 0.0232 | 0.0043 | 0.2494 |
| class mediator resulting in cell cycle arrest | | | | | | | | |
| neuromuscular process controlling balance | −0.0671 | 0.0683 | −0.0536 | 0 | −0.0821 | 0.0873 | 0.0131 | 0.2707 |
| cerebral cortex development | −0.0900 | 0.0084 | 0.0429 | 0 | −0.0509 | 0.0201 | 0.0212 | 0.3105 |
| chromatin organization | 0.1124 | −0.0253 | −0.0372 | 0 | 0.0583 | −0.0559 | −0.0242 | 0.3119 |
| glycosphingolipid metabolic process | −0.0694 | 0.1001 | −0.0406 | 0 | −0.0317 | 0.1313 | 0.0103 | 0.3123 |
| long-chain fatty-acyl-CoA biosynthetic process | −0.1165 | 0.0725 | 0.0822 | 0 | −0.0753 | 0.0499 | 0.0060 | 0.3479 |
| triglyceride biosynthetic process | −0.1165 | 0.0725 | 0.0822 | 0 | −0.0753 | 0.0499 | 0.0060 | 0.3479 |
| regulation of cellular amino acid metabolic process | −0.0253 | 0.0210 | 0.0286 | 0 | −0.0483 | 0.0377 | 0.0053 | 0.3793 |
| proteasome complex | −0.0294 | 0.0185 | 0.0286 | 0 | −0.0483 | 0.0377 | 0.0034 | 0.3822 |
| antioxidant activity | −0.0832 | 0.0735 | 0.0126 | 0 | −0.0284 | 0.0584 | −0.0300 | 0.3931 |
| positive regulation of ubiquitin-protein ligase | −0.0273 | 0.0193 | 0.0180 | 0 | −0.0470 | 0.0336 | −0.0116 | 0.4053 |
| activity involved in mitotic cell cycle | | | | | | | | |
| sphingolipid metabolic process | −0.0004 | 0.0974 | −0.0447 | 0 | −0.0778 | 0.1134 | −0.0008 | 0.4104 |
| organ regeneration | −0.0742 | 0.0793 | 0.0403 | 0 | −0.0346 | 0.0556 | −0.0494 | 0.4592 |
| regulation of alternative nuclear mRNA splicing, | 0.0798 | −0.0692 | −0.0266 | 0 | 0.0142 | −0.0588 | 0.0231 | 0.4625 |
| via spliceosome | | | | | | | | |
| chromatin DNA binding | 0.1894 | −0.0544 | −0.0460 | 0 | 0.0384 | −0.0232 | −0.0427 | 0.4708 |
| AU-rich element binding | 0.1265 | −0.0172 | −0.0241 | 0 | 0.0471 | −0.0167 | −0.1082 | 0.4826 |
| oxidative phosphorylation | 0.0229 | 0.0636 | −0.0853 | 0 | 0.0238 | 0.0762 | 0.0631 | 0.4834 |
| extrinsic to plasma membrane | −0.0251 | 0.1114 | −0.0378 | 0 | −0.0302 | 0.0340 | −0.1490 | 0.4999 |
| respiratory chain | 0.1530 | 0.0415 | −0.0414 | 0 | 0.0531 | 0.0066 | −0.1126 | 0.5045 |
| actin filament binding | −0.0745 | 0.0536 | −0.0167 | 0 | −0.0227 | 0.0244 | 0.0102 | 0.5343 |
| proteasome core complex, alpha-subunit complex | −0.0081 | 0.0604 | −0.0239 | 0 | −0.0596 | 0.0150 | −0.0361 | 0.5367 |
| nuclear chromatin | 0.1070 | −0.0256 | −0.0186 | 0 | 0.0415 | −0.0624 | 0.0375 | 0.5417 |
| binding of sperm to zona pellucida | −0.0760 | −0.0533 | 0.0545 | 0 | 0.0272 | −0.0177 | 0.0382 | 0.5812 |
| nucleosome assembly | 0.2323 | 0.0107 | −0.0460 | 0 | 0.0541 | −0.0525 | −0.0334 | 0.5920 |
| nuclear euchromatin | 0.1838 | −0.0875 | −0.0548 | 0 | 0.0585 | −0.0336 | 0.0436 | 0.6011 |
| cell body | −0.0816 | 0.0346 | 0.0302 | 0 | −0.0031 | 0.0092 | 0.0236 | 0.6066 |
| catalytic step 2 spliceosome | 0.0534 | −0.0448 | −0.0107 | 0 | 0.0002 | −0.0277 | 0.0050 | 0.6068 |
| cytochrome-c oxidase activity | 0.1105 | 0.0092 | −0.0635 | 0 | 0.0132 | 0.0153 | −0.0190 | 0.6407 |
| phosphate ion binding | −0.0613 | 0.0001 | 0.0791 | 0 | −0.0882 | 0.1059 | −0.0216 | 0.6553 |
| membrane organization | −0.0648 | 0.0390 | 0.0459 | 0 | −0.0170 | 0.0083 | 0.0057 | 0.6574 |
| cytosolic large ribosomal subunit | −0.0926 | −0.1471 | 0.0440 | 0 | 0.0624 | −0.0509 | 0.0204 | 0.6583 |
| GDP binding | −0.0094 | 0.0540 | 0.0012 | 0 | −0.0506 | 0.0165 | −0.0248 | 0.6690 |
| small ribosomal subunit | −0.1499 | −0.0633 | 0.0859 | 0 | 0.0576 | −0.0737 | 0.0071 | 0.6782 |
| translation elongation factor activity | −0.1778 | −0.1602 | 0.1270 | 0 | 0.0296 | −0.0584 | 0.0303 | 0.6960 |
| actin filament polymerization | −0.2064 | 0.0673 | 0.0156 | 0 | 0.0176 | 0.0591 | −0.0257 | 0.6981 |
| Z disc | −0.0552 | −0.0183 | 0.0394 | 0 | −0.0196 | 0.0035 | 0.0001 | 0.7103 |
| endoplasmic reticulum unfolded protein response | 0.0098 | 0.0267 | −0.0083 | 0 | −0.0295 | 0.0125 | 0.0067 | 0.7328 |
| translational elongation | −0.1143 | −0.1466 | 0.0610 | 0 | 0.0598 | −0.0375 | 0.0168 | 0.7511 |
| rRNA binding | −0.1135 | −0.1256 | 0.0460 | 0 | 0.0597 | −0.0239 | 0.0241 | 0.7556 |
| ruffle | −0.0776 | −0.0450 | 0.0215 | 0 | −0.0157 | 0.0169 | 0.0063 | 0.7595 |
| viral transcription | −0.1080 | −0.1466 | 0.0607 | 0 | 0.0598 | −0.0337 | 0.0168 | 0.7630 |
| chaperonin-containing T-complex | −0.0810 | −0.0653 | 0.0428 | 0 | 0.0229 | −0.0061 | 0.0190 | 0.7884 |
| sperm protein complex | −0.0810 | −0.0653 | 0.0428 | 0 | 0.0229 | −0.0061 | 0.0190 | 0.7884 |
| SRP-dependent cotranslational protein targeting to | −0.0926 | −0.1141 | 0.0447 | 0 | 0.0544 | −0.0221 | 0.0126 | 0.7962 |
| membrane | | | | | | | | |
| translational termination | −0.1075 | −0.1465 | 0.0552 | 0 | 0.0590 | −0.0264 | 0.0141 | 0.8017 |
| nuclear-transcribed mRNA catabolic process, | −0.1017 | −0.1255 | 0.0489 | 0 | 0.0536 | −0.0221 | 0.0141 | 0.8020 |
| nonsense-mediated decay | | | | | | | | |
| natural killer cell mediated cytotoxicity | −0.1795 | −0.1473 | 0.1505 | 0 | 0.1150 | −0.0569 | 0.0053 | 0.8098 |
| viral infectious cycle | −0.1058 | −0.1421 | 0.0552 | 0 | 0.0587 | −0.0221 | 0.0141 | 0.8180 |
| ribosomal small subunit biogenesis | −0.1175 | 0.1554 | 0.1020 | 0 | 0.0401 | −0.0075 | 0.0324 | 0.8247 |
| de novo' posttranslational protein folding | −0.0722 | 0.0462 | 0.0672 | 0 | 0.0095 | −0.0191 | 0.0198 | 0.8310 |
| respiratory electron transport chain | 0.0598 | 0.0192 | −0.0362 | 0 | 0.0061 | −0.0161 | −0.0058 | 0.8347 |
| platelet degranulation | −0.0640 | 0.0689 | 0.0388 | 0 | 0.0205 | 0.0161 | −0.0165 | 0.8570 |
| blood microparticle | −0.1046 | −0.0898 | 0.0689 | 0 | 0.0150 | −0.0190 | −0.0076 | 0.8754 |
| protein disulfide isomerase activity | 0.0917 | 0.0573 | −0.1152 | 0 | −0.0138 | 0.0157 | −0.0005 | 0.8895 |
| glycolysis | −0.1081 | −0.0693 | 0.0546 | 0 | −0.0146 | 0.0070 | −0.0566 | 0.9062 |
| positive regulation of protein insertion into | −0.0695 | 0.0411 | 0.0880 | 0 | 0.0210 | −0.0036 | 0.0101 | 0.9134 |
| mitochondrial membrane involved in apoptotic | | | | | | | | |
| signaling pathway | | | | | | | | |
| MHC class II protein complex binding | −0.1600 | −0.0124 | 0.1197 | 0 | 0.0905 | −0.0065 | 0.0106 | 0.9143 |
| cytosolic small ribosomal subunit | −0.1230 | −0.1266 | 0.0849 | 0 | 0.0690 | −0.0075 | −0.0010 | 0.9634 |
| protein polymerization | −0.1994 | 0.0203 | 0.1495 | 0 | 0.0768 | 0.0050 | −0.0002 | 0.9776 |
| microtubule-based process | −0.1498 | 0.0291 | 0.0908 | 0 | 0.0238 | −0.0308 | −0.0002 | 1 |
| cellular component movement | −0.0188 | 0.0209 | 0.0220 | 0 | 0.0293 | −0.0229 | −0.0067 | 1 |
| male gonad development | −0.1006 | 0.0477 | 0.0322 | 0 | 0.0125 | −0.0015 | −0.0080 | 1 |
| negative regulation of protein kinase activity | −0.1137 | 0.0293 | 0.0910 | 0 | 0.0457 | 0.0081 | −0.0852 | 1 |
| prefoldin complex | 0.0078 | −0.0066 | 0.1214 | 0 | −0.0901 | 0.0197 | −0.0170 | 1 |
| somitogenesis | 0.2031 | −0.0837 | −0.0527 | 0 | −0.0755 | 0.0226 | 0.0089 | 1 |

TABLE 4
Additional proteins that correlate significantly to the CDC protein markers

| | prot | Phase | pval | cor | qval | celltype |
|---|---|---|---|---|---|---|
| 1 | O00483 | G1 | 0.002768 | 0.169348 | 0.037656 | Melanoma |
| 2 | O14949 | G1 | 0.002344 | 0.147051 | 0.033665 | Melanoma |
| 3 | O14949 | S | 0.000517 | 0.204846 | 0.011391 | Melanoma |
| 4 | O14979 | G1 | 5.59E−07 | 0.215654 | 4.49E−05 | Melanoma |
| 5 | O15143 | S | 0.002802 | 0.098584 | 0.037903 | Melanoma |
| 6 | O43390 | G1 | 9.54E−06 | 0.172743 | 0.000516 | Melanoma |
| 7 | O43684 | G1 | 0.002057 | 0.165257 | 0.030478 | Melanoma |
| 8 | O43809 | G1 | 2.46E−05 | 0.178244 | 0.001021 | Melanoma |
| 9 | O60313 | S | 0.001803 | 0.163247 | 0.028151 | Melanoma |
| 10 | O60637 | G1 | 0.000187 | 0.211688 | 0.00515 | Melanoma |
| 11 | O60869 | G2 | 7.04E−08 | 0.238294 | 7.28E−06 | Melanoma |
| 12 | O60925 | G2 | 0.003253 | 0.131627 | 0.041701 | Melanoma |
| 13 | O75153 | G2 | 7.64E−24 | 0.544395 | 2.09E−21 | Melanoma |
| 14 | O75367 | G1 | 0.001815 | 0.138599 | 0.028238 | Melanoma |
| 15 | O75390 | G1 | 0.002781 | 0.07631 | 0.037731 | Melanoma |
| 16 | O75494 | G1 | 0.000787 | 0.226767 | 0.01602 | Melanoma |
| 17 | O75494 | S | 0.000949 | 0.175789 | 0.018477 | Melanoma |
| 18 | O94776 | S | 1.53E−05 | 0.161673 | 0.000741 | Melanoma |
| 19 | O95433 | S | 0.002129 | 0.138191 | 0.031154 | Melanoma |
| 20 | O95881 | S | 0.003864 | 0.13557 | 0.046574 | Melanoma |
| 21 | P00403 | G1 | 0.00131 | 0.16342 | 0.022751 | Melanoma |
| 22 | P00441 | S | 9.92E−59 | 0.600106 | 6.59E−56 | Melanoma |
| 23 | P00505 | G1 | 0.004013 | 0.137244 | 0.047995 | Melanoma |
| 24 | P02545 | G1 | 2.02E−11 | 0.329407 | 3.25E−09 | Melanoma |
| 25 | P04406 | G2 | 0.000387 | 0.168334 | 0.009187 | Melanoma |
| 26 | P04792 | G2 | 0.000401 | 0.133196 | 0.009432 | Melanoma |
| 27 | P06748 | G1 | 6.66E−09 | 0.342976 | 7.94E−07 | Melanoma |
| 28 | P07195 | G2 | 0.001065 | 0.13238 | 0.019987 | Melanoma |
| 29 | P07305 | G1 | 2.17E−09 | 0.299325 | 2.80E−07 | Melanoma |
| 30 | P07437 | G2 | 0.001428 | 0.158986 | 0.024079 | Melanoma |
| 31 | P07910 | G1 | 0.002543 | 0.195706 | 0.035751 | Melanoma |
| 32 | P08311 | S | 8.90E−05 | 0.123551 | 0.002857 | Melanoma |
| 33 | P08567 | G2 | 0.00035 | 0.214853 | 0.008699 | Melanoma |
| 34 | P08670 | G1 | 0.000915 | 0.197406 | 0.017885 | Melanoma |
| 35 | P09012 | S | 0.00289 | 0.092693 | 0.038308 | Melanoma |
| 36 | P09651 | G1 | 0.001176 | 0.148675 | 0.021288 | Melanoma |
| 37 | P09669 | G1 | 8.03E−09 | 0.213605 | 9.34E−07 | Melanoma |
| 38 | P10412 | G1 | 2.78E−27 | 0.388801 | 8.62E−25 | Melanoma |
| 39 | P10809 | G1 | 0.002616 | 0.099564 | 0.036551 | Melanoma |
| 40 | P11310 | S | 1.44E−29 | 0.429362 | 4.79E−27 | Melanoma |
| 41 | P11387 | G1 | 0.002001 | 0.114461 | 0.030142 | Melanoma |
| 42 | P11940 | G2 | 1.83E−05 | 0.167822 | 0.000811 | Melanoma |
| 43 | P12236 | G1 | 0.002655 | 0.06312 | 0.036771 | Melanoma |
| 44 | P12956 | G1 | 2.07E−05 | 0.218699 | 0.000907 | Melanoma |
| 45 | P13010 | G1 | 8.90E−05 | 0.272558 | 0.002857 | Melanoma |
| 46 | P13473 | S | 0.001462 | 0.140278 | 0.024566 | Melanoma |
| 47 | P13667 | G1 | 0.001112 | 0.148656 | 0.020607 | Melanoma |
| 48 | P14174 | G2 | 0.000326 | 0.162901 | 0.008246 | Melanoma |
| 49 | P14324 | G2 | 1.40E−54 | 0.597771 | 8.15E−52 | Melanoma |
| 50 | P14618 | G2 | 0.001114 | 0.149989 | 0.020607 | Melanoma |
| 51 | P14854 | G1 | 0.003012 | 0.075339 | 0.039477 | Melanoma |
| 52 | P14866 | G1 | 0.003711 | 0.123486 | 0.045559 | Melanoma |
| 53 | P14866 | S | 0.003516 | 0.069344 | 0.044212 | Melanoma |
| 54 | P14927 | G1 | 0.001216 | 0.164223 | 0.021908 | Melanoma |
| 55 | P15559 | G2 | 0.00043 | 0.161882 | 0.010054 | Melanoma |
| 56 | P16104 | G1 | 5.27E−40 | 0.546449 | 2.45E−37 | Melanoma |
| 57 | P16150 | S | 0.001531 | 0.189542 | 0.02535 | Melanoma |
| 58 | P16401 | G1 | 3.12E−213 | 0.883314 | 1.45E−209 | Melanoma |
| 59 | P16402 | G1 | 6.70E−14 | 0.295448 | 1.42E−11 | Melanoma |
| 60 | P16403 | G1 | 1.75E−102 | 0.729705 | 2.71E−99 | Melanoma |
| 61 | P16949 | G2 | 0.002363 | 0.118805 | 0.033828 | Melanoma |
| 62 | P17096 | G1 | 0.000472 | 0.112873 | 0.01076 | Melanoma |
| 63 | P17844 | G1 | 0.001129 | 0.173822 | 0.020607 | Melanoma |
| 64 | P17931 | G2 | 0.0017 | 0.140174 | 0.027269 | Melanoma |
| 65 | P19338 | G1 | 0.00288 | 0.213242 | 0.03829 | Melanoma |
| 66 | P19838 | S | 0.003787 | 0.292409 | 0.046019 | Melanoma |
| 67 | P19878 | G1 | 0.000867 | 0.317421 | 0.017094 | Melanoma |
| 68 | P20290 | G2 | 0.00304 | 0.150959 | 0.039737 | Melanoma |
| 69 | P20671 | G1 | 9.48E−14 | 0.500856 | 1.92E−11 | Melanoma |
| 70 | P20700 | G1 | 3.28E−06 | 0.161075 | 0.000206 | Melanoma |
| 71 | P20962 | G2 | 0.00033 | 0.139458 | 0.008247 | Melanoma |
| 72 | P21333 | G2 | 0.003579 | 0.09646 | 0.044644 | Melanoma |
| 73 | P22087 | G1 | 2.02E−11 | 0.317622 | 3.25E−09 | Melanoma |
| 74 | P22307 | S | 0.002059 | 0.135241 | 0.030478 | Melanoma |
| 75 | P22626 | G1 | 3.41E−09 | 0.242429 | 4.29E−07 | Melanoma |
| 76 | P23246 | G1 | 8.62E−07 | 0.287383 | 6.37E−05 | Melanoma |
| 77 | P24158 | S | 0.00052 | 0.223539 | 0.011414 | Melanoma |
| 78 | P24534 | G2 | 0.002112 | 0.156845 | 0.031004 | Melanoma |
| 79 | P24539 | G1 | 0.000123 | 0.14206 | 0.003755 | Melanoma |
| 80 | P25705 | G1 | 3.43E−06 | 0.137582 | 0.000213 | Melanoma |
| 81 | P25787 | S | 6.11E−06 | 0.232148 | 0.000351 | Melanoma |
| 82 | P26599 | G1 | 6.91E−06 | 0.212563 | 0.000392 | Melanoma |
| 83 | P27348 | G2 | 0.004145 | 0.112018 | 0.049308 | Melanoma |
| 84 | P27824 | S | 0.001765 | 0.112216 | 0.027844 | Melanoma |
| 85 | P28072 | S | 0.002875 | 0.111916 | 0.03829 | Melanoma |
| 86 | P30084 | S | 0.003094 | 0.144295 | 0.040217 | Melanoma |
| 87 | P30101 | G1 | 0.001226 | 0.141828 | 0.021945 | Melanoma |
| 88 | P31040 | G1 | 0.002479 | 0.112175 | 0.035167 | Melanoma |
| 89 | P31040 | S | 0.003773 | 0.106521 | 0.046019 | Melanoma |
| 90 | P33991 | G1 | 0.004082 | 0.080676 | 0.048706 | Melanoma |
| 91 | P35232 | G1 | 1.56E−08 | 0.180357 | 1.69E−06 | Melanoma |
| 92 | P35613 | S | 9.12E−38 | 0.472115 | 3.86E−35 | Melanoma |
| 93 | P36957 | G1 | 0.0002 | 0.105997 | 0.005429 | Melanoma |
| 94 | P37108 | G1 | 0.003654 | 0.141164 | 0.045217 | Melanoma |
| 95 | P38159 | G1 | 8.92E−10 | 0.269653 | 1.24E−07 | Melanoma |
| 96 | P38646 | G1 | 0.000359 | 0.09239 | 0.008844 | Melanoma |
| 97 | P40926 | G1 | 5.37E−06 | 0.187222 | 0.000312 | Melanoma |
| 98 | P43243 | G1 | 4.00E−11 | 0.289435 | 6.20E−09 | Melanoma |
| 99 | P43307 | S | 0.002922 | 0.145585 | 0.038623 | Melanoma |
| 100 | P47985 | G1 | 4.25E−05 | 0.184489 | 0.001585 | Melanoma |
| 101 | P48637 | S | 5.85E−30 | 0.45043 | 2.09E−27 | Melanoma |
| 102 | P49327 | G2 | 0.000834 | 0.156784 | 0.016799 | Melanoma |
| 103 | P49411 | G1 | 0.001028 | 0.128178 | 0.019522 | Melanoma |
| 104 | P50402 | G1 | 1.18E−05 | 0.139485 | 0.000603 | Melanoma |
| 105 | P50502 | G2 | 0.001378 | 0.147762 | 0.023458 | Melanoma |
| 106 | P51991 | G1 | 7.98E−06 | 0.224153 | 0.000447 | Melanoma |
| 107 | P52272 | G1 | 3.16E−06 | 0.15495 | 0.000202 | Melanoma |
| 108 | P52434 | G2 | 0.001978 | 0.147077 | 0.029984 | Melanoma |
| 109 | P56381 | G1 | 9.74E−07 | 0.199585 | 7.08E−05 | Melanoma |
| 110 | P56545 | G1 | 0.00163 | 0.185884 | 0.026524 | Melanoma |
| 111 | P61006 | S | 0.00238 | 0.199354 | 0.033969 | Melanoma |
| 112 | P61026 | G1 | 0.000757 | 0.10029 | 0.015576 | Melanoma |
| 113 | P61289 | G2 | 5.75E−63 | 0.597737 | 4.46E−60 | Melanoma |
| 114 | P61604 | S | 0.002164 | 0.110594 | 0.031562 | Melanoma |
| 115 | P62306 | S | 0.000839 | 0.217185 | 0.016835 | Melanoma |
| 116 | P62314 | G1 | 0.000358 | 0.142013 | 0.008844 | Melanoma |
| 117 | P62805 | G1 | 2.08E−179 | 0.870036 | 4.84E−176 | Melanoma |
| 118 | P62807 | G1 | 7.90E−84 | 0.693619 | 9.19E−81 | Melanoma |
| 119 | P62826 | G2 | 0.002741 | 0.171895 | 0.037622 | Melanoma |
| 120 | P63162 | S | 3.91E−05 | 0.18419 | 0.001516 | Melanoma |
| 121 | P68371 | G2 | 7.45E−05 | 0.153233 | 0.002494 | Melanoma |
| 122 | P78527 | G1 | 1.97E−14 | 0.257906 | 4.37E−12 | Melanoma |
| 123 | P84103 | G1 | 3.80E−07 | 0.228718 | 3.34E−05 | Melanoma |
| 124 | P99999 | G1 | 5.63E−05 | 0.194457 | 0.002016 | Melanoma |
| 125 | Q00325 | G1 | 6.44E−07 | 0.152629 | 4.99E−05 | Melanoma |
| 126 | Q00839 | G1 | 3.45E−15 | 0.379258 | 8.45E−13 | Melanoma |
| 127 | Q01130 | G1 | 0.001927 | 0.047233 | 0.029495 | Melanoma |
| 128 | Q03252 | G1 | 0.001716 | 0.104034 | 0.027345 | Melanoma |
| 129 | Q07666 | G1 | 9.47E−08 | 0.248984 | 9.58E−06 | Melanoma |
| 130 | Q07955 | G1 | 5.43E−05 | 0.113668 | 0.00196 | Melanoma |
| 131 | Q08211 | G1 | 0.000115 | 0.198862 | 0.003583 | Melanoma |
| 132 | Q13151 | G1 | 4.16E−05 | 0.170652 | 0.001574 | Melanoma |
| 133 | Q13243 | G2 | 4.04E−05 | 0.397253 | 0.001555 | Melanoma |
| 134 | Q13247 | G1 | 0.000722 | 0.192208 | 0.014991 | Melanoma |
| 135 | Q13257 | G2 | 1.54E−30 | 0.668233 | 5.99E−28 | Melanoma |
| 136 | Q13561 | G1 | 0.002575 | 0.152915 | 0.036091 | Melanoma |
| 137 | Q13595 | S | 0.001903 | 0.238034 | 0.029228 | Melanoma |
| 138 | Q14247 | G2 | 0.000234 | 0.181435 | 0.006153 | Melanoma |
| 139 | Q14677 | G2 | 0.002081 | 0.100842 | 0.030644 | Melanoma |
| 140 | Q14978 | G1 | 2.16E−05 | 0.214911 | 0.000931 | Melanoma |
| 141 | Q15233 | G1 | 0.000189 | 0.218454 | 0.005168 | Melanoma |
| 142 | Q15365 | S | 0.001026 | 0.147359 | 0.019522 | Melanoma |
| 143 | Q15370 | G2 | 0.003911 | 0.123677 | 0.047028 | Melanoma |
| 144 | Q15392 | G1 | 0.001652 | 0.112106 | 0.026777 | Melanoma |
| 145 | Q15424 | G1 | 0.001564 | 0.141457 | 0.025619 | Melanoma |
| 146 | Q15907 | G1 | 0.000561 | 0.1549 | 0.012129 | Melanoma |
| 147 | Q16778 | G1 | 1.80E−40 | 0.522673 | 9.32E−38 | Melanoma |
| 148 | Q16836 | G1 | 8.07E−05 | 0.123684 | 0.002662 | Melanoma |
| 149 | Q16891 | G1 | 0.001888 | 0.1483 | 0.029228 | Melanoma |
| 150 | Q5XPI4 | G1 | 0.003822 | 0.167665 | 0.04619 | Melanoma |
| 151 | Q71DI3 | G1 | 6.16E−66 | 0.598935 | 5.73E−63 | Melanoma |
| 152 | Q7Z434 | S | 1.62E−14 | 0.450597 | 3.77E−12 | Melanoma |
| 153 | Q86UE4 | S | 7.37E−05 | 0.12934 | 0.002485 | Melanoma |
| 154 | Q8TCJ2 | G1 | 0.002008 | 0.156284 | 0.030142 | Melanoma |
| 155 | Q8TER0 | S | 0.003351 | 0.344735 | 0.042724 | Melanoma |
| 156 | Q92522 | G1 | 1.54E−06 | 0.174952 | 0.000103 | Melanoma |
| 157 | Q92616 | S | 0.000648 | 0.131248 | 0.013753 | Melanoma |
| 158 | Q92945 | G1 | 0.000773 | 0.084775 | 0.015846 | Melanoma |
| 159 | Q92947 | G1 | 0.000232 | 0.247869 | 0.006131 | Melanoma |
| 160 | Q96AE4 | G1 | 0.002507 | 0.09384 | 0.03546 | Melanoma |
| 161 | Q96SU4 | S | 0.002725 | 0.121777 | 0.03751 | Melanoma |
| 162 | Q99623 | G1 | 8.36E−10 | 0.209161 | 1.22E−07 | Melanoma |
| 163 | Q99729 | G1 | 6.47E−11 | 0.298252 | 9.72E−09 | Melanoma |
| 164 | Q99848 | G1 | 5.01E−07 | 0.268132 | 4.17E−05 | Melanoma |
| 165 | Q99878 | G1 | 9.61E−25 | 0.371213 | 2.80E−22 | Melanoma |
| 166 | Q99880 | G1 | 6.92E−18 | 0.413637 | 1.79E−15 | Melanoma |
| 167 | Q9BQE3 | G2 | 0.00013 | 0.209228 | 0.003891 | Melanoma |
| 168 | Q9BVC6 | G1 | 5.35E−05 | 0.133219 | 0.001944 | Melanoma |
| 169 | Q9BZH6 | S | 0.000477 | 0.139022 | 0.01082 | Melanoma |
| 170 | Q9H773 | S | 0.000181 | 0.21258 | 0.005022 | Melanoma |
| 171 | Q9NVP1 | G1 | 0.000162 | 0.133282 | 0.004673 | Melanoma |
| 172 | Q9NX63 | G1 | 0.001629 | 0.129154 | 0.026524 | Melanoma |
| 173 | Q9NX63 | S | 0.00086 | 0.204211 | 0.017094 | Melanoma |
| 174 | Q9UBM7 | G1 | 1.13E−08 | 0.22183 | 1.25E−06 | Melanoma |
| 175 | Q9UKM9 | G1 | 1.53E−09 | 0.269349 | 2.04E−07 | Melanoma |
| 176 | Q9UMS4 | G1 | 1.44E−07 | 0.228575 | 1.42E−05 | Melanoma |
| 177 | Q9Y277 | S | 0.00175 | 0.144972 | 0.027758 | Melanoma |
| 178 | O14979 | G1 | 0.000277 | 0.125208 | 0.020463 | Monocyte |
| 179 | P00441 | S | 3.46E−101 | 0.73174 | 5.37E−98 | Monocyte |
| 180 | P00558 | G1 | 0.000114 | 0.176898 | 0.010231 | Monocyte |
| 181 | P04075 | G1 | 5.76E−08 | 0.216304 | 1.58E−05 | Monocyte |
| 182 | P05204 | G1 | 0.00078 | 0.151994 | 0.043739 | Monocyte |
| 183 | P06748 | G1 | 3.58E−05 | 0.185525 | 0.003964 | Monocyte |
| 184 | P07437 | G1 | 0.000189 | 0.189549 | 0.014438 | Monocyte |
| 185 | P09429 | G1 | 4.87E−08 | 0.202385 | 1.41E−05 | Monocyte |
| 186 | P09651 | G1 | 9.44E−05 | 0.137829 | 0.008872 | Monocyte |
| 187 | P10412 | G1 | 2.25E−08 | 0.165507 | 7.47E−06 | Monocyte |
| 188 | P11142 | G1 | 0.000732 | 0.148992 | 0.041553 | Monocyte |
| 189 | P11310 | S | 2.86E−30 | 0.531988 | 1.33E−27 | Monocyte |
| 190 | P12236 | G1 | 2.92E−05 | 0.14846 | 0.003393 | Monocyte |
| 191 | P16104 | G1 | 5.66E−12 | 0.243373 | 2.19E−09 | Monocyte |
| 192 | P16401 | G1 | 1.66E−150 | 0.7938 | 7.72E−147 | Monocyte |
| 193 | P16403 | G1 | 2.22E−38 | 0.46972 | 1.48E−35 | Monocyte |
| 194 | P17844 | G1 | 9.53E−05 | 0.16121 | 0.008872 | Monocyte |
| 195 | P18124 | G1 | 8.55E−06 | 0.175857 | 0.001326 | Monocyte |
| 196 | P19338 | G1 | 3.20E−07 | 0.210507 | 7.84E−05 | Monocyte |
| 197 | P22087 | G1 | 6.36E−06 | 0.172042 | 0.001138 | Monocyte |
| 198 | P22626 | G1 | 7.75E−06 | 0.167487 | 0.001265 | Monocyte |
| 199 | P23141 | S | 0.000433 | 0.134933 | 0.028954 | Monocyte |
| 200 | P23297 | S | 2.65E−05 | 0.146244 | 0.00316 | Monocyte |
| 201 | P24534 | G1 | 7.15E−05 | 0.142242 | 0.007078 | Monocyte |
| 202 | P26373 | G1 | 2.09E−05 | 0.155062 | 0.002669 | Monocyte |
| 203 | P29401 | G1 | 9.24E−06 | 0.19487 | 0.001344 | Monocyte |
| 204 | P30084 | S | 0.000672 | 0.125445 | 0.039092 | Monocyte |
| 205 | P31350 | G1 | 8.41E−05 | 0.168671 | 0.008152 | Monocyte |
| 206 | P31949 | G1 | 0.000419 | 0.077157 | 0.028698 | Monocyte |
| 207 | P35613 | S | 3.64E−54 | 0.551083 | 3.39E−51 | Monocyte |
| 208 | P39023 | G1 | 3.48E−05 | 0.144492 | 0.003954 | Monocyte |
| 209 | P46776 | G1 | 9.11E−06 | 0.175346 | 0.001344 | Monocyte |
| 210 | P46781 | G1 | 0.000458 | 0.120098 | 0.029581 | Monocyte |
| 211 | P48637 | S | 5.13E−35 | 0.501312 | 2.65E−32 | Monocyte |
| 212 | P49207 | G1 | 1.35E−05 | 0.168547 | 0.001798 | Monocyte |
| 213 | P49588 | S | 0.000842 | 0.177025 | 0.045668 | Monocyte |
| 214 | P51452 | S | 0.000106 | 0.326714 | 0.009684 | Monocyte |
| 215 | P52566 | G1 | 1.39E−07 | 0.203099 | 3.60E−05 | Monocyte |
| 216 | P60709 | G1 | 5.03E−06 | 0.187335 | 0.000971 | Monocyte |
| 217 | P61254 | G1 | 5.22E−06 | 0.177382 | 0.000971 | Monocyte |
| 218 | P62249 | G1 | 0.000307 | 0.138409 | 0.021943 | Monocyte |
| 219 | P62328 | G1 | 3.00E−06 | 0.200186 | 0.000635 | Monocyte |
| 220 | P62701 | G1 | 0.000484 | 0.116107 | 0.030857 | Monocyte |
| 221 | P62805 | G1 | 4.45E−115 | 0.702605 | 1.04E−111 | Monocyte |
| 222 | P62807 | G1 | 4.62E−46 | 0.446036 | 3.59E−43 | Monocyte |
| 223 | P62888 | G1 | 4.50E−05 | 0.180981 | 0.004873 | Monocyte |
| 224 | P62899 | G1 | 0.000668 | 0.117687 | 0.039092 | Monocyte |
| 225 | P84090 | G1 | 2.24E−05 | 0.190342 | 0.002747 | Monocyte |
| 226 | P84103 | G1 | 0.000231 | 0.154852 | 0.017342 | Monocyte |
| 227 | Q00839 | G1 | 0.000129 | 0.146843 | 0.011145 | Monocyte |
| 228 | Q01130 | G1 | 1.12E−05 | 0.162499 | 0.001545 | Monocyte |
| 229 | Q04941 | S | 0.00062 | 0.212157 | 0.037298 | Monocyte |
| 230 | Q13257 | G2 | 5.61E−64 | 0.902197 | 6.53E−61 | Monocyte |
| 231 | Q14247 | S | 0.000583 | 0.126623 | 0.035707 | Monocyte |
| 232 | Q16778 | G1 | 1.86E−22 | 0.320098 | 7.85E−20 | Monocyte |
| 233 | Q71DI3 | G1 | 1.24E−37 | 0.443394 | 7.20E−35 | Monocyte |
| 234 | Q7Z434 | S | 0.000144 | 0.339479 | 0.011545 | Monocyte |
| 235 | Q8IY50 | G1 | 0.000574 | 0.16569 | 0.035637 | Monocyte |
| 236 | Q8TER0 | S | 2.96E−06 | 0.474726 | 0.000635 | Monocyte |
| 237 | Q96I99 | S | 0.000987 | 0.128073 | 0.049918 | Monocyte |
| 238 | Q96KB5 | S | 0.000497 | 0.289828 | 0.031244 | Monocyte |
| 239 | Q99878 | G1 | 3.81E−11 | 0.223383 | 1.36E−08 | Monocyte |
| 240 | Q99880 | G1 | 2.93E−08 | 0.258416 | 9.08E−06 | Monocyte |
| 241 | Q9BQE3 | G1 | 0.000147 | 0.120295 | 0.011626 | Monocyte |
| 242 | Q9UIG0 | G1 | 0.000181 | 0.12043 | 0.014074 | Monocyte |
| 243 | Q9UMS4 | G1 | 7.88E−06 | 0.16617 | 0.001265 | Monocyte |

TABLE 5
Differentially abundant proteins
between melanoma sub-populations

| | pvals | prot | FC | qval | Condition |
|---|---|---|---|---|---|
| 1 | 3.12E−14 | E9PAV3 | −0.82568 | 2.83E−13 | Cluster A |
| 2 | 0.000425 | O00193 | −0.61361 | 0.001112 | Cluster A |
| 3 | 1.86E−05 | O00232 | 0.355623 | 6.46E−05 | Cluster B |
| 4 | 2.29E−16 | O00244 | −0.63274 | 2.46E−15 | Cluster A |
| 5 | 9.88E−09 | O00299 | −0.45768 | 4.96E−08 | Cluster A |
| 6 | 1.49E−07 | O00483 | 0.644639 | 6.53E−07 | Cluster B |
| 7 | 0.002487 | O00541 | 0.200772 | 0.00563 | Cluster B |
| 8 | 2.64E−06 | O00625 | −0.65861 | 1.03E−05 | Cluster A |
| 9 | 1.52E−08 | O14561 | −0.67198 | 7.45E−08 | Cluster A |
| 10 | 8.35E−05 | O14737 | −0.26512 | 0.000248 | Cluster A |
| 11 | 1.27E−05 | O14818 | 0.201452 | 4.56E−05 | Cluster B |
| 12 | 0.000841 | O14880 | 0.506075 | 0.002113 | Cluster B |
| 13 | 1.65E−06 | O14949 | 0.570996 | 6.51E−06 | Cluster B |
| 14 | 3.50E−07 | O14979 | 0.409702 | 1.49E−06 | Cluster B |
| 15 | 0.001232 | O15160 | 0.453257 | 0.003 | Cluster B |
| 16 | 0.000466 | O15258 | 0.258014 | 0.001213 | Cluster B |
| 17 | 2.00E−08 | O15427 | 0.452887 | 9.75E−08 | Cluster B |
| 18 | 0.001684 | O43143 | 0.151812 | 0.003953 | Cluster B |
| 19 | 6.33E−09 | O43149 | −0.8572 | 3.27E−08 | Cluster A |
| 20 | 6.21E−23 | O43175 | −0.7895 | 1.13E−21 | Cluster A |
| 21 | 2.09E−11 | O43390 | 0.478258 | 1.39E−10 | Cluster B |
| 22 | 0.000266 | O43615 | 0.263402 | 0.000723 | Cluster B |
| 23 | 0.000105 | O43660 | 0.532088 | 0.00031 | Cluster B |
| 24 | 6.30E−10 | O43707 | −0.35768 | 3.61E−09 | Cluster A |
| 25 | 6.35E−05 | O43719 | 0.669254 | 0.000196 | Cluster B |
| 26 | 2.23E−07 | O43776 | −0.70144 | 9.66E−07 | Cluster A |
| 27 | 0.000503 | O43809 | 0.312914 | 0.001301 | Cluster B |
| 28 | 9.07E−07 | O43852 | 0.2741 | 3.69E−06 | Cluster B |
| 29 | 6.31E−05 | O60506 | 0.12646 | 0.000195 | Cluster B |
| 30 | 7.20E−07 | O60637 | 0.673247 | 2.96E−06 | Cluster B |
| 31 | 0.001285 | O60701 | −0.37834 | 0.003113 | Cluster A |
| 32 | 5.28E−05 | O60762 | 0.487165 | 0.000168 | Cluster B |
| 33 | 3.36E−08 | O60869 | −0.5431 | 1.58E−07 | Cluster A |
| 34 | 1.34E−05 | O75083 | −0.26055 | 4.80E−05 | Cluster A |
| 35 | 2.08E−30 | O75347 | −1.54061 | 8.86E−29 | Cluster A |
| 36 | 7.55E−05 | O75367 | 0.281095 | 0.000228 | Cluster B |
| 37 | 0.004071 | O75368 | −0.35157 | 0.008915 | Cluster A |
| 38 | 0.000553 | O75390 | 0.182302 | 0.00142 | Cluster B |
| 39 | 5.39E−05 | O75396 | 0.284217 | 0.00017 | Cluster B |
| 40 | 3.19E−05 | O75475 | 0.517588 | 0.000106 | Cluster B |
| 41 | 1.17E−08 | O75494 | 0.382192 | 5.85E−08 | Cluster B |
| 42 | 0.000237 | O75533 | 0.246317 | 0.000646 | Cluster B |
| 43 | 7.48E−06 | O75822 | −0.4246 | 2.78E−05 | Cluster A |
| 44 | 4.88E−19 | O75874 | −0.61024 | 6.57E−18 | Cluster A |
| 45 | 5.63E−09 | O75937 | −0.59283 | 2.92E−08 | Cluster A |
| 46 | 0.001174 | O75947 | 0.244923 | 0.002865 | Cluster B |
| 47 | 0.000212 | O75952 | −0.40123 | 0.000586 | Cluster A |
| 48 | 4.09E−05 | O76021 | 0.292958 | 0.000133 | Cluster B |
| 49 | 0.000103 | O94776 | 0.184518 | 0.000305 | Cluster B |
| 50 | 0.004086 | O94905 | 0.207211 | 0.008934 | Cluster B |
| 51 | 5.88E−10 | O95831 | 0.52995 | 3.39E−09 | Cluster B |
| 52 | 0.001268 | O96000 | 0.423476 | 0.003082 | Cluster B |
| 53 | 2.11E−32 | P00338 | −1.0044 | 1.35E−30 | Cluster A |
| 54 | 0.000141 | P00390 | 0.31313 | 0.000409 | Cluster B |
| 55 | 1.12E−11 | P00403 | 0.549323 | 7.59E−11 | Cluster B |
| 56 | 4.74E−05 | P00491 | 0.558853 | 0.000152 | Cluster B |
| 57 | 1.60E−13 | P00505 | 0.416121 | 1.31E−12 | Cluster B |
| 58 | 3.74E−30 | P00558 | −0.73015 | 1.49E−28 | Cluster A |
| 59 | 1.37E−13 | P02545 | 0.349926 | 1.13E−12 | Cluster B |
| 60 | 0.002469 | P02746 | −0.41888 | 0.0056 | Cluster A |
| 61 | 0.000781 | P02786 | 0.312129 | 0.001973 | Cluster B |
| 62 | 6.88E−37 | P04075 | −1.07509 | 1.76E−34 | Cluster A |
| 63 | 3.12E−13 | P04080 | −0.55725 | 2.42E−12 | Cluster A |
| 64 | 2.18E−34 | P04406 | −1.10982 | 2.32E−32 | Cluster A |
| 65 | 1.21E−15 | P04792 | −0.49493 | 1.26E−14 | Cluster A |
| 66 | 3.10E−11 | P04843 | 0.354144 | 2.00E−10 | Cluster B |
| 67 | 4.50E−13 | P05023 | 0.486066 | 3.40E−12 | Cluster B |
| 68 | 7.75E−07 | P05141 | 0.31598 | 3.17E−06 | Cluster B |
| 69 | 0.000468 | P05198 | −0.24527 | 0.001218 | Cluster A |
| 70 | 0.003529 | P05387 | 0.325099 | 0.007811 | Cluster B |
| 71 | 1.16E−19 | P06396 | −1.06764 | 1.62E−18 | Cluster A |
| 72 | 2.53E−14 | P06454 | −0.93455 | 2.33E−13 | Cluster A |
| 73 | 0.000809 | P06576 | 0.228252 | 0.002036 | Cluster B |
| 74 | 3.62E−30 | P06703 | −1.28798 | 1.49E−28 | Cluster A |
| 75 | 1.53E−37 | P06733 | −1.36521 | 7.99E−35 | Cluster A |
| 76 | 0.001015 | P06737 | −0.22981 | 0.00251 | Cluster A |
| 77 | 7.10E−20 | P06744 | −0.605 | 1.03E−18 | Cluster A |
| 78 | 1.53E−05 | P06748 | 0.381867 | 5.39E−05 | Cluster B |
| 79 | 1.59E−05 | P06753 | −0.42719 | 5.61E−05 | Cluster A |
| 80 | 2.11E−29 | P07195 | −0.89401 | 7.51E−28 | Cluster A |
| 81 | 5.10E−14 | P07237 | 0.354456 | 4.53E−13 | Cluster B |
| 82 | 1.40E−07 | P07305 | 0.567936 | 6.19E−07 | Cluster B |
| 83 | 4.66E−10 | P07339 | 0.348277 | 2.77E−09 | Cluster B |
| 84 | 2.01E−08 | P07355 | −0.27484 | 9.75E−08 | Cluster A |
| 85 | 1.50E−29 | P07437 | −1.38465 | 5.49E−28 | Cluster A |
| 86 | 0.00053 | P07686 | 0.466031 | 0.001363 | Cluster B |
| 87 | 1.65E−18 | P07737 | −0.68596 | 2.15E−17 | Cluster A |
| 88 | 2.29E−18 | P07814 | −0.55223 | 2.92E−17 | Cluster A |
| 89 | 2.99E−37 | P07900 | −0.76978 | 9.56E−35 | Cluster A |
| 90 | 8.00E−14 | P07910 | 0.290417 | 6.87E−13 | Cluster B |
| 91 | 0.00023 | P07954 | 0.229223 | 0.000629 | Cluster B |
| 92 | 0.000404 | P08195 | 0.525502 | 0.001063 | Cluster B |
| 93 | 2.45E−31 | P08238 | −0.86512 | 1.20E−29 | Cluster A |
| 94 | 0.001004 | P08559 | 0.609111 | 0.002493 | Cluster B |
| 95 | 2.86E−05 | P08574 | 0.714068 | 9.55E−05 | Cluster B |
| 96 | 5.53E−05 | P08579 | 0.264134 | 0.000173 | Cluster B |
| 97 | 3.59E−05 | P08621 | 0.209994 | 0.000118 | Cluster B |
| 98 | 0.000951 | P08670 | 0.24358 | 0.002371 | Cluster B |
| 99 | 4.22E−07 | P08708 | −0.42354 | 1.76E−06 | Cluster A |
| 100 | 1.65E−05 | P08758 | −0.30339 | 5.78E−05 | Cluster A |
| 101 | 9.71E−08 | P09012 | 0.339135 | 4.37E−07 | Cluster B |
| 102 | 1.75E−05 | P09104 | −0.35185 | 6.13E−05 | Cluster A |
| 103 | 2.04E−18 | P09429 | −0.9781 | 2.64E−17 | Cluster A |
| 104 | 2.95E−13 | P09496 | −0.42384 | 2.32E−12 | Cluster A |
| 105 | 0.001377 | P09525 | 0.640199 | 0.003297 | Cluster B |
| 106 | 5.36E−10 | P09651 | 0.34817 | 3.15E−09 | Cluster B |
| 107 | 0.001055 | P09661 | 0.521305 | 0.002606 | Cluster B |
| 108 | 5.33E−13 | P09669 | 0.623897 | 3.99E−12 | Cluster B |
| 109 | 0.000377 | P0DJD0 | −0.64078 | 0.001002 | Cluster A |
| 110 | 3.06E−13 | P0DP25 | −0.56427 | 2.39E−12 | Cluster A |
| 111 | 0.000314 | P10412 | 0.278471 | 0.000844 | Cluster B |
| 112 | 7.55E−05 | P10599 | −0.28802 | 0.000228 | Cluster A |
| 113 | 5.57E−10 | P10768 | −0.5146 | 3.26E−09 | Cluster A |
| 114 | 6.40E−14 | P10809 | 0.384987 | 5.57E−13 | Cluster B |
| 115 | 2.75E−13 | P11021 | 0.353903 | 2.17E−12 | Cluster B |
| 116 | 3.45E−34 | P11142 | −0.5486 | 3.40E−32 | Cluster A |
| 117 | 3.40E−06 | P11177 | 0.374213 | 1.30E−05 | Cluster B |
| 118 | 0.003608 | P11310 | 0.272773 | 0.00797 | Cluster B |
| 119 | 0.001949 | P11387 | 0.177075 | 0.004515 | Cluster B |
| 120 | 1.10E−23 | P11413 | −0.88257 | 2.07E−22 | Cluster A |
| 121 | 6.61E−08 | P11586 | −0.3257 | 3.02E−07 | Cluster A |
| 122 | 1.50E−09 | P11766 | −0.51913 | 8.32E−09 | Cluster A |
| 123 | 4.24E−27 | P11940 | −0.59068 | 1.21E−25 | Cluster A |
| 124 | 1.35E−21 | P12236 | 0.483392 | 2.21E−20 | Cluster B |
| 125 | 7.03E−15 | P12268 | −0.49227 | 6.87E−14 | Cluster A |
| 126 | 0.000156 | P12955 | −0.3736 | 0.000442 | Cluster A |
| 127 | 0.000284 | P12956 | 0.27326 | 0.000767 | Cluster B |
| 128 | 0.001533 | P13010 | 0.129831 | 0.003644 | Cluster B |
| 129 | 6.19E−07 | P13073 | 0.409011 | 2.55E−06 | Cluster B |
| 130 | 0.001366 | P13473 | 0.43576 | 0.003283 | Cluster B |
| 131 | 5.60E−14 | P13489 | −0.58986 | 4.94E−13 | Cluster A |
| 132 | 1.53E−36 | P13639 | −0.86102 | 2.80E−34 | Cluster A |
| 133 | 8.30E−15 | P13667 | 0.443775 | 7.98E−14 | Cluster B |
| 134 | 2.12E−05 | P13797 | −0.46211 | 7.26E−05 | Cluster A |
| 135 | 1.23E−05 | P14174 | −0.8638 | 4.44E−05 | Cluster A |
| 136 | 0.000179 | P14314 | 0.226635 | 0.000501 | Cluster B |
| 137 | 1.29E−19 | P14324 | −0.72374 | 1.77E−18 | Cluster A |
| 138 | 2.22E−07 | P14550 | −0.43144 | 9.66E−07 | Cluster A |
| 139 | 3.52E−36 | P14618 | −1.03235 | 5.62E−34 | Cluster A |
| 140 | 7.86E−19 | P14625 | 0.436893 | 1.04E−17 | Cluster B |
| 141 | 0.000145 | P14854 | 0.370584 | 0.000418 | Cluster B |
| 142 | 1.37E−13 | P14866 | 0.351631 | 1.13E−12 | Cluster B |
| 143 | 0.000417 | P14868 | −0.20749 | 0.001092 | Cluster A |
| 144 | 6.46E−12 | P14927 | 0.919169 | 4.44E−11 | Cluster B |
| 145 | 2.76E−25 | P15559 | −0.89132 | 6.61E−24 | Cluster A |
| 146 | 6.51E−28 | P15880 | −0.86918 | 2.03E−26 | Cluster A |
| 147 | 4.15E−10 | P16104 | 0.430715 | 2.48E−09 | Cluster B |
| 148 | 6.71E−09 | P16401 | 0.359916 | 3.45E−08 | Cluster B |
| 149 | 1.29E−06 | P16402 | 0.440066 | 5.19E−06 | Cluster B |
| 150 | 2.54E−16 | P16403 | 0.483059 | 2.71E−15 | Cluster B |
| 151 | 3.17E−08 | P16615 | 0.452749 | 1.50E−07 | Cluster B |
| 152 | 2.36E−09 | P16949 | −0.33546 | 1.27E−08 | Cluster A |
| 153 | 2.68E−05 | P17813 | −0.95997 | 8.99E−05 | Cluster A |
| 154 | 0.001607 | P17844 | 0.112873 | 0.003791 | Cluster B |
| 155 | 5.70E−18 | P17931 | −0.60021 | 6.94E−17 | Cluster A |
| 156 | 0.002284 | P17987 | −0.14194 | 0.005217 | Cluster A |
| 157 | 8.91E−27 | P18077 | −0.74137 | 2.37E−25 | Cluster A |
| 158 | 3.07E−33 | P18124 | −0.68313 | 2.31E−31 | Cluster A |
| 159 | 6.40E−10 | P18621 | −0.46983 | 3.65E−09 | Cluster A |
| 160 | 2.43E−15 | P18669 | −0.71882 | 2.47E−14 | Cluster A |
| 161 | 6.23E−06 | P18859 | 0.582091 | 2.33E−05 | Cluster B |
| 162 | 2.17E−16 | P19105 | −0.98987 | 2.35E−15 | Cluster A |
| 163 | 8.96E−05 | P19623 | −0.32276 | 0.000265 | Cluster A |
| 164 | 5.22E−10 | P20042 | −0.55471 | 3.08E−09 | Cluster A |
| 165 | 5.82E−12 | P20290 | −0.66705 | 4.02E−11 | Cluster A |
| 166 | 0.001081 | P20340 | 0.288453 | 0.00266 | Cluster B |
| 167 | 8.78E−11 | P20674 | 0.331045 | 5.40E−10 | Cluster B |
| 168 | 4.25E−08 | P20700 | 0.425694 | 1.97E−07 | Cluster B |
| 169 | 4.53E−12 | P20962 | −0.66494 | 3.17E−11 | Cluster A |
| 170 | 0.000227 | P21281 | −0.27116 | 0.000624 | Cluster A |
| 171 | 0.001157 | P21333 | −0.0888 | 0.002829 | Cluster A |
| 172 | 5.54E−11 | P22061 | −0.83965 | 3.50E−10 | Cluster A |
| 173 | 3.64E−09 | P22087 | 0.419214 | 1.92E−08 | Cluster B |
| 174 | 9.74E−09 | P22102 | −0.40844 | 4.90E−08 | Cluster A |
| 175 | 6.00E−17 | P22234 | −0.87472 | 6.92E−16 | Cluster A |
| 176 | 0.004433 | P22307 | 0.391144 | 0.00961 | Cluster B |
| 177 | 2.60E−05 | P22314 | −0.31986 | 8.76E−05 | Cluster A |
| 178 | 1.79E−15 | P22392 | −0.75493 | 1.85E−14 | Cluster A |
| 179 | 8.40E−11 | P22626 | 0.264726 | 5.19E−10 | Cluster B |
| 180 | 0.00022 | P22695 | 0.274443 | 0.000607 | Cluster B |
| 181 | 0.000281 | P23193 | −0.34296 | 0.000759 | Cluster A |
| 182 | 1.08E−19 | P23246 | 0.453158 | 1.52E−18 | Cluster B |
| 183 | 1.06E−06 | P23284 | 0.235905 | 4.28E−06 | Cluster B |
| 184 | 8.46E−09 | P23297 | −0.54046 | 4.28E−08 | Cluster A |
| 185 | 9.02E−23 | P23396 | −0.59555 | 1.63E−21 | Cluster A |
| 186 | 1.34E−13 | P23526 | −0.41283 | 1.11E−12 | Cluster A |
| 187 | 5.06E−36 | P23528 | −1.37736 | 7.20E−34 | Cluster A |
| 188 | 0.000206 | P23588 | −0.23232 | 0.000573 | Cluster A |
| 189 | 3.10E−31 | P24534 | −1.09398 | 1.47E−29 | Cluster A |
| 190 | 2.42E−18 | P24539 | 0.727454 | 3.06E−17 | Cluster B |
| 191 | 2.65E−11 | P24752 | 0.382243 | 1.76E−10 | Cluster B |
| 192 | 4.12E−06 | P24941 | −0.30691 | 1.57E−05 | Cluster A |
| 193 | 1.57E−06 | P25398 | −0.50855 | 6.23E−06 | Cluster A |
| 194 | 9.47E−23 | P25705 | 0.57104 | 1.68E−21 | Cluster B |
| 195 | 2.50E−25 | P26038 | −0.5567 | 6.15E−24 | Cluster A |
| 196 | 0.000155 | P26368 | 0.502116 | 0.000442 | Cluster B |
| 197 | 1.67E−30 | P26373 | −0.72597 | 7.38E−29 | Cluster A |
| 198 | 0.004603 | P26447 | 0.174892 | 0.009962 | Cluster B |
| 199 | 0.00353 | P26583 | −0.25724 | 0.007811 | Cluster A |
| 200 | 8.52E−06 | P26639 | −0.24982 | 3.12E−05 | Cluster A |
| 201 | 2.88E−26 | P26641 | −0.75723 | 7.36E−25 | Cluster A |
| 202 | 9.34E−14 | P27635 | −0.45446 | 7.87E−13 | Cluster A |
| 203 | 4.22E−13 | P27797 | 0.382461 | 3.22E−12 | Cluster B |
| 204 | 3.36E−18 | P27824 | 0.554246 | 4.21E−17 | Cluster B |
| 205 | 3.71E−14 | P29401 | −0.44459 | 3.35E−13 | Cluster A |
| 206 | 4.57E−11 | P29692 | −0.51322 | 2.91E−10 | Cluster A |
| 207 | 1.69E−09 | P30040 | 0.385868 | 9.30E−09 | Cluster B |
| 208 | 6.97E−09 | P30041 | −0.51789 | 3.55E−08 | Cluster A |
| 209 | 3.63E−05 | P30048 | 0.418534 | 0.000119 | Cluster B |
| 210 | 1.10E−22 | P30050 | −0.60264 | 1.89E−21 | Cluster A |
| 211 | 6.81E−09 | P30084 | 0.542131 | 3.48E−08 | Cluster B |
| 212 | 0.003624 | P30086 | −0.22016 | 0.007987 | Cluster A |
| 213 | 7.07E−11 | P30101 | 0.338817 | 4.45E−10 | Cluster B |
| 214 | 2.01E−09 | P30533 | 0.429159 | 1.09E−08 | Cluster B |
| 215 | 0.000109 | P30626 | −0.26781 | 0.000319 | Cluster A |
| 216 | 8.40E−13 | P31040 | 0.920718 | 6.21E−12 | Cluster B |
| 217 | 2.20E−05 | P31939 | −0.40174 | 7.49E−05 | Cluster A |
| 218 | 3.94E−06 | P31942 | 0.227399 | 1.51E−05 | Cluster B |
| 219 | 3.10E−06 | P31943 | 0.330107 | 1.20E−05 | Cluster B |
| 220 | 2.00E−21 | P31946 | −0.69249 | 3.24E−20 | Cluster A |
| 221 | 2.06E−24 | P31947 | −1.56597 | 4.31E−23 | Cluster A |
| 222 | 3.24E−24 | P31949 | −0.79482 | 6.48E−23 | Cluster A |
| 223 | 1.24E−21 | P32119 | −0.79627 | 2.06E−20 | Cluster A |
| 224 | 3.62E−08 | P32969 | −0.3943 | 1.69E−07 | Cluster A |
| 225 | 1.38E−05 | P33991 | 0.241691 | 4.91E−05 | Cluster B |
| 226 | 2.16E−14 | P34897 | 0.429394 | 2.00E−13 | Cluster B |
| 227 | 1.07E−06 | P34932 | −0.23008 | 4.33E−06 | Cluster A |
| 228 | 6.22E−28 | P35232 | 0.587405 | 1.99E−26 | Cluster B |
| 229 | 0.000458 | P35241 | −0.35663 | 0.001196 | Cluster A |
| 230 | 1.31E−07 | P35268 | −0.31236 | 5.85E−07 | Cluster A |
| 231 | 0.001121 | P35580 | −0.22232 | 0.002748 | Cluster A |
| 232 | 5.55E−05 | P35610 | 0.530629 | 0.000174 | Cluster B |
| 233 | 5.75E−07 | P35613 | 0.403417 | 2.39E−06 | Cluster B |
| 234 | 5.63E−14 | P35637 | −0.71123 | 4.94E−13 | Cluster A |
| 235 | 0.002035 | P35659 | 0.246953 | 0.004682 | Cluster B |
| 236 | 3.16E−10 | P36542 | 0.398698 | 1.89E−09 | Cluster B |
| 237 | 0.001967 | P36551 | 0.312979 | 0.004541 | Cluster B |
| 238 | 2.22E−20 | P36578 | −0.5004 | 3.37E−19 | Cluster A |
| 239 | 7.20E−05 | P36776 | 0.347853 | 0.000219 | Cluster B |
| 240 | 2.23E−13 | P36957 | 0.57224 | 1.78E−12 | Cluster B |
| 241 | 3.31E−05 | P37108 | 0.324531 | 0.000109 | Cluster B |
| 242 | 2.46E−06 | P37802 | −0.38857 | 9.60E−06 | Cluster A |
| 243 | 1.50E−05 | P37837 | −0.28877 | 5.33E−05 | Cluster A |
| 244 | 1.99E−10 | P38159 | 0.289836 | 1.21E−09 | Cluster B |
| 245 | 2.98E−08 | P38606 | −0.2953 | 1.43E−07 | Cluster A |
| 246 | 2.16E−12 | P38646 | 0.23637 | 1.53E−11 | Cluster B |
| 247 | 0.000357 | P38919 | −0.19906 | 0.000951 | Cluster A |
| 248 | 3.57E−27 | P39019 | −0.80608 | 1.04E−25 | Cluster A |
| 249 | 4.57E−27 | P39023 | −0.65706 | 1.27E−25 | Cluster A |
| 250 | 5.33E−08 | P39656 | 0.348306 | 2.46E−07 | Cluster B |
| 251 | 2.05E−31 | P40121 | −1.2558 | 1.09E−29 | Cluster A |
| 252 | 2.99E−06 | P40227 | −0.38208 | 1.16E−05 | Cluster A |
| 253 | 5.88E−18 | P40429 | −0.53509 | 7.10E−17 | Cluster A |
| 254 | 3.75E−07 | P40925 | −0.29951 | 1.58E−06 | Cluster A |
| 255 | 4.12E−22 | P40926 | 0.480023 | 7.02E−21 | Cluster B |
| 256 | 2.65E−13 | P40939 | 0.421507 | 2.11E−12 | Cluster B |
| 257 | 0.00032 | P41091 | −0.44608 | 0.000858 | Cluster A |
| 258 | 2.63E−05 | P41250 | −0.25185 | 8.84E−05 | Cluster A |
| 259 | 3.34E−09 | P42677 | −0.55661 | 1.77E−08 | Cluster A |
| 260 | 4.95E−14 | P42704 | 0.392109 | 4.43E−13 | Cluster B |
| 261 | 0.000342 | P42765 | 0.359394 | 0.000914 | Cluster B |
| 262 | 2.33E−24 | P42766 | −0.88902 | 4.73E−23 | Cluster A |
| 263 | 1.56E−12 | P43243 | 0.402815 | 1.12E−11 | Cluster B |
| 264 | 0.003746 | P43246 | 0.446104 | 0.008233 | Cluster B |
| 265 | 3.86E−08 | P43304 | 0.566857 | 1.80E−07 | Cluster B |
| 266 | 1.51E−05 | P43307 | 0.488912 | 5.34E−05 | Cluster B |
| 267 | 8.24E−11 | P43487 | −0.88924 | 5.12E−10 | Cluster A |
| 268 | 0.000224 | P46013 | 0.234344 | 0.000617 | Cluster B |
| 269 | 0.001727 | P46060 | 0.431159 | 0.004038 | Cluster B |
| 270 | 0.000167 | P46087 | 0.257009 | 0.000472 | Cluster B |
| 271 | 4.32E−26 | P46776 | −0.67854 | 1.08E−24 | Cluster A |
| 272 | 1.04E−16 | P46777 | −0.54192 | 1.16E−15 | Cluster A |
| 273 | 2.92E−11 | P46778 | −0.47598 | 1.90E−10 | Cluster A |
| 274 | 8.21E−21 | P46779 | −0.51359 | 1.30E−19 | Cluster A |
| 275 | 4.30E−32 | P46781 | −0.9441 | 2.62E−30 | Cluster A |
| 276 | 6.49E−17 | P46782 | −0.8983 | 7.41E−16 | Cluster A |
| 277 | 2.49E−07 | P46977 | 0.592938 | 1.08E−06 | Cluster B |
| 278 | 9.13E−14 | P47813 | −0.61921 | 7.78E−13 | Cluster A |
| 279 | 1.43E−24 | P47914 | −0.98786 | 3.09E−23 | Cluster A |
| 280 | 0.000178 | P47985 | 0.401999 | 0.0005 | Cluster B |
| 281 | 0.000145 | P48643 | −0.20277 | 0.000418 | Cluster A |
| 282 | 0.000406 | P49189 | −0.29647 | 0.001066 | Cluster A |
| 283 | 6.18E−31 | P49207 | −0.75898 | 2.82E−29 | Cluster A |
| 284 | 0.000147 | P49321 | −0.24422 | 0.000421 | Cluster A |
| 285 | 1.56E−37 | P49327 | −0.97639 | 7.99E−35 | Cluster A |
| 286 | 8.69E−06 | P49368 | −0.22257 | 3.18E−05 | Cluster A |
| 287 | 1.96E−15 | P49411 | 0.405932 | 2.00E−14 | Cluster B |
| 288 | 0.002496 | P49588 | −0.17698 | 0.005641 | Cluster A |
| 289 | 0.0007 | P49736 | 0.233924 | 0.00178 | Cluster B |
| 290 | 8.36E−15 | P49755 | 0.921753 | 7.98E−14 | Cluster B |
| 291 | 3.58E−16 | P49773 | −0.78305 | 3.78E−15 | Cluster A |
| 292 | 3.34E−05 | P50213 | 0.295064 | 0.00011 | Cluster B |
| 293 | 4.81E−13 | P50395 | −0.48282 | 3.62E−12 | Cluster A |
| 294 | 7.08E−09 | P50402 | 0.35498 | 3.59E−08 | Cluster B |
| 295 | 0.001295 | P50454 | 0.377681 | 0.003132 | Cluster B |
| 296 | 1.75E−16 | P50502 | −0.67741 | 1.92E−15 | Cluster A |
| 297 | 0.002327 | P50552 | 0.392631 | 0.005306 | Cluster B |
| 298 | 2.84E−07 | P50990 | −0.31324 | 1.22E−06 | Cluster A |
| 299 | 3.73E−05 | P50991 | −0.30816 | 0.000121 | Cluster A |
| 300 | 0.000605 | P51148 | 0.40263 | 0.001551 | Cluster B |
| 301 | 7.50E−17 | P51149 | 0.519755 | 8.41E−16 | Cluster B |
| 302 | 0.000621 | P51159 | 0.356667 | 0.001588 | Cluster B |
| 303 | 2.75E−09 | P51572 | 0.454622 | 1.47E−08 | Cluster B |
| 304 | 0.00015 | P51610 | 0.301292 | 0.000429 | Cluster B |
| 305 | 0.000183 | P51659 | 0.512309 | 0.000512 | Cluster B |
| 306 | 1.79E−09 | P51991 | 0.456262 | 9.80E−09 | Cluster B |
| 307 | 2.90E−08 | P52209 | −0.34196 | 1.40E−07 | Cluster A |
| 308 | 5.64E−12 | P52272 | 0.289145 | 3.92E−11 | Cluster B |
| 309 | 7.29E−05 | P52565 | −0.60092 | 0.000221 | Cluster A |
| 310 | 0.00094 | P52566 | −0.22612 | 0.002348 | Cluster A |
| 311 | 1.15E−07 | P52907 | −0.39994 | 5.12E−07 | Cluster A |
| 312 | 1.22E−09 | P53396 | −0.33979 | 6.81E−09 | Cluster A |
| 313 | 8.31E−05 | P53801 | 0.631089 | 0.000248 | Cluster B |
| 314 | 7.81E−05 | P54136 | −0.27424 | 0.000234 | Cluster A |
| 315 | 8.88E−06 | P54819 | 0.374041 | 3.23E−05 | Cluster B |
| 316 | 1.83E−12 | P55060 | −0.44626 | 1.30E−11 | Cluster A |
| 317 | 0.000366 | P55072 | −0.17802 | 0.000973 | Cluster A |
| 318 | 6.50E−15 | P55084 | 0.557281 | 6.39E−14 | Cluster B |
| 319 | 0.001008 | P55265 | 0.392475 | 0.002498 | Cluster B |
| 320 | 5.65E−05 | P55327 | −0.50409 | 0.000176 | Cluster A |
| 321 | 3.51E−05 | P55809 | 0.491307 | 0.000115 | Cluster B |
| 322 | 5.47E−09 | P55854 | −0.42826 | 2.84E−08 | Cluster A |
| 323 | 3.06E−14 | P56381 | 0.582334 | 2.80E−13 | Cluster B |
| 324 | 0.001718 | P57103 | −0.63399 | 0.004025 | Cluster A |
| 325 | 4.61E−24 | P58546 | −1.60773 | 9.07E−23 | Cluster A |
| 326 | 0.000166 | P59998 | −0.23955 | 0.00047 | Cluster A |
| 327 | 1.02E−09 | P60174 | −0.32861 | 5.74E−09 | Cluster A |
| 328 | 0.001307 | P60228 | −0.47963 | 0.003154 | Cluster A |
| 329 | 8.44E−20 | P60660 | −0.70355 | 1.21E−18 | Cluster A |
| 330 | 5.21E−09 | P60709 | −0.2329 | 2.72E−08 | Cluster A |
| 331 | 1.41E−19 | P60842 | −0.61683 | 1.92E−18 | Cluster A |
| 332 | 1.19E−12 | P60866 | −0.69578 | 8.72E−12 | Cluster A |
| 333 | 1.45E−06 | P61009 | 0.429563 | 5.74E−06 | Cluster B |
| 334 | 1.21E−11 | P61026 | 0.425535 | 8.13E−11 | Cluster B |
| 335 | 1.88E−05 | P61081 | −0.27211 | 6.50E−05 | Cluster A |
| 336 | 1.08E−05 | P61088 | −0.82012 | 3.90E−05 | Cluster A |
| 337 | 2.51E−28 | P61247 | −0.65198 | 8.23E−27 | Cluster A |
| 338 | 1.33E−34 | P61254 | −0.78173 | 1.55E−32 | Cluster A |
| 339 | 1.20E−17 | P61313 | −0.5487 | 1.42E−16 | Cluster A |
| 340 | 2.25E−21 | P61353 | −0.57239 | 3.60E−20 | Cluster A |
| 341 | 9.83E−15 | P61604 | 0.417969 | 9.32E−14 | Cluster B |
| 342 | 4.87E−15 | P61927 | −0.61789 | 4.83E−14 | Cluster A |
| 343 | 1.06E−17 | P61956 | −0.93005 | 1.26E−16 | Cluster A |
| 344 | 3.30E−13 | P61970 | −0.81314 | 2.54E−12 | Cluster A |
| 345 | 2.24E−33 | P61981 | −0.89703 | 1.79E−31 | Cluster A |
| 346 | 7.20E−30 | P62081 | −0.84394 | 2.71E−28 | Cluster A |
| 347 | 2.35E−31 | P62241 | −0.87734 | 1.20E−29 | Cluster A |
| 348 | 3.18E−23 | P62244 | −0.92185 | 5.90E−22 | Cluster A |
| 349 | 3.97E−30 | P62249 | −1.05765 | 1.54E−28 | Cluster A |
| 350 | 2.13E−27 | P62258 | −1.24916 | 6.34E−26 | Cluster A |
| 351 | 4.17E−08 | P62263 | −0.38402 | 1.94E−07 | Cluster A |
| 352 | 1.00E−11 | P62266 | −0.59355 | 6.81E−11 | Cluster A |
| 353 | 7.44E−12 | P62269 | −0.88374 | 5.09E−11 | Cluster A |
| 354 | 1.32E−06 | P62273 | −1.55548 | 5.29E−06 | Cluster A |
| 355 | 2.84E−25 | P62277 | −0.57524 | 6.61E−24 | Cluster A |
| 356 | 9.86E−33 | P62280 | −1.1753 | 7.01E−31 | Cluster A |
| 357 | 1.61E−20 | P62424 | −0.48845 | 2.51E−19 | Cluster A |
| 358 | 6.15E−22 | P62701 | −0.53683 | 1.04E−20 | Cluster A |
| 359 | 2.06E−12 | P62750 | −0.30544 | 1.46E−11 | Cluster A |
| 360 | 2.11E−32 | P62753 | −1.22349 | 1.35E−30 | Cluster A |
| 361 | 2.24E−10 | P62805 | 0.32429 | 1.35E−09 | Cluster B |
| 362 | 8.13E−11 | P62807 | 0.421736 | 5.08E−10 | Cluster B |
| 363 | 0.000296 | P62820 | 0.139936 | 0.000796 | Cluster B |
| 364 | 2.20E−33 | P62826 | −1.2103 | 1.79E−31 | Cluster A |
| 365 | 2.84E−25 | P62829 | −0.70939 | 6.61E−24 | Cluster A |
| 366 | 3.08E−08 | P62834 | 0.572888 | 1.46E−07 | Cluster B |
| 367 | 3.57E−18 | P62847 | −0.92265 | 4.44E−17 | Cluster A |
| 368 | 2.61E−20 | P62851 | −0.74325 | 3.93E−19 | Cluster A |
| 369 | 4.31E−18 | P62854 | −0.97447 | 5.31E−17 | Cluster A |
| 370 | 1.71E−20 | P62857 | −0.81644 | 2.64E−19 | Cluster A |
| 371 | 0.001284 | P62861 | −0.34391 | 0.003113 | Cluster A |
| 372 | 1.45E−08 | P62888 | −0.36215 | 7.21E−08 | Cluster A |
| 373 | 8.51E−29 | P62899 | −0.74303 | 2.86E−27 | Cluster A |
| 374 | 1.36E−24 | P62906 | −0.62127 | 2.99E−23 | Cluster A |
| 375 | 1.79E−09 | P62910 | −0.37741 | 9.80E−09 | Cluster A |
| 376 | 6.18E−05 | P62913 | −0.31267 | 0.000192 | Cluster A |
| 377 | 1.18E−26 | P62917 | −0.5499 | 3.08E−25 | Cluster A |
| 378 | 1.92E−27 | P62937 | −1.14452 | 5.86E−26 | Cluster A |
| 379 | 0.001416 | P62942 | −0.25551 | 0.003379 | Cluster A |
| 380 | 5.74E−25 | P62987 | −0.66072 | 1.31E−23 | Cluster A |
| 381 | 0.004039 | P62995 | 0.244584 | 0.008862 | Cluster B |
| 382 | 5.78E−32 | P63104 | −1.00792 | 3.36E−30 | Cluster A |
| 383 | 2.54E−06 | P63162 | 0.338502 | 9.90E−06 | Cluster B |
| 384 | 2.67E−08 | P63173 | −0.50742 | 1.29E−07 | Cluster A |
| 385 | 1.02E−23 | P63220 | −2.4649 | 1.95E−22 | Cluster A |
| 386 | 7.01E−36 | P63241 | −1.71532 | 8.96E−34 | Cluster A |
| 387 | 4.34E−06 | P63244 | −0.3341 | 1.65E−05 | Cluster A |
| 388 | 0.00262 | P67809 | −0.20563 | 0.005889 | Cluster A |
| 389 | 3.64E−07 | P67812 | 0.585149 | 1.54E−06 | Cluster B |
| 390 | 6.75E−25 | P68036 | −0.72305 | 1.52E−23 | Cluster A |
| 391 | 5.48E−08 | P68363 | −1.7886 | 2.52E−07 | Cluster A |
| 392 | 6.97E−27 | P68371 | −1.04751 | 1.90E−25 | Cluster A |
| 393 | 0.000403 | P68400 | 0.252293 | 0.001063 | Cluster B |
| 394 | 0.001061 | P69849 | 0.426735 | 0.002614 | Cluster B |
| 395 | 8.35E−15 | P78371 | −0.58565 | 7.98E−14 | Cluster A |
| 396 | 0.00137 | P78417 | −0.13674 | 0.003287 | Cluster A |
| 397 | 5.34E−17 | P78527 | 0.284545 | 6.20E−16 | Cluster B |
| 398 | 0.000511 | P80723 | 0.728923 | 0.001321 | Cluster B |
| 399 | 9.99E−16 | P83731 | −0.59037 | 1.05E−14 | Cluster A |
| 400 | 3.61E−11 | P83881 | −0.39767 | 2.32E−10 | Cluster A |
| 401 | 2.04E−24 | P84098 | −1.28032 | 4.31E−23 | Cluster A |
| 402 | 6.49E−08 | P99999 | 0.336349 | 2.97E−07 | Cluster B |
| 403 | 0.000267 | Q00059 | 0.300231 | 0.000726 | Cluster B |
| 404 | 8.24E−06 | Q00325 | 0.320047 | 3.04E−05 | Cluster B |
| 405 | 2.30E−08 | Q00610 | −0.26091 | 1.11E−07 | Cluster A |
| 406 | 4.15E−07 | Q00688 | −0.66944 | 1.74E−06 | Cluster A |
| 407 | 2.87E−15 | Q00839 | 0.453312 | 2.86E−14 | Cluster B |
| 408 | 0.002542 | Q01130 | −0.24878 | 0.005724 | Cluster A |
| 409 | 2.60E−15 | Q01469 | −0.69946 | 2.62E−14 | Cluster A |
| 410 | 1.31E−12 | Q01518 | −0.63043 | 9.46E−12 | Cluster A |
| 411 | 0.001352 | Q01650 | 0.336574 | 0.003256 | Cluster B |
| 412 | 0.000124 | Q01813 | −0.34486 | 0.000361 | Cluster A |
| 413 | 0.001632 | Q01844 | 0.432603 | 0.003845 | Cluster B |
| 414 | 0.001832 | Q02218 | 0.322724 | 0.00426 | Cluster B |
| 415 | 3.55E−20 | Q02543 | −0.44844 | 5.27E−19 | Cluster A |
| 416 | 8.71E−05 | Q02790 | −0.23755 | 0.000259 | Cluster A |
| 417 | 2.95E−29 | Q02878 | −0.64358 | 1.02E−27 | Cluster A |
| 418 | 0.002913 | Q02978 | 0.280501 | 0.00649 | Cluster B |
| 419 | 2.43E−05 | Q03252 | 0.262842 | 8.27E−05 | Cluster B |
| 420 | 3.17E−05 | Q04837 | 0.284435 | 0.000105 | Cluster B |
| 421 | 1.15E−36 | Q06830 | −1.96378 | 2.44E−34 | Cluster A |
| 422 | 9.35E−14 | Q07020 | −0.40355 | 7.87E−13 | Cluster A |
| 423 | 0.000203 | Q07021 | 0.910802 | 0.000563 | Cluster B |
| 424 | 4.39E−06 | Q07666 | 0.429952 | 1.66E−05 | Cluster B |
| 425 | 5.50E−05 | Q07955 | 0.241926 | 0.000173 | Cluster B |
| 426 | 1.53E−14 | Q08211 | 0.345416 | 1.43E−13 | Cluster B |
| 427 | 0.001605 | Q08380 | 0.341092 | 0.003791 | Cluster B |
| 428 | 0.002762 | Q08722 | 0.479918 | 0.006187 | Cluster B |
| 429 | 0.002531 | Q08945 | 0.316821 | 0.00571 | Cluster B |
| 430 | 0.00156 | Q09028 | 0.203954 | 0.003701 | Cluster B |
| 431 | 1.40E−06 | Q09666 | −0.3841 | 5.59E−06 | Cluster A |
| 432 | 3.00E−08 | Q12906 | 0.301276 | 1.43E−07 | Cluster B |
| 433 | 3.08E−05 | Q12931 | 0.151581 | 0.000103 | Cluster B |
| 434 | 4.82E−05 | Q13011 | 0.290689 | 0.000155 | Cluster B |
| 435 | 1.93E−05 | Q13151 | 0.287588 | 6.64E−05 | Cluster B |
| 436 | 0.00417 | Q13242 | 0.478419 | 0.009054 | Cluster B |
| 437 | 3.61E−07 | Q13247 | 0.298732 | 1.53E−06 | Cluster B |
| 438 | 1.95E−06 | Q13263 | 0.285788 | 7.68E−06 | Cluster B |
| 439 | 0.001934 | Q13347 | −0.26185 | 0.004489 | Cluster A |
| 440 | 2.62E−09 | Q13404 | −0.46985 | 1.41E−08 | Cluster A |
| 441 | 0.000194 | Q13409 | −0.25841 | 0.000539 | Cluster A |
| 442 | 7.22E−10 | Q13428 | 0.433707 | 4.08E−09 | Cluster B |
| 443 | 8.33E−05 | Q13435 | 0.215308 | 0.000248 | Cluster B |
| 444 | 2.96E−07 | Q13442 | −0.52039 | 1.27E−06 | Cluster A |
| 445 | 0.000937 | Q13451 | 0.191705 | 0.002344 | Cluster B |
| 446 | 1.85E−07 | Q13637 | 0.616847 | 8.09E−07 | Cluster B |
| 447 | 2.11E−05 | Q13838 | −0.35218 | 7.25E−05 | Cluster A |
| 448 | 0.000645 | Q13951 | −0.27726 | 0.001642 | Cluster A |
| 449 | 9.62E−14 | Q14019 | −0.68323 | 8.04E−13 | Cluster A |
| 450 | 8.44E−34 | Q14247 | −1.06257 | 7.71E−32 | Cluster A |
| 451 | 2.80E−05 | Q14376 | −0.33939 | 9.38E−05 | Cluster A |
| 452 | 6.52E−13 | Q14444 | −0.44996 | 4.85E−12 | Cluster A |
| 453 | 6.41E−05 | Q14566 | 0.312227 | 0.000197 | Cluster B |
| 454 | 1.40E−07 | Q14697 | 0.22257 | 6.19E−07 | Cluster B |
| 455 | 4.26E−07 | Q14956 | 0.629553 | 1.78E−06 | Cluster B |
| 456 | 1.28E−11 | Q14978 | 0.360392 | 8.59E−11 | Cluster B |
| 457 | 9.27E−06 | Q14980 | 0.252161 | 3.37E−05 | Cluster B |
| 458 | 0.00076 | Q15008 | −0.17083 | 0.001929 | Cluster A |
| 459 | 6.09E−20 | Q15056 | −0.6192 | 8.96E−19 | Cluster A |
| 460 | 0.004127 | Q15061 | 0.394754 | 0.008993 | Cluster B |
| 461 | 6.75E−17 | Q15084 | 0.446317 | 7.64E−16 | Cluster B |
| 462 | 0.000172 | Q15149 | 0.212718 | 0.000486 | Cluster B |
| 463 | 7.77E−05 | Q15181 | −0.37909 | 0.000234 | Cluster A |
| 464 | 2.78E−11 | Q15233 | 0.35504 | 1.83E−10 | Cluster B |
| 465 | 1.64E−13 | Q15293 | 0.466244 | 1.33E−12 | Cluster B |
| 466 | 5.44E−05 | Q15363 | 0.582913 | 0.000171 | Cluster B |
| 467 | 9.02E−08 | Q15366 | −0.57619 | 4.08E−07 | Cluster A |
| 468 | 3.17E−09 | Q15370 | −0.39154 | 1.69E−08 | Cluster A |
| 469 | 1.83E−05 | Q15382 | −0.72495 | 6.34E−05 | Cluster A |
| 470 | 1.48E−08 | Q15392 | 0.457466 | 7.29E−08 | Cluster B |
| 471 | 3.15E−06 | Q15393 | 0.297394 | 1.21E−05 | Cluster B |
| 472 | 0.000522 | Q15417 | −0.51015 | 0.001345 | Cluster A |
| 473 | 3.63E−13 | Q15424 | 0.400138 | 2.78E−12 | Cluster B |
| 474 | 7.97E−06 | Q15459 | 0.279085 | 2.95E−05 | Cluster B |
| 475 | 5.57E−19 | Q15691 | −0.73942 | 7.43E−18 | Cluster A |
| 476 | 1.17E−12 | Q15696 | −0.82614 | 8.59E−12 | Cluster A |
| 477 | 6.81E−05 | Q15717 | 0.227067 | 0.000208 | Cluster B |
| 478 | 0.000338 | Q15758 | 0.290283 | 0.000906 | Cluster B |
| 479 | 1.71E−13 | Q15907 | 0.34981 | 1.38E−12 | Cluster B |
| 480 | 6.50E−05 | Q16181 | −0.31381 | 0.000199 | Cluster A |
| 481 | 0.002789 | Q16543 | −0.28875 | 0.006235 | Cluster A |
| 482 | 0.002045 | Q16629 | 0.321038 | 0.004695 | Cluster B |
| 483 | 4.03E−09 | Q16778 | 0.376063 | 2.11E−08 | Cluster B |
| 484 | 4.66E−06 | Q16836 | 0.320968 | 1.76E−05 | Cluster B |
| 485 | 5.78E−10 | Q16891 | 0.382721 | 3.35E−09 | Cluster B |
| 486 | 2.04E−06 | Q1KMD3 | 0.457295 | 8.02E−06 | Cluster B |
| 487 | 0.000145 | Q2TAY7 | 0.49664 | 0.000418 | Cluster B |
| 488 | 4.39E−06 | Q3ZCQ8 | 0.417587 | 1.66E−05 | Cluster B |
| 489 | 0.002826 | Q5SSJ5 | 0.234401 | 0.006307 | Cluster B |
| 490 | 0.000773 | Q5T3I0 | 0.455183 | 0.001958 | Cluster B |
| 491 | 1.87E−37 | Q5VTE0 | −1.81353 | 7.99E−35 | Cluster A |
| 492 | 0.000138 | Q5XKP0 | 0.461118 | 0.000401 | Cluster B |
| 493 | 2.23E−09 | Q71DI3 | 0.369699 | 1.21E−08 | Cluster B |
| 494 | 2.95E−17 | Q7KZF4 | −0.44767 | 3.46E−16 | Cluster A |
| 495 | 0.001585 | Q86UE4 | 0.260247 | 0.003753 | Cluster B |
| 496 | 0.001963 | Q86XP3 | 0.43685 | 0.004541 | Cluster B |
| 497 | 1.20E−05 | Q8N5M9 | 0.625843 | 4.33E−05 | Cluster B |
| 498 | 2.52E−05 | Q8N7Z2 | 0.75849 | 8.53E−05 | Cluster B |
| 499 | 1.03E−09 | Q8NBS9 | 0.452789 | 5.74E−09 | Cluster B |
| 500 | 5.31E−05 | Q8NBX0 | 0.509982 | 0.000169 | Cluster B |
| 501 | 0.001807 | Q8NC51 | −0.23208 | 0.00421 | Cluster A |
| 502 | 7.14E−10 | Q8TCJ2 | 0.682638 | 4.06E−09 | Cluster B |
| 503 | 1.46E−07 | Q8TCT9 | 0.587904 | 6.42E−07 | Cluster B |
| 504 | 0.000107 | Q8TDN6 | 0.625323 | 0.000315 | Cluster B |
| 505 | 0.000176 | Q8WU90 | −0.56266 | 0.000494 | Cluster A |
| 506 | 3.68E−05 | Q8WW12 | −0.404 | 0.00012 | Cluster A |
| 507 | 1.31E−05 | Q8WXH0 | 0.557275 | 4.68E−05 | Cluster B |
| 508 | 0.000219 | Q8WYA6 | 0.496705 | 0.000604 | Cluster B |
| 509 | 1.78E−05 | Q8WYQ5 | −1.25789 | 6.20E−05 | Cluster A |
| 510 | 0.000172 | Q92522 | 0.291841 | 0.000486 | Cluster B |
| 511 | 6.09E−07 | Q92598 | −0.41817 | 2.52E−06 | Cluster A |
| 512 | 2.79E−11 | Q92734 | −0.56871 | 1.83E−10 | Cluster A |
| 513 | 5.11E−05 | Q96AG4 | 0.242719 | 0.000163 | Cluster B |
| 514 | 3.69E−09 | Q96C19 | −0.3718 | 1.94E−08 | Cluster A |
| 515 | 5.71E−06 | Q96I99 | 0.388862 | 2.15E−05 | Cluster B |
| 516 | 0.000228 | Q96IX5 | 0.299308 | 0.000625 | Cluster B |
| 517 | 0.00238 | Q96SB4 | 0.162596 | 0.005416 | Cluster B |
| 518 | 1.08E−22 | Q99497 | −0.62536 | 1.89E−21 | Cluster A |
| 519 | 7.05E−14 | Q99536 | −0.57329 | 6.10E−13 | Cluster A |
| 520 | 6.20E−05 | Q99613 | −0.37232 | 0.000192 | Cluster A |
| 521 | 9.36E−20 | Q99623 | 0.434747 | 1.33E−18 | Cluster B |
| 522 | 5.92E−06 | Q99714 | 0.318625 | 2.22E−05 | Cluster B |
| 523 | 7.43E−08 | Q99729 | 0.415402 | 3.38E−07 | Cluster B |
| 524 | 0.004616 | Q99757 | 0.313102 | 0.009973 | Cluster B |
| 525 | 0.000393 | Q99829 | −0.19002 | 0.001038 | Cluster A |
| 526 | 0.004116 | Q99832 | −0.11634 | 0.008983 | Cluster A |
| 527 | 1.75E−05 | Q99848 | 0.624443 | 6.12E−05 | Cluster B |
| 528 | 8.48E−06 | Q99873 | −0.34667 | 3.12E−05 | Cluster A |
| 529 | 0.000866 | Q99878 | 0.206655 | 0.002171 | Cluster B |
| 530 | 0.000379 | Q99986 | 1.57746 | 0.001005 | Cluster B |
| 531 | 1.47E−31 | Q9BQE3 | −1.13268 | 8.19E−30 | Cluster A |
| 532 | 1.22E−08 | Q9BQG0 | 0.426151 | 6.07E−08 | Cluster B |
| 533 | 2.84E−11 | Q9BVC6 | 0.455681 | 1.85E−10 | Cluster B |
| 534 | 0.002663 | Q9BVP2 | 0.59797 | 0.005975 | Cluster B |
| 535 | 3.14E−08 | Q9BXS5 | −0.46504 | 1.49E−07 | Cluster A |
| 536 | 0.00225 | Q9BY44 | 0.589738 | 0.005149 | Cluster B |
| 537 | 0.000135 | Q9BZH6 | 0.44511 | 0.000392 | Cluster B |
| 538 | 0.001396 | Q9C0B1 | −0.25084 | 0.003336 | Cluster A |
| 539 | 0.000108 | Q9GZT3 | 0.474864 | 0.000316 | Cluster B |
| 540 | 3.41E−07 | Q9GZZ1 | −0.45864 | 1.46E−06 | Cluster A |
| 541 | 0.001676 | Q9H0A0 | 0.628824 | 0.003941 | Cluster B |
| 542 | 2.17E−05 | Q9H1E3 | −0.56292 | 7.41E−05 | Cluster A |
| 543 | 2.10E−24 | Q9H299 | −1.08255 | 4.34E−23 | Cluster A |
| 544 | 6.84E−05 | Q9H2W6 | 0.66679 | 0.000208 | Cluster B |
| 545 | 1.65E−10 | Q9H3K6 | −0.40339 | 1.01E−09 | Cluster A |
| 546 | 0.000966 | Q9H3N1 | 0.398583 | 0.002404 | Cluster B |
| 547 | 0.000486 | Q9H444 | 0.407015 | 0.00126 | Cluster B |
| 548 | 4.36E−05 | Q9H9B4 | 0.57824 | 0.000141 | Cluster B |
| 549 | 9.96E−07 | Q9HAV0 | 0.434474 | 4.04E−06 | Cluster B |
| 550 | 0.000147 | Q9HAV7 | 0.238317 | 0.000422 | Cluster B |
| 551 | 1.43E−07 | Q9HB71 | −0.58267 | 6.29E−07 | Cluster A |
| 552 | 0.00027 | Q9HC38 | 0.239842 | 0.000732 | Cluster B |
| 553 | 0.002439 | Q9NQP4 | 0.676201 | 0.00554 | Cluster B |
| 554 | 1.31E−12 | Q9NR30 | 0.333618 | 9.46E−12 | Cluster B |
| 555 | 1.96E−08 | Q9NR45 | −1.9634 | 9.59E−08 | Cluster A |
| 556 | 0.001092 | Q9NRV9 | −0.15657 | 0.002681 | Cluster A |
| 557 | 0.001773 | Q9NRX4 | −0.60578 | 0.004138 | Cluster A |
| 558 | 9.88E−08 | Q9NSD9 | −0.46788 | 4.44E−07 | Cluster A |
| 559 | 8.40E−10 | Q9NTK5 | −0.32783 | 4.73E−09 | Cluster A |
| 560 | 5.35E−05 | Q9NVI7 | 0.230967 | 0.000169 | Cluster B |
| 561 | 6.91E−06 | Q9NVP1 | 0.472615 | 2.58E−05 | Cluster B |
| 562 | 3.80E−11 | Q9NX63 | 0.41873 | 2.43E−10 | Cluster B |
| 563 | 0.001504 | Q9NYF8 | 0.343114 | 0.003582 | Cluster B |
| 564 | 0.003628 | Q9NZR2 | 0.382122 | 0.007987 | Cluster B |
| 565 | 5.23E−10 | Q9UBM7 | 0.386689 | 3.08E−09 | Cluster B |
| 566 | 7.50E−11 | Q9UHD8 | −0.55872 | 4.70E−10 | Cluster A |
| 567 | 8.96E−08 | Q9UII2 | 0.334059 | 4.06E−07 | Cluster B |
| 568 | 7.92E−05 | Q9UJ41 | −0.57763 | 0.000237 | Cluster A |
| 569 | 3.25E−07 | Q9UJZ1 | 0.388886 | 1.39E−06 | Cluster B |
| 570 | 2.50E−05 | Q9UK45 | −0.94633 | 8.47E−05 | Cluster A |
| 571 | 6.04E−05 | Q9UK76 | −0.6992 | 0.000188 | Cluster A |
| 572 | 1.05E−14 | Q9UKM9 | 0.444614 | 9.83E−14 | Cluster B |
| 573 | 0.004138 | Q9UKV3 | 0.331258 | 0.009002 | Cluster B |
| 574 | 1.30E−06 | Q9UKY7 | −0.59835 | 5.22E−06 | Cluster A |
| 575 | 0.000638 | Q9ULV4 | −0.28439 | 0.001628 | Cluster A |
| 576 | 0.001995 | Q9ULZ3 | 0.260849 | 0.004596 | Cluster B |
| 577 | 6.83E−05 | Q9UM00 | 0.35684 | 0.000208 | Cluster B |
| 578 | 4.45E−05 | Q9UMS4 | 0.374463 | 0.000144 | Cluster B |
| 579 | 0.002089 | Q9UMX5 | 0.332133 | 0.004789 | Cluster B |
| 580 | 0.003481 | Q9UQ35 | 0.211948 | 0.007729 | Cluster B |
| 581 | 7.16E−24 | Q9UQ80 | −0.86166 | 1.39E−22 | Cluster A |
| 582 | 7.24E−07 | Q9Y237 | −0.74523 | 2.97E−06 | Cluster A |
| 583 | 5.74E−10 | Q9Y261 | −2.39187 | 3.34E−09 | Cluster A |
| 584 | 1.48E−16 | Q9Y266 | −0.62088 | 1.63E−15 | Cluster A |
| 585 | 2.23E−10 | Q9Y277 | 0.559255 | 1.35E−09 | Cluster B |
| 586 | 3.87E−12 | Q9Y2S6 | −0.76764 | 2.72E−11 | Cluster A |
| 587 | 4.71E−05 | Q9Y2W1 | −0.27429 | 0.000152 | Cluster A |
| 588 | 0.003295 | Q9Y2X3 | 0.259104 | 0.007329 | Cluster B |
| 589 | 0.000108 | Q9Y3B4 | 0.616503 | 0.000317 | Cluster B |
| 590 | 4.99E−05 | Q9Y4G6 | −1.13097 | 0.00016 | Cluster A |
| 591 | 7.81E−06 | Q9Y4L1 | 0.249345 | 2.90E−05 | Cluster B |
| 592 | 0.000787 | Q9Y5B9 | 0.185761 | 0.001986 | Cluster B |

TABLE 6
Protein set enrichment analysis between melanoma sub-populations

| | | | numberOf | fractionOfDB_{—} | Cond1med_{—} | Cond2med_{—} | | |
|---|---|---|---|---|---|---|---|---|
| | GO_term | pVal | Matches | Observed | int | int | qVal | dif |
| 1 | symporter activity | 2.76E−09 | 3 | 0.0625 | 0.0705 | 0.3146 | 8.14E−09 | −0.2441 |
| 2 | primary cilium | 1.34E−09 | 5 | 0.1190 | 0.0025 | 0.4329 | 4.04E−09 | −0.4303 |
| 3 | core promoter binding | 9.06E−15 | 7 | 0.1346 | −0.0026 | 0.2972 | 3.97E−14 | 0.2998 |
| 4 | placenta development | 3.49E−07 | 5 | 0.0820 | −0.1133 | −0.4332 | 8.48E−07 | 0.3199 |
| 5 | extrinsic to internal side of plasma membrane | 7.00E−17 | 8 | 0.1600 | 0.1004 | −0.2319 | 3.39E−16 | 0.3324 |
| 6 | positive regulation of neuron differentiation | 5.39E−09 | 9 | 0.0600 | 0.0397 | −0.2022 | 1.57E−08 | 0.2418 |
| 7 | MAPK cascade | 2.62E−11 | 12 | 0.0845 | 0.1048 | −0.1794 | 8.99E−11 | 0.2842 |
| 8 | activation of MAPK activity | 7.62E−06 | 13 | 0.1040 | 0.0473 | −0.2264 | 1.65E−05 | 0.2737 |
| 9 | positive regulation of epithelial cell proliferation | 5.10E−08 | 6 | 0.0714 | 0.3063 | −0.1582 | 1.34E−07 | 0.4645 |
| 10 | Rho GTPase activator activity | 1.33E−45 | 7 | 0.2333 | −0.0459 | −1.1085 | 1.87E−44 | 1.0626 |
| 11 | cytoskeleton organization | 2.16E−17 | 20 | 0.1515 | 0.0852 | −0.3638 | 1.07E−16 | 0.4491 |
| 12 | positive regulation of Rho GTPase activity | 6.73E−34 | 7 | 0.1321 | 0.0019 | −1.0512 | 6.72E−33 | 1.0531 |
| 13 | phagocytic vesicle membrane | 2.28E−16 | 13 | 0.1111 | −0.0679 | 0.2775 | 1.07E−15 | −0.3454 |
| 14 | intermediate filament | 1.85E−09 | 7 | 0.0446 | 0.3323 | 0.6053 | 5.55E−09 | −0.2730 |
| 15 | chloride transport | 7.93E−09 | 5 | 0.0735 | 0.0165 | −0.3134 | 2.26E−08 | 0.3299 |
| 16 | transmembrane transporter activity | 4.13E−63 | 12 | 0.1846 | 0.0942 | 0.4025 | 8.79E−62 | 0.3083 |
| 17 | odontogenesis of dentin-containing tooth | 4.94E−06 | 7 | 0.0588 | 0.0441 | 0.2580 | 1.08E−05 | 0.2139 |
| 18 | endonuclease activity | 1.32E−16 | 10 | 0.1786 | 0.0616 | −0.2292 | 6.26E−16 | 0.2908 |
| 19 | peroxidase activity | 1.18E−17 | 8 | 0.2000 | 0.2018 | −0.0631 | 6.02E−17 | 0.2649 |
| 20 | hydrogen peroxide catabolic process | 4.21E−25 | 9 | 0.4500 | 0.1843 | −0.1840 | 3.20E−24 | 0.3683 |
| 21 | acid-amino acid ligase activity | 2.91E−36 | 7 | 0.0560 | 0.1301 | −0.5181 | 3.14E−35 | 0.6482 |
| 22 | triglyceride biosynthetic process | 6.55E−35 | 6 | 0.1132 | 0.2813 | −0.2853 | 6.66E−34 | 0.5666 |
| 23 | long-chain fatty-acyl-CoA biosynthetic process | 6.55E−35 | 6 | 0.3529 | 0.2813 | −0.2853 | 6.66E−34 | 0.5666 |
| 24 | cellular lipid metabolic process | 2.27E−12 | 34 | 0.2048 | 0.1012 | 0.2368 | 8.51E−12 | −0.1356 |
| 25 | skeletal system development | 1.87E−20 | 14 | 0.0915 | 0.3169 | −0.0447 | 1.09E−19 | 0.3617 |
| 26 | extracellular matrix disassembly | 4.74E−05 | 22 | 0.1930 | −0.0140 | 0.1576 | 9.46E−05 | −0.1715 |
| 27 | adipose tissue development | 3.63E−05 | 6 | 0.1622 | 0.0206 | 0.1627 | 7.34E−05 | −0.1420 |
| 28 | negative regulation of cell growth | 2.60E−05 | 16 | 0.0994 | 0.0812 | −0.0636 | 5.32E−05 | 0.1447 |
| 29 | axonogenesis | 1.41E−10 | 17 | 0.1360 | 0.0515 | −0.0893 | 4.57E−10 | 0.1409 |
| 30 | negative regulation of microtubule polymerization | 2.02E−29 | 6 | 0.4286 | 0.0403 | −0.2787 | 1.78E−28 | 0.3190 |
| 31 | positive regulation of cellular component movement | 3.26E−77 | 4 | 0.2667 | 0.0403 | 0.4593 | 9.88E−76 | 0.4996 |
| 32 | glutathione metabolic process | 5.29E−09 | 10 | 0.1449 | −0.0257 | −0.3265 | 1.54E−08 | 0.3007 |
| 33 | pyridoxal phosphate binding | 1.63E−06 | 11 | 0.0679 | 0.1065 | 0.1870 | 3.77E−06 | −0.0805 |
| 34 | ubiquitin-ubiquitin ligase activity | 2.77E−05 | 4 | 0.3333 | 0.0054 | 0.2534 | 5.65E−05 | −0.2481 |
| 35 | integral to endoplasmic reticulum membrane | 2.34E−05 | 10 | 0.1064 | −0.0036 | 0.2176 | 4.80E−05 | 0.2212 |
| 36 | response to oxidative stress | 5.26E−10 | 20 | 0.1136 | 0.1467 | −0.0623 | 1.63E−09 | 0.2090 |
| 37 | learning or memory | 2.92E−24 | 5 | 0.0694 | −0.0350 | −0.5563 | 2.16E−23 | 0.5213 |
| 38 | ATP-dependent protein binding | 3.55E−06 | 3 | 0.2727 | 0.0082 | −0.1710 | 7.92E−06 | 0.1793 |
| 39 | negative regulation of sequence-specific DNA binding transcription | 3.44E−16 | 8 | 0.1270 | 0.0093 | −0.2079 | 1.59E−15 | 0.2172 |
| | factor activity | | | | | | | |
| 40 | negative regulation of T cell receptor signaling pathway | 1.93E−06 | 4 | 0.1905 | 0.2498 | −0.1641 | 4.44E−06 | 0.4139 |
| 41 | cytoskeletal protein binding | 2.09E−07 | 15 | 0.1485 | −0.0332 | −0.3650 | 5.22E−07 | 0.3318 |
| 42 | extrinsic to membrane | 2.92E−13 | 13 | 0.1912 | −0.0156 | −0.2922 | 1.18E−12 | 0.2766 |
| 43 | double-stranded RNA binding | 6.74E−12 | 24 | 0.3000 | 0.0125 | 0.0008 | 2.43E−11 | 0.0117 |
| 44 | single-stranded RNA binding | 4.12E−15 | 5 | 0.1190 | 0.0031 | 0.2632 | 1.83E−14 | −0.2601 |
| 45 | helicase activity | 7.06E−11 | 15 | 0.1095 | 0.0705 | −0.3055 | 2.32E−10 | 0.3760 |
| 46 | response to virus | 1.17E−82 | 31 | 0.2039 | 0.0444 | −0.2977 | 4.11E−81 | 0.3421 |
| 47 | actin filament organization | 2.04E−07 | 8 | 0.0899 | −0.1308 | −0.8892 | 5.13E−07 | 0.7584 |
| 48 | multicellular organism growth | 1.18E−11 | 6 | 0.0462 | 0.1847 | 0.6057 | 4.19E−11 | −0.4210 |
| 49 | cilium | 1.04E−14 | 23 | 0.1494 | 0.1024 | 0.2633 | 4.49E−14 | 0.3657 |
| 50 | aspartic-type endopeptidase activity | 2.04E−08 | 4 | 0.0488 | 0.0474 | 0.3875 | 5.62E−08 | −0.3400 |
| 51 | intermediate filament cytoskeleton | 3.32E−05 | 13 | 0.1238 | 0.0687 | 0.2199 | 6.73E−05 | −0.1512 |
| 52 | glucose homeostasis | 2.25E−77 | 6 | 0.0451 | 0.1080 | −0.7183 | 6.94E−76 | 0.8263 |
| 53 | anion transport | 1.84E−08 | 3 | 0.0714 | −0.0281 | 0.1523 | 5.09E−08 | 0.1804 |
| 54 | voltage-gated anion channel activity | 1.84E−08 | 3 | 0.2000 | −0.0281 | 0.1523 | 5.09E−08 | −0.1804 |
| 55 | PML body | 3.61E−14 | 13 | 0.1215 | 0.1276 | 0.1615 | 1.51E−13 | 0.2891 |
| 56 | apoptotic signaling pathway | 2.39E−28 | 19 | 0.1557 | 0.1409 | −0.4552 | 2.04E−27 | 0.5961 |
| 57 | positive regulation of apoptotic signaling pathway | 2.51E−14 | 4 | 0.0784 | 0.1245 | −0.2836 | 1.06E−13 | 0.4081 |
| 58 | protein export from nucleus | 3.48E−64 | 14 | 0.3111 | 0.0809 | −0.2336 | 7.50E−63 | 0.3146 |
| 59 | embryo implantation | 2.87E−06 | 4 | 0.0435 | 0.0101 | −0.1593 | 6.49E−06 | 0.1694 |
| 60 | ATP-dependent DNA helicase activity | 3.91E−14 | 14 | 0.2692 | −0.0052 | 0.1367 | 1.63E−13 | −0.1419 |
| 61 | DNA duplex unwinding | 3.27E−07 | 18 | 0.2169 | 0.0299 | 0.1222 | 7.98E−07 | −0.0923 |
| 62 | cellular protein modification process | 3.25E−19 | 20 | 0.1274 | 0.0780 | −0.0953 | 1.80E−18 | 0.1734 |
| 63 | antigen processing and presentation | 2.30E−20 | 7 | 0.1129 | 0.0029 | 0.4295 | 1.33E−19 | −0.4265 |
| 64 | proteolysis involved in cellular protein catabolic process | 8.16E−06 | 5 | 0.1190 | −0.1262 | 0.0621 | 1.76E−05 | 0.1883 |
| 65 | antigen processing and presentation of peptide antigen via MHC class I | 3.02E−08 | 57 | 0.3202 | −0.0286 | 0.0329 | 8.12E−08 | 0.0615 |
| 66 | peptide antigen binding | 2.20E−08 | 5 | 0.0420 | −0.0958 | 0.1983 | 5.99E−08 | 0.2941 |
| 67 | protein peptidyl-prolyl isomerization | 1.67E−20 | 16 | 0.1720 | 0.0082 | 0.2224 | 9.90E−20 | 0.2306 |
| 68 | peptidyl-prolyl cis-trans isomerase activity | 1.67E−20 | 16 | 0.1720 | 0.0082 | −0.2224 | 9.90E−20 | 0.2306 |
| 69 | nucleoside diphosphate kinase activity | 3.61E−13 | 5 | 0.1111 | 0.2514 | −0.2527 | 1.44E−12 | 0.5041 |
| 70 | nucleoside diphosphate phosphorylation | 3.61E−13 | 5 | 0.1136 | 0.2514 | −0.2527 | 1.44E−12 | 0.5041 |
| 71 | GTP biosynthetic process | 3.52E−14 | 3 | 0.0833 | 0.2785 | 0.2801 | 1.47E−13 | 0.5585 |
| 72 | UTP biosynthetic process | 6.81E−11 | 4 | 0.1081 | 0.1666 | −0.2017 | 2.25E−10 | 0.3682 |
| 73 | CTP biosynthetic process | 1.41E−12 | 4 | 0.1081 | 0.1841 | −0.2017 | 5.42E−12 | 0.3858 |
| 74 | tRNA binding | 2.68E−07 | 15 | 0.3000 | −0.0021 | −0.1247 | 6.63E−07 | 0.1227 |
| 75 | endosome to lysosome transport | 6.22E−12 | 5 | 0.1471 | 0.1312 | 0.4981 | 2.25E−11 | −0.3669 |
| 76 | skeletal muscle cell differentiation | 2.30E−06 | 4 | 0.0625 | 0.1282 | 0.4153 | 5.21E−06 | 0.2871 |
| 77 | cellular response to organic cyclic compound | 2.24E−08 | 5 | 0.0746 | −0.0754 | −0.3551 | 6.08E−08 | 0.2796 |
| 78 | spindle assembly | 1.88E−31 | 4 | 0.0548 | 0.1006 | −0.9763 | 1.72E−30 | 1.0769 |
| 79 | regulation of heart rate by cardiac conduction | 6.04E−40 | 4 | 0.1667 | 0.3252 | −0.6161 | 7.21E−39 | 0.9413 |
| 80 | negative regulation of retinoic acid receptor signaling pathway | 5.52E−10 | 3 | 0.0652 | −0.2428 | 0.1134 | 1.71E−09 | −0.3563 |
| 81 | response to antibiotic | 2.32E−05 | 3 | 0.0612 | 0.0337 | 0.1976 | 4.77E−05 | −0.1640 |
| 82 | hippo signaling cascade | 2.57E−64 | 6 | 0.1667 | 0.2426 | −0.5479 | 5.70E−63 | 0.7905 |
| 83 | retina homeostasis | 2.21E−20 | 9 | 0.2250 | 0.0474 | −0.2762 | 1.28E−19 | 0.3236 |
| 84 | cell cortex | 1.90E−12 | 26 | 0.1436 | 0.1600 | −0.0661 | 7.17E−12 | 0.2262 |
| 85 | blood microparticle | 4.94E−52 | 22 | 0.1358 | 0.0237 | −0.3317 | 8.11E−51 | 0.3553 |
| 86 | lipid particle organization | 1.40E−71 | 4 | 0.3333 | 0.2428 | −0.8938 | 3.77E−70 | 1.1365 |
| 87 | viral infectious cycle | 0 | 89 | 0.7542 | 0.0755 | −0.5257 | 0 | 0.6012 |
| 88 | membrane organization | 1.16E−51 | 58 | 0.3946 | 0.0756 | −0.1968 | 1.88E−50 | 0.2724 |
| 89 | ruffle | 1.86E−62 | 32 | 0.2270 | 0.0847 | −0.2427 | 3.90E−61 | 0.3273 |
| 90 | neuron differentiation | 1.09E−05 | 9 | 0.0938 | 0.1505 | 0.0565 | 2.30E−05 | 0.0940 |
| 91 | nucleosome | 1.45E−68 | 18 | 0.2169 | 0.0272 | 0.3488 | 3.64E−67 | −0.3216 |
| 92 | nucleosome assembly | 5.01E−45 | 33 | 0.1823 | 0.0206 | 0.2396 | 6.75E−44 | −0.2190 |
| 93 | nuclear-transcribed mRNA catabolic process, deadenylation-dependent | 7.76E−24 | 20 | 0.3636 | 0.0383 | −0.2885 | 5.66E−23 | 0.3268 |
| | decay | | | | | | | |
| 94 | nuclear-transcribed mRNA poly(A) tail shortening | 2.24E−35 | 9 | 0.3000 | 0.0401 | −0.3240 | 2.35E−34 | 0.3641 |
| 95 | regulation of translation | 6.57E−53 | 19 | 0.2568 | 0.0870 | −0.3151 | 1.11E−51 | 0.4020 |
| 96 | estrogen receptor binding | 1.15E−14 | 8 | 0.2759 | 0.0776 | 0.3261 | 4.96E−14 | −0.2486 |
| 97 | gene silencing by RNA | 2.66E−10 | 9 | 0.2727 | 0.1104 | −0.2671 | 8.35E−10 | 0.3775 |
| 98 | negative regulation of intracellular estrogen receptor signaling pathway | 3.15E−20 | 4 | 0.3333 | 0.1520 | 0.5860 | 1.81E−19 | −0.4340 |
| 99 | negative regulation of catalytic activity | 1.78E−14 | 19 | 0.1397 | 0.0388 | −0.1225 | 7.60E−14 | 0.1613 |
| 100 | DNA binding, bending | 2.22E−08 | 12 | 0.2034 | −0.0230 | −0.1468 | 6.05E−08 | 0.1238 |
| 101 | regulation of cell proliferation | 2.17E−06 | 21 | 0.1214 | 0.0683 | 0.2382 | 4.96E−06 | −0.1699 |
| 102 | autophagic vacuole assembly | 9.32E−07 | 5 | 0.0877 | 0.0031 | 0.2057 | 2.19E−06 | −0.2026 |
| 103 | synaptic vesicle | 8.40E−07 | 9 | 0.0581 | 0.0120 | 0.2800 | 1.98E−06 | −0.2681 |
| 104 | calcium-dependent protein binding | 5.47E−12 | 19 | 0.2235 | −0.0247 | −0.1651 | 1.99E−11 | 0.1404 |
| 105 | establishment of cell polarity | 2.46E−101 | 4 | 0.0784 | 0.3553 | −0.9167 | 1.38E−99 | 1.2720 |
| 106 | single fertilization | 2.02E−15 | 5 | 0.0962 | 0.1055 | −0.3123 | 9.03E−15 | 0.4178 |
| 107 | NADP binding | 1.94E−36 | 13 | 0.1625 | 0.1179 | −0.3299 | 2.12E−35 | 0.4477 |
| 108 | histone deacetylase activity | 6.89E−06 | 5 | 0.1064 | −0.0406 | 0.1368 | 1.49E−05 | −0.1774 |
| 109 | cytoplasmic microtubule | 5.79E−82 | 11 | 0.1310 | 0.1127 | −0.6804 | 1.98E−80 | 0.7931 |
| 110 | histone deacetylation | 1.29E−25 | 7 | 0.1186 | 0.0518 | 0.3953 | 9.95E−25 | −0.3436 |
| 111 | ubiquitin binding | 8.62E−07 | 13 | 0.2500 | 0.0313 | −0.2052 | 2.03E−06 | 0.2365 |
| 112 | beta-tubulin binding | 1.57E−10 | 9 | 0.1800 | 0.0572 | 0.3093 | 5.04E−10 | −0.2521 |
| 113 | Rab GTPase activator activity | 1.46E−19 | 4 | 0.0303 | −0.0263 | −0.4140 | 8.21E−19 | 0.3877 |
| 114 | positive regulation of Rab GTPase activity | 1.46E−19 | 4 | 0.0303 | −0.0263 | −0.4140 | 8.21E−19 | 0.3877 |
| 115 | methyltransferase activity | 3.45E−19 | 12 | 0.0645 | 0.0690 | −0.2851 | 1.91E−18 | 0.3541 |
| 116 | regulation of growth | 3.09E−07 | 12 | 0.2308 | 0.0290 | −0.1444 | 7.61E−07 | 0.1734 |
| 117 | negative regulation of viral genome replication | 3.83E−08 | 9 | 0.2143 | 0.0102 | 0.2127 | 1.02E−07 | −0.2025 |
| 118 | cytosolic small ribosomal subunit | 0 | 33 | 0.8049 | 0.0993 | −0.6353 | 0 | 0.7347 |
| 119 | chromatin | 1.24E−06 | 24 | 0.2182 | 0.0045 | −0.0739 | 2.88E−06 | 0.0784 |
| 120 | cysteine-type endopeptidase activity | 1.99E−06 | 10 | 0.0926 | −0.0747 | 0.1546 | 4.57E−06 | −0.2293 |
| 121 | regulation of translational fidelity | 2.64E−08 | 6 | 0.2727 | 0.0441 | −0.1194 | 7.13E−08 | 0.1635 |
| 122 | nuclear pore | 1.33E−32 | 30 | 0.2655 | 0.1103 | −0.2307 | 1.26E−31 | 0.3410 |
| 123 | mRNA transport | 2.18E−06 | 21 | 0.3000 | 0.0471 | 0.1606 | 4.96E−06 | 0.1134 |
| 124 | microtubule cytoskeleton | 1.54E−24 | 30 | 0.1563 | 0.1194 | −0.1106 | 1.16E−23 | 0.2300 |
| 125 | negative regulation of Wnt receptor signaling pathway | 1.77E−07 | 4 | 0.0656 | 0.0312 | −0.2562 | 4.49E−07 | 0.2875 |
| 126 | post-embryonic development | 1.95E−10 | 12 | 0.0774 | 0.1640 | 0.4913 | 6.23E−10 | −0.3273 |
| 127 | regulation of alternative nuclear mRNA splicing, via spliceosome | 3.71E−08 | 12 | 0.4000 | 0.0220 | 0.1497 | 9.86E−08 | 0.1278 |
| 128 | antioxidant activity | 1.03E−12 | 4 | 0.1905 | 0.1147 | −0.2135 | 4.01E−12 | 0.3282 |
| 129 | mRNA binding | 7.48E−56 | 36 | 0.3333 | 0.0451 | −0.1553 | 1.36E−54 | 0.2004 |
| 130 | sequence-specific DNA binding RNA polymerase II transcription factor | 9.11E−34 | 3 | 0.0288 | 0.2147 | 0.8005 | 8.97E−33 | −0.5858 |
| | activity | | | | | | | |
| 131 | purine base metabolic process | 4.56E−11 | 17 | 0.4722 | 0.0051 | −0.1525 | 1.52E−10 | 0.1576 |
| 132 | sodium:potassium-exchanging ATPase complex | 3.24E−11 | 3 | 0.1154 | 0.0680 | 0.5054 | 1.10E−10 | −0.4373 |
| 133 | apical part of cell | 2.53E−09 | 14 | 0.0946 | 0.0169 | −0.1250 | 7.49E−09 | 0.1420 |
| 134 | microtubule organizing center | 3.38E−07 | 27 | 0.1765 | 0.0101 | −0.0960 | 8.24E−07 | 0.1062 |
| 135 | negative regulation of NF-kappaB transcription factor activity | 6.85E−06 | 8 | 0.1039 | −0.0904 | 0.0420 | 1.49E−05 | −0.1324 |
| 136 | tumor necrosis factor-mediated signaling pathway | 4.27E−05 | 3 | 0.0667 | −0.0463 | 0.1898 | 8.56E−05 | −0.2362 |
| 137 | spliceosomal complex | 4.85E−26 | 54 | 0.4655 | 0.0109 | 0.1803 | 3.78E−25 | −0.1694 |
| 138 | structural constituent of cytoskeleton | 1.17E−70 | 30 | 0.1935 | 0.0011 | −0.3164 | 3.01E−69 | 0.3175 |
| 139 | microtubule-based process | 1.19E−115 | 12 | 0.1188 | 0.0609 | −0.8660 | 7.48E−114 | 0.9269 |
| 140 | protein polymerization | 1.20E−139 | 8 | 0.1702 | 0.0704 | −1.0025 | 9.42E−138 | 1.0729 |
| 141 | rRNA processing | 4.27E−184 | 45 | 0.3846 | 0.0652 | −0.4179 | 4.48E−182 | 0.4830 |
| 142 | ribonucleoprotein complex | 2.02E−156 | 66 | 0.4314 | 0.0591 | −0.1540 | 1.99E−154 | 0.2132 |
| 143 | dendritic spine | 1.92E−08 | 15 | 0.1200 | 0.1852 | −0.0085 | 5.29E−08 | 0.1937 |
| 144 | somitogenesis | 1.43E−08 | 5 | 0.0676 | 0.0916 | 0.3582 | 4.01E−08 | −0.2666 |
| 145 | glycolysis | 1.13E−306 | 22 | 0.1667 | 0.1840 | −0.4889 | 1.62E−304 | 0.6729 |
| 146 | response to ischemia | 4.89E−05 | 3 | 0.0882 | −0.0610 | 0.2024 | 9.73E−05 | −0.2634 |
| 147 | RNA processing | 7.99E−24 | 30 | 0.2290 | −0.0006 | 0.1877 | 5.80E−23 | −0.1883 |
| 148 | regulation of cell morphogenesis | 1.27E−52 | 5 | 0.2632 | 0.2686 | −0.7196 | 2.10E−51 | 0.9882 |
| 149 | transcription regulatory region sequence-specific DNA binding | 9.40E−09 | 4 | 0.0482 | 0.0375 | 0.3156 | 2.67E−08 | −0.2780 |
| 150 | translation initiation factor activity | 8.02E−41 | 34 | 0.2267 | −0.0037 | 0.2885 | 9.80E−40 | 0.2848 |
| 151 | immune system process | 2.17E−08 | 9 | 0.2093 | 0.1911 | −0.1314 | 5.97E−08 | 0.3224 |
| 152 | negative regulation of type I interferon production | 1.22E−23 | 5 | 0.1429 | 0.0887 | −0.4462 | 8.81E−23 | 0.5349 |
| 153 | cellular response to interferon-gamma | 2.00E−71 | 4 | 0.1379 | 0.1112 | −0.3624 | 5.26E−70 | 0.4737 |
| 154 | cellular response to interleukin-1 | 3.45E−09 | 4 | 0.0909 | −0.0024 | 0.2461 | 1.01E−08 | −0.2485 |
| 155 | negative regulation of protein serine/threonine kinase activity | 3.37E−55 | 6 | 0.1935 | 0.0660 | −0.3827 | 5.96E−54 | 0.4487 |
| 156 | spindle pole | 9.18E−07 | 26 | 0.1857 | 0.0174 | 0.2370 | 2.16E−06 | −0.2196 |
| 157 | cerebral cortex development | 3.88E−32 | 11 | 0.1375 | 0.1873 | −0.3623 | 3.60E−31 | 0.5496 |
| 158 | spindle | 3.96E−09 | 35 | 0.2397 | 0.0818 | −0.0456 | 1.16E−08 | 0.1274 |
| 159 | proton-transporting V-type ATPase, V1 domain | 1.62E−12 | 4 | 0.2500 | 0.1335 | −0.1283 | 6.17E−12 | 0.2617 |
| 160 | hydrogen ion transporting ATP synthase activity, rotational mechanism | 3.79E−65 | 8 | 0.3077 | 0.1019 | 0.4455 | 8.53E−64 | −0.3436 |
| 161 | proton-transporting ATPase activity, rotational mechanism | 7.99E−07 | 12 | 0.4000 | 0.1049 | 0.2277 | 1.89E−06 | −0.1228 |
| 162 | Rho GTPase binding | 3.68E−08 | 11 | 0.2075 | −0.0131 | 0.1994 | 9.81E−08 | 0.1863 |
| 163 | DNA-directed DNA polymerase activity | 4.94E−05 | 6 | 0.0674 | −0.0117 | 0.3337 | 9.81E−05 | −0.3454 |
| 164 | DNA-dependent DNA replication | 8.35E−08 | 8 | 0.0870 | 0.0605 | 0.3330 | 2.16E−07 | −0.2725 |
| 165 | melanosome | 9.96E−10 | 73 | 0.7157 | 0.0963 | 0.0679 | 3.02E−09 | 0.0283 |
| 166 | bone mineralization | 1.57E−07 | 3 | 0.0652 | −0.0597 | 0.3057 | 3.98E−07 | −0.3654 |
| 167 | cytoplasmic vesicle membrane | 1.17E−92 | 30 | 0.2521 | 0.1624 | −0.3644 | 5.76E−91 | 0.5268 |
| 168 | amino acid transport | 4.65E−08 | 5 | 0.1282 | −0.1159 | 0.1735 | 1.23E−07 | −0.2894 |
| 169 | regulation of release of sequestered calcium ion into cytosol by | 5.57E−07 | 3 | 0.2143 | 0.0933 | −0.0929 | 1.33E−06 | 0.1862 |
| | sarcoplasmic reticulum | | | | | | | |
| 170 | sarcoplasmic reticulum membrane | 4.37E−11 | 7 | 0.2333 | 0.0937 | 0.3509 | 1.46E−10 | −0.2572 |
| 171 | liver development | 5.72E−13 | 13 | 0.0813 | 0.0131 | 0.2189 | 2.25E−12 | −0.2059 |
| 172 | RNA polymerase II distal enhancer sequence-specific DNA binding | 4.38E−06 | 14 | 0.3111 | −0.0408 | 0.0626 | 9.67E−06 | −0.1034 |
| 173 | neural crest cell migration | 1.77E−92 | 4 | 0.0702 | 0.3660 | −0.8998 | 8.44E−91 | 1.2658 |
| 174 | developmental growth | 1.98E−12 | 4 | 0.0769 | 0.0295 | −0.4418 | 7.45E−12 | 0.4713 |
| 175 | ruffle membrane | 1.09E−27 | 23 | 0.2473 | 0.0407 | −0.3348 | 9.12E−27 | 0.3755 |
| 176 | protein self-association | 1.12E−14 | 9 | 0.1800 | 0.1385 | 0.5579 | 4.82E−14 | −0.4194 |
| 177 | regulation of cell shape | 3.37E−13 | 23 | 0.1250 | 0.0178 | −0.2560 | 1.36E−12 | 0.2738 |
| 178 | DNA catabolic process, endonucleolytic | 5.09E−13 | 9 | 0.1011 | 0.0426 | −0.1945 | 2.00E−12 | 0.2370 |
| 179 | glucose metabolic process | 3.84E−115 | 50 | 0.3311 | 0.1273 | −0.2688 | 2.33E−113 | 0.3961 |
| 180 | postsynaptic density | 3.50E−29 | 13 | 0.0677 | 0.0640 | −0.3200 | 3.05E−28 | 0.3841 |
| 181 | p53 binding | 1.91E−10 | 8 | 0.1290 | −0.0494 | 0.2327 | 6.11E−10 | −0.2822 |
| 182 | cellular response to hydrogen peroxide | 6.94E−08 | 8 | 0.1250 | 0.0778 | −0.1775 | 1.81E−07 | 0.2553 |
| 183 | recycling endosome | 4.27E−16 | 12 | 0.1463 | 0.0088 | 0.3471 | 1.96E−15 | −0.3382 |
| 184 | response to toxin | 2.05E−07 | 10 | 0.0901 | 0.0741 | −0.0994 | 5.15E−07 | 0.1734 |
| 185 | response to cytokine stimulus | 3.45E−08 | 5 | 0.0556 | 0.0000 | 0.2740 | 9.22E−08 | −0.2740 |
| 186 | chloride channel activity | 2.42E−10 | 6 | 0.0968 | 0.0219 | −0.3337 | 7.64E−10 | 0.3556 |
| 187 | ATPase activity, coupled | 4.34E−14 | 7 | 0.2500 | 0.0624 | −0.3040 | 1.79E−13 | 0.3664 |
| 188 | chaperone mediated protein folding requiring cofactor | 1.70E−38 | 4 | 0.1212 | 0.0926 | −0.4196 | 1.94E−37 | 0.5122 |
| 189 | G2/M transition of mitotic cell cycle | 2.23E−99 | 48 | 0.3179 | 0.0944 | −0.2645 | 1.21E−97 | 0.3589 |
| 190 | regulation of ion transmembrane transport | 1.86E−05 | 11 | 0.0618 | 0.0070 | −0.1937 | 3.87E−05 | 0.2007 |
| 191 | chloride channel complex | 1.10E−08 | 5 | 0.0862 | 0.0071 | −0.3284 | 3.09E−08 | 0.3355 |
| 192 | protein complex assembly | 7.43E−19 | 27 | 0.1971 | 0.0411 | −0.1599 | 3.98E−18 | 0.2009 |
| 193 | cortical cytoskeleton | 1.01E−10 | 10 | 0.2941 | −0.0269 | −0.1778 | 3.30E−10 | 0.1509 |
| 194 | JNK cascade | 6.36E−17 | 4 | 0.0667 | 0.0967 | −0.4574 | 3.09E−16 | 0.5540 |
| 195 | telomere maintenance | 1.80E−11 | 24 | 0.3038 | 0.0521 | 0.2155 | 6.25E−11 | −0.1634 |
| 196 | protein sumoylation | 2.10E−06 | 9 | 0.2813 | 0.1265 | −0.0576 | 4.79E−06 | 0.1841 |
| 197 | M band | 5.72E−91 | 10 | 0.3571 | 0.1855 | −0.5734 | 2.51E−89 | 0.7589 |
| 198 | I band | 3.67E−48 | 4 | 0.1600 | 0.1431 | −0.7377 | 5.61E−47 | 0.8808 |
| 199 | cytosolic large ribosomal subunit | 0 | 46 | 0.8214 | 0.0575 | −0.4983 | 0 | 0.5558 |
| 200 | protein heterooligomerization | 6.72E−15 | 15 | 0.1049 | 0.1137 | −0.1556 | 2.96E−14 | 0.2693 |
| 201 | mitochondrial proton-transporting ATP synthase complex, coupling | 1.11E−27 | 5 | 0.4545 | 0.1134 | 0.5065 | 9.30E−27 | −0.3931 |
| | factor F(o) | | | | | | | |
| 202 | protein localization | 5.79E−07 | 12 | 0.1379 | 0.1527 | 0.4441 | 1.38E−06 | −0.2915 |
| 203 | T cell activation | 1.08E−07 | 6 | 0.0938 | 0.0074 | 0.1496 | 2.77E−07 | −0.1422 |
| 204 | mitochondrial respiratory chain complex I | 3.97E−06 | 9 | 0.1579 | 0.1040 | 0.4186 | 8.78E−06 | −0.3146 |
| 205 | NAD metabolic process | 4.83E−21 | 3 | 0.1765 | 0.2828 | −0.2346 | 2.99E−20 | 0.5174 |
| 206 | cellular carbohydrate metabolic process | 9.11E−40 | 7 | 0.1373 | 0.2311 | −0.1412 | 1.06E−38 | 0.3723 |
| 207 | NAD binding | 9.83E−21 | 21 | 0.2414 | 0.1693 | −0.0252 | 6.03E−20 | 0.1945 |
| 208 | kinesin complex | 7.66E−22 | 15 | 0.0932 | 0.2500 | −0.3960 | 5.08E−21 | 0.6461 |
| 209 | protein serine/threonine phosphatase activity | 7.16E−10 | 17 | 0.2615 | 0.0799 | 0.2206 | 2.20E−09 | −0.1407 |
| 210 | positive regulation of DNA binding | 4.81E−43 | 7 | 0.2414 | −0.0171 | −0.4702 | 6.21E−42 | 0.4530 |
| 211 | positive regulation of protein phosphorylation | 8.74E−07 | 17 | 0.1012 | 0.0585 | −0.1902 | 2.06E−06 | 0.2487 |
| 212 | protein disulfide oxidoreductase activity | 6.18E−13 | 7 | 0.1795 | −0.0757 | −0.2602 | 2.42E−12 | 0.1845 |
| 213 | cell redox homeostasis | 1.59E−12 | 20 | 0.1342 | −0.0338 | 0.1726 | 6.07E−12 | −0.2065 |
| 214 | small ribosomal subunit | 0 | 13 | 0.2826 | 0.1058 | −0.7565 | 0 | 0.8623 |
| 215 | rRNA binding | 9.03E−240 | 12 | 0.3000 | 0.0760 | −0.5321 | 1.09E−237 | 0.6081 |
| 216 | protein methylation | 6.24E−14 | 5 | 0.0847 | 0.0592 | −0.2891 | 2.56E−13 | 0.3483 |
| 217 | fatty acid biosynthetic process | 4.48E−12 | 7 | 0.0875 | 0.4321 | −0.1686 | 1.64E−11 | 0.6007 |
| 218 | U1 snRNP | 5.40E−17 | 12 | 0.3750 | 0.0210 | 0.1874 | 2.63E−16 | −0.2084 |
| 219 | mRNA splice site selection | 1.67E−17 | 8 | 0.1818 | 0.0310 | 0.3085 | 8.45E−17 | −0.2775 |
| 220 | RS domain binding | 2.91E−07 | 5 | 0.3846 | −0.0064 | 0.2875 | 7.18E−07 | −0.2939 |
| 221 | nuclear periphery | 8.78E−06 | 5 | 0.4167 | −0.1023 | 0.3065 | 1.88E−05 | −0.4087 |
| 222 | protein import into nucleus | 5.08E−09 | 11 | 0.1774 | 0.0532 | −0.0212 | 1.48E−08 | 0.0744 |
| 223 | cell projection assembly | 2.82E−24 | 3 | 0.2000 | 0.2165 | −0.4015 | 2.11E−23 | 0.6180 |
| 224 | response to insulin stimulus | 4.21E−17 | 6 | 0.0583 | 0.0756 | 0.3782 | 2.07E−16 | −0.3026 |
| 225 | positive regulation of protein binding | 2.21E−78 | 7 | 0.1296 | 0.0653 | −0.6790 | 6.96E−77 | 0.7443 |
| 226 | protein targeting to mitochondrion | 2.07E−10 | 25 | 0.4032 | 0.0849 | 0.2611 | 6.58E−10 | −0.1762 |
| 227 | cell leading edge | 8.20E−06 | 10 | 0.1515 | 0.1216 | −0.0921 | 1.76E−05 | 0.2137 |
| 228 | mast cell granule | 1.73E−76 | 4 | 0.1333 | 0.3477 | −0.6160 | 5.14E−75 | 0.9637 |
| 229 | positive regulation of insulin secretion | 3.97E−05 | 6 | 0.0984 | 0.1055 | 0.4181 | 7.98E−05 | −0.3126 |
| 230 | myosin complex | 1.30E−11 | 12 | 0.1111 | 0.1370 | −0.1418 | 4.59E−11 | 0.2788 |
| 231 | regulation of gene expression | 1.96E−05 | 8 | 0.0860 | 0.1581 | −0.1596 | 4.06E−05 | 0.3177 |
| 232 | positive regulation of proteasomal ubiquitin-dependent protein catabolic | 1.00E−21 | 9 | 0.1607 | 0.0718 | −0.2466 | 6.49E−21 | 0.3184 |
| | process | | | | | | | |
| 233 | condensed nuclear chromosome | 4.75E−13 | 11 | 0.3056 | 0.0478 | 0.3658 | 1.88E−12 | −0.3180 |
| 234 | nuclear inner membrane | 2.88E−40 | 10 | 0.2857 | 0.0419 | 0.3551 | 3.46E−39 | −0.3132 |
| 235 | formation of translation preinitiation complex | 6.07E−09 | 13 | 0.5200 | −0.0183 | −0.2185 | 1.75E−08 | 0.2002 |
| 236 | eukaryotic translation initiation factor 3 complex | 1.80E−08 | 14 | 0.2090 | −0.0189 | −0.1858 | 5.02E−08 | 0.1669 |
| 237 | regulation of translational initiation | 1.34E−21 | 27 | 0.5000 | 0.0030 | −0.2245 | 8.64E−21 | 0.2275 |
| 238 | eukaryotic 43S preinitiation complex | 6.07E−09 | 13 | 0.5200 | −0.0183 | −0.2185 | 1.75E−08 | 0.2002 |
| 239 | eukaryotic 48S preinitiation complex | 6.07E−09 | 13 | 0.5417 | −0.0183 | −0.2185 | 1.75E−08 | 0.2002 |
| 240 | protein N-linked glycosylation | 3.23E−07 | 3 | 0.0909 | 0.0314 | 0.3826 | 7.93E−07 | −0.3513 |
| 241 | Rho protein signal transduction | 9.20E−44 | 13 | 0.2167 | 0.0497 | −0.8038 | 1.20E−42 | 0.8536 |
| 242 | DNA metabolic process | 8.58E−12 | 5 | 0.1000 | 0.0711 | −0.2524 | 3.07E−11 | 0.3235 |
| 243 | DNA-dependent ATPase activity | 3.04E−05 | 5 | 0.0794 | −0.1703 | 0.0236 | 6.18E−05 | −0.1939 |
| 244 | regulation of actin cytoskeleton organization | 4.17E−21 | 10 | 0.1370 | −0.0419 | −0.6609 | 2.59E−20 | 0.6190 |
| 245 | response to hormone stimulus | 1.37E−35 | 10 | 0.1724 | 0.0156 | 0.3757 | 1.46E−34 | −0.3601 |
| 246 | lipoprotein metabolic process | 1.87E−16 | 3 | 0.0417 | −0.0719 | 0.2379 | 8.83E−16 | −0.3097 |
| 247 | negative regulation of protein kinase activity | 2.11E−88 | 12 | 0.1290 | 0.0908 | −0.5506 | 8.31E−87 | 0.6414 |
| 248 | fatty acid metabolic process | 2.22E−13 | 6 | 0.0952 | 0.2441 | −0.1549 | 9.01E−13 | 0.3989 |
| 249 | muscle cell homeostasis | 8.67E−19 | 4 | 0.1250 | 0.0726 | −0.5939 | 4.63E−18 | 0.6664 |
| 250 | ESC/E(Z) complex | 3.65E−06 | 4 | 0.1429 | −0.1091 | 0.1234 | 8.10E−06 | −0.2325 |
| 251 | establishment of mitotic spindle orientation | 3.77E−05 | 3 | 0.0882 | 0.0530 | 0.3136 | 7.61E−05 | −0.2606 |
| 252 | inner cell mass cell proliferation | 2.67E−05 | 3 | 0.1250 | 0.0106 | 0.2412 | 5.47E−05 | −0.2307 |
| 253 | retrograde vesicle-mediated transport, Golgi to ER | 4.27E−09 | 15 | 0.4545 | 0.0158 | 0.1946 | 1.25E−08 | −0.1788 |
| 254 | regulation of cell adhesion | 1.15E−06 | 12 | 0.2182 | 0.0974 | −0.0750 | 2.68E−06 | 0.1724 |
| 255 | bone resorption | 2.43E−11 | 4 | 0.1081 | 0.0514 | 0.3704 | 8.36E−11 | −0.3190 |
| 256 | uropod | 3.71E−14 | 4 | 0.2857 | −0.0786 | −0.2817 | 1.55E−13 | 0.2031 |
| 257 | cellular component movement | 7.40E−44 | 35 | 0.3241 | −0.0436 | −0.3191 | 9.72E−43 | 0.2756 |
| 258 | myosin II complex | 1.88E−21 | 3 | 0.2727 | 0.1398 | −0.1386 | 1.20E−20 | 0.2783 |
| 259 | regulation of blood pressure | 2.04E−05 | 9 | 0.1200 | −0.0237 | 0.2003 | 4.22E−05 | −0.2240 |
| 260 | DNA damage response, signal transduction resulting in induction of | 9.15E−11 | 7 | 0.0769 | 0.0628 | −0.0451 | 2.98E−10 | 0.1079 |
| | apoptosis | | | | | | | |
| 261 | DNA-dependent DNA replication initiation | 1.26E−10 | 8 | 0.2222 | −0.1097 | 0.0908 | 4.08E−10 | −0.2005 |
| 262 | MCM complex | 4.30E−11 | 7 | 0.3684 | −0.1105 | 0.0900 | 1.44E−10 | −0.2005 |
| 263 | cell division | 1.33E−50 | 21 | 0.1963 | 0.0561 | −0.2698 | 2.12E−49 | 0.3259 |
| 264 | damaged DNA binding | 3.44E−06 | 20 | 0.1587 | 0.0462 | −0.0431 | 7.69E−06 | 0.0893 |
| 265 | double-strand break repair via nonhomologous end joining | 5.49E−09 | 7 | 0.2500 | 0.1137 | 0.3252 | 1.59E−08 | −0.2115 |
| 266 | ADP binding | 1.06E−05 | 14 | 0.3590 | 0.0403 | 0.1046 | 2.24E−05 | −0.0643 |
| 267 | flavin adenine dinucleotide binding | 9.40E−29 | 16 | 0.0851 | 0.0363 | 0.3354 | 8.14E−28 | −0.2992 |
| 268 | keratinocyte differentiation | 9.44E−22 | 8 | 0.1212 | 0.1356 | −0.4606 | 6.17E−21 | 0.5962 |
| 269 | fatty-acyl-CoA binding | 2.57E−34 | 7 | 0.2414 | 0.0747 | 0.5318 | 2.58E−33 | −0.4571 |
| 270 | embryo development | 2.37E−05 | 13 | 0.0935 | 0.0505 | 0.1017 | 4.87E−05 | −0.0512 |
| 271 | response to starvation | 8.38E−12 | 5 | 0.0877 | 0.0057 | 0.3514 | 3.00E−11 | −0.3457 |
| 272 | hippocampus development | 4.08E−08 | 7 | 0.1045 | 0.2322 | −0.1195 | 1.08E−07 | 0.3518 |
| 273 | ion channel binding | 1.29E−29 | 16 | 0.1600 | 0.0685 | −0.1936 | 1.14E−28 | 0.2622 |
| 274 | brush border | 4.95E−05 | 5 | 0.1250 | 0.0005 | −0.0916 | 9.82E−05 | 0.0922 |
| 275 | F-actin capping protein complex | 2.75E−61 | 5 | 0.3333 | 0.3471 | −0.4389 | 5.63E−60 | 0.7860 |
| 276 | ribosome binding | 5.66E−10 | 19 | 0.3654 | 0.0026 | −0.1109 | 1.74E−09 | 0.1135 |
| 277 | response to amino acid stimulus | 6.97E−53 | 4 | 0.0800 | 0.2121 | −0.4568 | 1.17E−51 | 0.6689 |
| 278 | positive regulation of translation | 4.31E−22 | 19 | 0.3115 | 0.0426 | −0.2001 | 2.90E−21 | 0.2428 |
| 279 | positive regulation of stress fiber assembly | 6.98E−24 | 5 | 0.1020 | 0.0674 | −0.5145 | 5.12E−23 | 0.5818 |
| 280 | positive regulation of protein kinase B signaling cascade | 5.37E−06 | 7 | 0.0761 | 0.0084 | 0.2691 | 1.17E−05 | −0.2608 |
| 281 | cell body | 1.64E−78 | 20 | 0.2299 | 0.0095 | −0.2873 | 5.27E−77 | 0.2969 |
| 282 | ATP biosynthetic process | 2.99E−06 | 9 | 0.3750 | 0.0703 | 0.2771 | 6.74E−06 | 0.2068 |
| 283 | response to activity | 4.85E−08 | 10 | 0.1923 | 0.0652 | 0.2565 | 1.28E−07 | −0.1912 |
| 284 | positive regulation of neuron apoptotic process | 1.61E−24 | 8 | 0.1194 | 0.1132 | −0.3507 | 1.21E−23 | 0.4639 |
| 285 | positive regulation of blood pressure | 2.91E−10 | 3 | 0.1304 | 0.0913 | −0.2747 | 9.15E−10 | 0.3660 |
| 286 | Rac GTPase binding | 8.81E−08 | 11 | 0.2558 | −0.0332 | −0.0232 | 2.28E−07 | −0.0100 |
| 287 | regulation of the force of heart contraction | 1.75E−17 | 3 | 0.1111 | −0.0033 | 0.4506 | 8.80E−17 | 0.4539 |
| 288 | positive regulation of cell growth | 1.81E−17 | 18 | 0.2045 | 0.1953 | 0.0290 | 9.07E−17 | 0.1663 |
| 289 | removal of superoxide radicals | 1.28E−54 | 5 | 0.2083 | 0.3803 | −0.3708 | 2.24E−53 | 0.7511 |
| 290 | internal side of plasma membrane | 1.88E−07 | 8 | 0.1333 | 0.0899 | −0.3034 | 4.73E−07 | 0.3933 |
| 291 | positive regulation of NF-kappaB transcription factor activity | 4.12E−14 | 23 | 0.1533 | 0.1065 | −0.0644 | 1.70E−13 | 0.1709 |
| 292 | fructose 6-phosphate metabolic process | 1.46E−10 | 5 | 0.1923 | −0.0239 | −0.3041 | 4.69E−10 | 0.2802 |
| 293 | anterior/posterior pattern specification | 2.11E−05 | 6 | 0.0462 | 0.0220 | 0.3121 | 4.37E−05 | −0.2901 |
| 294 | Z disc | 5.83E−18 | 23 | 0.1456 | 0.0807 | −0.1475 | 3.02E−17 | 0.2282 |
| 295 | epithelial cell differentiation | 8.91E−06 | 22 | 0.2750 | −0.0205 | −0.1203 | 1.91E−05 | 0.0998 |
| 296 | toll-like receptor signaling pathway | 5.27E−06 | 18 | 0.1565 | 0.1001 | −0.0696 | 1.15E−05 | 0.1698 |
| 297 | transcriptional repressor complex | 1.90E−26 | 12 | 0.1538 | 0.0582 | −0.2754 | 1.52E−25 | 0.3336 |
| 298 | nuclear chromatin | 4.09E−23 | 29 | 0.1667 | 0.0082 | 0.1644 | 2.86E−22 | −0.1562 |
| 299 | iron-sulfur cluster binding | 5.24E−08 | 3 | 0.0811 | 0.0714 | −0.3253 | 1.37E−07 | 0.3967 |
| 300 | secretory granule membrane | 9.28E−13 | 3 | 0.0833 | −0.1127 | 0.3309 | 3.61E−12 | −0.4436 |
| 301 | maternal placenta development | 1.83E−09 | 3 | 0.1667 | −0.0974 | 0.4196 | 5.50E−09 | −0.5170 |
| 302 | nuclear envelope lumen | 9.16E−81 | 3 | 0.2500 | 0.0464 | −0.8702 | 3.01E−79 | 0.9166 |
| 303 | regulation of insulin secretion | 3.83E−16 | 18 | 0.2000 | 0.0009 | 0.2089 | 1.77E−15 | −0.2080 |
| 304 | microtubule cytoskeleton organization | 2.19E−27 | 14 | 0.0859 | 0.1694 | −0.1871 | 1.80E−26 | 0.3565 |
| 305 | Ran GTPase binding | 1.13E−07 | 13 | 0.1970 | 0.0276 | −0.1731 | 2.89E−07 | 0.2007 |
| 306 | repressing transcription factor binding | 1.36E−23 | 6 | 0.1500 | 0.0962 | −0.3316 | 9.75E−23 | 0.4278 |
| 307 | ER-associated protein catabolic process | 1.85E−20 | 8 | 0.1905 | −0.0070 | 0.2512 | 1.09E−19 | −0.2582 |
| 308 | regulation of neuron apoptotic process | 4.08E−58 | 4 | 0.1538 | 0.1807 | −0.5285 | 7.75E−57 | 0.7091 |
| 309 | pseudopodium | 3.64E−05 | 7 | 0.2917 | 0.0529 | −0.1218 | 7.34E−05 | 0.1747 |
| 310 | CenH3-containing nucleosome assembly at centromere | 1.46E−06 | 9 | 0.3600 | 0.0744 | 0.2157 | 3.40E−06 | −0.1413 |
| 311 | NADH dehydrogenase (ubiquinone) activity | 9.29E−06 | 8 | 0.1194 | 0.1258 | 0.4901 | 1.98E−05 | −0.3642 |
| 312 | replication fork | 3.80E−11 | 4 | 0.1000 | 0.0559 | 0.4037 | 1.28E−10 | −0.3478 |
| 313 | chromatin DNA binding | 8.30E−26 | 9 | 0.1304 | 0.0410 | 0.3417 | 6.41E−25 | −0.3007 |
| 314 | cellular response to heat | 1.55E−11 | 7 | 0.1591 | −0.0221 | 0.2861 | 5.43E−11 | −0.3082 |
| 315 | histone H3 deacetylation | 9.53E−15 | 6 | 0.2400 | 0.0265 | 0.3381 | 4.16E−14 | −0.3116 |
| 316 | ER to Golgi vesicle-mediated transport | 4.95E−13 | 16 | 0.1495 | 0.0244 | 0.1880 | 1.96E−12 | −0.1635 |
| 317 | protein secretion | 5.77E−36 | 6 | 0.2222 | 0.0689 | 0.4599 | 6.18E−35 | −0.3911 |
| 318 | protein maturation | 3.42E−15 | 3 | 0.1154 | −0.0181 | 0.3479 | 1.52E−14 | −0.3660 |
| 319 | double-strand break repair | 7.32E−14 | 20 | 0.2500 | 0.0539 | 0.2068 | 2.99E−13 | −0.1529 |
| 320 | response to ionizing radiation | 1.42E−07 | 4 | 0.0667 | 0.0726 | 0.4400 | 3.62E−07 | −0.3674 |
| 321 | translation elongation factor activity | 1.05E−275 | 10 | 0.1961 | 0.1009 | −0.7616 | 1.38E−273 | 0.8625 |
| 322 | translational elongation | 0 | 84 | 0.6512 | 0.0734 | −0.5621 | 0 | 0.6355 |
| 323 | cell chemotaxis | 5.91E−06 | 6 | 0.0845 | −0.1442 | −0.4587 | 1.28E−05 | 0.3145 |
| 324 | activating transcription factor binding | 9.12E−06 | 4 | 0.1538 | −0.0098 | 0.2681 | 1.95E−05 | −0.2779 |
| 325 | substantia nigra development | 4.81E−15 | 20 | 0.4348 | 0.1008 | −0.0618 | 2.13E−14 | 0.1626 |
| 326 | positive regulation of phagocytosis | 1.11E−16 | 5 | 0.1563 | −0.1615 | 0.2038 | 5.31E−16 | −0.3653 |
| 327 | brush border membrane | 7.79E−38 | 4 | 0.0588 | −0.1205 | −0.8762 | 8.71E−37 | 0.7556 |
| 328 | tRNA aminoacylation for protein translation | 5.63E−22 | 28 | 0.4058 | 0.0034 | −0.1856 | 3.74E−21 | 0.1890 |
| 329 | actomyosin structure organization | 2.20E−06 | 6 | 0.1935 | 0.0949 | −0.1093 | 4.99E−06 | 0.2043 |
| 330 | proteasome core complex, alpha-subunit complex | 3.44E−07 | 7 | 0.2692 | −0.0375 | 0.0570 | 8.36E−07 | 0.0946 |
| 331 | protein disulfide isomerase activity | 2.95E−70 | 10 | 0.3226 | −0.0087 | 0.3645 | 7.50E−69 | −0.3732 |
| 332 | COPI-coated vesicle | 1.88E−18 | 3 | 0.2727 | 0.0380 | 0.7951 | 9.89E−18 | −0.7571 |
| 333 | syntaxin binding | 5.26E−11 | 5 | 0.0962 | −0.0756 | 0.2779 | 1.75E−10 | −0.3535 |
| 334 | DNA damage checkpoint | 2.71E−11 | 4 | 0.0678 | 0.1067 | 0.5327 | 9.29E−11 | −0.4259 |
| 335 | negative regulation of DNA replication | 1.01E−14 | 5 | 0.1667 | −0.1111 | −0.7643 | 4.39E−14 | 0.6532 |
| 336 | tricarboxylic acid cycle | 4.85E−35 | 21 | 0.4200 | 0.0544 | 0.2481 | 5.00E−34 | −0.1937 |
| 337 | isocitrate metabolic process | 2.15E−09 | 3 | 0.2727 | 0.0465 | −0.3325 | 6.37E−09 | 0.3791 |
| 338 | positive regulation of nitric oxide biosynthetic process | 6.44E−86 | 4 | 0.1000 | 0.0667 | −0.6094 | 2.42E−84 | 0.6761 |
| 339 | negative regulation of ryanodine-sensitive calcium-release channel | 1.05E−08 | 3 | 0.2727 | 0.1002 | −0.0999 | 2.98E−08 | 0.2001 |
| | activity | | | | | | | |
| 340 | microvillus | 2.33E−14 | 12 | 0.2308 | 0.0411 | −0.1549 | 9.86E−14 | 0.1960 |
| 341 | adult locomotory behavior | 3.46E−19 | 11 | 0.1264 | 0.1305 | −0.1355 | 1.91E−18 | 0.2660 |
| 342 | transcription from mitochondrial promoter | 9.32E−06 | 3 | 0.2727 | 0.1532 | 0.4923 | 1.99E−05 | −0.3391 |
| 343 | aerobic respiration | 1.56E−11 | 7 | 0.2800 | −0.0184 | 0.2265 | 5.45E−11 | −0.2449 |
| 344 | cytokine binding | 3.84E−21 | 5 | 0.2000 | 0.0944 | −0.4401 | 2.40E−20 | 0.5345 |
| 345 | negative regulation of proteolysis | 6.15E−10 | 5 | 0.1163 | 0.2571 | −0.1700 | 1.89E−09 | 0.4270 |
| 346 | translational termination | 0 | 78 | 0.7800 | 0.0735 | −0.5473 | 0 | 0.6207 |
| 347 | T cell differentiation in thymus | 1.03E−09 | 5 | 0.0893 | 0.0537 | 0.3345 | 3.13E−09 | −0.2808 |
| 348 | regulation of cyclin-dependent protein kinase activity | 1.83E−33 | 6 | 0.0674 | 0.0772 | −0.6234 | 1.76E−32 | 0.7006 |
| 349 | mitochondrial electron transport, NADH to ubiquinone | 8.49E−06 | 9 | 0.1800 | 0.0644 | 0.2295 | 1.82E−05 | −0.1651 |
| 350 | neuron apoptotic process | 1.43E−07 | 9 | 0.1429 | 0.2412 | −0.1299 | 3.63E−07 | 0.3711 |
| 351 | intrinsic apoptotic signaling pathway | 1.81E−81 | 19 | 0.3167 | 0.1540 | −0.3102 | 6.09E−80 | 0.4642 |
| 352 | isomerase activity | 5.45E−08 | 6 | 0.2069 | −0.0341 | 0.2653 | 1.43E−07 | −0.2994 |
| 353 | de novo' IMP biosynthetic process | 2.14E−26 | 6 | 0.6000 | 0.0453 | −0.3111 | 1.69E−25 | 0.3564 |
| 354 | cerebellum development | 1.07E−08 | 4 | 0.0784 | 0.0427 | −0.2844 | 3.02E−08 | 0.3271 |
| 355 | tetrahydrofolate biosynthetic process | 1.55E−14 | 3 | 0.2500 | 0.0551 | −0.3561 | 6.65E−14 | 0.4112 |
| 356 | small-subunit processome | 7.06E−43 | 4 | 0.2105 | 0.1071 | −0.6545 | 8.97E−42 | 0.7616 |
| 357 | cellular response to oxidative stress | 1.23E−40 | 9 | 0.2045 | 0.1776 | −0.2669 | 1.49E−39 | 0.4445 |
| 358 | glycogen biosynthetic process | 3.17E−07 | 6 | 0.1538 | 0.0696 | −0.1749 | 7.79E−07 | 0.2445 |
| 359 | mitochondrial transport | 2.58E−54 | 3 | 0.1034 | 0.2583 | −0.4483 | 4.47E−53 | 0.7065 |
| 360 | acyl-CoA dehydrogenase activity | 4.47E−06 | 3 | 0.0857 | 0.0335 | 0.3614 | 9.84E−06 | −0.3279 |
| 361 | DNA topoisomerase (ATP-hydrolyzing) activity | 2.49E−05 | 3 | 0.2727 | −0.1182 | 0.0638 | 5.11E−05 | −0.1819 |
| 362 | DNA topological change | 8.13E−06 | 5 | 0.1136 | −0.0845 | −0.1469 | 1.75E−05 | 0.0624 |
| 363 | transcription cofactor activity | 4.19E−08 | 10 | 0.1163 | 0.1380 | −0.1365 | 1.11E−07 | 0.2745 |
| 364 | RNA helicase activity | 2.35E−07 | 8 | 0.6667 | 0.0344 | 0.1927 | 5.84E−07 | −0.1584 |
| 365 | binding of sperm to zona pellucida | 2.82E−47 | 12 | 0.2308 | 0.0028 | −0.2384 | 4.20E−46 | 0.2413 |
| 366 | ossification | 1.83E−08 | 7 | 0.0745 | 0.0846 | −0.2926 | 5.08E−08 | 0.3772 |
| 367 | cyclin-dependent protein kinase activity | 4.72E−05 | 3 | 0.0698 | 0.1909 | −0.0634 | 9.43E−05 | 0.2543 |
| 368 | circadian rhythm | 1.66E−11 | 9 | 0.1154 | 0.0715 | 0.2641 | 5.79E−11 | −0.1926 |
| 369 | GDP binding | 1.53E−28 | 21 | 0.3818 | −0.0091 | 0.3117 | 1.32E−27 | −0.3208 |
| 370 | skeletal muscle tissue development | 3.26E−21 | 4 | 0.0533 | 0.0849 | −0.4791 | 2.06E−20 | 0.5640 |
| 371 | pentose-phosphate shunt | 1.41E−71 | 7 | 0.2258 | 0.0774 | −0.3483 | 3.77E−70 | 0.4257 |
| 372 | blood vessel development | 1.96E−12 | 5 | 0.0685 | 0.3305 | 0.7654 | 7.39E−12 | −0.4349 |
| 373 | NuRD complex | 1.62E−10 | 7 | 0.1944 | −0.0583 | 0.1386 | 5.20E−10 | −0.1969 |
| 374 | mRNA catabolic process | 1.43E−12 | 3 | 0.1765 | 0.0213 | −0.3699 | 5.46E−12 | 0.3911 |
| 375 | gluconeogenesis | 1.97E−91 | 22 | 0.3188 | 0.1681 | −0.2531 | 8.88E−90 | 0.4212 |
| 376 | adenyl nucleotide binding | 5.55E−12 | 3 | 0.0698 | 0.0760 | −0.3502 | 2.01E−11 | 0.4262 |
| 377 | clathrin coat of trans-Golgi network vesicle | 4.68E−22 | 4 | 0.2222 | 0.1513 | −0.1391 | 3.14E−21 | 0.2903 |
| 378 | clathrin coat of coated pit | 9.81E−22 | 5 | 0.2381 | 0.1484 | −0.1391 | 6.39E−21 | 0.2874 |
| 379 | receptor tyrosine kinase binding | 3.62E−24 | 8 | 0.2105 | 0.0284 | −0.3162 | 2.67E−23 | 0.3446 |
| 380 | positive regulation of peptidyl-serine phosphorylation | 4.29E−05 | 9 | 0.1552 | 0.1396 | −0.0602 | 8.60E−05 | 0.1997 |
| 381 | cell periphery | 2.98E−08 | 6 | 0.1579 | 0.1888 | −0.2996 | 8.02E−08 | 0.4884 |
| 382 | DNA ligation involved in DNA repair | 4.84E−30 | 3 | 0.3000 | −0.0353 | −0.6013 | 4.33E−29 | 0.5660 |
| 383 | copper ion transport | 1.17E−20 | 3 | 0.1071 | 0.1457 | −0.2765 | 7.11E−20 | 0.4222 |
| 384 | membrane protein ectodomain proteolysis | 2.08E−10 | 5 | 0.2000 | 0.0071 | 0.1713 | 6.61E−10 | −0.1642 |
| 385 | signal peptide processing | 3.72E−07 | 3 | 0.1304 | −0.0387 | 0.3418 | 9.00E−07 | 0.3805 |
| 386 | serine-type peptidase activity | 6.33E−12 | 9 | 0.0938 | −0.0842 | 0.4508 | 2.28E−11 | 0.5350 |
| 387 | sperm protein complex | 1.77E−61 | 9 | 0.4091 | 0.0030 | −0.2561 | 3.68E−60 | 0.2591 |
| 388 | chaperonin-containing T-complex | 1.60E−59 | 9 | 0.4286 | 0.0019 | −0.2561 | 3.16E−58 | 0.2580 |
| 389 | positive regulation of ATPase activity | 3.97E−07 | 6 | 0.2000 | 0.0197 | −0.1975 | 9.59E−07 | 0.2171 |
| 390 | single-stranded DNA binding | 6.04E−07 | 29 | 0.2900 | −0.0186 | 0.1155 | 1.44E−06 | −0.1341 |
| 391 | nucleocytoplasmic transport | 2.10E−16 | 7 | 0.1346 | 0.1384 | 0.0175 | 9.89E−16 | 0.1209 |
| 392 | oxaloacetate metabolic process | 1.76E−14 | 6 | 0.3158 | 0.1843 | 0.4418 | 7.52E−14 | −0.2575 |
| 393 | apoptotic cell clearance | 1.89E−27 | 3 | 0.1111 | −0.0353 | 0.4007 | 1.56E−26 | −0.4360 |
| 394 | Golgi organization | 3.45E−11 | 13 | 0.2167 | 0.0217 | 0.3457 | 1.17E−10 | −0.3241 |
| 395 | barbed-end actin filament capping | 1.63E−20 | 5 | 0.3333 | 0.3442 | −0.2589 | 9.71E−20 | 0.6031 |
| 396 | oxidoreductase activity, acting on NADH or NADPH | 4.51E−11 | 3 | 0.1304 | 0.0556 | 0.4292 | 1.51E−10 | 0.3735 |
| 397 | hemidesmosome | 6.25E−06 | 5 | 0.2500 | −0.0297 | 0.1078 | 1.36E−05 | −0.1375 |
| 398 | positive regulation of dendrite morphogenesis | 3.96E−05 | 3 | 0.0811 | −0.0059 | −0.2831 | 7.96E−05 | 0.2772 |
| 399 | negative regulation of endothelial cell proliferation | 1.24E−19 | 6 | 0.1818 | 0.0610 | 0.4194 | 7.05E−19 | −0.3583 |
| 400 | malate metabolic process | 2.09E−07 | 3 | 0.1364 | 0.1559 | 0.3094 | 5.22E−07 | −0.1535 |
| 401 | protein tetramerization | 1.87E−07 | 9 | 0.2432 | −0.0171 | −0.2124 | 4.71E−07 | 0.1953 |
| 402 | regulation of circadian rhythm | 3.64E−06 | 6 | 0.2000 | 0.0341 | 0.2330 | 8.09E−06 | −0.1989 |
| 403 | spindle assembly involved in mitosis | 1.14E−05 | 4 | 0.3636 | −0.0301 | −0.1648 | 2.40E−05 | 0.1347 |
| 404 | positive regulation of fibroblast proliferation | 1.52E−66 | 6 | 0.0769 | 0.2888 | −0.4795 | 3.52E−65 | 0.7683 |
| 405 | protein kinase C binding | 3.74E−19 | 17 | 0.3036 | 0.0308 | −0.1962 | 2.05E−18 | 0.2270 |
| 406 | cotranslational protein targeting to membrane | 1.10E−08 | 4 | 0.3333 | −0.0479 | 0.3067 | 3.09E−08 | −0.3546 |
| 407 | glycoprotein binding | 7.60E−27 | 13 | 0.1494 | −0.0545 | 0.2138 | 6.15E−26 | −0.2683 |
| 408 | dendrite cytoplasm | 3.59E−07 | 4 | 0.1739 | −0.0001 | 0.2307 | 8.70E−07 | −0.2309 |
| 409 | apolipoprotein binding | 2.81E−18 | 3 | 0.1364 | 0.0455 | 0.5615 | 1.48E−17 | −0.5160 |
| 410 | chaperone-mediated protein folding | 7.74E−13 | 16 | 0.3902 | −0.0193 | 0.1316 | 3.02E−12 | −0.1509 |
| 411 | cofactor binding | 7.50E−11 | 4 | 0.1250 | 0.0767 | −0.2231 | 2.46E−10 | 0.2998 |
| 412 | response to heat | 5.06E−07 | 10 | 0.1695 | −0.1153 | 0.1835 | 1.21E−06 | −0.2988 |
| 413 | positive regulation of protein import into nucleus, translocation | 1.16E−24 | 5 | 0.3125 | 0.0410 | −0.5842 | 8.80E−24 | 0.5432 |
| 414 | nuclear chromosome | 1.29E−07 | 11 | 0.3667 | 0.0063 | 0.1873 | 3.30E−07 | −0.1810 |
| 415 | chromatin remodeling | 9.33E−06 | 19 | 0.1959 | 0.0114 | 0.1211 | 1.99E−05 | 0.1097 |
| 416 | cellular response to interleukin-4 | 2.88E−21 | 7 | 0.2000 | −0.0347 | −0.4376 | 1.82E−20 | 0.4030 |
| 417 | neuron projection development | 1.80E−36 | 12 | 0.0795 | 0.1699 | −0.2523 | 1.98E−35 | 0.4222 |
| 418 | lung development | 1.26E−06 | 11 | 0.0840 | 0.1057 | 0.3106 | 2.92E−06 | −0.2048 |
| 419 | translation initiation factor binding | 2.84E−17 | 5 | 0.1724 | −0.0021 | 0.2707 | 1.41E−16 | 0.2686 |
| 420 | cytoplasmic membrane-bounded vesicle | 1.04E−05 | 23 | 0.1783 | −0.0414 | −0.2561 | 2.21E−05 | 0.2147 |
| 421 | ATP metabolic process | 1.14E−12 | 3 | 0.0909 | 0.1667 | −0.1389 | 4.39E−12 | 0.3055 |
| 422 | response to ethanol | 6.04E−09 | 15 | 0.0932 | 0.1773 | −0.0286 | 1.75E−08 | 0.2058 |
| 423 | regulation of acetyl-CoA biosynthetic process from pyruvate | 5.32E−11 | 8 | 0.4000 | −0.0338 | 0.2211 | 1.77E−10 | 0.2549 |
| 424 | Ras GTPase binding | 3.44E−06 | 4 | 0.2000 | −0.0695 | 0.4286 | 7.69E−06 | −0.4981 |
| 425 | fatty acid transport | 1.99E−14 | 3 | 0.1429 | 0.2694 | 0.6519 | 8.49E−14 | −0.3825 |
| 426 | positive regulation of protein serine/threonine kinase activity | 3.01E−18 | 6 | 0.2308 | −0.0602 | −0.5967 | 1.58E−17 | 0.5364 |
| 427 | dolichyl-diphosphooligosaccharide-protein glycotransferase activity | 2.91E−17 | 6 | 0.4286 | 0.0258 | 0.3661 | 1.43E−16 | −0.3403 |
| 428 | regulation of sodium ion transport | 2.14E−05 | 3 | 0.1667 | 0.0245 | 0.3275 | 4.42E−05 | −0.3030 |
| 429 | peptide binding | 2.03E−09 | 11 | 0.2200 | 0.0219 | −0.0439 | 6.04E−09 | 0.0658 |
| 430 | androgen receptor signaling pathway | 1.97E−35 | 10 | 0.2083 | 0.0951 | −0.4147 | 2.08E−34 | 0.5098 |
| 431 | protein neddylation | 1.62E−08 | 3 | 0.2727 | 0.0431 | −0.2579 | 4.53E−08 | 0.3010 |
| 432 | tubulin binding | 1.21E−71 | 5 | 0.0980 | 0.0539 | −0.6659 | 3.34E−70 | 0.7198 |
| 433 | protein N-terminus binding | 3.10E−45 | 21 | 0.1810 | 0.0777 | −0.1761 | 4.28E−44 | 0.2538 |
| 434 | osteoblast differentiation | 6.53E−40 | 31 | 0.2366 | 0.0419 | 0.1884 | 7.68E−39 | −0.1464 |
| 435 | striated muscle cell differentiation | 4.02E−35 | 4 | 0.2000 | 0.4791 | −0.6384 | 4.16E−34 | 1.1175 |
| 436 | negative regulation of release of cytochrome c from mitochondria | 4.84E−07 | 5 | 0.2778 | 0.2515 | 0.5898 | 1.17E−06 | −0.3383 |
| 437 | tropomyosin binding | 1.91E−38 | 4 | 0.1667 | 0.4163 | −0.5945 | 2.17E−37 | 1.0108 |
| 438 | ubiquitin ligase complex | 4.10E−14 | 9 | 0.1184 | 0.0643 | −0.2040 | 1.70E−13 | 0.2683 |
| 439 | male germ cell nucleus | 7.53E−07 | 3 | 0.0938 | 0.0681 | 0.2913 | 1.79E−06 | −0.2232 |
| 440 | regulation of synaptic plasticity | 1.47E−22 | 7 | 0.0959 | 0.1049 | −0.4518 | 9.99E−22 | 0.5567 |
| 441 | 2 iron, 2 sulfur cluster binding | 5.41E−06 | 5 | 0.1515 | 0.1634 | 0.5588 | 1.18E−05 | 0.3954 |
| 442 | sodium:potassium-exchanging ATPase activity | 3.24E−11 | 3 | 0.2500 | 0.0680 | 0.5054 | 1.10E−10 | −0.4373 |
| 443 | response to gamma radiation | 2.29E−05 | 5 | 0.1220 | 0.0846 | 0.3212 | 4.72E−05 | −0.2367 |
| 444 | RNA polymerase II transcription factor binding | 2.35E−11 | 6 | 0.1277 | −0.0183 | 0.1702 | 8.10E−11 | 0.1884 |
| 445 | peroxisomal matrix | 1.17E−12 | 10 | 0.2778 | 0.1076 | −0.1400 | 4.50E−12 | 0.2477 |
| 446 | cellular senescence | 2.02E−08 | 5 | 0.2381 | −0.1977 | 0.1491 | 5.56E−08 | −0.3468 |
| 447 | positive regulation of extrinsic apoptotic signaling pathway | 2.64E−08 | 5 | 0.1190 | −0.0587 | 0.2038 | 7.13E−08 | 0.2624 |
| 448 | cellular calcium ion homeostasis | 6.91E−16 | 15 | 0.1327 | −0.0501 | 0.2832 | 3.14E−15 | 0.3332 |
| 449 | response to salt stress | 1.48E−23 | 3 | 0.1200 | −0.1103 | −0.8640 | 1.06E−22 | 0.7538 |
| 450 | defense response to Gram-positive bacterium | 1.65E−06 | 7 | 0.0959 | 0.0195 | 0.2875 | 3.81E−06 | −0.2679 |
| 451 | chromosome segregation | 1.09E−05 | 17 | 0.1932 | −0.0449 | 0.0671 | 2.30E−05 | 0.1120 |
| 452 | nuclear heterochromatin | 1.22E−11 | 8 | 0.2105 | 0.0910 | 0.2700 | 4.30E−11 | 0.1790 |
| 453 | protein localization to nucleus | 1.54E−06 | 5 | 0.1111 | 0.0195 | 0.2002 | 3.56E−06 | −0.1807 |
| 454 | late endosome | 2.39E−10 | 16 | 0.1345 | 0.0090 | 0.2374 | 7.56E−10 | −0.2284 |
| 455 | polysome | 1.50E−17 | 11 | 0.2973 | −0.0253 | −0.2169 | 7.62E−17 | 0.1915 |
| 456 | negative regulation of translation | 2.40E−32 | 18 | 0.2903 | 0.0404 | −0.1847 | 2.23E−31 | 0.2250 |
| 457 | DNA damage response, signal transduction by p53 class mediator | 3.41E−13 | 7 | 0.1842 | 0.0781 | 0.3064 | 1.37E−12 | −0.2283 |
| | resulting in induction of apoptosis | | | | | | | |
| 458 | regulation of angiogenesis | 1.52E−17 | 3 | 0.1000 | −0.0264 | −0.5445 | 7.72E−17 | 0.5182 |
| 459 | activation of cysteine-type endopeptidase activity involved in apoptotic | 5.25E−18 | 16 | 0.1702 | −0.0254 | 0.1415 | 2.73E−17 | −0.1668 |
| | process | | | | | | | |
| 460 | endocytic vesicle | 7.50E−06 | 10 | 0.1515 | −0.0763 | 0.2451 | 1.62E−05 | −0.3214 |
| 461 | respiratory electron transport chain | 1.86E−147 | 44 | 0.4190 | 0.1104 | 0.4538 | 1.72E−145 | −0.3435 |
| 462 | regulation of signal transduction | 5.61E−45 | 3 | 0.0968 | 0.1135 | −0.6958 | 7.49E−44 | 0.8093 |
| 463 | DNA unwinding involved in replication | 4.26E−12 | 7 | 0.4667 | −0.1421 | 0.0555 | 1.56E−11 | −0.1976 |
| 464 | histone binding | 3.66E−10 | 27 | 0.3375 | 0.0399 | 0.1738 | 1.14E−09 | 0.1340 |
| 465 | cellular response to glucose starvation | 1.63E−18 | 5 | 0.2083 | 0.0077 | 0.3064 | 8.67E−18 | −0.2987 |
| 466 | fatty acid beta-oxidation | 9.00E−48 | 12 | 0.2727 | 0.0709 | 0.4462 | 1.35E−46 | 0.3753 |
| 467 | positive regulation of interferon-alpha production | 3.61E−11 | 3 | 0.1429 | −0.0389 | 0.2760 | 1.22E−10 | −0.3149 |
| 468 | positive regulation of T cell activation | 2.16E−19 | 4 | 0.1667 | −0.0518 | 0.2760 | 1.20E−18 | −0.3278 |
| 469 | endoplasmic reticulum unfolded protein response | 1.01E−23 | 26 | 0.2796 | 0.0182 | 0.2047 | 7.28E−23 | −0.1865 |
| 470 | positive regulation of protein secretion | 4.62E−28 | 6 | 0.1200 | 0.2172 | −0.4498 | 3.93E−27 | 0.6669 |
| 471 | microtubule plus-end binding | 8.72E−20 | 5 | 0.2273 | 0.1441 | −0.3441 | 4.98E−19 | 0.4882 |
| 472 | neutrophil chemotaxis | 1.07E−05 | 4 | 0.0755 | 0.1694 | −0.1674 | 2.26E−05 | 0.3368 |
| 473 | membrane protein intracellular domain proteolysis | 9.41E−09 | 3 | 0.1500 | 0.1662 | 0.5931 | 2.67E−08 | −0.4269 |
| 474 | TOR signaling cascade | 1.34E−95 | 4 | 0.2353 | 0.1135 | −1.0843 | 7.06E−94 | 1.1978 |
| 475 | negative regulation of cell adhesion | 3.58E−06 | 9 | 0.2195 | −0.1855 | 0.6847 | 7.98E−06 | 0.4992 |
| 476 | alternative nuclear mRNA splicing, via spliceosome | 3.13E−39 | 5 | 0.3125 | 0.0372 | 0.3437 | 3.60E−38 | −0.3065 |
| 477 | nuclear speck | 3.07E−25 | 69 | 0.3651 | 0.0382 | 0.1823 | 2.35E−24 | −0.1442 |
| 478 | cortical actin cytoskeleton | 1.02E−20 | 10 | 0.2222 | −0.0259 | 0.7477 | 6.24E−20 | 0.7218 |
| 479 | glucose binding | 4.66E−28 | 5 | 0.2273 | 0.1165 | −0.4140 | 3.95E−27 | 0.5305 |
| 480 | response to steroid hormone stimulus | 1.03E−17 | 3 | 0.1200 | −0.0259 | −0.4611 | 5.28E−17 | 0.4352 |
| 481 | response to endoplasmic reticulum stress | 5.97E−49 | 13 | 0.2321 | −0.0108 | 0.3529 | 9.31E−48 | −0.3637 |
| 482 | cellular response to reactive oxygen species | 4.76E−05 | 6 | 0.3529 | −0.0968 | 0.1602 | 9.48E−05 | 0.2571 |
| 483 | reactive oxygen species metabolic process | 1.41E−06 | 7 | 0.2258 | −0.0740 | 0.1367 | 3.27E−06 | −0.2107 |
| 484 | NADH metabolic process | 1.20E−08 | 4 | 0.3636 | 0.1700 | 0.4043 | 3.37E−08 | −0.2343 |
| 485 | succinate metabolic process | 1.65E−15 | 5 | 0.4167 | 0.0385 | 0.4135 | 7.41E−15 | 0.3750 |
| 486 | galactose catabolic process | 1.23E−05 | 4 | 0.4000 | 0.0839 | −0.1210 | 2.56E−05 | 0.2049 |
| 487 | positive regulation of interleukin-6 production | 1.74E−16 | 3 | 0.0526 | −0.0463 | 0.2715 | 8.24E−16 | −0.3178 |
| 488 | negative regulation of actin filament polymerization | 2.82E−14 | 5 | 0.2632 | 0.0237 | −0.4420 | 1.19E−13 | 0.4657 |
| 489 | oxidative phosphorylation | 6.47E−28 | 8 | 0.4706 | 0.0053 | 0.2990 | 5.46E−27 | −0.2936 |
| 490 | androgen receptor binding | 9.79E−31 | 10 | 0.2564 | 0.0745 | −0.1461 | 8.87E−30 | 0.2206 |
| 491 | mitochondrial intermembrane space | 8.59E−37 | 19 | 0.2468 | 0.0463 | 0.3382 | 9.53E−36 | −0.2919 |
| 492 | semaphorin receptor binding | 1.72E−27 | 4 | 0.1905 | −0.0150 | −1.0965 | 1.42E−26 | 1.0815 |
| 493 | melanosome transport | 5.35E−10 | 4 | 0.1481 | −0.0625 | 0.2929 | 1.66E−09 | −0.3554 |
| 494 | B cell proliferation | 3.64E−12 | 3 | 0.0769 | −0.0416 | 0.3223 | 1.34E−11 | −0.3639 |
| 495 | ion transmembrane transporter activity | 5.94E−16 | 5 | 0.4167 | 0.2073 | −0.4874 | 2.71E−15 | 0.6947 |
| 496 | receptor internalization | 4.92E−06 | 6 | 0.1538 | 0.0608 | −0.0820 | 1.08E−05 | 0.1429 |
| 497 | positive regulation of type I interferon production | 3.09E−06 | 18 | 0.2609 | 0.0849 | 0.2182 | 6.95E−06 | −0.1333 |
| 498 | Hsp70 protein binding | 5.95E−07 | 10 | 0.4167 | 0.1214 | −0.1565 | 1.42E−06 | 0.2779 |
| 499 | cytoplasmic stress granule | 1.92E−17 | 18 | 0.3673 | 0.0417 | −0.1715 | 9.59E−17 | 0.2132 |
| 500 | enoyl-CoA hydratase activity | 4.15E−44 | 4 | 0.5000 | 0.0995 | 0.6085 | 5.49E−43 | −0.5090 |
| 501 | long-chain-enoyl-CoA hydratase activity | 6.35E−40 | 3 | 0.5000 | 0.0729 | 0.5234 | 7.52E−39 | −0.4505 |
| 502 | cleavage furrow | 2.25E−11 | 14 | 0.2979 | 0.0795 | −0.1091 | 7.79E−11 | 0.1887 |
| 503 | stress fiber | 2.36E−14 | 20 | 0.3390 | 0.0405 | −0.1974 | 9.97E−14 | 0.2380 |
| 504 | respiratory chain | 1.58E−20 | 3 | 0.2727 | 0.1732 | 0.6190 | 9.45E−20 | −0.4458 |
| 505 | I-kappaB kinase/NF-kappaB cascade | 1.26E−20 | 4 | 0.0833 | 0.1142 | −0.4574 | 7.60E−20 | 0.5715 |
| 506 | regulation of heart contraction | 7.41E−12 | 5 | 0.1087 | 0.1342 | 0.2101 | 2.66E−11 | 0.3443 |
| 507 | protein targeting | 1.59E−55 | 13 | 0.2203 | 0.1083 | −0.3452 | 2.85E−54 | 0.4535 |
| 508 | myosin V binding | 1.10E−11 | 3 | 0.2727 | −0.0476 | 0.3582 | 3.93E−11 | −0.4058 |
| 509 | microtubule plus end | 6.41E−11 | 6 | 0.3333 | 0.1223 | −0.2645 | 2.12E−10 | 0.3867 |
| 510 | spindle organization | 1.46E−08 | 5 | 0.1923 | 0.1745 | 0.3029 | 4.08E−08 | 0.4773 |
| 511 | neural tube development | 2.19E−32 | 4 | 0.1429 | 0.3628 | −0.3263 | 2.05E−31 | 0.6892 |
| 512 | membrane depolarization | 1.21E−33 | 3 | 0.1304 | 0.1498 | −0.4710 | 1.19E−32 | 0.6207 |
| 513 | cholesterol binding | 8.05E−07 | 6 | 0.1463 | −0.0711 | 0.0877 | 1.91E−06 | −0.1588 |
| 514 | mitochondrial fusion | 2.75E−11 | 4 | 0.2353 | 0.1136 | 0.5389 | 9.41E−11 | −0.4252 |
| 515 | germ cell programmed cell death | 1.04E−10 | 3 | 0.2308 | 0.0994 | 0.3617 | 3.38E−10 | −0.2623 |
| 516 | response to radiation | 1.00E−07 | 5 | 0.1786 | −0.0510 | −0.3620 | 2.59E−07 | 0.3109 |
| 517 | rough endoplasmic reticulum | 5.22E−11 | 8 | 0.1739 | 0.0781 | 0.3728 | 1.74E−10 | −0.2947 |
| 518 | protein oligomerization | 5.07E−10 | 12 | 0.2667 | 0.1420 | 0.3705 | 1.58E−09 | 0.2285 |
| 519 | phosphatidylinositol-4,5-bisphosphate binding | 1.51E−18 | 8 | 0.1270 | 0.0498 | −0.2914 | 8.02E−18 | 0.3411 |
| 520 | release of cytochrome c from mitochondria | 5.53E−07 | 8 | 0.2105 | 0.1631 | 0.0397 | 1.32E−06 | 0.1234 |
| 521 | B cell activation | 1.01E−07 | 6 | 0.2069 | −0.0367 | 0.2404 | 2.59E−07 | −0.2771 |
| 522 | negative regulation of gene expression | 1.54E−05 | 9 | 0.0783 | 0.0684 | −0.1719 | 3.22E−05 | 0.2402 |
| 523 | negative regulation of extrinsic apoptotic signaling pathway | 9.13E−10 | 8 | 0.1538 | 0.1847 | −0.0916 | 2.79E−09 | 0.2763 |
| 524 | MyD88-dependent toll-like receptor signaling pathway | 4.06E−10 | 17 | 0.1932 | 0.0747 | −0.1016 | 1.27E−09 | 0.1763 |
| 525 | nitric oxide metabolic process | 5.43E−27 | 4 | 0.1818 | 0.1779 | −0.3707 | 4.41E−26 | 0.5486 |
| 526 | cytochrome-c oxidase activity | 2.42E−53 | 9 | 0.1698 | 0.1961 | 0.6031 | 4.14E−52 | −0.4070 |
| 527 | mitochondrial respiratory chain | 3.20E−11 | 4 | 0.1600 | 0.1569 | 0.5555 | 1.09E−10 | −0.3986 |
| 528 | nuclear outer membrane | 1.60E−33 | 9 | 0.3000 | 0.1319 | 0.4995 | 1.55E−32 | −0.3675 |
| 529 | mitochondrion transport along microtubule | 2.11E−14 | 4 | 0.2667 | 0.0147 | 0.3741 | 8.97E−14 | −0.3594 |
| 530 | MyD88-independent toll-like receptor signaling pathway | 2.19E−08 | 13 | 0.1646 | 0.0883 | −0.1830 | 5.97E−08 | 0.2713 |
| 531 | toll-like receptor 3 signaling pathway | 2.19E−08 | 13 | 0.1605 | 0.0883 | −0.1830 | 5.97E−08 | 0.2713 |
| 532 | potassium channel regulator activity | 3.52E−33 | 5 | 0.1020 | 0.3392 | −0.6883 | 3.36E−32 | 1.0274 |
| 533 | SRP-dependent cotranslational protein targeting to membrane | 0 | 95 | 0.8051 | 0.0670 | −0.4935 | 0 | 0.5605 |
| 534 | coated pit | 1.88E−12 | 18 | 0.3000 | −0.0156 | 0.3177 | 7.10E−12 | −0.3332 |
| 535 | fibroblast growth factor binding | 5.26E−48 | 4 | 0.1481 | 0.1213 | −0.5611 | 7.97E−47 | 0.6824 |
| 536 | ribosomal large subunit biogenesis | 4.95E−89 | 11 | 0.6875 | 0.0282 | −0.4485 | 2.00E−87 | 0.4767 |
| 537 | phosphoprotein binding | 5.13E−86 | 9 | 0.2093 | 0.1394 | −0.3112 | 1.97E−84 | 0.4506 |
| 538 | voltage-gated chloride channel activity | 7.84E−09 | 4 | 0.1429 | 0.0072 | −0.3284 | 2.24E−08 | 0.3356 |
| 539 | establishment of protein localization | 3.49E−06 | 5 | 0.1852 | −0.0355 | −0.1767 | 7.80E−06 | 0.1412 |
| 540 | trans-Golgi network membrane | 2.56E−16 | 10 | 0.1923 | 0.1430 | −0.1841 | 1.19E−15 | 0.3271 |
| 541 | positive regulation of epithelial cell migration | 1.66E−19 | 5 | 0.2000 | 0.0793 | −0.5088 | 9.28E−19 | 0.5880 |
| 542 | chemoattractant activity | 2.38E−57 | 5 | 0.2174 | 0.1378 | −0.4689 | 4.47E−56 | 0.6067 |
| 543 | positive chemotaxis | 8.98E−18 | 8 | 0.2424 | 0.0035 | −0.5299 | 4.61E−17 | 0.5334 |
| 544 | toll-like receptor 10 signaling pathway | 5.75E−23 | 15 | 0.2273 | 0.1128 | −0.2384 | 3.96E−22 | 0.3512 |
| 545 | T cell receptor signaling pathway | 1.10E−06 | 15 | 0.1163 | 0.0726 | −0.1098 | 2.57E−06 | 0.1825 |
| 546 | early endosome to late endosome transport | 1.73E−11 | 4 | 0.2353 | 0.0218 | 0.1553 | 6.04E−11 | −0.1335 |
| 547 | clathrin coat | 2.12E−19 | 4 | 0.2857 | 0.1426 | −0.1375 | 1.18E−18 | 0.2801 |
| 548 | platelet degranulation | 2.11E−36 | 23 | 0.2805 | 0.0247 | −0.2830 | 2.29E−35 | 0.3077 |
| 549 | calcium-mediated signaling using intracellular calcium source | 1.84E−07 | 3 | 0.2143 | 0.2178 | 0.6084 | 4.66E−07 | −0.3906 |
| 550 | negative regulation of DNA damage response, signal transduction by p53 | 1.03E−12 | 4 | 0.2857 | 0.2809 | −0.0904 | 3.99E−12 | 0.3713 |
| | class mediator | | | | | | | |
| 551 | regulation of phosphoprotein phosphatase activity | 7.57E−11 | 4 | 0.2500 | 0.1542 | 0.5613 | 2.47E−10 | −0.4071 |
| 552 | positive regulation of calcium ion import | 8.18E−10 | 3 | 0.1875 | 0.3561 | −0.1168 | 2.50E−09 | 0.4729 |
| 553 | intrinsic apoptotic signaling pathway in response to endoplasmic | 2.60E−07 | 5 | 0.1136 | 0.0024 | 0.3731 | 6.45E−07 | −0.3706 |
| | reticulum stress | | | | | | | |
| 554 | protein N-linked glycosylation via asparagine | 1.18E−39 | 26 | 0.2653 | 0.0140 | 0.2756 | 1.37E−38 | −0.2616 |
| 555 | protein destabilization | 1.92E−06 | 3 | 0.1200 | 0.1061 | 0.3400 | 4.43E−06 | −0.2339 |
| 556 | glucose 6-phosphate metabolic process | 2.44E−57 | 4 | 0.2500 | 0.0972 | −0.5205 | 4.52E−56 | 0.6177 |
| 557 | regulation of nitric-oxide synthase activity | 3.14E−26 | 5 | 0.2381 | 0.1766 | −0.3690 | 2.46E−25 | 0.5456 |
| 558 | 3-hydroxyacyl-CoA dehydrogenase activity | 2.64E−42 | 5 | 0.5000 | 0.0480 | 0.4523 | 3.30E−41 | −0.4042 |
| 559 | stress-activated MAPK cascade | 2.74E−12 | 12 | 0.2034 | 0.0882 | −0.2384 | 1.02E−11 | 0.3266 |
| 560 | cytokine production | 3.81E−45 | 3 | 0.0698 | 0.2413 | −0.5954 | 5.18E−44 | 0.8367 |
| 561 | positive regulation of heart rate | 1.01E−07 | 3 | 0.1765 | −0.0014 | 0.4307 | 2.60E−07 | −0.4321 |
| 562 | endoplasmic reticulum-Golgi intermediate compartment | 1.26E−42 | 24 | 0.4068 | −0.0246 | 0.3250 | 1.58E−41 | −0.3496 |
| 563 | COPII vesicle coating | 8.84E−18 | 6 | 0.2857 | 0.0762 | 0.7531 | 4.55E−17 | −0.6769 |
| 564 | innate immune response in mucosa | 3.36E−06 | 5 | 0.2500 | 0.0207 | 0.2668 | 7.52E−06 | −0.2460 |
| 565 | mRNA 3′-end processing | 7.51E−08 | 27 | 0.6279 | 0.0236 | 0.1923 | 1.95E−07 | −0.1687 |
| 566 | positive regulation of actin filament depolymerization | 5.82E−68 | 4 | 0.3333 | 0.3196 | −0.8686 | 1.43E−66 | 1.1882 |
| 567 | enzyme inhibitor activity | 3.13E−06 | 5 | 0.1351 | −0.0511 | 0.1614 | 7.05E−06 | −0.2125 |
| 568 | superoxide dismutase activity | 7.26E−10 | 3 | 0.1875 | 0.1272 | −0.2807 | 2.22E−09 | 0.4079 |
| 569 | V(D)J recombination | 1.13E−16 | 4 | 0.3077 | 0.0288 | −0.2561 | 5.42E−16 | 0.2849 |
| 570 | ankyrin binding | 7.80E−08 | 8 | 0.3478 | 0.0428 | 0.2823 | 2.02E−07 | −0.2395 |
| 571 | ATP-dependent chromatin remodeling | 4.46E−06 | 14 | 0.5600 | −0.0526 | 0.0623 | 9.84E−06 | −0.1149 |
| 572 | oligosaccharyltransferase complex | 2.91E−17 | 6 | 0.3529 | 0.0258 | 0.3661 | 1.43E−16 | −0.3403 |
| 573 | purine ribonucleoside monophosphate biosynthetic process | 5.68E−30 | 10 | 0.7143 | 0.0336 | −0.2795 | 5.06E−29 | 0.3131 |
| 574 | chromatin organization | 4.28E−13 | 25 | 0.2066 | 0.0119 | 0.1804 | 1.70E−12 | −0.1685 |
| 575 | activation of cysteine-type endopeptidase activity involved in apoptotic | 3.16E−09 | 3 | 0.2500 | 0.2076 | 0.4005 | 9.30E−09 | −0.1929 |
| | process by cytochrome c | | | | | | | |
| 576 | mannose binding | 4.57E−12 | 6 | 0.2500 | 0.0070 | 0.3630 | 1.67E−11 | −0.3560 |
| 577 | chaperone-mediated protein complex assembly | 3.97E−13 | 8 | 0.5000 | 0.0984 | −0.1251 | 1.58E−12 | 0.2235 |
| 578 | neutral amino acid transmembrane transporter activity | 2.61E−08 | 3 | 0.2308 | 0.1580 | 0.1654 | 7.08E−08 | −0.3234 |
| 579 | intracellular estrogen receptor signaling pathway | 2.51E−10 | 5 | 0.2500 | −0.0274 | 0.3119 | 7.91E−10 | −0.3394 |
| 580 | cell adhesion molecule binding | 4.85E−07 | 6 | 0.1277 | −0.0786 | −0.3765 | 1.17E−06 | 0.2978 |
| 581 | natural killer cell mediated cytotoxicity | 8.13E−83 | 5 | 0.2632 | 0.2181 | −0.8636 | 2.91E−81 | 1.0816 |
| 582 | inclusion body | 9.03E−11 | 4 | 0.2105 | −0.0725 | −0.3216 | 2.95E−10 | 0.2491 |
| 583 | mitochondrial nucleoid | 5.92E−94 | 23 | 0.5610 | 0.0572 | 0.3425 | 3.01E−92 | 0.2853 |
| 584 | negative regulation of nuclear mRNA splicing, via spliceosome | 7.05E−32 | 12 | 0.7500 | 0.0118 | 0.3004 | 6.50E−31 | −0.2885 |
| 585 | MHC class I protein binding | 1.53E−46 | 4 | 0.2667 | 0.0942 | −0.1965 | 2.25E−45 | 0.2907 |
| 586 | fatty acid binding | 1.33E−31 | 4 | 0.2222 | 0.0805 | −0.4035 | 1.22E−30 | 0.4841 |
| 587 | negative regulation of epidermal growth factor receptor signaling | 4.37E−23 | 13 | 0.2889 | 0.0799 | −0.2671 | 3.05E−22 | 0.3471 |
| | pathway | | | | | | | |
| 588 | negative regulation of proteasomal ubiquitin-dependent protein catabolic | 1.70E−58 | 5 | 0.2778 | −0.1116 | −0.9291 | 3.31E−57 | 0.8174 |
| | process | | | | | | | |
| 589 | RNA-induced silencing complex | 9.07E−07 | 4 | 0.2222 | 0.1843 | −0.1633 | 2.13E−06 | 0.3477 |
| 590 | insulin-like growth factor receptor binding | 1.96E−26 | 3 | 0.1579 | 0.1049 | −0.6143 | 1.56E−25 | 0.7192 |
| 591 | FK506 binding | 7.64E−07 | 7 | 0.3889 | −0.0333 | −0.2172 | 1.81E−06 | 0.1839 |
| 592 | AU-rich element binding | 1.07E−09 | 6 | 0.3750 | −0.0208 | 0.2254 | 3.24E−09 | −0.2462 |
| 593 | toll-like receptor 4 signaling pathway | 1.84E−15 | 17 | 0.1753 | 0.0750 | −0.2384 | 8.26E−15 | 0.3134 |
| 594 | de novo' posttranslational protein folding | 7.88E−127 | 27 | 0.6923 | 0.0090 | −0.3270 | 5.40E−125 | 0.3360 |
| 595 | glycerophospholipid biosynthetic process | 1.33E−22 | 11 | 0.1209 | 0.0842 | 0.5178 | 9.04E−22 | −0.4336 |
| 596 | cellular respiration | 3.63E−08 | 3 | 0.1667 | 0.1375 | 0.3671 | 9.67E−08 | −0.2297 |
| 597 | monosaccharide binding | 7.64E−34 | 3 | 0.2000 | −0.0313 | −0.4969 | 7.57E−33 | 0.4657 |
| 598 | negative regulation of androgen receptor signaling pathway | 2.89E−24 | 3 | 0.2143 | 0.1146 | 0.5411 | 2.15E−23 | −0.4265 |
| 599 | toll-like receptor 9 signaling pathway | 5.75E−23 | 15 | 0.2055 | 0.1128 | −0.2384 | 3.96E−22 | 0.3512 |
| 600 | positive regulation of binding | 1.10E−06 | 3 | 0.1304 | 0.0814 | −0.2157 | 2.57E−06 | 0.2970 |
| 601 | protein refolding | 3.60E−21 | 9 | 0.6000 | 0.0992 | −0.2652 | 2.26E−20 | 0.3644 |
| 602 | GTP-dependent protein binding | 3.17E−09 | 8 | 0.4211 | −0.0394 | 0.4241 | 9.34E−09 | −0.4635 |
| 603 | DNA-(apurinic or apyrimidinic site) lyase activity | 4.59E−17 | 3 | 0.1579 | 0.0633 | −0.2605 | 2.25E−16 | 0.3237 |
| 604 | proline-rich region binding | 1.37E−16 | 5 | 0.2174 | 0.0403 | −0.4542 | 6.53E−16 | 0.4945 |
| 605 | potassium ion binding | 5.09E−16 | 3 | 0.1579 | 0.1615 | −0.3096 | 2.33E−15 | 0.4712 |
| 606 | RNA polymerase II repressing transcription factor binding | 2.71E−05 | 5 | 0.1429 | −0.0406 | 0.1277 | 5.54E−05 | −0.1683 |
| 607 | protein K63-linked ubiquitination | 7.18E−19 | 4 | 0.1333 | 0.1562 | −0.3582 | 3.86E−18 | 0.5144 |
| 608 | mitochondrial inner membrane presequence translocase complex | 8.99E−22 | 3 | 0.1304 | 0.1451 | 0.5932 | 5.90E−21 | −0.4481 |
| 609 | ameboidal cell migration | 5.03E−19 | 3 | 0.1875 | −0.0162 | −0.6165 | 2.71E−18 | 0.6003 |
| 610 | mRNA stabilization | 1.95E−09 | 4 | 0.3077 | 0.0127 | 0.2751 | 5.83E−09 | 0.2878 |
| 611 | nuclear-transcribed mRNA catabolic process, nonsense-mediated decay | 0 | 95 | 0.7983 | 0.0689 | −0.5303 | 0 | 0.5993 |
| 612 | actin-dependent ATPase activity | 1.89E−20 | 6 | 0.5000 | 0.1090 | −0.1451 | 1.11E−19 | 0.2541 |
| 613 | snRNA binding | 9.69E−10 | 3 | 0.1579 | −0.0265 | 0.2451 | 2.95E−09 | −0.2717 |
| 614 | small nuclear ribonucleoprotein complex | 1.77E−18 | 11 | 0.5500 | −0.0379 | 0.2145 | 9.34E−18 | −0.2524 |
| 615 | Golgi to plasma membrane protein transport | 4.93E−06 | 5 | 0.2000 | 0.1017 | 0.3734 | 1.08E−05 | −0.2716 |
| 616 | positive regulation of cell cycle arrest | 1.01E−06 | 3 | 0.1579 | −0.0066 | 0.3840 | 2.36E−06 | 0.3906 |
| 617 | RAGE receptor binding | 4.09E−08 | 5 | 0.3846 | −0.2398 | 0.5446 | 1.08E−07 | 0.3048 |
| 618 | Cajal body | 2.11E−13 | 19 | 0.4222 | 0.0507 | 0.2010 | 8.58E−13 | 0.1503 |
| 619 | transferrin transport | 2.70E−05 | 13 | 0.3939 | 0.0785 | −0.0243 | 5.50E−05 | 0.1027 |
| 620 | regulated secretory pathway | 4.42E−33 | 3 | 0.2308 | 0.0000 | 0.4514 | 4.20E−32 | −0.4514 |
| 621 | mitotic spindle organization | 1.01E−22 | 11 | 0.6471 | 0.0613 | −0.3467 | 6.91E−22 | 0.4080 |
| 622 | nucleobase-containing small molecule metabolic process | 9.97E−12 | 37 | 0.4744 | −0.0085 | −0.1280 | 3.56E−11 | 0.1195 |
| 623 | toll-like receptor 2 signaling pathway | 3.97E−23 | 16 | 0.2192 | 0.1129 | −0.2384 | 2.79E−22 | 0.3513 |
| 624 | TRIF-dependent toll-like receptor signaling pathway | 2.19E−08 | 13 | 0.1711 | 0.0883 | −0.1830 | 5.97E−08 | 0.2713 |
| 625 | toll-like receptor TLR1:TLR2 signaling pathway | 3.97E−23 | 16 | 0.2254 | 0.1129 | −0.2384 | 2.79E−22 | 0.3513 |
| 626 | toll-like receptor TLR6:TLR2 signaling pathway | 3.97E−23 | 16 | 0.2254 | 0.1129 | −0.2384 | 2.79E−22 | 0.3513 |
| 627 | platelet alpha granule lumen | 1.65E−10 | 6 | 0.1250 | 0.0331 | −0.4666 | 5.28E−10 | 0.4997 |
| 628 | anatomical structure morphogenesis | 2.58E−06 | 12 | 0.1304 | 0.1693 | −0.1574 | 5.82E−06 | 0.3267 |
| 629 | endocytic vesicle membrane | 3.37E−12 | 9 | 0.1343 | 0.0869 | −0.3210 | 1.25E−11 | 0.4079 |
| 630 | establishment of endothelial barrier | 7.09E−08 | 4 | 0.2667 | −0.0493 | −0.1888 | 1.85E−07 | 0.1395 |
| 631 | translation factor activity, nucleic acid binding | 2.73E−66 | 12 | 0.5000 | 0.0201 | −0.4813 | 6.24E−65 | 0.5014 |
| 632 | lysosomal lumen | 2.26E−12 | 16 | 0.2254 | −0.0233 | 0.2468 | 8.47E−12 | −0.2701 |
| 633 | regulation of neuron differentiation | 1.39E−11 | 4 | 0.2105 | 0.0224 | −0.4443 | 4.90E−11 | 0.4666 |
| 634 | RNA splicing, via transesterification reactions | 1.99E−09 | 15 | 0.6000 | −0.0025 | 0.1758 | 5.93E−09 | −0.1783 |
| 635 | nucleobase-containing small molecule interconversion | 1.05E−05 | 13 | 0.7222 | 0.0100 | −0.0867 | 2.24E−05 | 0.0967 |
| 636 | protein K11-linked ubiquitination | 2.23E−30 | 4 | 0.1538 | 0.1179 | −0.5181 | 2.01E−29 | 0.6360 |
| 637 | vitamin metabolic process | 7.29E−15 | 17 | 0.1977 | 0.1952 | −0.1572 | 3.20E−14 | 0.3524 |
| 638 | water-soluble vitamin metabolic process | 7.29E−15 | 17 | 0.2152 | 0.1952 | −0.1572 | 3.20E−14 | 0.3524 |
| 639 | generation of precursor metabolites and energy | 6.60E−43 | 13 | 0.2500 | 0.0888 | 0.4378 | 8.45E−42 | −0.3491 |
| 640 | regulation of interferon-gamma-mediated signaling pathway | 2.34E−16 | 7 | 0.4375 | −0.0797 | −0.5310 | 1.09E−15 | 0.4513 |
| 641 | regulation of type I interferon-mediated signaling pathway | 1.89E−17 | 5 | 0.1852 | −0.0785 | −0.5369 | 9.44E−17 | 0.4584 |
| 642 | COPI coating of Golgi vesicle | 1.85E−07 | 12 | 0.9231 | 0.0015 | 0.1628 | 4.66E−07 | −0.1613 |
| 643 | activation of signaling protein activity involved in unfolded protein | 7.12E−35 | 21 | 0.3231 | 0.0183 | 0.2687 | 7.19E−34 | −0.2503 |
| | response | | | | | | | |
| 644 | S100 protein binding | 2.31E−46 | 8 | 0.7273 | 0.1083 | −0.3835 | 3.37E−45 | 0.4918 |
| 645 | response to unfolded protein | 2.22E−26 | 19 | 0.3800 | 0.0547 | −0.1920 | 1.75E−25 | 0.2467 |
| 646 | toll-like receptor 5 signaling pathway | 5.75E−23 | 15 | 0.2308 | 0.1128 | −0.2384 | 3.96E−22 | 0.3512 |
| 647 | nuclear telomere cap complex | 5.87E−06 | 3 | 0.2500 | 0.1305 | 0.3069 | 1.28E−05 | −0.1764 |
| 648 | monocyte chemotaxis | 3.08E−58 | 3 | 0.1765 | 0.2268 | −0.3659 | 5.92E−57 | 0.5927 |
| 649 | nucleotide-binding domain, leucine rich repeat containing receptor | 2.63E−56 | 9 | 0.2143 | 0.0098 | −0.4285 | 4.81E−55 | 0.4383 |
| | signaling pathway | | | | | | | |
| 650 | regulation of transcription from RNA polymerase II promoter in response | 5.22E−18 | 5 | 0.1923 | 0.1116 | −0.2351 | 2.72E−17 | 0.3467 |
| | to hypoxia | | | | | | | |
| 651 | branched chain family amino acid catabolic process | 1.16E−12 | 9 | 0.5000 | −0.0376 | 0.2211 | 4.47E−12 | −0.2587 |
| 652 | IkappaB kinase complex | 2.15E−11 | 4 | 0.3636 | −0.0952 | 0.2555 | 7.44E−11 | −0.3507 |
| 653 | nucleotide-binding oligomerization domain containing signaling pathway | 1.72E−45 | 4 | 0.1600 | 0.1392 | −0.4623 | 2.40E−44 | 0.6016 |
| 654 | protein transmembrane transport | 2.22E−06 | 5 | 0.4167 | 0.0503 | 0.3942 | 5.04E−06 | −0.3439 |
| 655 | striated muscle contraction | 9.16E−51 | 3 | 0.2000 | 0.1439 | −0.8200 | 1.47E−49 | 0.9639 |
| 656 | actin filament capping | 4.49E−05 | 6 | 0.3750 | 0.0840 | −0.1424 | 8.97E−05 | 0.2264 |
| 657 | Set1C/COMPASS complex | 1.23E−05 | 3 | 0.3333 | −0.1002 | 0.2421 | 2.57E−05 | −0.3423 |
| 658 | endoplasmic reticulum-Golgi intermediate compartment membrane | 1.41E−26 | 10 | 0.3448 | 0.0715 | 0.4562 | 1.14E−25 | −0.3847 |
| 659 | muscle filament sliding | 1.12E−05 | 8 | 0.2105 | 0.1088 | −0.0931 | 2.37E−05 | 0.2019 |
| 660 | interaction with host | 9.25E−09 | 10 | 0.2941 | 0.1047 | −0.0943 | 2.63E−08 | 0.1990 |
| 661 | mitotic nuclear envelope disassembly | 4.71E−16 | 19 | 0.5135 | 0.1425 | 0.4297 | 2.16E−15 | −0.2872 |
| 662 | U12-type spliceosomal complex | 1.38E−05 | 16 | 0.6667 | −0.0146 | 0.1053 | 2.88E−05 | −0.1199 |
| 663 | site of double-strand break | 2.36E−06 | 5 | 0.3125 | 0.0194 | 0.1003 | 5.33E−06 | −0.0809 |
| 664 | catalytic step 2 spliceosome | 3.96E−67 | 55 | 0.6875 | 0.0000 | 0.2034 | 9.45E−66 | −0.2034 |
| 665 | cellular aldehyde metabolic process | 7.45E−09 | 4 | 0.3333 | 0.1663 | −0.1128 | 2.14E−08 | 0.2790 |
| 666 | positive regulation of protein insertion into mitochondrial membrane | 6.37E−146 | 11 | 0.4074 | 0.1797 | −0.5360 | 5.57E−144 | 0.7157 |
| | involved in apoptotic signaling pathway | | | | | | | |
| 667 | termination of RNA polymerase II transcription | 7.51E−08 | 27 | 0.5870 | 0.0236 | 0.1923 | 1.95E−07 | −0.1687 |
| 668 | low-density lipoprotein particle receptor binding | 2.50E−21 | 3 | 0.2308 | 0.0657 | 0.4366 | 1.59E−20 | −0.3709 |
| 669 | cytoskeletal anchoring at plasma membrane | 1.87E−05 | 5 | 0.3571 | −0.0337 | −0.1178 | 3.89E−05 | 0.0841 |
| 670 | antibacterial humoral response | 4.13E−06 | 6 | 0.2609 | 0.0225 | 0.2650 | 9.14E−06 | −0.2425 |
| 671 | phagocytic vesicle | 2.18E−10 | 9 | 0.3103 | −0.0844 | 0.2957 | 6.92E−10 | −0.3801 |
| 672 | Sin3 complex | 2.56E−07 | 4 | 0.3333 | −0.0578 | 0.1446 | 6.34E−07 | −0.2025 |
| 673 | DNA strand elongation involved in DNA replication | 6.80E−09 | 19 | 0.6129 | −0.0807 | 0.0730 | 1.95E−08 | −0.1537 |
| 674 | mitotic nuclear envelope reassembly | 4.97E−14 | 7 | 0.7000 | 0.1580 | 0.4892 | 2.04E−13 | −0.3312 |
| 675 | NADPH binding | 3.13E−07 | 5 | 0.5000 | 0.2438 | −0.1276 | 7.70E−07 | 0.3714 |
| 676 | mitochondrial proton-transporting ATP synthase complex | 6.76E−72 | 16 | 0.7619 | 0.0830 | 0.3891 | 1.94E−70 | −0.3061 |
| 677 | mitochondrial ATP synthesis coupled proton transport | 4.83E−67 | 14 | 0.8750 | 0.0893 | 0.3971 | 1.14E−65 | −0.3079 |
| 678 | clathrin-coated endocytic vesicle membrane | 2.64E−13 | 7 | 0.1628 | 0.1477 | −0.1274 | 1.07E−12 | 0.2751 |
| 679 | virus-host interaction | 3.75E−19 | 4 | 0.3333 | 0.0776 | 0.4858 | 2.05E−18 | −0.4083 |
| 680 | pantothenate metabolic process | 5.91E−68 | 3 | 0.2500 | 0.5473 | −0.4194 | 1.43E−66 | 0.9668 |
| 681 | lamellipodium membrane | 2.40E−22 | 4 | 0.2500 | 0.2027 | −0.4494 | 1.62E−21 | 0.6521 |
| 682 | glutamate metabolic process | 9.23E−10 | 3 | 0.2500 | 0.1404 | 0.4504 | 2.81E−09 | −0.3099 |
| 683 | positive regulation of blood vessel endothelial cell migration | 5.55E−21 | 3 | 0.1765 | 0.1120 | −0.4174 | 3.41E−20 | 0.5293 |
| 684 | MHC class II protein complex binding | 2.90E−231 | 7 | 0.4118 | 0.1567 | −0.6323 | 3.26E−229 | 0.7891 |
| 685 | viral transcription | 0 | 76 | 0.9268 | 0.0748 | −0.5495 | 0 | 0.6243 |
| 686 | 3′-UTR-mediated mRNA stabilization | 5.32E−14 | 7 | 0.5833 | −0.0525 | 0.2277 | 2.18E−13 | −0.2802 |
| 687 | ribosomal small subunit biogenesis | 0 | 12 | 0.9231 | 0.1003 | −0.8253 | 0 | 0.9256 |
| 688 | RNA polymerase II transcription regulatory region sequence-specific | 1.44E−09 | 4 | 0.4444 | 0.1225 | 0.5360 | 4.33E−09 | −0.4135 |
| | DNA binding transcription factor activity involved in negative regulation | | | | | | | |
| | of transcription | | | | | | | |

### REFERENCES

1. Regev A, Teichmann S A, Lander E S, Amit I, Benoist C, Birney E, Bodenmiller B, Campbell P, Carninci P, Clatworthy M, Clevers H, Deplancke B, Dunham I, Eberwine J, Eils R, Enard W, Farmer A, Fugger L, Gottgens B, Hacohen N, Haniffa M, Hemberg M, Kim S, Klenerman P, Kriegstein A, Lein E, Linnarsson S, Lundberg E, Lundeberg J, Majumder P, Marioni J C, Merad M, Mhlanga M, Nawijn M, Netea M, Nolan G, Pe′er D, Phillipakis A, Ponting C P, Quake S, Reik W, Rozenblatt-Rosen O, Sanes J, Satija R, Schumacher T N, Shalek A, Shapiro E, Sharma P, Shin J W, Stegle O, Stratton M, Stubbington M J T, Theis F J, Uhlen M, van Oudenaarden A, Wagner A, Watt F, Weissman J, Wold B, Xavier R, Yosef N; Human Cell Atlas Meeting Participants. The Human Cell Atlas. Elife. 2017 Dec. 5; 6:e27041.
2. Specht H, Slavov N. Transformative Opportunities for Single-Cell Proteomics. J Proteome Res. 2018 Aug. 3; 17(8):2565-2571.
3. Ziegenhain C, Vieth B, Parekh S, Reinius B, Guillaumet-Adkins A, Smets M, Leonhardt H, Heyn H, Hellmann I, Enard W. Comparative Analysis of Single-Cell RNA Sequencing Methods. Mol Cell. 2017 Feb. 16; 65(4):631-643.e4.
4. Slavov N. Unpicking the proteome in single cells. Science. 2020 Jan. 31; 367(6477):512-513.
5 Slavov N. Learning from natural variation across the proteomes of single cells. PLOS Biol. 2022 Jan. 5; 20(1):e3001512.
6. Shaffer S M, Dunagin M C, Torborg S R, Torre E A, Emert B, Krepler C, Beqiri M, Sproesser K, Brafford P A, Xiao M, Eggan E, Anastopoulos I N, Vargas-Garcia C A, Singh A, Nathanson K L, Herlyn M, Raj A. Rare cell variability and drug-induced reprogramming as a mode of cancer drug resistance. Nature. 2017 Jun. 15; 546(7658):431-435.
7. Emert B L, Cote C J, Torre E A, Dardani I P, Jiang C L, Jain N, Shaffer S M, Raj A. Variability within rare cell states enables multiple paths toward drug resistance. Nat Biotechnol. 2021 July;39(7):865-876.
8. Mahdessian D, Cesnik A J, Gnann C, Danielsson F, Stenström L, Arif M, Zhang C, Le T, Johansson F, Shutten R, Bäckström A, Axelsson U, Thul P, Cho N H, Carja O, Uhlén M, Mardinoglu A, Stadler C, Lindskog C, Ayoglu B, Leonetti M D, Pontén F, Sullivan D P, Lundberg E. Spatiotemporal dissection of the cell cycle with single-cell proteogenomics. Nature. 2021 February;590(7847):649-654.
9. Slavov N. Scaling Up Single-Cell Proteomics. Mol Cell Proteomics. 2022 Jan;21(1): 100179.
10. Slavov N. Single-cell protein analysis by mass spectrometry. Curr Opin Chem Biol. 2021 Feb;60:1-9.
11. Vanderaa C, Gatto L. Replication of single-cell proteomics data reveals important computational challenges. Expert Rev Proteomics. 2021 October; 18(10):835-843.
12. Kelly R T. Single-cell Proteomics: Progress and Prospects. Mol Cell Proteomics. 2020 Nov; 19(11):1739-1748.
13. Specht H, Emmott E, Petelski A A, Huffman R G, Perlman D H, Serra M, Kharchenko P, Koller A, Slavov N. Single-cell proteomic and transcriptomic analysis of macrophage heterogeneity using SCOPE2. Genome Biol. 2021 Jan. 27; 22(1):50.
14. Klein A M, Mazutis L, Akartuna I, Tallapragada N, Veres A, Li V, Peshkin L, Weitz D A, Kirschner M W. Droplet barcoding for single-cell transcriptomics applied to embryonic stem cells. Cell. 2015 May 21;161(5):1187-1201.
15. Macosko E Z, Basu A, Satija R, Nemesh J, Shekhar K, Goldman M, Tirosh I, Bialas A R, Kamitaki N, Martersteck E M, Trombetta J J, Weitz D A, Sanes J R, Shalek A K, Regev A, McCarroll S A. Highly Parallel Genome-wide Expression Profiling of Individual Cells Using Nanoliter Droplets. Cell. 2015 May 21; 161(5): 1202-1214.
16. Harrison Specht, Guillaume Harmange, David H. Perlman, Edward Emmott, Zachary Niziolek, Bogdan Budnik, Nikolai Slavov. Automated sample preparation for high-throughput single-cell proteomics. bioRxiv. Aug. 25, 2018. 399774 (doi.org/10.1101/399774)
17. Petelski A A, Emmott E, Leduc A, Huffman R G, Specht H, Perlman D H, Slavov N. Multiplexed single-cell proteomics using SCOPE2. Nat Protoc. 2021 December; 16(12):5398-5425.
18. Marx V. A dream of single-cell proteomics. Nat Methods. 2019 September; 16(9):809-812.
19. R Gray Huffman, Andrew Leduc, Christoph Wichmann, Marco di Gioia, Francesco Borriello, Harrison Specht, Jason Derks, Saad Khan, Edward Emmott, Aleksandra A. Petelski, David H Perlman, Jürgen Cox, Ivan Zanoni, Nikolai Slavov. Prioritized single-cell proteomics reveals molecular and functional polarization across primary macrophages, bioRxiv Mar. 16, 2022. 484655 (doi.org/10.1101/2022.03.16.484655).
20. Giurgiu M, Reinhard J, Brauner B, Dunger-Kaltenbach I, Fobo G, Frishman G, Montrone C, Ruepp A. CORUM: the comprehensive resource of mammalian protein complexes-2019. Nucleic Acids Res. 2019 Jan. 8; 47(D1):D559-D563.
21. Fallahi-Sichani M, Becker V, Izar B, Baker G J, Lin J R, Boswell S A, Shah P, Rotem A, Garraway L A, Sorger P K. Adaptive resistance of melanoma cells to RAF inhibition via reversible induction of a slowly dividing de-differentiated state. Mol Syst Biol. 2017 Jan. 9; 13(1):905.
22. Dahlén E, Veitonmäki N, Norlén P. Bispecific antibodies in cancer immunotherapy. Ther Adv Vaccines Immunother. 2018 February;6(1):3-17.
23. Gebauer F, Wicklein D, Horst J, Sundermann P, Maar H, Streichert T, Tachezy M, Izbicki J R, Bockhorn M, Schumacher U. Carcinoembryonic antigen-related cell adhesion molecules (CEACAM) 1, 5 and 6 as biomarkers in pancreatic cancer. PLOS One. 2014 Nov. 19; 9(11):e113023.
24. Slavov N. Increasing proteomics throughput. Nat Biotechnol. 2021 July;39(7):809-810.
25. Hughes C S, Foehr S, Garfield D A, Furlong E E, Steinmetz L M, Krijgsveld J. Ultrasensitive proteome analysis using paramagnetic bead technology. Mol Syst Biol. 2014 Oct. 30; 10(10):757.
26. Hughes C S, Moggridge S, Müller T, Sorensen P H, Morin G B, Krijgsveld J. Single-pot, solid-phase-enhanced sample preparation for proteomics experiments. Nat Protoc. 2019 Jan; 14(1):68-85.
27. Kulak N A, Pichler G, Paron I, Nagaraj N, Mann M. Minimal, encapsulated proteomic-sample processing applied to copy-number estimation in eukaryotic cells. Nat Methods. 2014 Mar; 11(3):319-24.
28. Cooper S. The synchronization manifesto: a critique of whole-culture synchronization. FEBS J. 2019 December; 286(23):4650-4656.
29. Specht H, Slavov N. Optimizing Accuracy and Depth of Protein Quantification in Experiments Using Isobaric Carriers. J Proteome Res. 2021 Jan. 1; 20(1):880-887.
30. Charif, D. & Lobry, J. SeqinR 1.0-2: a contributed package to the R project for statistical computing devoted to biological sequences retrieval and analysis. Biological and Medical Physics, Biomedical Engineering (eds Bastolla, U., Porto, M., Roman, H. & Vendruscolo, M.) ISBN: 978-3-540-35305-8, 207-232 (2007).
31. Jason Derks, Andrew Leduc, R. Gray Huffman, Harrison Specht, Markus Ralser, Vadim Demichev, Nikolai Slavov. Increasing the throughput of sensitive proteomics by plexDIA. bioRxiv Nov. 3, 2021. 467007 (doi.org/10.1101/2021.11.03.467007).
32. Cox J, Mann M. MaxQuant enables high peptide identification rates, individualized p.p.b.-range mass accuracies and proteome-wide protein quantification. Nat Biotechnol. 2008 Dec;26(12):1367-72.
33. Cox J, Neuhauser N, Michalski A, Scheltema R A, Olsen J V, Mann M. Andromeda: a peptide search engine integrated into the MaxQuant environment. J Proteome Res. 2011 Apr. 1; 10(4):1794-805.
34. Tyanova S, Temu T, Cox J. The MaxQuant computational platform for mass spectrometry-based shotgun proteomics. Nat Protoc. 2016 December; 11(12):2301-2319.
35. Demichev V, Messner C B, Vernardis S I, Lilley K S, Ralser M. DIA-NN: neural networks and interference correction enable deep proteome coverage in high throughput. Nat Methods. 2020 January; 17(1): 41-44.
36. Vanderaa C, Gatto L. Replication of single-cell proteomics data reveals important computational challenges. Expert Rev Proteomics. 2021 October; 18(10):835-843. (Previously: Christophe Vanderaa, Laurent Gatto. Utilizing Scp for the analysis and replication of single-cell proteomics data. bioRxiv. Apr. 12, 2021. 439408 (doi.org/10.1101/2021.04.12.439408).
37. Vanderaa, C. & Gatto, L. Mass Spectrometry-Based Single-Cell Proteomics Data Analysis. Bioconductor, 10.18129/B9.bioc.scp (2020).
38. Specht H, Emmott E, Petelski A A, Huffman R G, Perlman D H, Serra M, Kharchenko P, Koller A, Slavov N. Single-cell proteomic and transcriptomic analysis of macrophage heterogeneity using SCOPE2. Genome Biol. 2021 Jan. 27; 22(1):50.
39. Franks A, Airoldi E, Slavov N. Post-transcriptional regulation across human tissues. PLOS Comput Biol. 2017 May 8; 13(5):e1005535.
40. Gray Huffman, Harrison Specht, Albert Chen, Nikolai Slavov. DO-MS: Data-Driven Optimization of Mass Spectrometry Methods bioRxiv. Jan. 6, 2019. 512152 (doi.org/10.1101/512152).
41. Chen A T, Franks A, Slavov N. DART-ID increases single-cell proteome coverage. PLOS Comput Biol. 2019 Jul. 1; 15(7):e1007082 (previously bioRxiv. Aug. 23, 2018. 399121 (https://doi.org/10.1101/399121)).
42. Budnik B, Levy E, Harmange G, Slavov N. SCOPE-MS: mass spectrometry of single mammalian cells quantifies proteome heterogeneity during cell differentiation. Genome Biol. 2018 Oct. 22; 19(1): 161. (previously bioRxiv. Jan. 24, 2017. 102681 (doi.org/10.1101/102681)).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of forming a single-cell proteomic sample, said method comprising:
a) dispensing n droplets of lysis buffer onto a substantially planar solid surface, wherein n≥2;
b) dispensing a single cell into each of the n droplets of lysis buffer to produce n droplets, each comprising a lysed single cell;
c) dispensing digestion buffer into each of the n droplets to digest proteins from each lysed single cell to produce n droplets comprising peptides;
d) dispensing a chemical tag into each of the n droplets comprising the peptides to produce labeled peptides, wherein at least one droplet of the n droplets receives a different chemical tag from at least one other droplet of the n droplets, thereby enabling the labeled peptides in the at least one droplet to be distinguishable from the labeled peptides in the at least one other droplet; and
e) applying a fluid to merge at least a subset of the n droplets into a combined droplet on the substantially planar surface, thereby combining the labeled peptides to form a single-cell proteomic sample.

2. The method of claim 1, wherein each of the n droplets in step a), b), c), and/or d) has a volume of about 25 nanoliters (nl) or less.

3. The method of claim 1, wherein each of the n droplets in step a), b), c) and d) has a volume of about 25 nanoliters (nl) or less.

4. The method of claim 1, wherein the substantially planar solid surface is provided by a uniform glass slide.

5. The method of claim 1, wherein the substantially planar solid surface is etched with a geometric pattern.

6. The method of claim 1, wherein the substantially planar solid surface is fluorocarbon-coated.

7. The method of claim 1, wherein n is ≥10.

8. The method of claim 1, wherein the lysis buffer comprises about 4-8 nanoliters of 90-100% dimethyl sulfoxide (DMSO).

9. The method of claim 1, wherein step b) comprises dispensing the single cell in a cell suspension buffer with a volume of about 100-1,000 picoliters.

10. The method of claim 9, wherein step b) comprises dispensing the single cell in a cell suspension buffer with a volume of about 300 picoliters.

11. The method of claim 1, wherein the single cell is lysed in a total volume of about 4-10 nl for about 10-20 minutes.

12. The method of claim 1, wherein step c) comprises:
dispensing about 15-25 nl of about 120 ng/μl trypsin to each of the n droplets; and
digesting the proteins from each lysed single cell at about 1ºC above the dew point and a relative humidity of about 75% for about 4-5 hours.

13. The method of claim 1, wherein the chemical tag comprises a “light” version of TMT label reagents dissolved in DMSO.

14. The method of claim 1, wherein the chemical tag comprises a “heavy” version of TMT label reagents dissolved in DMSO.

15. The method of claim 1, wherein step d) comprises:
dispensing about 18-22 nl of a chemical tag into each of the n droplets comprising the peptides; and enabling the chemical tag to react with the peptides at room temperature and a relative humidity of about 75% for about 1 hour to produce the labeled peptides.

16. The method of claim 1, wherein the fluid is water.

17. The method of claim 1, wherein the fluid has a volume of about 1 μl.

18. The method of claim 1, wherein steps a) to e) are repeated at least once to form two or more single-cell proteomic samples on the substantially planar solid surface.

19. The method of claim 18, wherein at least 100 droplets of lysis buffer are dispensed onto the substantially planar solid surface.

20. The method of claim 19, wherein at least 500-3,000 droplets of lysis buffer are dispensed onto the substantially planar solid surface.

21. The method of claim 18, wherein the two or more single-cell proteomic samples comprises peptides from at least 100 cells.

22. The method of claim 21, wherein the two or more single-cell proteomic samples comprises peptides from about 100-10,000 cells.

23. The method of claim 1, wherein each droplet of the n droplets receives a unique chemical tag, thereby enabling the labeled peptides in each droplet to be distinguishable from the labeled peptides in each other droplet.

24. A method of performing a proteomic analysis comprising analyzing a single-cell proteomic sample formed by the method of claim 1.

25. The method of claim 24, wherein the analyzing comprises identifying and/or quantifying protein covariation across the single cells.

26. A single-cell proteomic sample formed by the method of claim 1.
